(19) United States
(12) Patent Application Publication
(10) Pub. No.: US 2012/0122840 A1
(43) Pub. Date: May 17, 2012

(54) COMPOUNDS AND THERAPEUTIC USES THEREOF

(75) Inventors: Dange Vijay Kumar, Salt Lake City, UT (US); Ian A. McAlexander, Salt Lake City, UT (US); Matthew Gregory Bursavich, Needham, MA (US); Christophe Hoarau, Pleasant Hill, CA (US); Paul M. Slattum, Salt Lake City, UT (US); David A. Gerrish, Lakewood, OH (US); Jeffrey W. Lockman, Princeton Junction, NJ (US); Weston R. Judd, Farmington, UT (US); Michael Saunders, Sandy, UT (US); Daniel P. Parker, Salt Lake City, UT (US); Daniel Feodore Zigar, Salt Lake City, UT (US); In Chul Kim, Sandy, UT (US); J. Adam Willardsen, Riverton, UT (US); Kraig M. Yager, Salt Lake City, UT (US); Mark D. Shenderovich, Salt Lake City, UT (US); Brandi L. Williams, Holladay, UT (US); Keith D. Tardif, Riverton, UT (US)

(73) Assignee: Myrexis, Inc., Salt Lake City, UT (US)

(21) Appl. No.: 13/243,876

(22) Filed: Sep. 23, 2011

Related U.S. Application Data
(63) Continuation of application No. PCT/US2010/028251, filed on Mar. 24, 2010.
(60) Provisional application No. 61/162,974, filed on Mar. 24, 2009.
(60) Provisional application No. 61/262,065, filed on Nov. 17, 2009.

(51) Int. Cl. A61K 31/541; C07D 473/32; G01N 33/567; A61K 31/5377; A61P 35/00; C07D 473/16; A61K 31/52

(52) U.S. Cl. 514/210.21; 544/277; 544/118; 544/58.2; 544/82; 514/263.4; 514/263.2; 514/263.24; 514/234.2; 514/263.22; 514/252.16; 514/228.5; 514/232.5; 435/7.23; 435/7.21

(57) ABSTRACT

The invention relates to compounds, pharmaceutical compositions, and uses thereof, including therapeutic uses thereof, such as methods useful for treating cancer.

### CROSS-REFERENCE TO RELATED APPLICATIONS

This application is a continuation of International Patent Application No. PCT/US2010/028521, filed Mar. 24, 2010, which claims the benefit of U.S. provisional application Ser. No. 61/162,974, filed Mar. 24, 2009, and U.S. provisional application Ser. No. 61/262,065, filed Nov. 17, 2009, the contents of each of which are hereby incorporated by reference herein in their entirety.

### FIELD OF THE INVENTION

The present invention relates generally to compounds, pharmaceutical compositions, and uses thereof, including therapeutic uses thereof, such as methods useful for treating diseases, particularly neoplastic diseases such as cancer.

### BACKGROUND OF THE INVENTION

Cancer is prevalent: there were an estimated 1.4 million new cases and 565,000 deaths in 2008. American Cancer Society, Cancer Facts & Figures 2008, 1-2 (2008). Although the five-year survival rate for cancer is now 66%, up from about 50% in the mid-nineteen seventies, cancer is still deadly. Id. at 2. In the United States in 2005, over half a million people died of cancer, representing 22.8% of all deaths. Although numerous treatments are available for various cancers, the fact remains that many cancers remain uncurable, untreatable, and/or become resistant to standard therapies. Bernadine Healy, We Need a New War on Cancer, U.S. NEWS & WORLD REPORT (Jun. 12, 2008). Thus, there is a need for new cancer treatments.

### BRIEF SUMMARY OF THE INVENTION

The present invention generally relates to compounds useful for treating neoplastic diseases, particularly cancer. Specifically, the present invention provides compounds with a structure according to Formula I

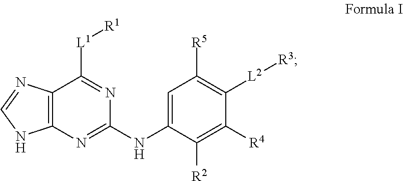

and pharmaceutically acceptable salts thereof,
wherein R¹, R², R³, R⁴, R⁵, L¹, and L² are as defined herein below.

The present invention also provides compounds with a structure according to Formula II

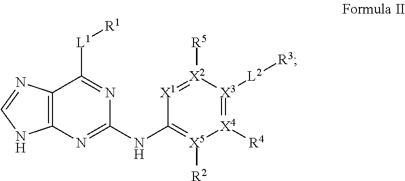

and pharmaceutically acceptable salts thereof,
wherein R¹, R², R³, R⁴, R⁵, X¹, X², X³, X⁴, X⁵, L¹, and L² are as defined herein below.

The compounds of the present invention are selective TTK inhibitors and are useful in treating cancer. Thus, in a related aspect, the present invention also provides a method for treating cancer by administering to a patient in need of such treatment a therapeutically effective amount of a compound of the present invention.

Also provided is the use of a compound of Formula I or II for the manufacture of a medicament useful for therapy, particularly for cancer. In addition, the present invention also provides a pharmaceutical composition having a compound of Formula I or II and one or more pharmaceutically acceptable excipients. A method for treating cancer by administering to a patient in need of the treatment the pharmaceutical composition is also encompassed.

In addition, the present invention further provides methods for treating or delaying the onset of the symptoms associated with cancer comprising administering an effective amount of a compound of the present invention, preferably in a pharmaceutical composition or medicament, to an individual having cancer.

The compounds of the present invention can be used in combination therapies. Thus, combination therapy methods are also provided for treating or delaying the onset of the symptoms associated with cancer. Such methods comprise administering to a patient in need thereof a compound of the present invention and, together or separately, at least one other anti-cancer therapy. For the convenience of combination therapy, the compound of the present invention can be administered together in the same formulation with another anti-cancer composition. Thus, the present invention also provides a pharmaceutical composition or medicament for the combination therapy, comprising an effective amount of a first compound according to the present invention and an effective amount of at least one anti-cancer composition, which is different from the first compound. Examples of anti-cancer compositions include, but are not limited to, chemotherapeutics, and protein kinase inhibitors.

The foregoing and other advantages and features of the invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### BRIEF DESCRIPTION OF THE DRAWINGS

FIG. 1 depicts the activity and selectivity of N6-cyclohexyl-N2-(2-methyl-4-morpholino-phenyl)-9H-purine-2,6-diamine (“Compound A”)—an exemplary TTK inhibitor of the present invention.

FIGS. 2A and 2B depict chromosome segregation defects caused by Compound A.

FIGS. 3A through 3C depict cell cycle effects of Compound A.

FIGS. 4A through 4C depict p53 pathway activation in response to Compound A.

FIGS. 5A and 5B depict induced phosphorylation of p53 and H2AX caused by Compound A.

FIGS. 6A through 6C depict ATR kinase phosphorylation of p53 and H2AX caused by Compound A.

FIGS. 7A through 7C depict the effect of caspase inhibition on responses induced by Compound A.

FIG. 8 depicts how overexpression of TTK induces the phosphorylation of a 90 kDa protein.

FIG. 9 depicts the identification of the phosphorylated 90 kDa protein as Hsp90.

FIG. 10 depicts the in vitro phosphorylation of Hsp90 by recombinant TTK.

FIG. 11 depicts the identification of the TTK phosphorylation site on Hsp90.

FIG. 12 depicts the co-precipitation of TTK and Hsp90 from lysates of cells overexpressing TTK.

### DETAILED DESCRIPTION OF THE INVENTION

### 1. Therapeutic Compounds

The present invention generally relates to compounds useful for treating cancer. Specifically, the present invention provides compounds of Formula I

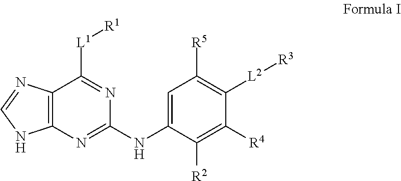

and pharmaceutically acceptable salts and solvates thereof.

In Formula I, R¹ is an optionally substituted carbocycle, heterocycle, aryl, or heteraryl.

In Formula I, R² is chosen from the group consisting of: halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle.

In Formula I, R³ is a group chosen from: hydro, haloalkyl, —R^{c}, —N(R^{b})C(═O)R^{c}, -alkylene-N(R)C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle.

In Formula I, R⁴ and R⁵ are independently chosen from: hydro, halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl.

In Formula I, L¹ is direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl (e.g., methyl), alkoxyl, carboxyl, or carbocycle.

In Formula I, L² is direct bond or a linker chosen from: —O—, —S—, (C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, —S(═O)₂—, -alkylene-, alkynylene (e.g., ethynylene, 1-propynylene, 2-propynylene), aryl (e.g., phenyl), heteroaryl, heterocycle (e.g., pyrrolidine, piperidine, piperazine, morpholine, and thiomorpholine), arylalkyl, heteroarylalkyl, and heterocyclylalkyl and —O-alkylene-; wherein R^{f} is chosen from hydro and C₁₋₄ alkyl.

In some embodiments, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments, R¹ is a substituted or unsubstituted C₃₋₆ cycloalkyl, heterocycle, C₃₋₆ cycloalkylalkyl, or heterocycloalkyl.

In some embodiments, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments, R¹ is chosen from the group consisting of:

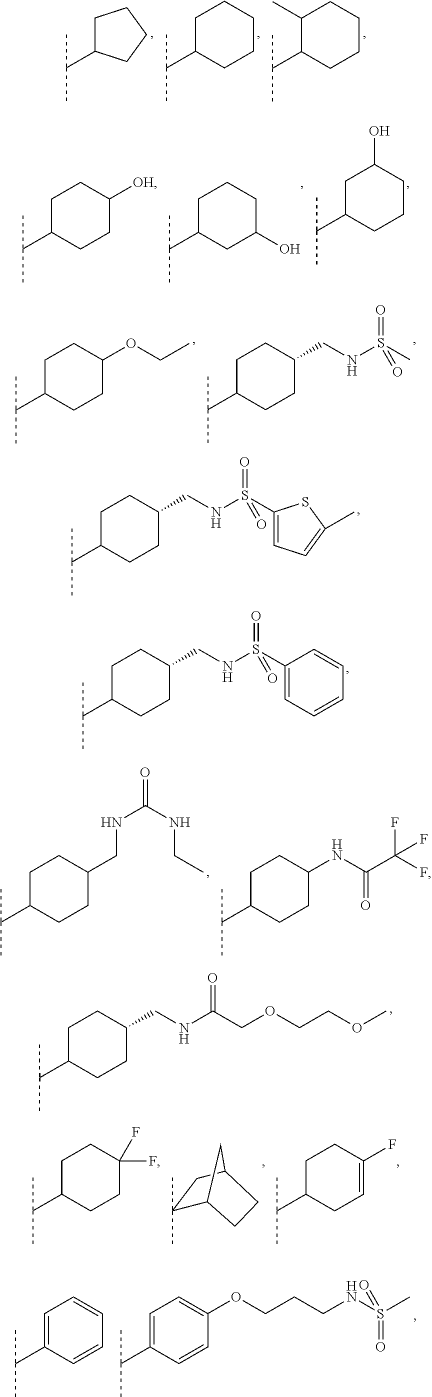

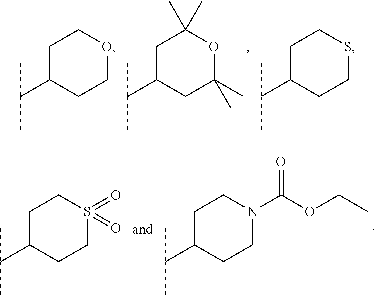

In some embodiments R² is halo, methyl optionally substituted with halo, ethyl optionally substituted with halo, methylthio, ethylthio, methoxy, or ethoxy.

In some embodiments R² is methyl, methoxy, ethoxy, Cl, trifluoromethyl.

In some embodiments R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino. In some embodiments R³ is hydro only when R⁴ or R⁵ is not hydro.

In some embodiments R³ is chosen from the group consisting of: methyl, methylene, trifluoromethyl, ethyl, ethylene, propyl, propylene, pentyl, pentylene,

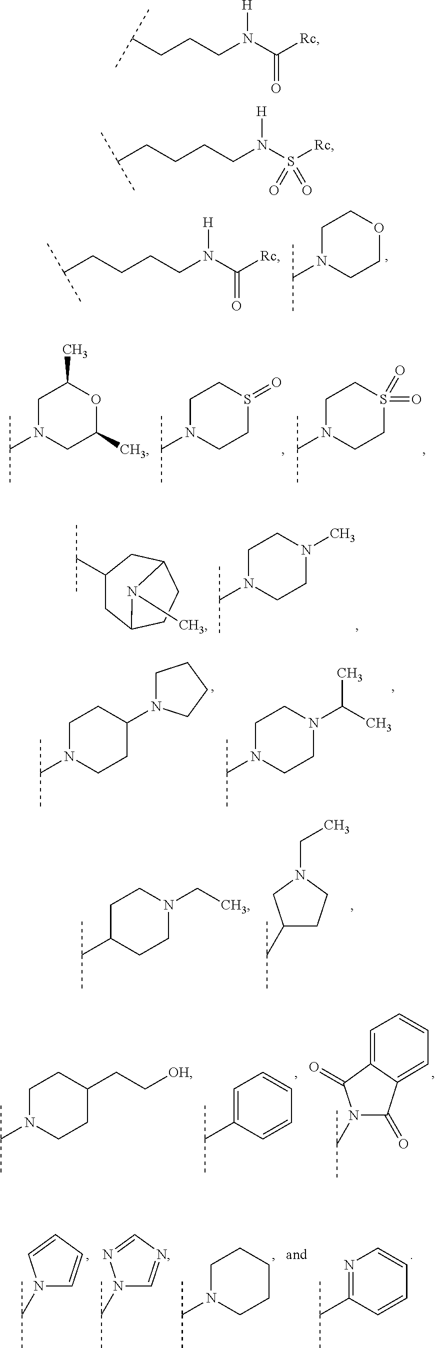

In some embodiments R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments, R^{c} is chosen from the group consisting of:

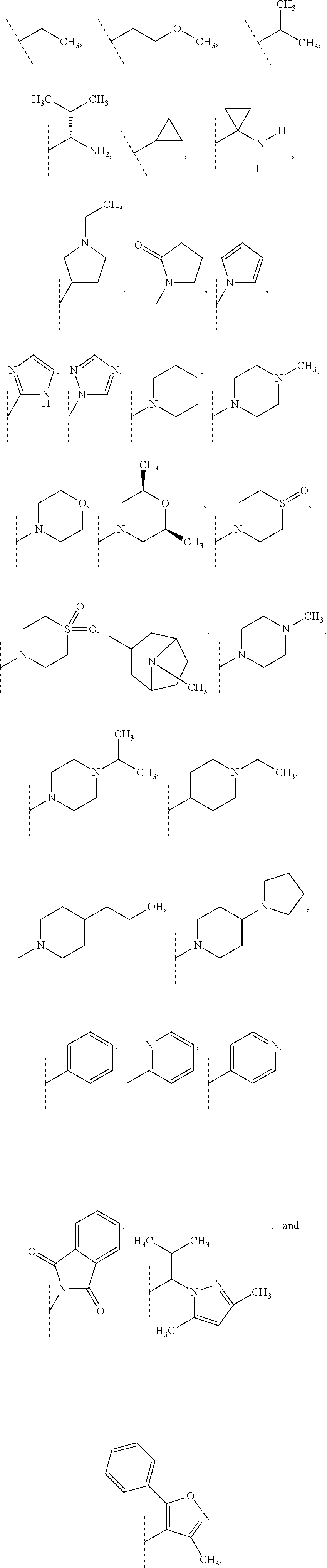

In some embodiments, R³ is —R⁶—R⁷, wherein R⁶ is: is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{f}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

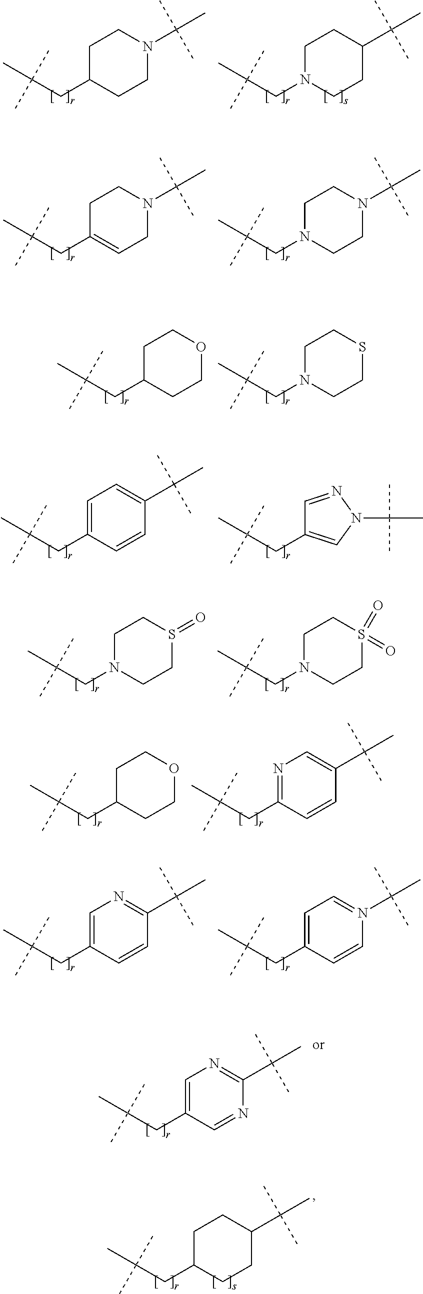

wherein r is 0, 1, or 2; and
wherein s is 0, 1, −1; and,
wherein R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is one of:

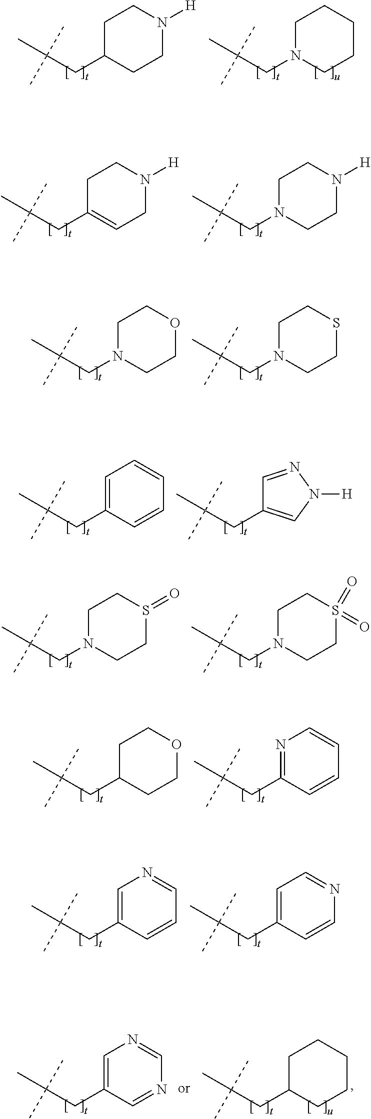

wherein t is 0, 1, or 2; and
wherein u is 0, 1, −1.

In some embodiments, r is 0.

In some embodiments, s is 1.

In some embodiments, t is 0.

In some embodiments, u is 1.

In some embodiments R² and R⁴, together with the carbon atoms to which they are attached, form the following ring structure:

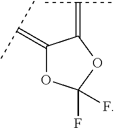

In some embodiments, R⁴, R⁵, or both, are Hydrogen.

In some embodiments, L¹ is direct bond or a linker chosen from: —O—, —S—, —S(═O)—, —S(═O)₂—, —N(R^{a})—, —CH(R^{a})—, —(CH₂)ₙ— wherein n is 1, 2 or 3, —C(═O)—, —C(═O)N(R^{a})—, wherein R^{a} is hydro or C₁₋₆ alkyl (e.g., methyl).

In some embodiments, L¹ is direct bond, —N(R^{a})— wherein R^{a} is hydro or C₁₋₃ alkyl (e.g., methyl or ethyl), —(CH₂)ₙ— wherein n is 1, 2 or 3, or —C(═O)—.

In some embodiments, L² is direct bond, or a linker chosen from: —O—, —O-alkylene-, —C(═O)—, —C(═O)N(R^{a})— wherein R^{a} is hydro or C₁₋₃ alkyl (e.g., methyl or ethyl), alkylene, alkynylene.

In some embodiments, L² is direct bond, or a linker chosen from: —O—, —O—(CH₂)ₙ— wherein n is 1, 2 or 3, —C(═O)—, —C(═O)N(R^{a})— wherein R^{a} is hydro or C₁₋₃ alkyl (e.g., methyl or ethyl), —(CH₂)ₙ— wherein n is 1, 2 or 3, —(CH₂)ₚ—C≡C—(CH₂)_{q}— wherein p and q are each independently 0, 1, 2 or 3.

In some embodiments, L² is alkynylene or:

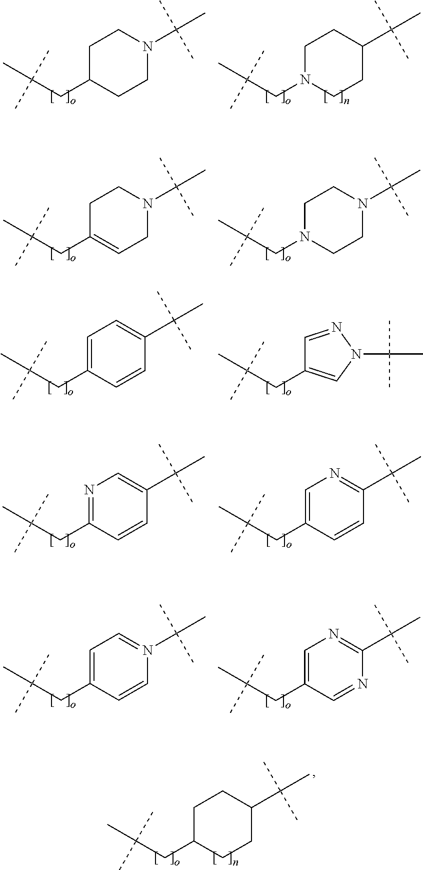

wherein o is 0, 1, or 2; and
wherein n is 0, 1, or −1.

In some embodiments, the compounds of Formula I are the compounds of Formula Ia

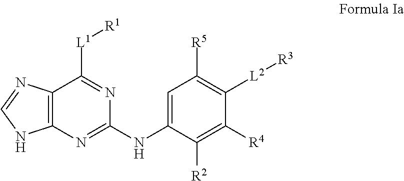

and pharmaceutically acceptable salts and solvates thereof.

In Formula Ia, R¹ is an optionally substituted carbocycle, heterocycle, aryl, or heteraryl.

In Formula Ia, R² is chosen from the group consisting of: halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle.

In Formula Ia, R³ is a group chosen from: hydro, haloalkyl, —R^{c}, —NH(C═O)—R^{c}, -alkylene-NH(C═O)—R^{c}, —(C═O)NH—R^{c}, -alkylene-(C═O)NH—R^{c}, —NH—S(═O)₂—R^{c}, -alkylene-NH—S(═O)₂—R^{c}, —S(═O)₂NH—R^{c}, -alkylene-S(═O)₂NH—R^{c}, and —N(R^{d})(R^{e})—; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino; and wherein R^{d} and R^{e} are each independently chosen from hydro and C₁₋₄ alkyl.

In Formula Ia, R⁴ and R⁵ are independently chosen from: hydro, halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a carbocycle, heterocycle, aryl or heteroaryl.

In Formula Ia, L¹ is direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl (e.g., methyl), alkoxyl, carboxyl, or carbocycle.

In Formula Ia, L² is direct bond or a linker chosen from: —O—, —S—, —(C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, and —S(═O)₂—, -alkylene-, and —O-alkylene-; wherein R^{f} is chosen from hydro and C₁₋₄ alkyl.

In some embodiments of Formula Ia, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ia, is a substituted or unsubstituted C₃₋₆ cycloalkyl, heterocycle, C₃₋₆ cycloalkylalkyl, or heterocycloalkyl.

In some embodiments of Formula Ia, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ia, R¹ is chosen from the group consisting of:

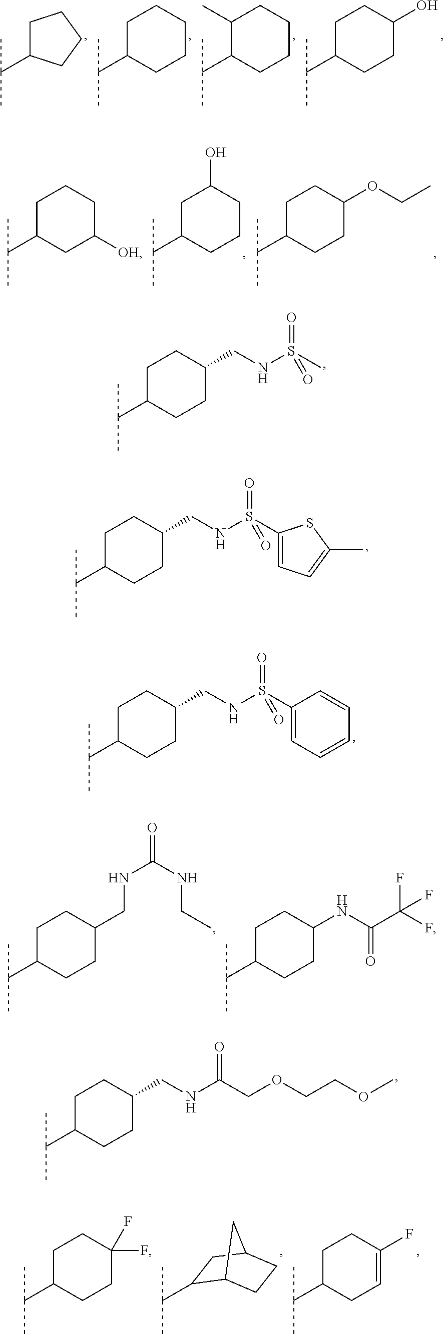

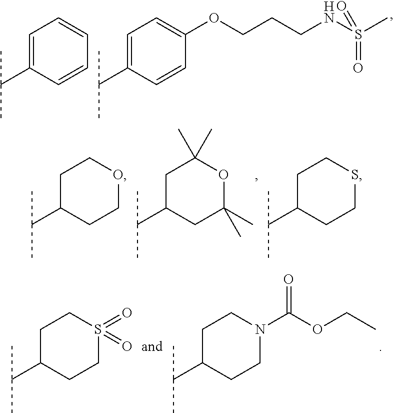

In some embodiments of Formula Ia, R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino. In some embodiments R³ is hydro only when R⁴ or R⁵ is not hydro.

In some embodiments of Formula Ia, R³ is chosen from the group consisting of: methyl, methylene, trifluoromethyl, ethyl, ethylene, propyl, propylene, pentyl, pentylene,

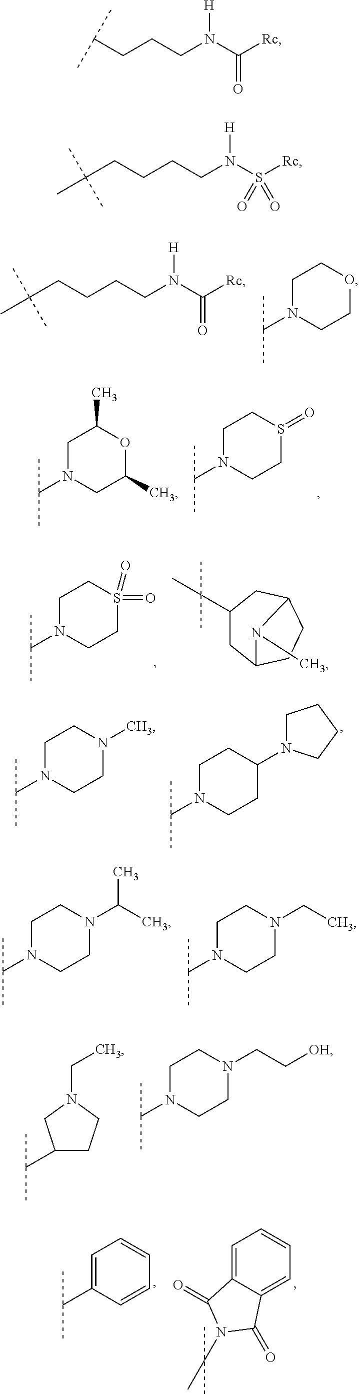

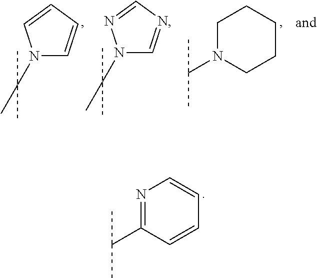

In some embodiments of Formula Ia, R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments of Formula Ia, R^{c} is chosen from the group consisting of:

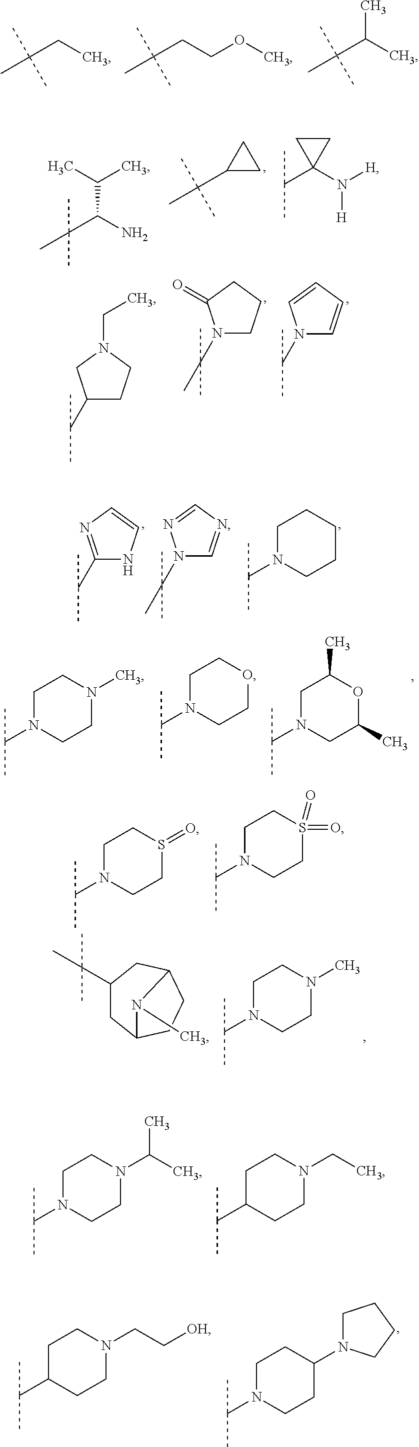

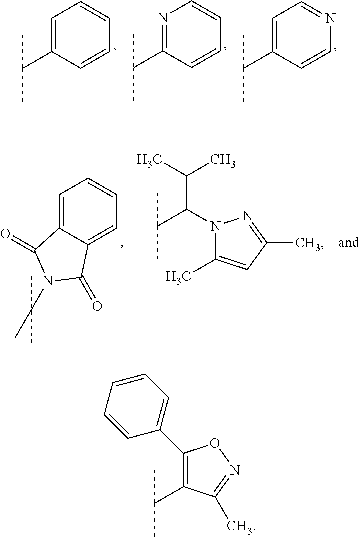

In some embodiments of Formula Ia, R² and R⁴, together with the carbon atoms to which they are attached, form the following ring structure:

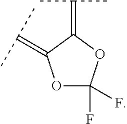

In some embodiments of Formula Ia, R⁴, R⁵, or both, are Hydrogen.

In some embodiments of Formula Ia, L¹ is direct bond or a linker chosen from: —O—, —S—, —S(═O)—, —S(═O)₂—, —N(R^{a})—, —CH(R^{a})—, —(CH₂)ₙ— wherein n is 1, 2 or 3, —C(═O)—, —C(═O)N(R^{a})—, wherein R^{a} is hydro or C₁₋₆ alkyl (e.g., methyl).

In some embodiments of Formula Ia, L¹ is direct bond, —N(R^{a})— wherein R^{a} is hydro or C₁₋₃ alkyl (e.g., methyl or ethyl), —(CH₂)ₙ— wherein n is 1, 2 or 3, or —C(═O)—.

In some embodiments of Formula Ia, L¹ is —N(H)—.

In some embodiments of Formula Ia, L² is direct bond, or a linker chosen from: —O—, —O-alkylene-, —C(═O)—, —C(═O)N(R^{a})— wherein R^{a} is hydro or C₁₋₃ alkyl (e.g., methyl or ethyl), alkylene, alkynylene.

In some embodiments of Formula Ia, L² is direct bond, or a linker chosen from: —O—, —O—(CH₂)ₙ— wherein n is 1, 2 or 3, —C(═O)—, —C(═O)N(R^{a})— wherein R^{a} is hydro or C₁₋₃ alkyl (e.g., methyl or ethyl), —(CH₂)ₙ— wherein n is 1, 2 or 3, —(CH₂)ₚ—C≡C—(CH₂)_{q}— wherein p and q are each independently 0, 1, 2 or 3.

In some embodiments, the compounds of Formula Ia are the compounds of Formula Ia1:

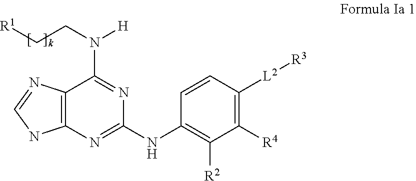

and pharmaceutically acceptable salts and solvates thereof.

In Formula Ia1, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ia1, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ia1, R¹ is chosen from the group consisting of:

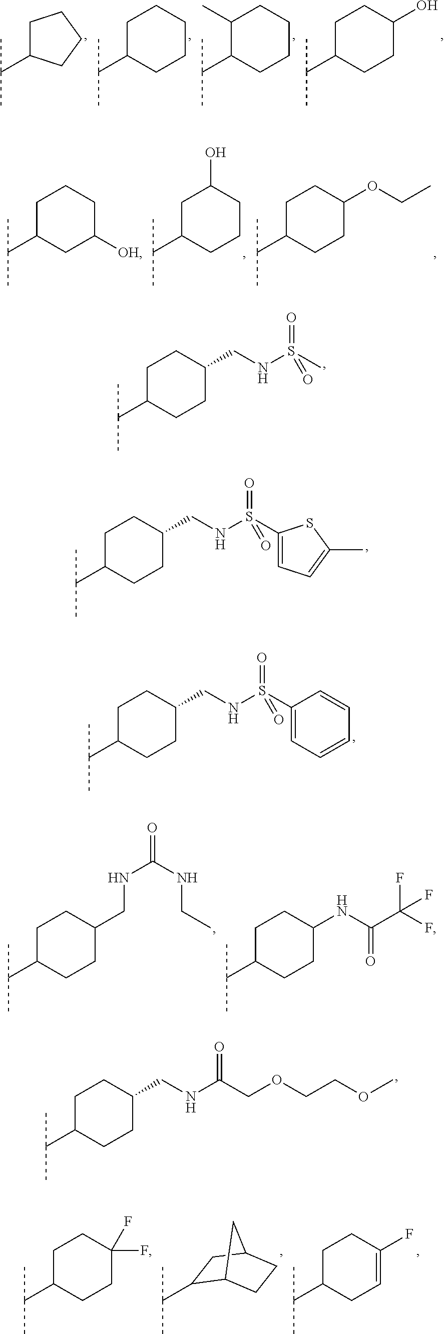

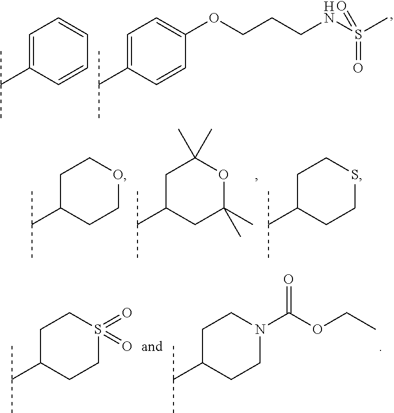

In Formula Ia1, k is −1, 0, 1, or 2.

In some embodiments of Formula Ia1, k is −1.

In Formula Ia1, R² is a group chosen from halo (e.g., Cl, Br, I), C₁₋₆ alkyl (preferably C₁₋₃ alkyl, e.g., methyl, ethyl, propyl, isopropyl, trifluoromethyl), C₂₋₆ alkenyl (preferably C₂₋₃ alkenyl, e.g., ethenyl), C₂₋₆ alkynyl (preferably C₂₋₃ alkynyl, e.g., ethynyl, propynyl), C₁₋₆ alkoxy (preferably C₁₋₃ alkoxy, e.g., methoxy, ethoxy, trifluoromethoxy), C₂₋₆ alkynyloxy (e.g., ethynyloxy), C₁₋₆ alkylthio (preferably C₁₋₃ alkylthio, e.g., methylthio, ethylthio), C₃₋₆ cycloalkyl (e.g., cyclopropyl), amino (e.g., —NH₂, methylamino, dimethylamino), (C₁₋₃ alkoxy)C₁₋₃ alkyl (e.g., methoxymethyl), sulfonyl (e.g., methylsulfonyl or ethylsulfonyl), and sulfonyloxy (e.g., methylsulfonyloxy).

In some embodiments of Formula Ia1, R² is methyl, methoxy, ethoxy, Cl, trifluoromethyl.

In Formula Ia1, R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino. In some embodiments of Formula Ia1, R³ is chosen from the group consisting of: methyl, methylene, trifluoromethyl, ethyl, ethylene, propyl, propylene, pentyl, pentylene,

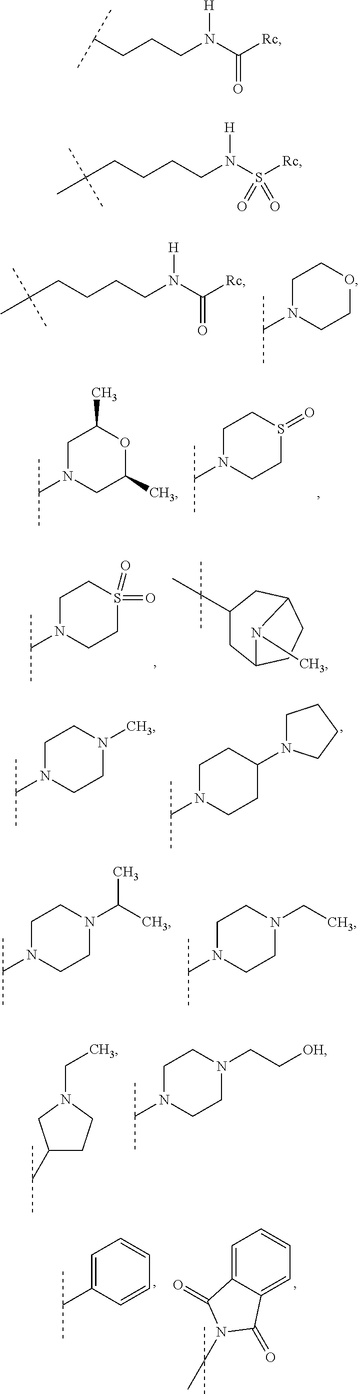

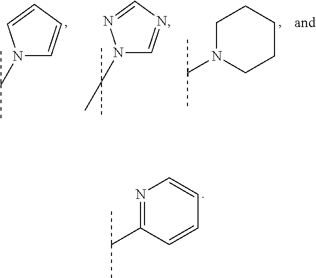

In some embodiments of Formula Ia1, R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments of Formula Ia1, R^{c} is chosen from the group consisting of:

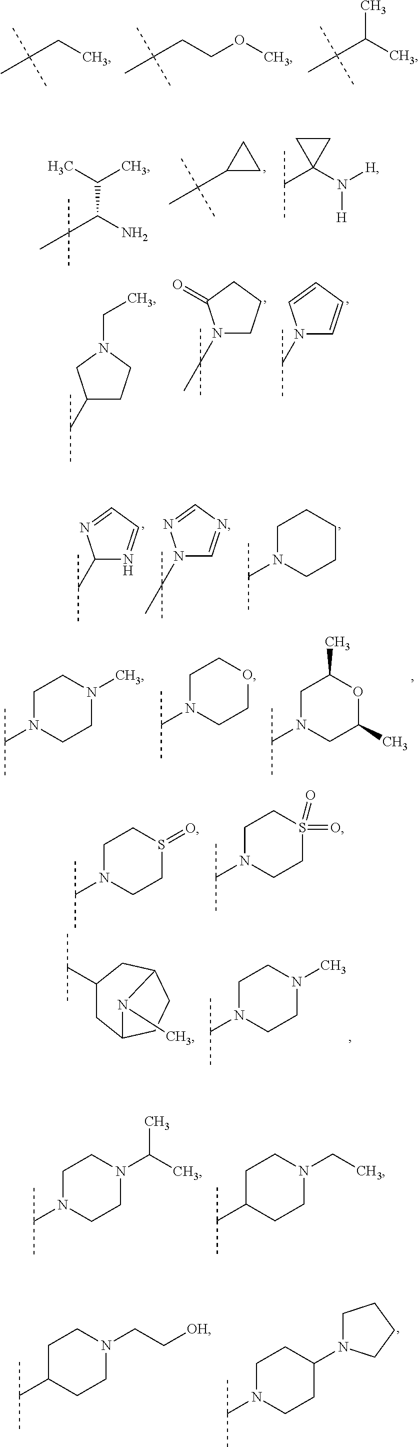

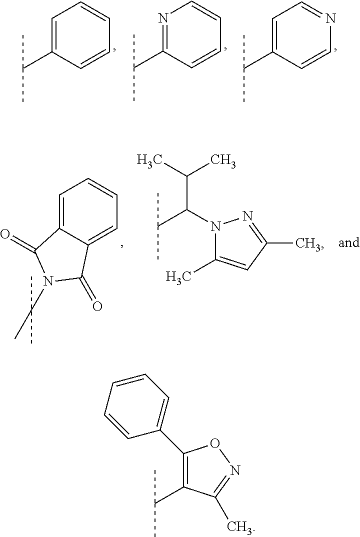

In Formula Ia1, L² is direct bond, or a linker chosen from: —O—, —O—alkylene-, —C(═O)—, —C(═O)N(R^{a})— wherein R^{a} is hydro or C₁₋₃ alkyl (e.g., methyl or ethyl), alkylene, alkynylene.

In some embodiments of Formula Ia1, L² is direct bond, or a linker chosen from: —O—, —O—(CH₂)ₙ— wherein n is 1, 2 or 3, —C(═O)—, —C(═O)N(R^{a})— wherein R^{a} is hydro or C₁₋₃ alkyl (e.g., methyl or ethyl), —(CH₂)ₙ— wherein n is 1, 2 or 3, —(CH₂)ₚ—C≡C—(CH₂)_{q}— wherein p and q are each independently 0, 1, 2 or 3.

In some embodiments, the compounds of Formula Ia are the compounds of Formula Ia2:

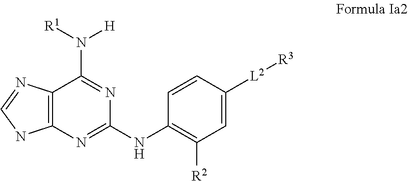

and pharmaceutically acceptable salts and solvates thereof.

In Formula Ia2, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ia2, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to the link to the core of the molecule) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ia2, R¹ is chosen from the group consisting of:

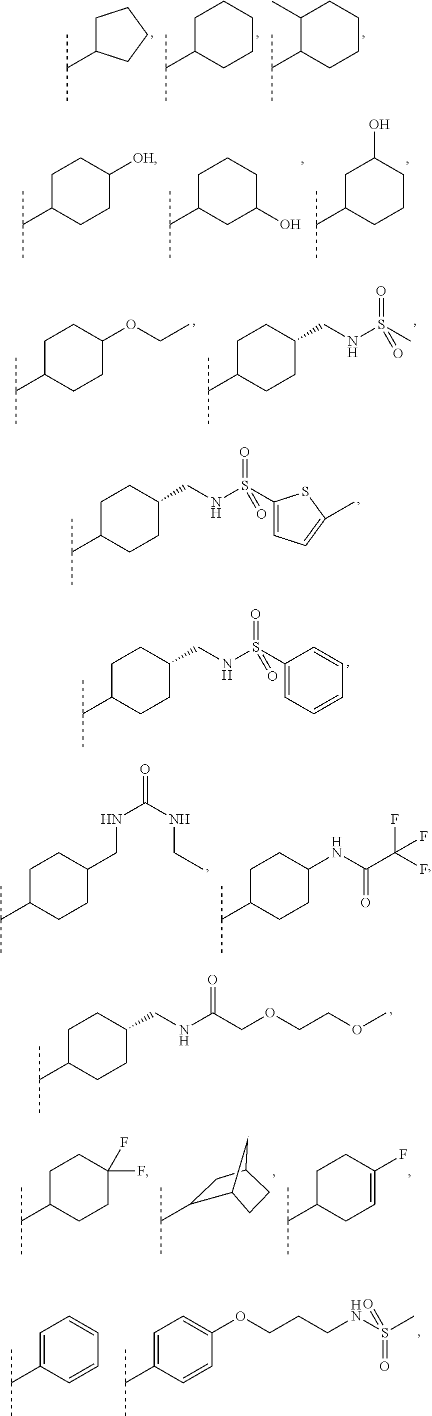

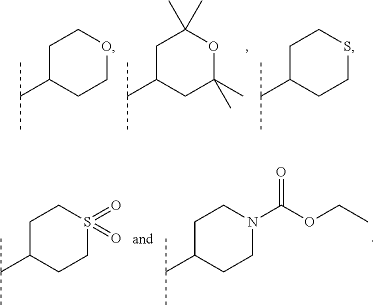

In Formula Ia2, R² is methyl, methoxy, ethoxy, Cl, trifluoromethyl.

In Formula Ia2, R³ is chosen from the group consisting of: methyl, methylene, trifluoromethyl, ethyl, ethylene, propyl, propylene, pentyl, pentylene,

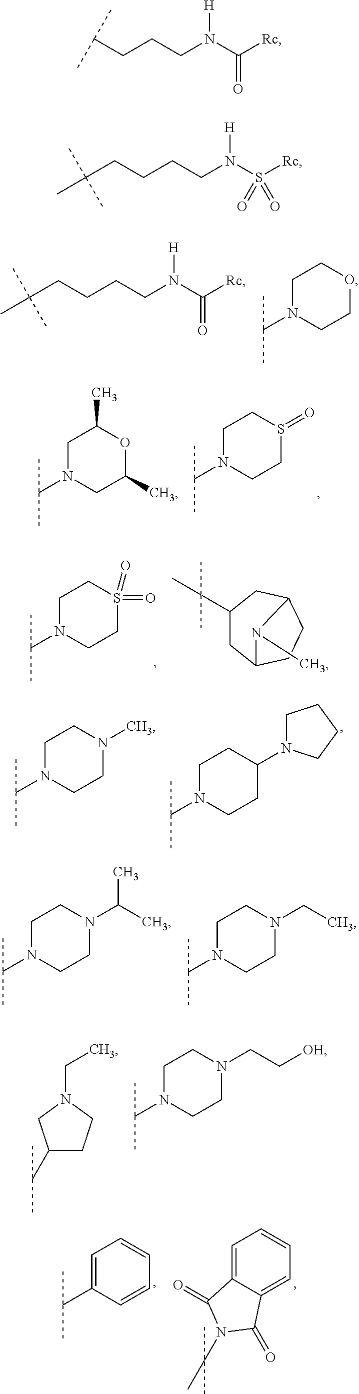

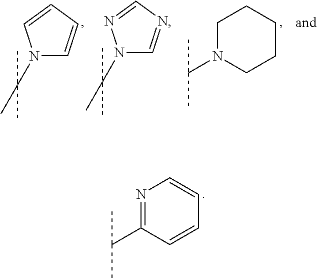

In Formula Ia2, R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole; or R^{c} is chosen from the group consisting of:

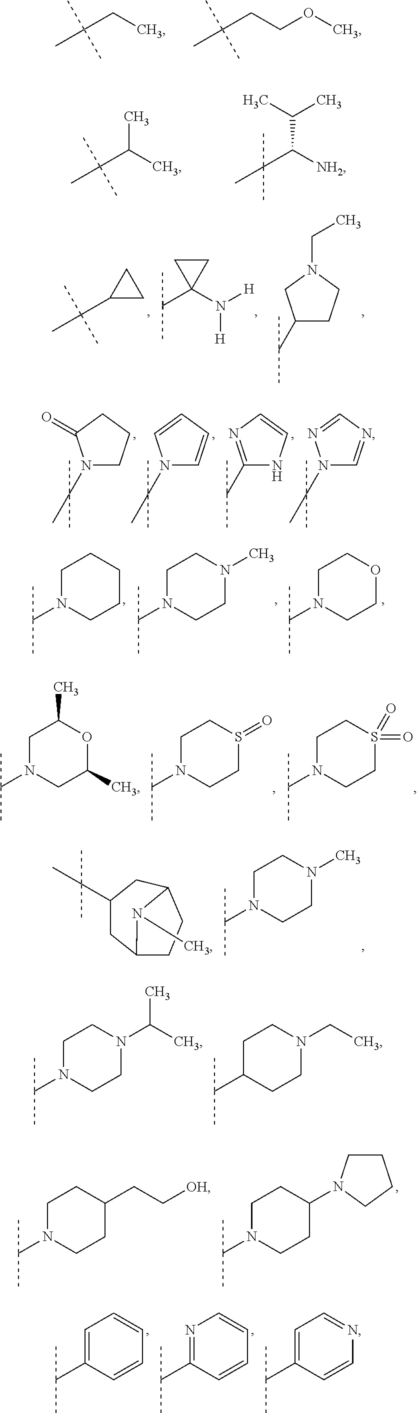

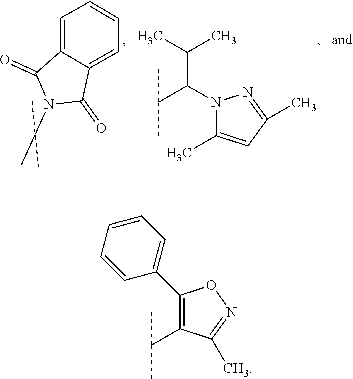

In Formula Ia2, L² is direct bond, or a linker chosen from: —O—, —O—(CH₂)ₙ— wherein n is 1, 2 or 3, —C(═O)—, —C(═O)N(R^{a})— wherein R^{a} is hydro or C₁₋₃ alkyl (e.g., methyl or ethyl), —(CH₂)ₙ— wherein n is 1, 2 or 3, —(CH₂)ₚ—C≡C—(CH₂)_{q}— wherein p and q are each independently 0, 1, 2 or 3.

In some embodiments, the compounds of Formula I are compounds of Formula Ib:

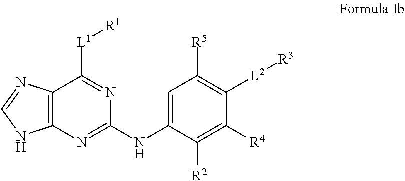

and pharmaceutically acceptable salts and solvates thereof.

In Formula Ib, R¹ is an optionally substituted carbocycle or heterocycle.

In Formula Ib, R² is chosen from the group consisting of: halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle.

In Formula Ib, R³ is a group chosen from: hydro, hydroxy, haloalkyl, —R^{c}, —C(═O)R^{c}, -alkylene-C(═O)R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, N(R^{b})S(O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})R^{e}; wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, heterocyclylalkyl, aryl, substituted or unsubstituted heteroaryl, nitro, hydroxy, halo, aminoalkyl, —C(═O)N(R^{dd})R^{ee} and —N(R^{dd})(R^{ee}); or R^{b} and R^{c}, when attached to the same atom, together with the atom to which they are bound form a heterocycle or carbocycle optionally substituted with alkyl, hydroxyl, or amino; wherein R^{d} and R^{e} are each independently chosen from hydro, hydroxyl, and C₁₋₄ alkyl, or R^{d} and R^{e} together with the nitrogen atom to which they are bound form a heterocycle optionally substituted with alkyl, hydroxyl, or amino; and wherein R^{dd} and R^{ee} are each independently chosen from hydro, hydroxyl, and C₁₋₄ alkyl, or R^{dd} and R^{ee} together with the nitrogen atom to which they are bound form a heterocycle optionally substituted with alkyl, hydroxyl, or amino.

In Formula Ib, R⁴ and R⁵ are independently chosen from: hydro, halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl.

In Formula Ib, L¹ is direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl (e.g., methyl), alkoxyl, carboxyl, or carbocycle.

In Formula Ib, L² is direct bond or a linker chosen from: alkynylene (e.g., ethynylene, 1-propynylene, 2-propynylene), aryl (e.g., phenyl), heterocycle (e.g., pyrrolidine, piperidine, piperazine, morpholine, and thiomorpholine), heteroaryl, heteroarylalkyl, arylalkyl, and heterocyclylalkyl.

In some embodiments of Formula Ib, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ib, is a substituted or unsubstituted C₃₋₆ cycloalkyl, heterocycle, C₃₋₆ cycloalkylalkyl, or heterocycloalkyl.

In some embodiments of Formula Ib, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ib, R¹ is chosen from the group consisting of:

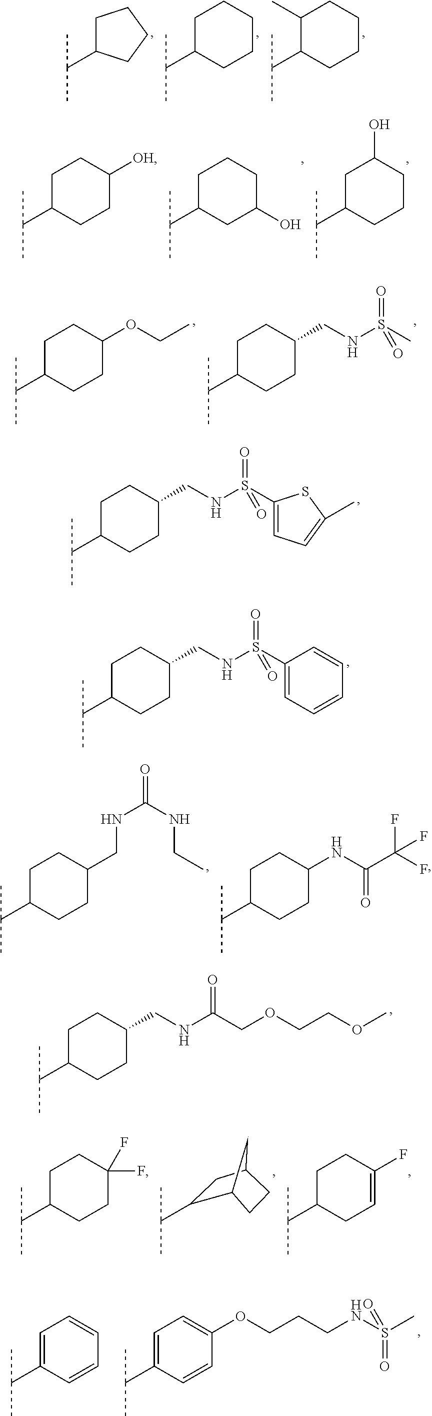

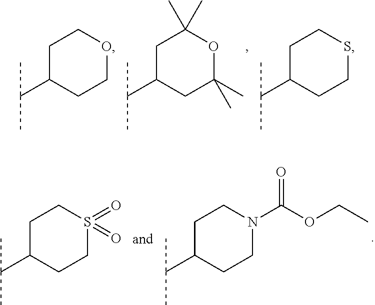

In some embodiments of Formula Ib, R² is halo, methyl optionally substituted with halo, ethyl optionally substituted with halo, methylthio, ethylthio, methoxy, or ethoxy.

In some embodiments of Formula Ib, R² is methyl, methoxy, ethoxy, Cl, or trifluoromethyl.

In some embodiments of Formula Ib, R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino.

In some embodiments of Formula Ib, R³ is hydro only when R⁴ or R⁵ is not hydro.

In some embodiments of Formula Ib, R³ is chosen from the group consisting of: methyl, methylene, trifluoromethyl, ethyl, ethylene, propyl, propylene, pentyl, pentylene,

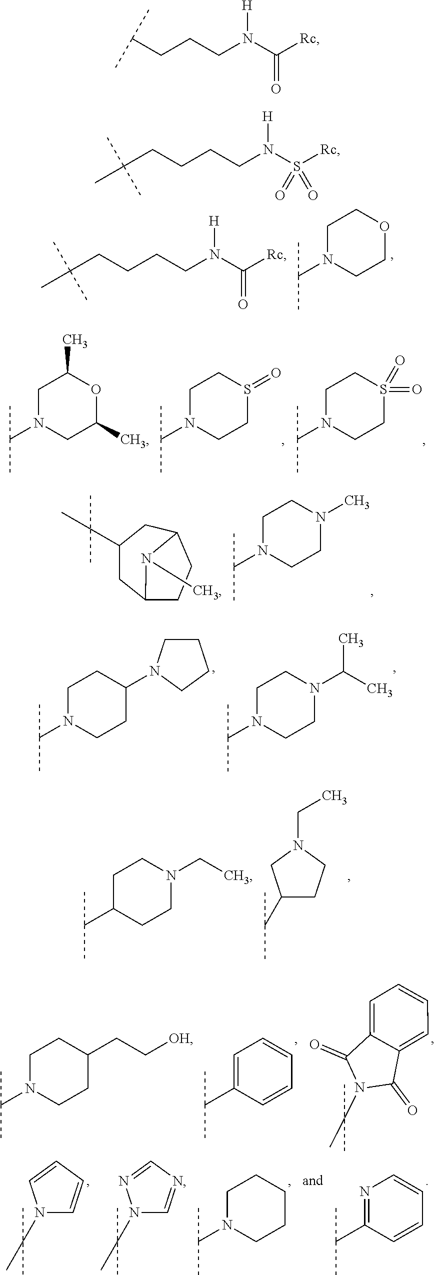

In some embodiments of Formula Ib R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments of Formula Ib R^{c} is chosen from the group consisting of:

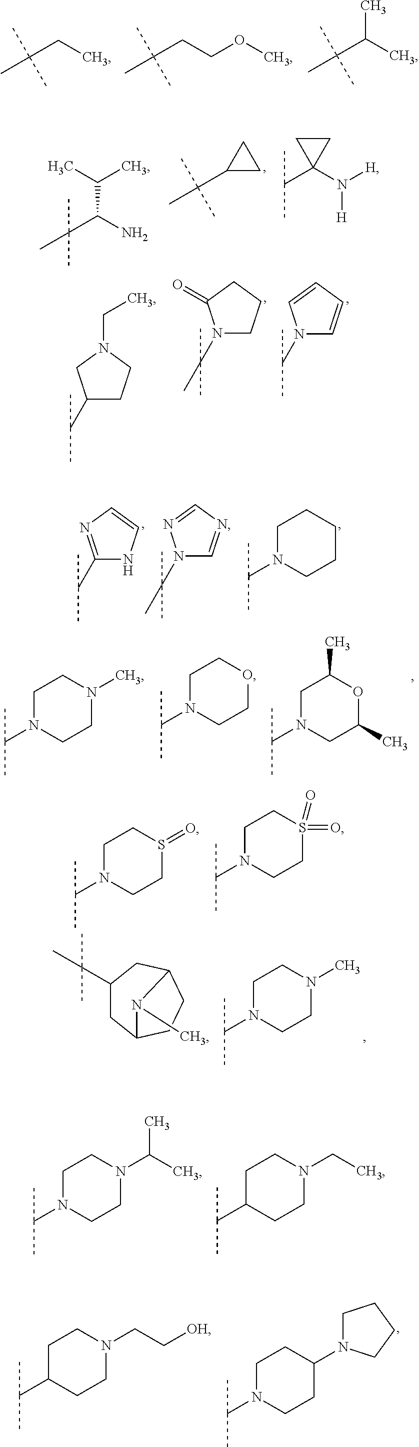

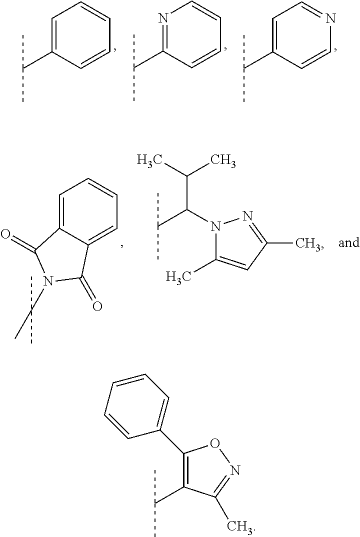

In some embodiments of Formula Ib, R³ is —R⁶—R⁷, wherein R⁶ is: is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

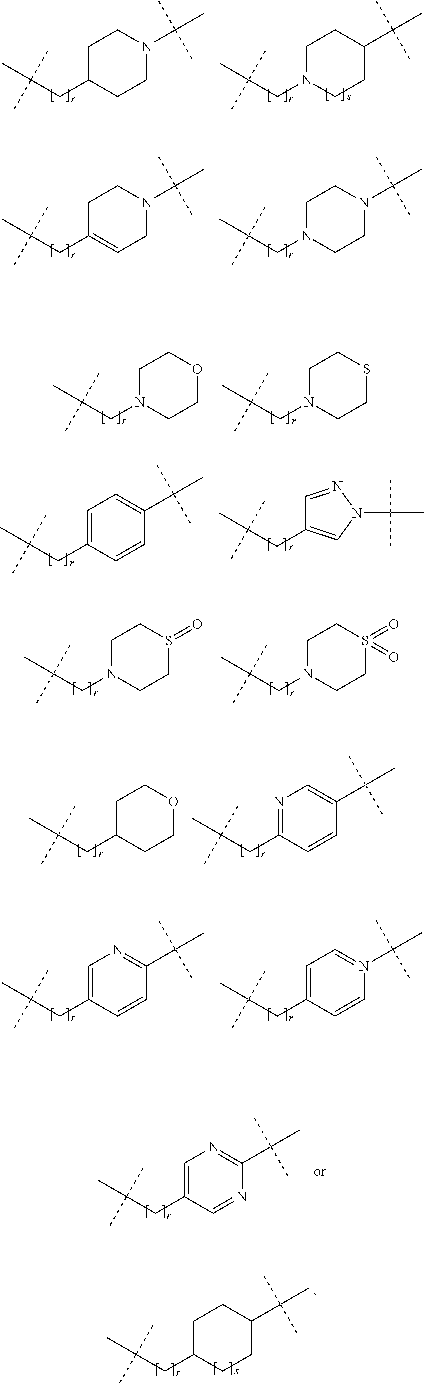

wherein r is 0, 1, or 2; and
wherein s is 0, 1, −1; and,

wherein R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is one of:

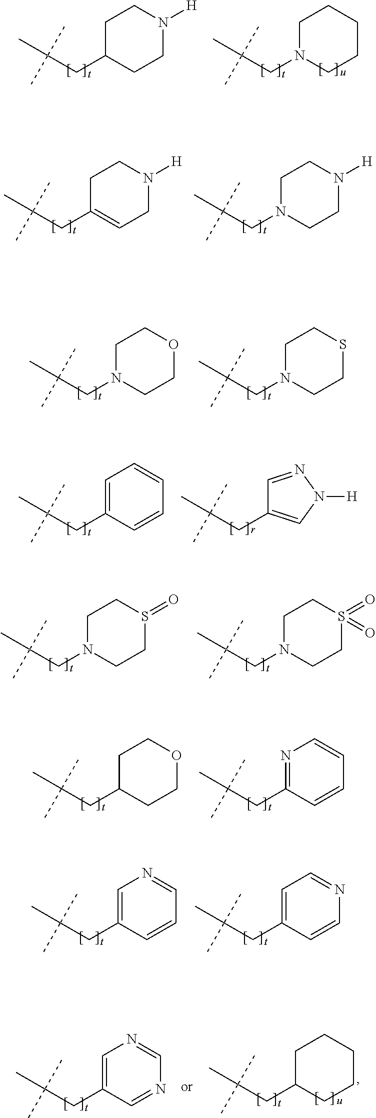

wherein t is 0, 1, or 2;
wherein u is 0, 1, −1.

In some embodiments of Formula Ib, r is 0.

In some embodiments of Formula Ib, s is 1.

In some embodiments of Formula Ib, t is 0.

In some embodiments of Formula Ib, u is 1.

In some embodiments of Formula Ib, R⁶ is C₁₋₃ alkyl, hydroxy, hydroxy-C₁₋₃ alkylene, halo-C₁₋₃ alkylene, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

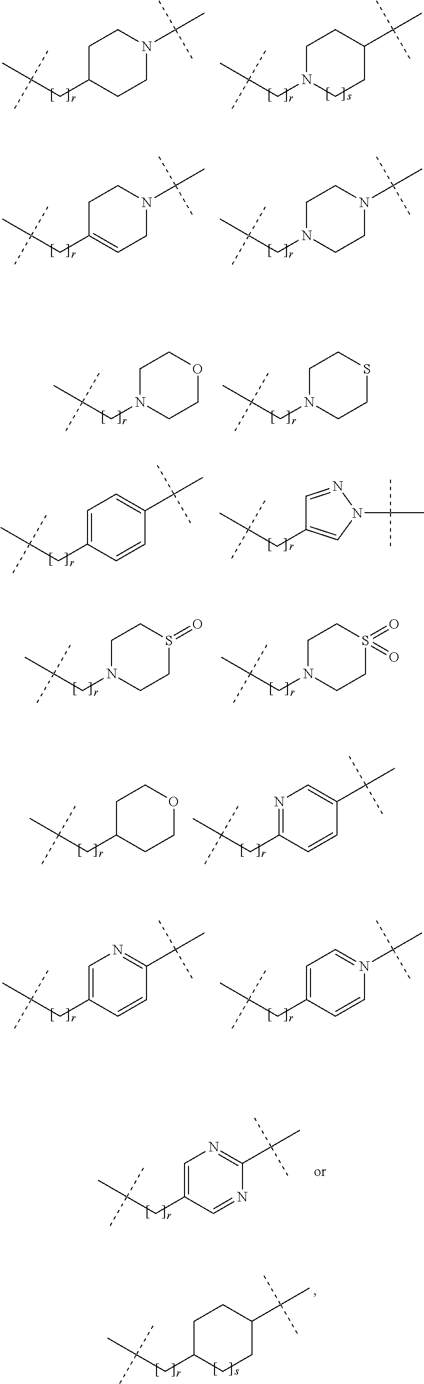

wherein r is 0, 1, or 2; and wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, —N(R^{h})Rⁱ—, wherein

R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or

R⁷ is one of:

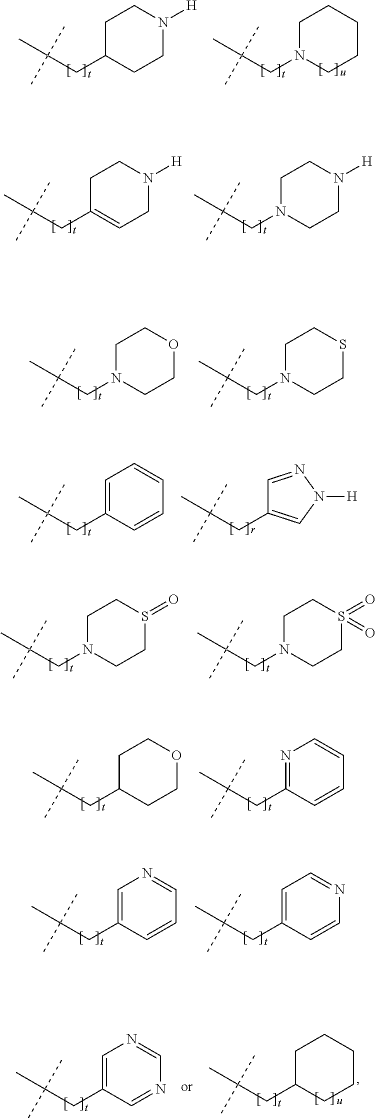

wherein t is 0, 1, or 2; and
wherein u is 0, 1, −1.

In some embodiments of Formula Ib, R⁶ is C₁₋₃ alkyl, hydroxy, hydroxy-C₁₋₃ alkylene, halo-C₁₋₃ alkylene, —C₁₋₃ alkylene-C(═O)—, —N(R^{f})C(═O)—, -alkylene-N(R^{f})C(═O)—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

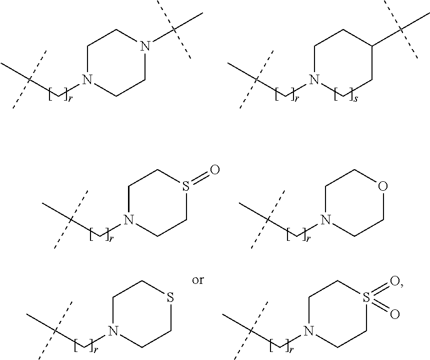

wherein r is 0, 1, or 2; and
wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is one of:

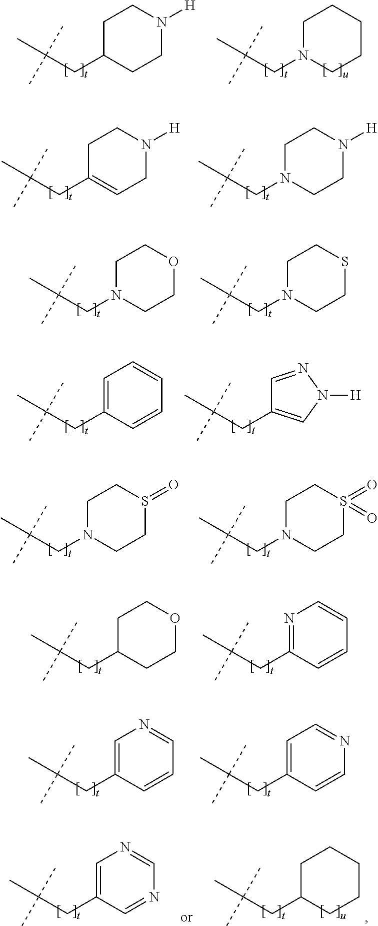

wherein t is 0, 1, or 2; and
wherein u is 0, 1, −1.

In some embodiments of Formula Ib, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —C(═O)N(R^{f})R^{f}—, —alkylene-C(═O)N(R^{f})R^{g}—, —S(═O)₂N(R^{f})R^{g}—, -alkylene-S(═O)₂N(R^{f})R^{g}—, —S(═O)₂—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, or amino-C₁₋₃ alkylene.

In some embodiments of Formula Ib, R⁶ is C₁₋₃ alkyl, hydroxy, hydroxy-C₁₋₃ alkylene, or halo-C₁₋₃ alkylene. In some embodiments of these embodiments, R⁷ is not present, is hydro, or is —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

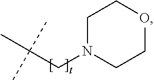

wherein t is 0, 1, or 2. In some of these embodiments of Formula Ib, t is 2.

In some embodiments of Formula Ib, R² and R⁴, together with the carbon atoms to which they are attached, form the following ring structure:

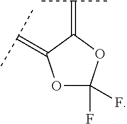

In some embodiments of Formula Ib, R⁴, R⁵, or both, are Hydrogen.

In some embodiments of Formula Ib, L¹ is direct bond or a linker chosen from: —O—, —S—, —S(═O)—, —S(═O)₂—, —N(R^{a})—, —CH(R^{a})—, —(CH₂)ₙ— wherein n is 1, 2 or 3, —C(═O)—, —C(═O)N(R^{a})—, wherein R^{a} is hydro or C₁₋₆ alkyl (e.g., methyl).

In some embodiments of Formula Ib, L¹ is direct bond, —N(R^{a})— wherein R^{a} is hydro or C₁₋₃ alkyl (e.g., methyl or ethyl), —(CH₂)ₙ— wherein n is 1, 2 or 3, or —C(═O)—.

In some embodiments of Formula Ib, L¹ is —N(H)—.

In some embodiments of Formula Ib, L² is alkynylene, aryl, arylalkyl, heteraryl, heteroarylalkyl, or

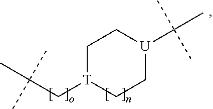

wherein T is carbon or nitrogen, U is carbon, nitrogen, sulfur, or oxygen, n is 0, 1, or −1, o is 0, 1, or 2, and there is optionally at least one ring carbon-ring carbon double bond.

In some embodiments of Formula Ib, L² is alkynylene or

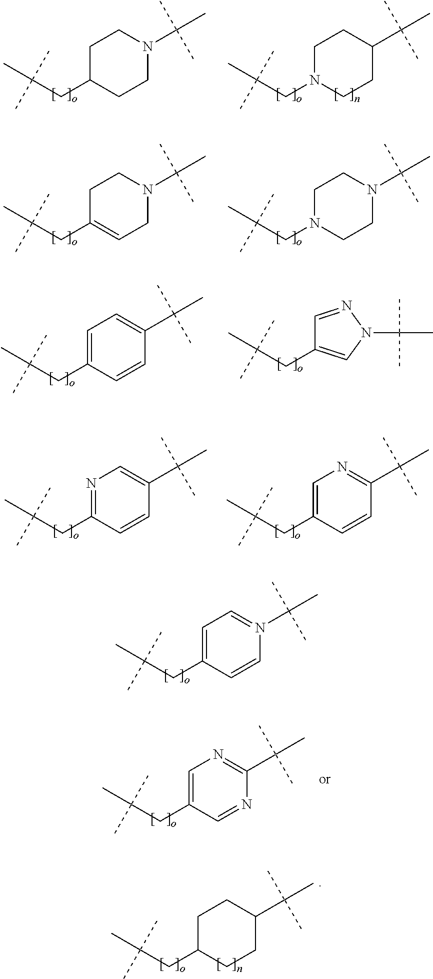

In some embodiments, the compounds of Formula Ib are compounds of Formula Ib1:

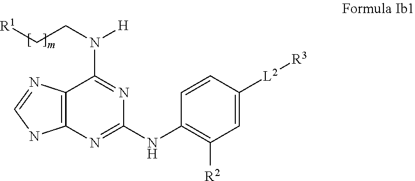

and pharmaceutically acceptable salts and solvates thereof.

In Formula Ib1, R¹ is an optionally substituted carbocycle or heterocycle.

In Formula Ib1, m is 0, 1, or −1.

In Formula Ib1, R² is halo, methyl optionally substituted with halo, ethyl optionally substituted with halo, methylthio, ethylthio, methoxy, or ethoxy.

In Formula Ib1, R³ is a group chosen from: hydro, hydroxy, haloalkyl, —R^{c}, —C(═O)R^{c}, -alkylene-C(═O)R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})R^{e}; wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, heterocyclylalkyl, aryl, substituted or unsubstituted heteroaryl, nitro, hydroxy, halo, aminoalkyl, —C(═O)N(R^{dd})R^{ee} and —N(R^{dd})(R^{ee}); or R^{b} and R^{c}, when attached to the same atom, together with the atom to which they are bound form a heterocycle or carbocycle optionally substituted with alkyl, hydroxyl, or amino; wherein R^{d} and R^{e} are each independently chosen from hydro, hydroxyl, and C₁₋₄ alkyl, or R^{d} and R^{e} together with the nitrogen atom to which they are bound form a heterocycle optionally substituted with alkyl, hydroxyl, or amino; and wherein R^{dd} and R^{ee} are each independently chosen from hydro, hydroxyl, and C₁₋₄ alkyl, or R^{dd} and R^{ee} together with the nitrogen atom to which they are bound form a heterocycle optionally substituted with alkyl, hydroxyl, or amino.

In Formula Ib1, L² is direct bond or a linker chosen from: alkynylene (e.g., ethynylene, 1-propynylene, 2-propynylene), aryl (e.g., phenyl), heterocycle (e.g., pyrrolidine, piperidine, piperazine, morpholine, and thiomorpholine), heteroaryl, heteroarylalkyl, arylalkyl, and heterocyclylalkyl.

In some embodiments of Formula Ib1, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ib1, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ib1, R¹ is chosen from the group consisting of:

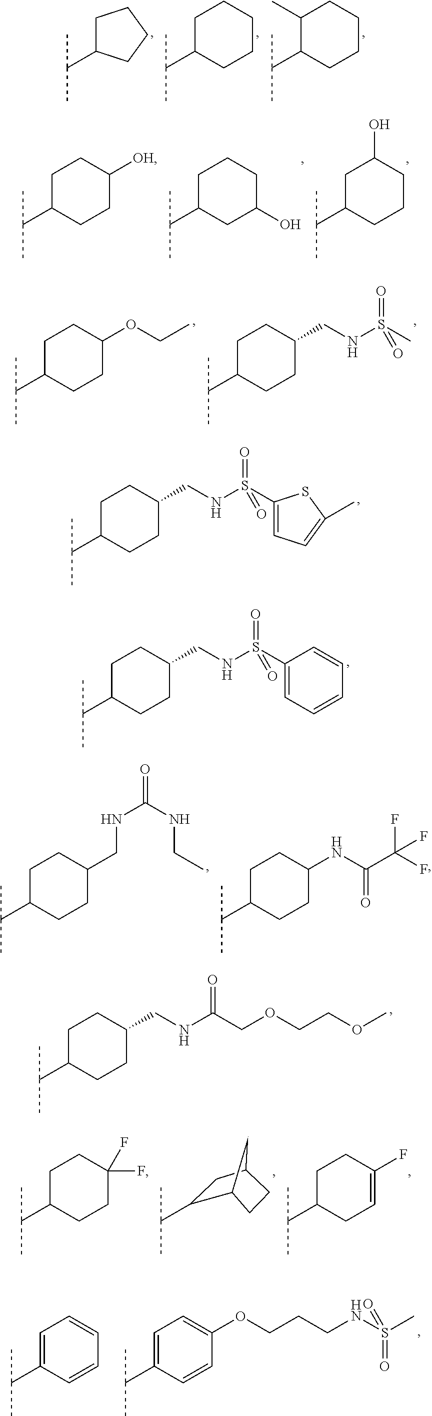

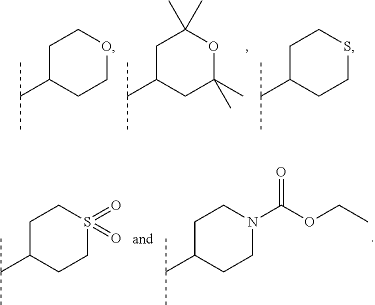

In some embodiments of Formula Ib1, R¹ is cyclobutyl, cyclopentyl, cyclohexyl, or oxane.

In some embodiments of Formula Ib1, m is −1.

In some embodiments of Formula Ib1, R² is methyl, methoxy, ethoxy, Cl, or trifluoromethyl.

In some embodiments of Formula Ib1, R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino.

In some embodiments of Formula Ib1, R³ is chosen from the group consisting of: methyl, methylene, trifluoromethyl, ethyl, ethylene, propyl, propylene, pentyl, pentylene,

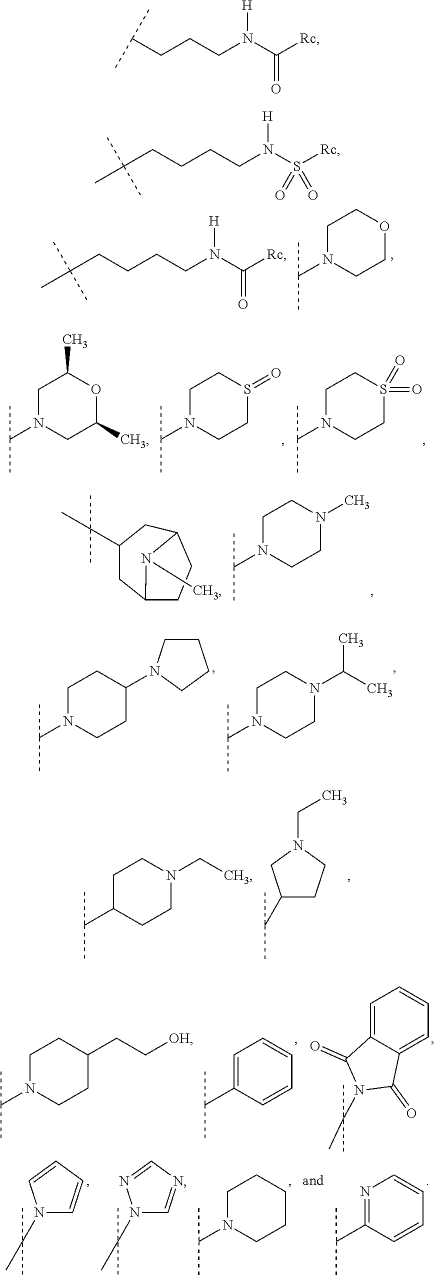

In some embodiments of Formula Ib1, R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments of Formula Ib1, R^{c} is chosen from the group consisting of:

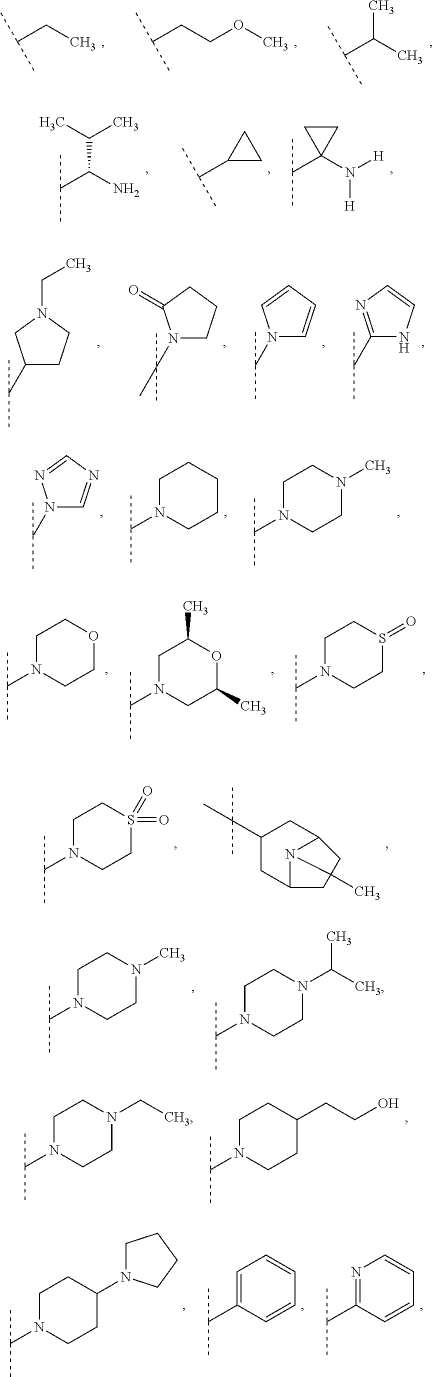

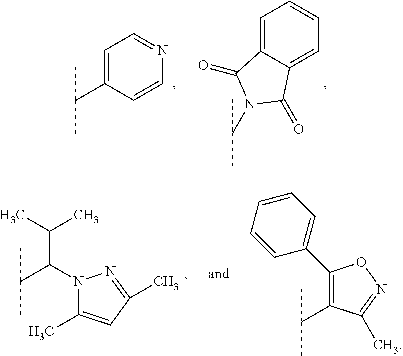

In some embodiments of Formula Ib1, R³ is —R⁶—R⁷, wherein R⁶ is: is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

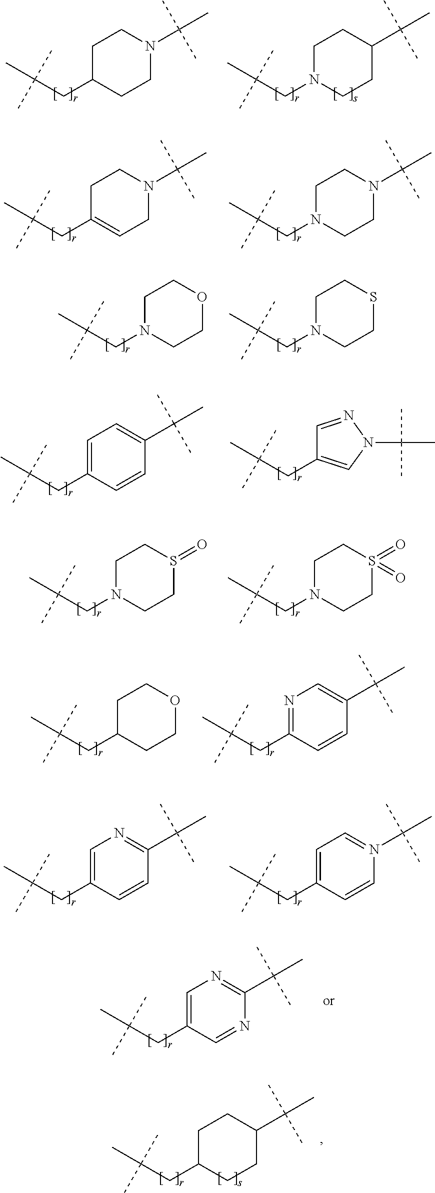

wherein r is 0, 1, or 2; and
wherein s is 0, 1, −1; and,

wherein R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

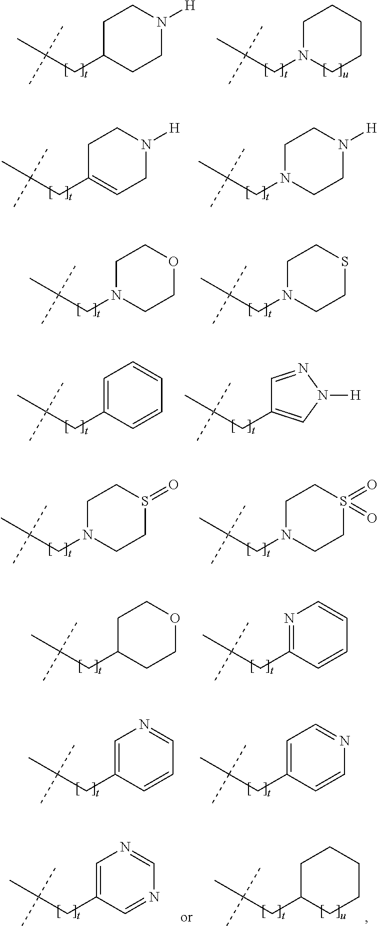

wherein t is 0, 1, or 2; and
wherein u is 0, 1, −1.

In some embodiments of Formula Ib1, r is 0.

In some embodiments of Formula Ib1, s is 1.

In some embodiments of Formula Ib1, t is 0.

In some embodiments of Formula Ib1, u is 1.

In some embodiments of Formula Ib1, R⁶ is C₁₋₃ alkyl, hydroxy, hydroxy-C₁₋₃ alkylene, halo-C₁₋₃ alkylene, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

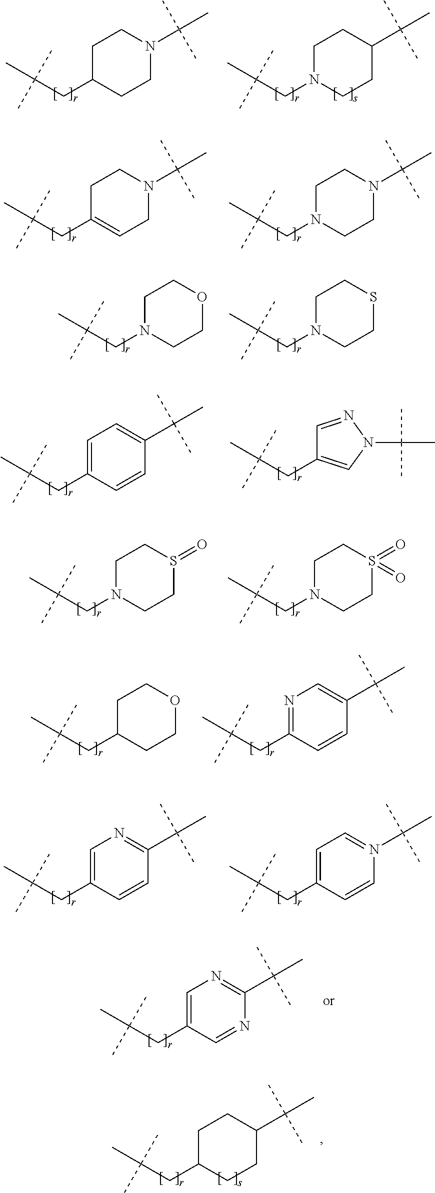

wherein r is 0, 1, or 2; and
wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or

R⁷ is:

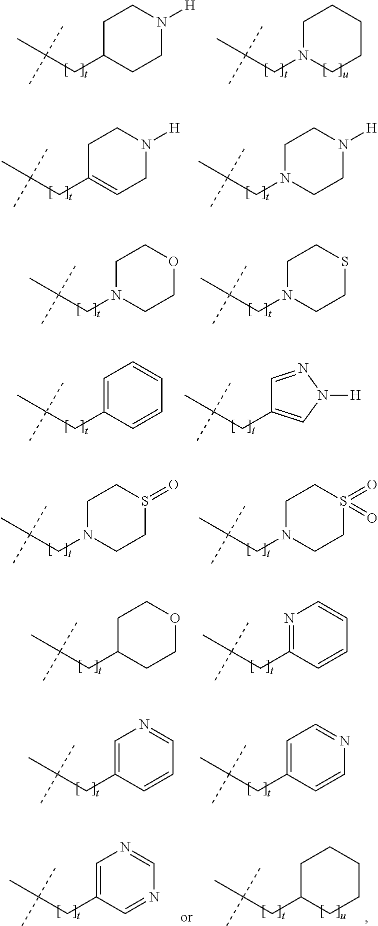

wherein t is 0, 1, or 2; and
wherein u is 0, 1, −1.

In some embodiments of Formula Ib1, R⁶ is C₁₋₃ alkyl, hydroxy, hydroxy-C₁₋₃ alkylene, halo-C₁₋₃ alkylene, —C₁₋₃ alkylene-C(═O)—, —N(R^{f})C(═O)—, -alkylene-N(R^{f})C(═O)—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

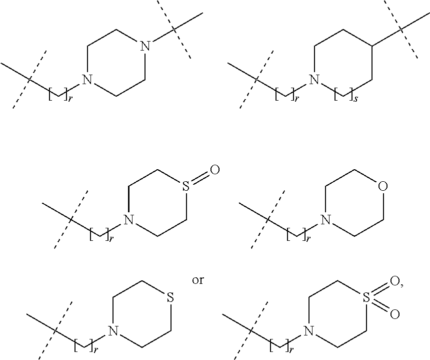

wherein r is 0, 1, or 2; and
wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is:

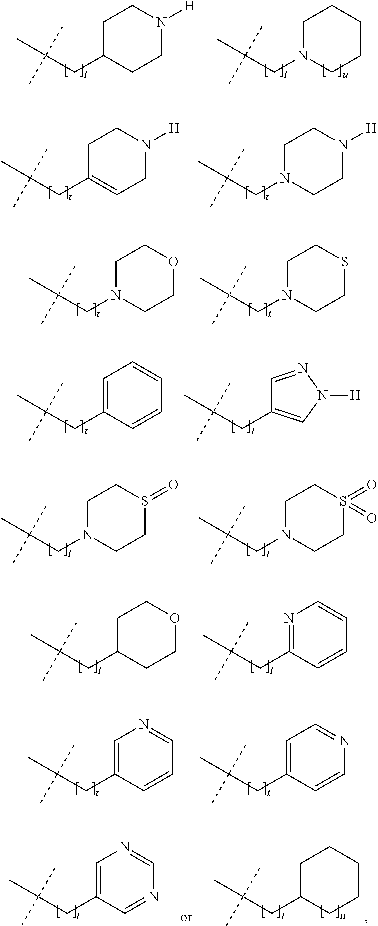

wherein t is 0, 1, or 2; and
wherein u is 0, 1, −1.

In some embodiments of Formula Ib1, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —C(═O)N(R^{f})R^{f}—, -alkylene-C(═O)N(R^{f})R^{g}—, —S(═O)₂N(R^{f})R^{g}—, -alkylene-S(═O)₂N(R^{f})R^{g}—, —S(═O)₂—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, or amino-C₁₋₃ alkylene.

In some embodiments of Formula Ib1, R⁶ is C₁₋₃ alkyl, hydroxy, hydroxy-C₁₋₃ alkylene, or halo-C₁₋₃ alkylene. In some embodiments of these embodiments, R⁷ is not present, is hydro, or is —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

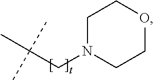

wherein t is 0, 1, or 2. In some of these embodiments of Formula Ib1, t is 2.

In some embodiments of Formula Ib1, L² is alkynylene, aryl, arylalkyl, heteraryl, heteroarylalkyl, or

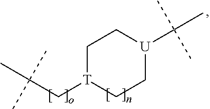

wherein T is carbon or nitrogen, U is carbon, nitrogen, sulfur, or oxygen, n is 0, 1, or −1, o is 0, 1, or 2, and there is optionally at least one ring carbon-ring carbon double bond.

In some embodiments of Formula Ib1, L² is alkynylene or:

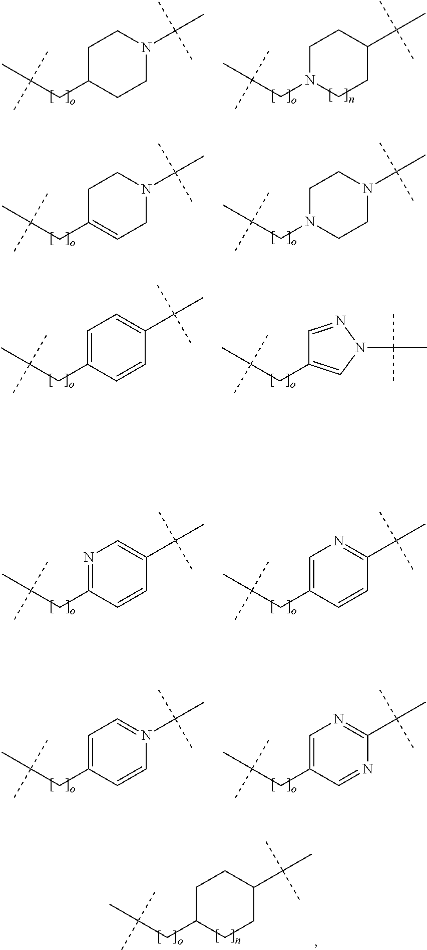

wherein o is 0, 1, or 2; and
wherein n is 0, 1, or −1.

In some embodiments, the compounds of Formula Ib are compounds of Formula Ib2:

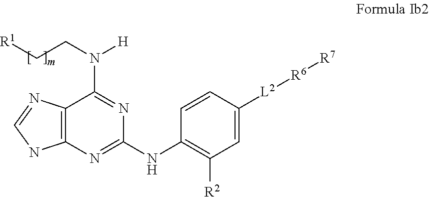

and pharmaceutically acceptable salts and solvates thereof.

In Formula Ib2, R¹ is an optionally substituted carbocycle or heterocycle.

In Formula Ib2, m is 0, 1, or −1.

In Formula Ib2, R² is halo, methyl optionally substituted with halo, ethyl optionally substituted with halo, methylthio, ethylthio, methoxy, or ethoxy.

In Formula Ib2, L² is direct bond or a linker chosen from: alkynylene (e.g., ethynylene, 1-propynylene, 2-propynylene), aryl (e.g., phenyl), heterocycle (e.g., pyrrolidine, piperidine, piperazine, morpholine, and thiomorpholine), heteroaryl, heteroarylalkyl, arylalkyl, and heterocyclylalkyl.

In Formula Ib2, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

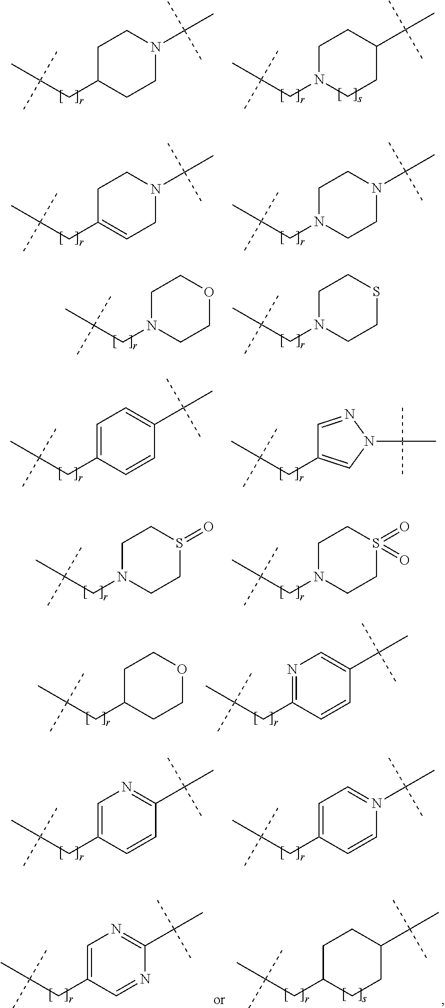

wherein r is 0, 1, or 2; and
wherein s is 0, 1, −1.

In Formula Ib2, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃alkylene-, or R^{h} and Rⁱ together with the nitrogen atom to which they are bound form a heterocycle heteroaryl optionally substituted with methyl, hydroxyl, or amino; or R⁷ is:

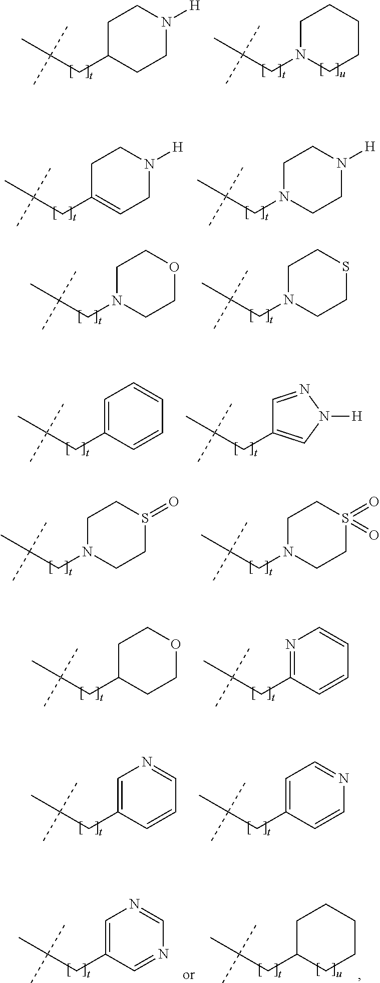

wherein t is 0, 1, or 2;
wherein u is 0, 1, −1.

In some embodiments of Formula Ib2, r is 0.

In some embodiments of Formula Ib2, s is 1.

In some embodiments of Formula Ib2, t is 0.

In some embodiments of Formula Ib2, u is 1.

In some embodiments of Formula Ib2, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula Ib2, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula Ib2, R¹ is chosen from the group consisting of:

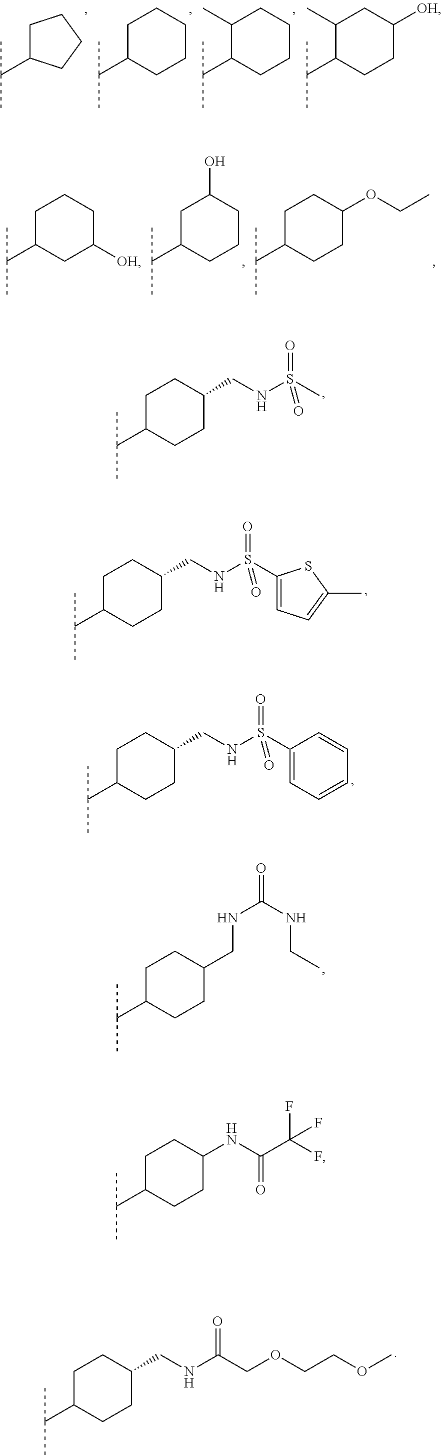

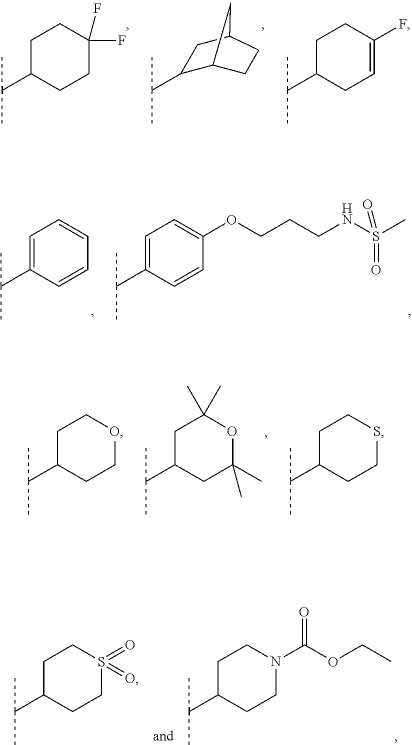

In some embodiments of Formula Ib2, R¹ is cyclobutyl, cyclopentyl, cyclohexyl, or oxane.

In some embodiments of Formula Ib2, m is −1.

In some embodiments of Formula Ib2, R² is methyl, methoxy, ethoxy, Cl, or trifluoromethyl.

In some embodiments of Formula Ib2, L² is alkynylene, or

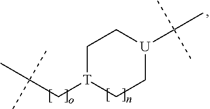

wherein T is carbon or nitrogen, U is carbon, nitrogen, sulfur, or oxygen, n is 0, 1, or −1, o is 0, 1, or 2, and there is optionally at least one ring carbon-ring carbon double bond.

In some embodiments of Formula Ib2, L² is alkynylene or:

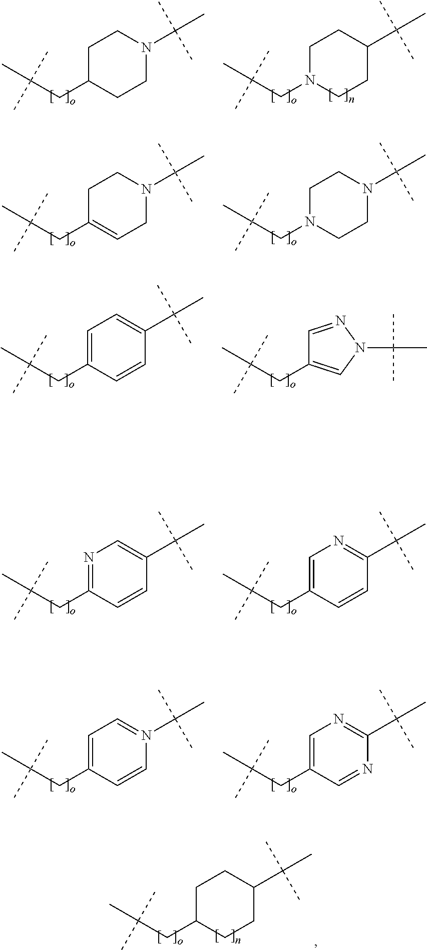

wherein o is 0, 1, or 2; and
wherein n is 0, 1, or −1.

In some embodiments, the compounds of Formula Ib are compounds of Formula Ib3:

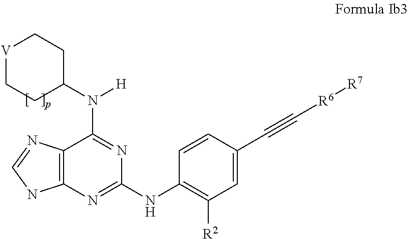

and pharmaceutically acceptable salts and solvates thereof.

In Formula Ib3, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib3, p is 0, 1, or −1.

In Formula Ib3, R² is halo, methyl optionally substituted with halo, ethyl optionally substituted with halo, methylthio, ethylthio, methoxy, or ethoxy.

In Formula Ib3, R⁶ is C₁₋₃ alkyl, hydroxy, hydroxy-C₁₋₃ alkylene, halo-C₁₋₃ alkylene, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

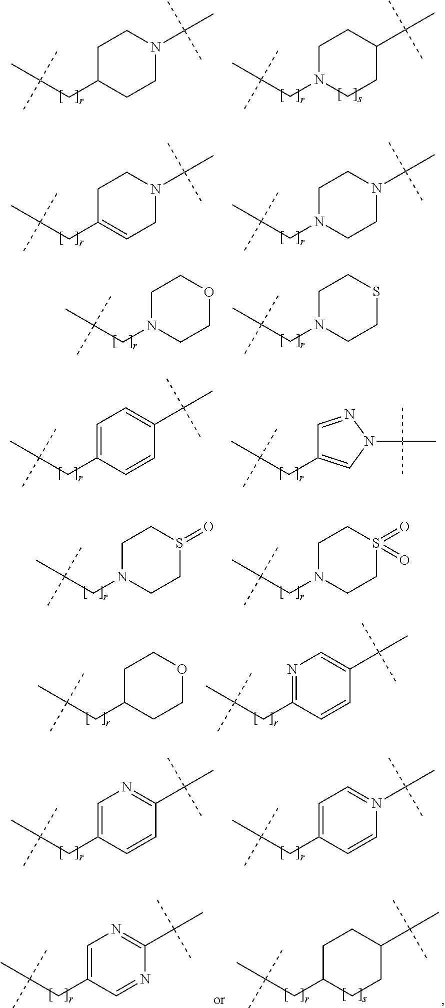

wherein r is 0, 1, or 2; and
wherein s is 0, 1, −1.

In Formula Ib3, R⁷ is not present, is hydro, or is one or more of: C₁₋₃ alkyl, C₃₋₆ cycloalkyl, hydroxy, hydroxy-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is:

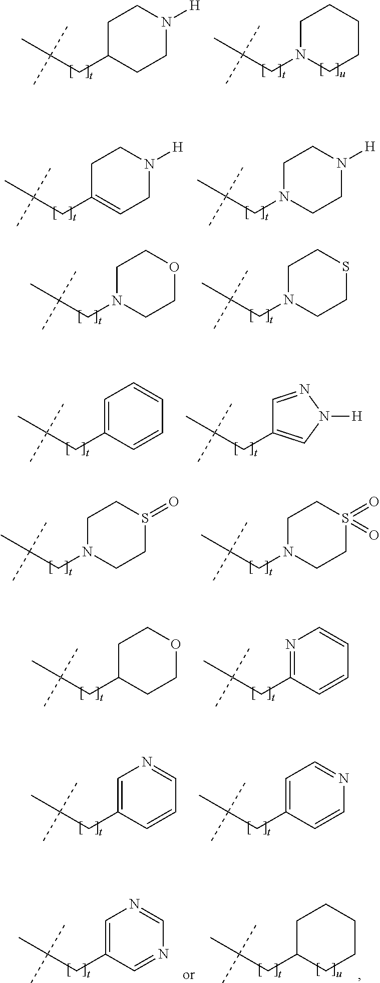

wherein t is 0, 1, or 2;
wherein u is 0, 1, −1.

In some embodiments of Formula Ib3, r is 0.

In some embodiments of Formula Ib3, s is 1.

In some embodiments of Formula Ib3, t is 0.

In some embodiments of Formula Ib3, u is 1.

In some embodiments of Formula Ib3, V is carbon optionally substituted with hydroxyl.

In some embodiments of Formula Ib3, V is oxygen.

In some embodiments of Formula Ib3, p is 1.

In some embodiments of Formula Ib3, R² is methyl, methoxy, ethoxy, Cl, or trifluoromethyl.

In some embodiments, the compounds of Formula Ib are compounds of Formula Ib4:

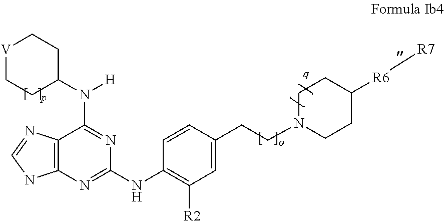

and pharmaceutically acceptable salts and solvates thereof.

In Formula Ib4, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib4, p is 0, 1, or −1.

In Formula Ib4, R² is halo, methyl optionally substituted with halo, ethyl optionally substituted with halo, methylthio, ethylthio, methoxy, or ethoxy.

In Formula Ib4, o is 0, 1, or −1.

In Formula Ib4, q is 0, 1, or −1.

In Formula Ib4, R⁶ is C₁₋₃ alkyl, hydroxy, hydroxy-C₁₋₃ alkylene, halo-C₁₋₃ alkylene, —C₁₋₃ alkylene-C(═O)—, —N(R^{f})C(═O)—, -alkylene-N(R^{f})C(═O)—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

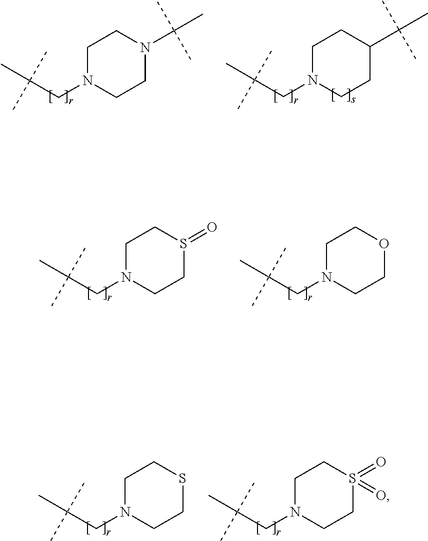

wherein r is 0, 1, or 2; and
wherein s is 0, 1, −1.

In Formula Ib4, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is:

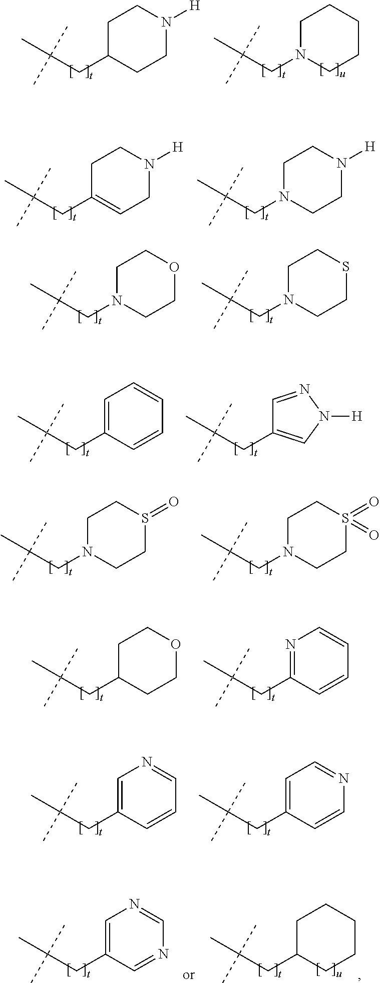

wherein t is 0, 1, or 2;
wherein u is 0, 1, −1.

In some embodiments of Formula Ib4, r is 0.

In some embodiments of Formula Ib4, s is 1.

In some embodiments of Formula Ib4, t is 0.

In some embodiments of Formula Ib4, u is 1.

In some embodiments of Formula Ib4, o is −1.

In some embodiments of Formula Ib4, q is 1.

In some embodiments of Formula Ib4, V is carbon optionally substituted with hydroxyl.

In some embodiments of Formula Ib4, V is oxygen.

In some embodiments of Formula Ib4, p is 1.

In some embodiments of Formula Ib4, R² is methyl, methoxy, ethoxy, Cl, or trifluoromethyl.

In some embodiments, the compounds of Formula Ib are compounds of Formula Ib5:

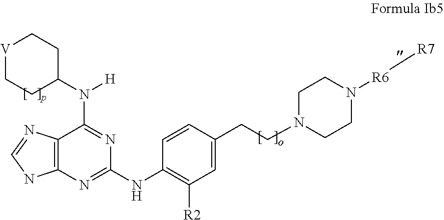

and pharmaceutically acceptable salts and solvates thereof.

In Formula Ib5, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib5, p is 0, 1, or −1.

In Formula Ib5, R² is halo, methyl optionally substituted with halo, ethyl optionally substituted with halo, methylthio, ethylthio, methoxy, or ethoxy.

In Formula Ib5, o is 0, 1, or −1.

In Formula Ib5, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

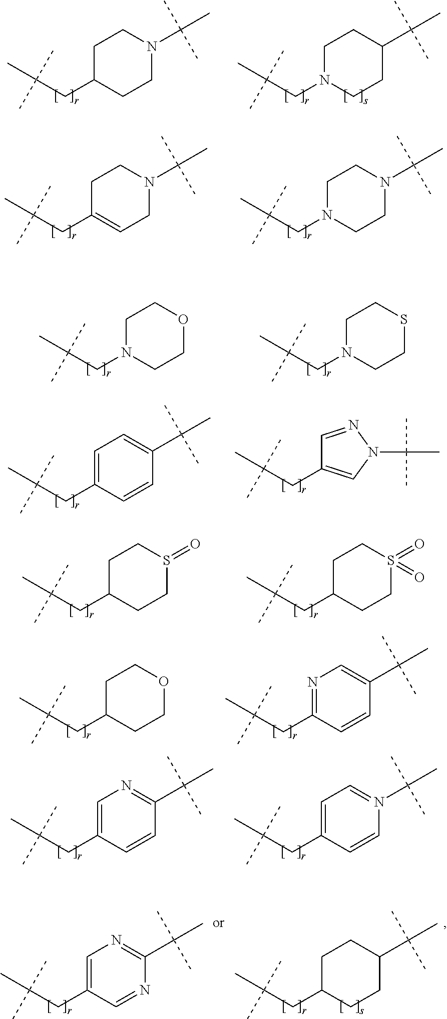

wherein r is 0, 1, or 2; and
wherein s is 0, 1, −1.

In Formula Ib5, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃alkylene-, or R^{h} and Rⁱ together with the nitrogen atom to which they are bound form a heterocycle heteroaryl optionally substituted with methyl, hydroxyl, or amino; or R⁷ is selected from:

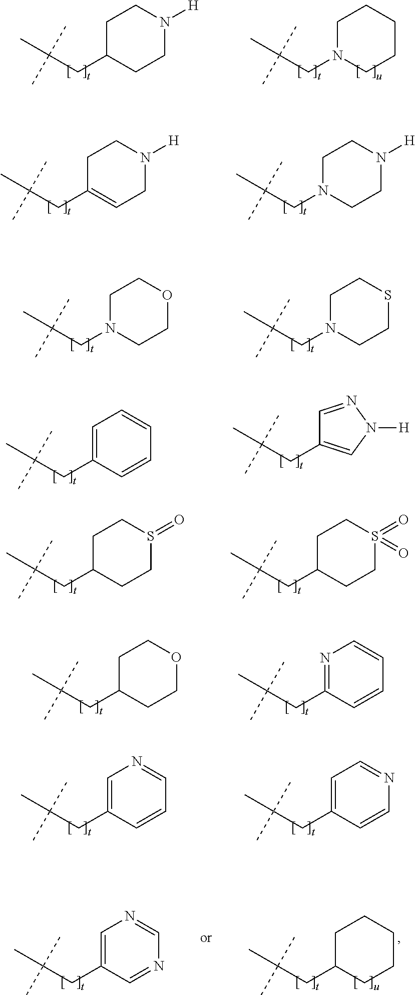

wherein t is 0, 1, or 2;
wherein u is 0, 1, −1.

In some embodiments of Formula Ib5, r is 0.

In some embodiments of Formula Ib5, s is 1.

In some embodiments of Formula Ib5, t is 0.

In some embodiments of Formula Ib5, u is 1.

In some embodiments of Formula Ib5, o is −1.

In some embodiments of Formula Ib5, V is carbon optionally substituted with hydroxyl.

In some embodiments of Formula Ib5, V is oxygen.

In some embodiments of Formula Ib5, p is 1.

In some embodiments of Formula Ib5, R² is methyl, methoxy, ethoxy, Cl, or trifluoromethyl.

In some embodiments, the compounds of Formula Ib are compounds of Formula Ib6:

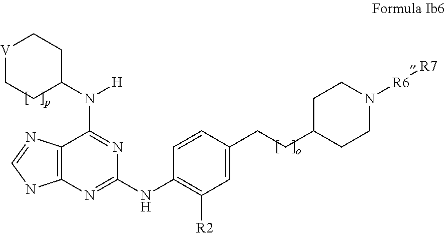

and pharmaceutically acceptable salts and solvates thereof.

In Formula Ib6, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib6, p is 0, 1, or −1.

In Formula Ib6, R² is halo, methyl optionally substituted with halo, ethyl optionally substituted with halo, methylthio, ethylthio, methoxy, or ethoxy.

In Formula Ib6, o is 0, 1, or −1.

In Formula Ib6, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

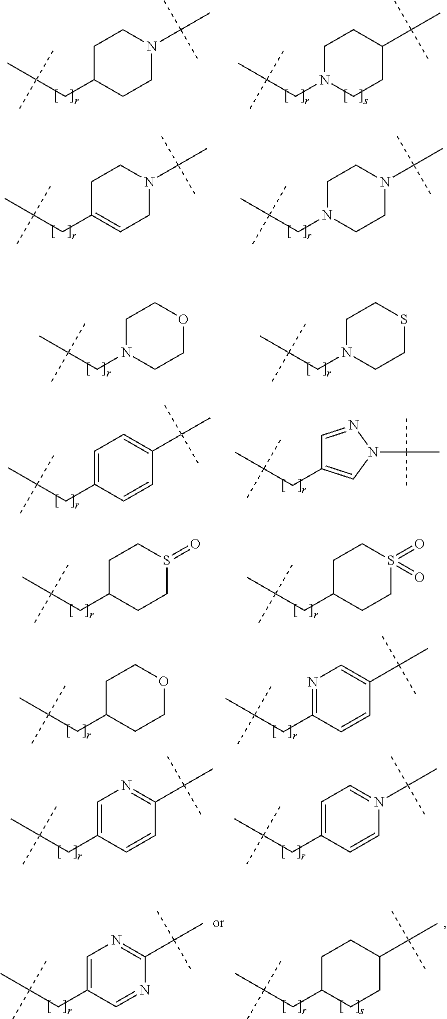

wherein r is 0, 1, or 2; and
wherein s is 0, 1, −1.

In Formula Ib6, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is one of:

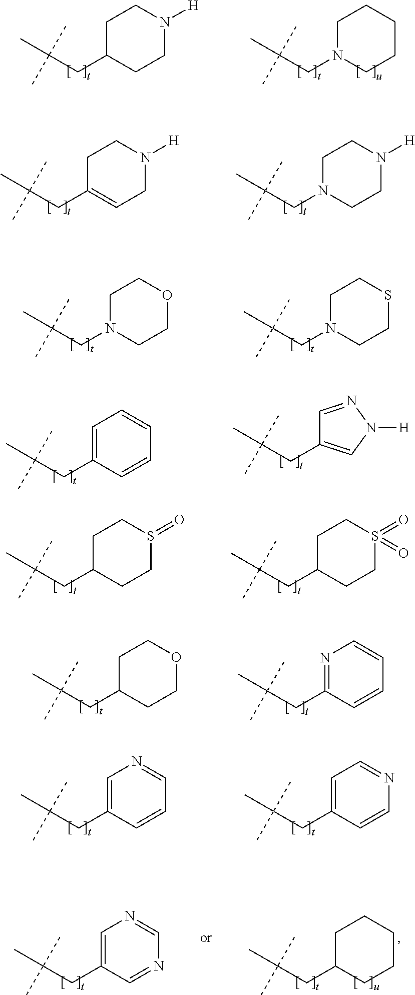

wherein t is 0, 1, or 2;
wherein u is 0, 1, −1.

In some embodiments of Formula Ib6, r is 0.

In some embodiments of Formula Ib6, s is 1.

In some embodiments of Formula Ib6, t is 0.

In some embodiments of Formula Ib6, u is 1.

In some embodiments of Formula Ib6, o is −1.

In some embodiments of Formula Ib6, V is carbon optionally substituted with hydroxyl.

In some embodiments of Formula Ib6, V is oxygen.

In some embodiments of Formula Ib6, p is 1.

In some embodiments of Formula Ib6, R² is methyl, methoxy, ethoxy, Cl, or trifluoromethyl.

In some embodiments, the compounds of Formula Ib are compounds of Formula Ib7:

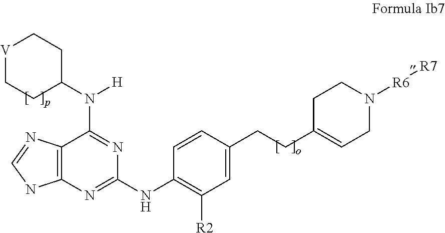

and pharmaceutically acceptable salts and solvates thereof.

In Formula Ib7, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib7, p is 0, 1, or −1.

In Formula Ib7, R² is halo, methyl optionally substituted with halo, ethyl optionally substituted with halo, methylthio, ethylthio, methoxy, or ethoxy.

In Formula Ib7, o is 0, 1, or −1.

In Formula Ib7, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

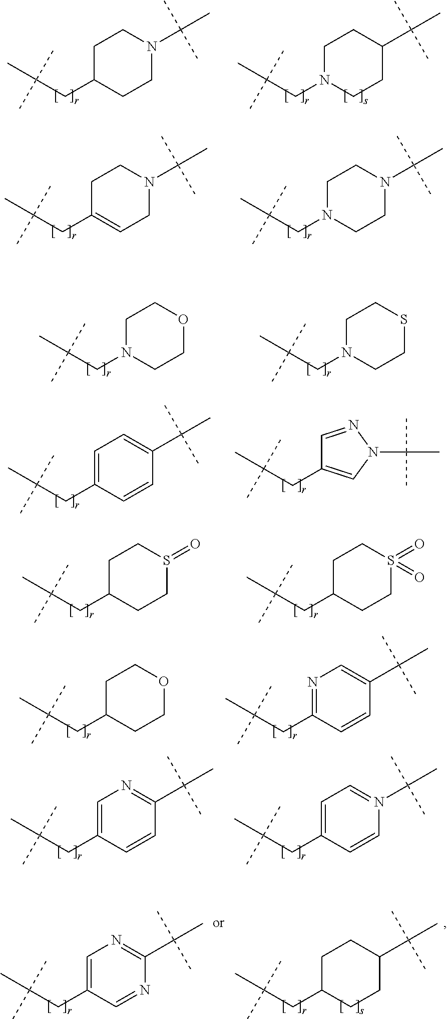

wherein r is 0, 1, or 2; and
wherein s is 0, 1, −1.

In Formula Ib7, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

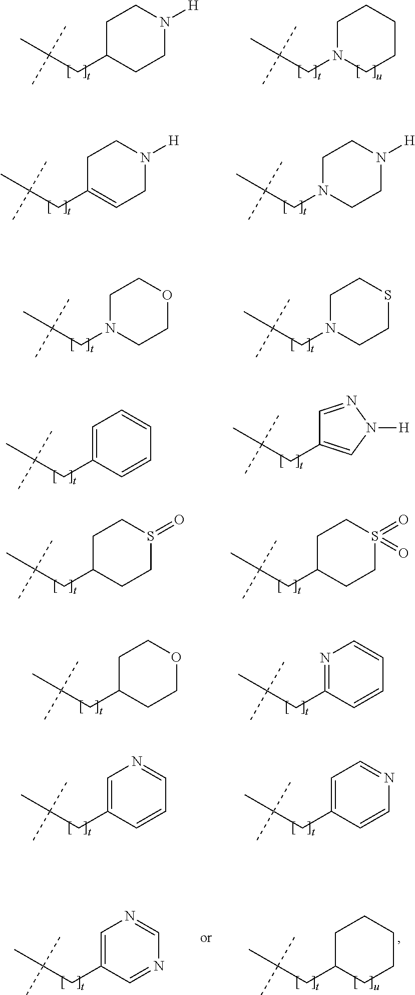

wherein t is 0, 1, or 2;
wherein u is 0, 1, −1.

In some embodiments of Formula Ib7, r is 0.

In some embodiments of Formula Ib7, s is 1.

In some embodiments of Formula Ib7, t is 0.

In some embodiments of Formula Ib7, u is 1.

In some embodiments of Formula Ib7, o is −1.

In some embodiments of Formula Ib7, V is carbon optionally substituted with hydroxyl.

In some embodiments of Formula Ib7, V is oxygen.

In some embodiments of Formula Ib7, p is 1.

In some embodiments of Formula Ib7, R² is methyl, methoxy, ethoxy, Cl, or trifluoromethyl.

In some embodiments, the compounds of Formula Ib are compounds of Formula Ib8:

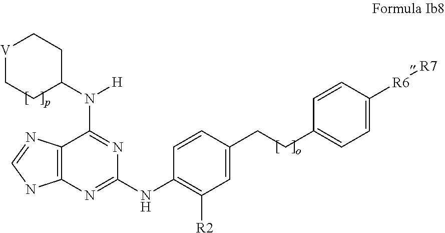

and pharmaceutically acceptable salts and solvates thereof.

In Formula Ib8, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib8, p is 0, 1, or −1.

In Formula Ib8, R² is halo, methyl optionally substituted with halo, ethyl optionally substituted with halo, methylthio, ethylthio, methoxy, or ethoxy.

In Formula Ib8, o is 0, 1, or −1.

In Formula Ib8, q is 0, 1, or −1.

In Formula Ib8, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —C(═O)N(R^{f})R^{f}—, -alkylene-C(═O)N(R^{f})R^{g}—, —S(═O)₂N(R^{f})R^{g}—, -alkylene-S(═O)₂N(R^{f})R^{g}—, —S(═O)₂—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷.

In Formula Ib8, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, or amino-C₁₋₃ alkylene.

In some embodiments of Formula Ib8, V is carbon optionally substituted with hydroxyl.

In some embodiments of Formula Ib8, V is oxygen.

In some embodiments of Formula Ib8, p is 1.

In some embodiments of Formula Ib8, R² is methyl, methoxy, ethoxy, Cl, or trifluoromethyl.

In some embodiments, the compounds of Formula Ib are compounds of Formula Ib9:

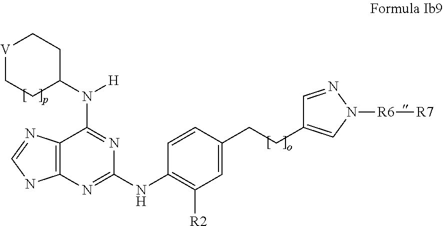

and pharmaceutically acceptable salts and solvates thereof.

In Formula Ib9, V is carbon, oxygen, nitrogen, or sulfur; when V is carbon it is optionally substituted with hydroxyl, —C₁₋₃ alkylene-hydroxyl, or —C₁₋₃ alkylene-amino; when V is nitrogen it is optionally substituted with —S(═O)₂C₁₋₃ alkyl or C₁₋₃ alkyl.

In Formula Ib9, p is 0, 1, or −1.

In Formula Ib9, R² is halo, methyl optionally substituted with halo, ethyl optionally substituted with halo, methylthio, ethylthio, methoxy, or ethoxy.

In Formula Ib9, o is 0, 1, or −1.

In Formula Ib9, R⁶ is C₁₋₃ alkyl, hydroxy, hydroxy-C₁₋₃ alkylene, or halo-C₁₋₃ alkylene.

In Formula Ib9, R⁷ is not present, is hydro, or is —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

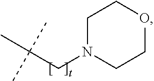

wherein t is 0, 1, or 2.

In some embodiments of Formula Ib9, t is 2.

In some embodiments of Formula Ib9, V is carbon optionally substituted with hydroxyl.

In some embodiments of Formula Ib9, V is oxygen.

In some embodiments of Formula Ib9, p is 1.

In some embodiments of Formula Ib9, R² is methyl, methoxy, ethoxy, Cl, or trifluoromethyl.

The present invention also provides compounds of Formula II

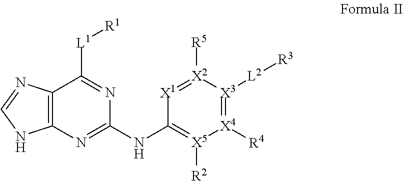

and pharmaceutically acceptable salts and solvates thereof.

In Formula II, R¹ is an optionally substituted carbocycle, heterocycle, aryl, or heteraryl.

In Formula II, R² is chosen from the group consisting of: halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle.

In Formula II, R³ is a group chosen from: hydro, haloalkyl, —R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle.

In Formula II, R⁴ and R⁵ are independently chosen from: hydro, halo (e.g. Cl, Br), hydroxyl, alkyl (e.g., C₁₋₆ alkyl), alkynyl, alkoxy (e.g., methoxy, ethoxy), alkynyloxy, haloalkyl (e.g., trifluoromethyl), haloalkoxy (e.g., trifluoromethoxy), cycloalkyloxy, heterocycle-alkoxy, cycloalkoxy, heterocycloxy, alkoxyalkyl, alkylthio, alkanoyl, amino (e.g., alkylamino), aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl.

In Formula II, X¹ is chosen from N, CH, or is not present.

In Formula II, X², X³, X⁴, and X⁵ are each independently chosen from N and C.

In Formula II, L¹ is direct bond or a linker chosen from: —O—, —S—, —S(═O), —S(═O)₂—, —NR^{a}—, —CH(—R^{a})—, —(CH₂)ₙ—, —N(—R^{a})—(CH₂)ₙ—, —(CH₂), —N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl (e.g., methyl), alkoxyl, carboxyl, or carbocycle.

In Formula II, L² is direct bond or a linker chosen from: —O—, —S—, (C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, —S(═O)₂—, -alkylene-, alkynylene (e.g., ethynylene, 1-propynylene, 2-propynylene), aryl (e.g., phenyl), heteroaryl, heterocycle (e.g., pyrrolidine, piperidine, piperazine, morpholine, and thiomorpholine), arylalkyl, heteroarylalkyl, and heterocyclylalkyl and —O-alkylene-; wherein R^{f} is chosen from hydro and C₁₋₄ alkyl.

In some embodiments of Formula II, when X², X³, or X⁴ are N, then there is no substituent at the N.

In some embodiments of Formula II, R¹ is a substituted or unsubstituted C₃₋₆ (preferably C₅₋₆) cycloalkyl (cyclopropyl, cyclopentyl or cyclohexyl) or heterocycle (e.g., tetrahydropyranyl, thianyl, piperidinyl or morpholinyl).

In some embodiments of Formula II, R¹ is a C₃₋₆ (preferably C₅₋₆) carbocycle (including cycloalkyl, e.g., cyclopropyl, cyclopentyl or cyclohexyl) or heterocycle (e.g., tetrahydropyranyl, thianyl, piperidinyl or morpholinyl) optionally substituted with one or more (e.g., 1, 2, 3 or 4) substituents (preferably at meta- and/or para-position relative to L₁) independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

In some embodiments of Formula II, R¹ is chosen from the group consisting of:

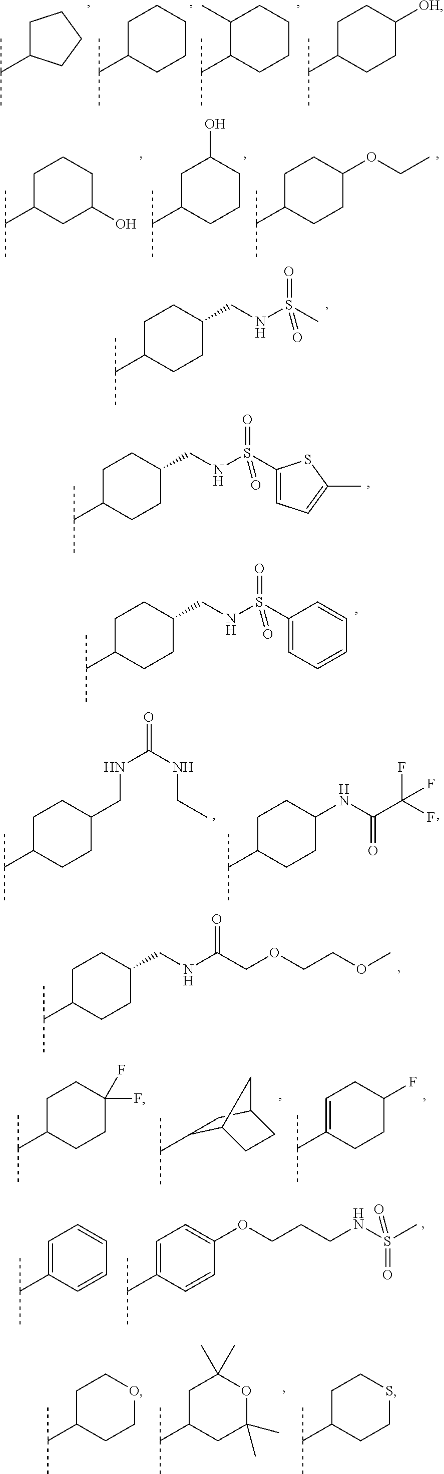

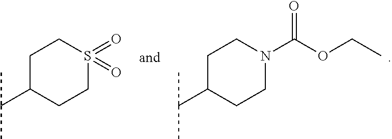

In Formula II, R² is halo, methyl optionally substituted with halo, ethyl optionally substituted with halo, methylthio, ethylthio, methoxy, or ethoxy.

In some embodiments of Formula II, R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino. In some embodiments R³ is hydro only when R⁴ or R⁵ is not hydro.

In some embodiments of Formula II, R³ is chosen from the group consisting of: methyl, methylene, trifluoromethyl, ethyl, ethylene, propyl, propylene, pentyl, pentylene,

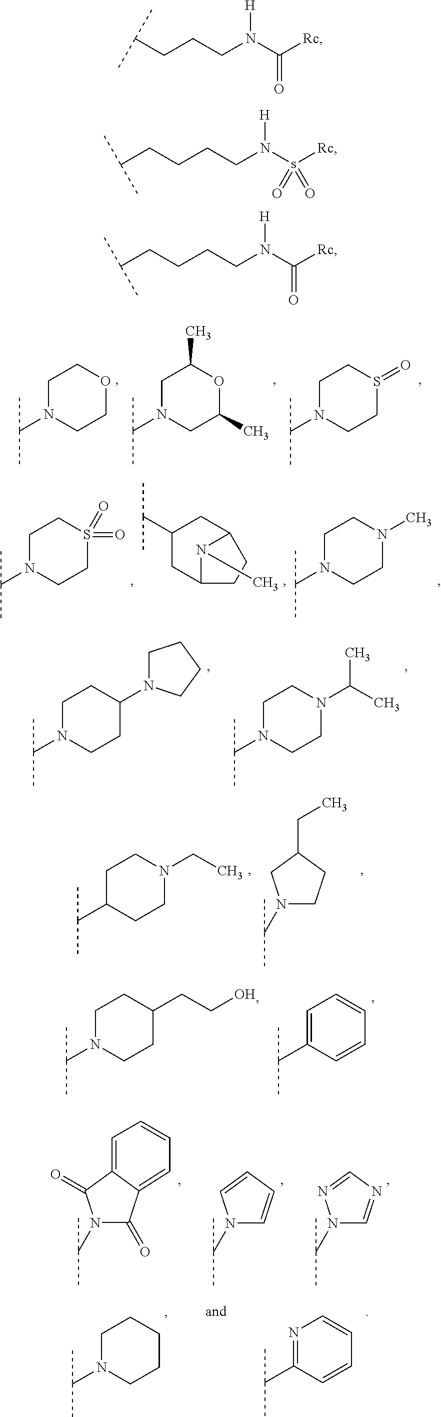

In some embodiments of Formula II, R^{c} is chosen from the group consisting of: C₁₋₆ alkyl (e.g., ethyl, isopropyl), C₁₋₆ alkoxy, C₃₋₆ cycloalkyl (e.g., cyclopropyl), benzyl, morpholino, pyrrolidinyl, piperidinyl, piperazinyl, bicyclic heterocycle, imidazole, pyrrole, pyridine, and triazole.

In some embodiments of Formula II, R^{c} is chosen from the group consisting of:

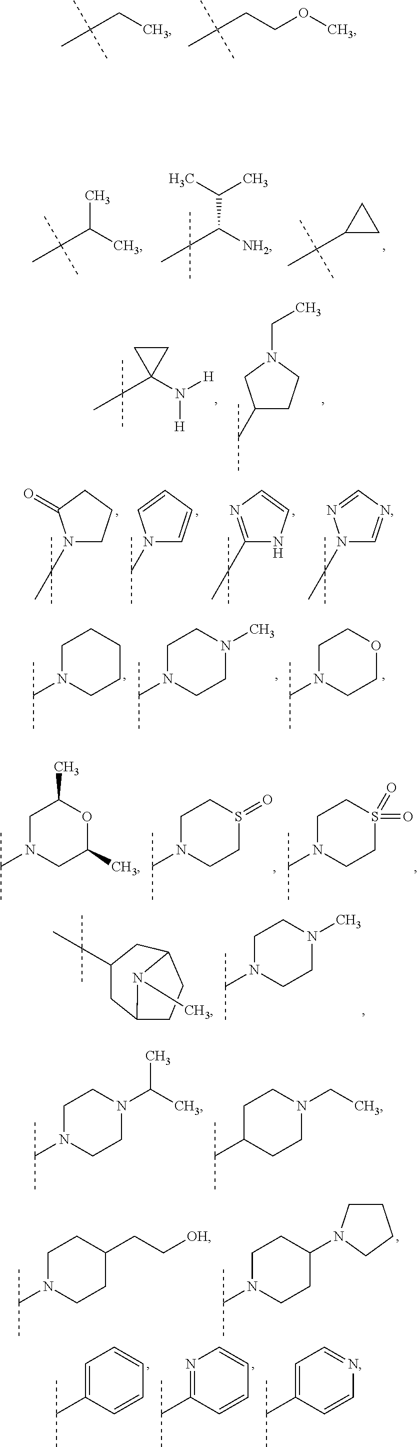

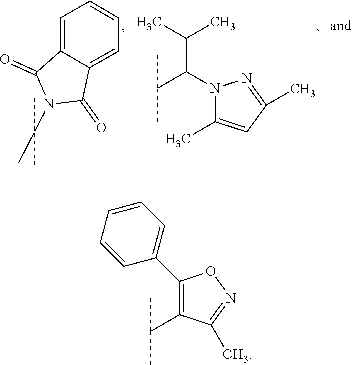

In some embodiments of Formula II, R³ is —R⁶—R⁷, wherein R⁶ is: C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

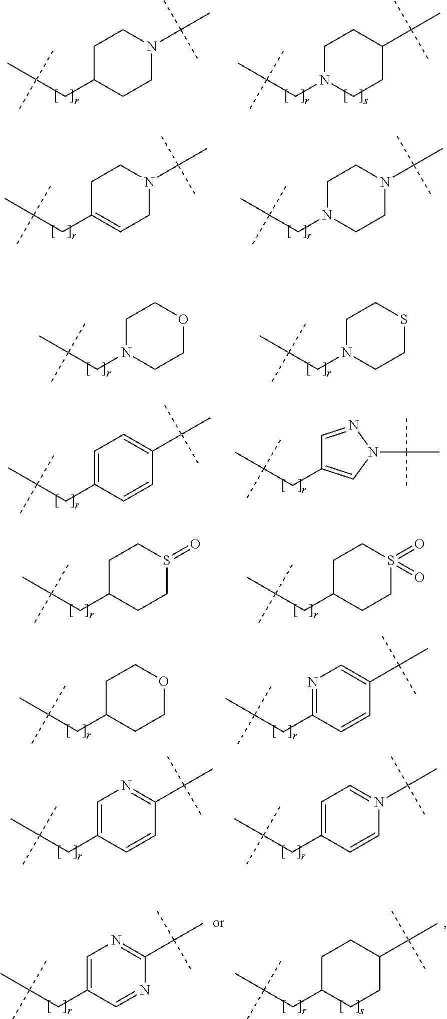

wherein r is 0, 1, or 2; and
wherein s is 0, 1, −1; and,

wherein R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, -alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

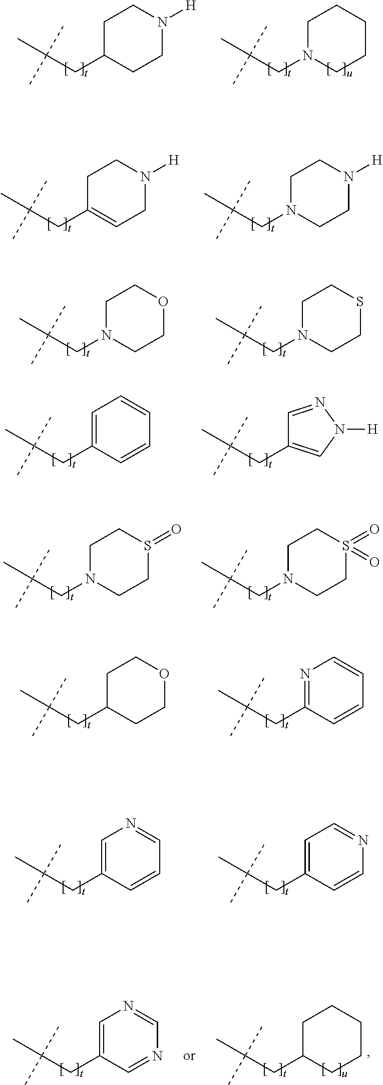

wherein t is 0, 1, or 2;
wherein u is 0, 1, −1.

In some embodiments of Formula II, r is 0.

In some embodiments of Formula II, s is 1.

In some embodiments of Formula II, t is 0.

In some embodiments of Formula II, u is 1.

In some embodiments of Formula II, R⁶ is C₁₋₃ alkyl, hydroxy, hydroxy-C₁₋₃ alkylene, halo-C₁₋₃ alkylene, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

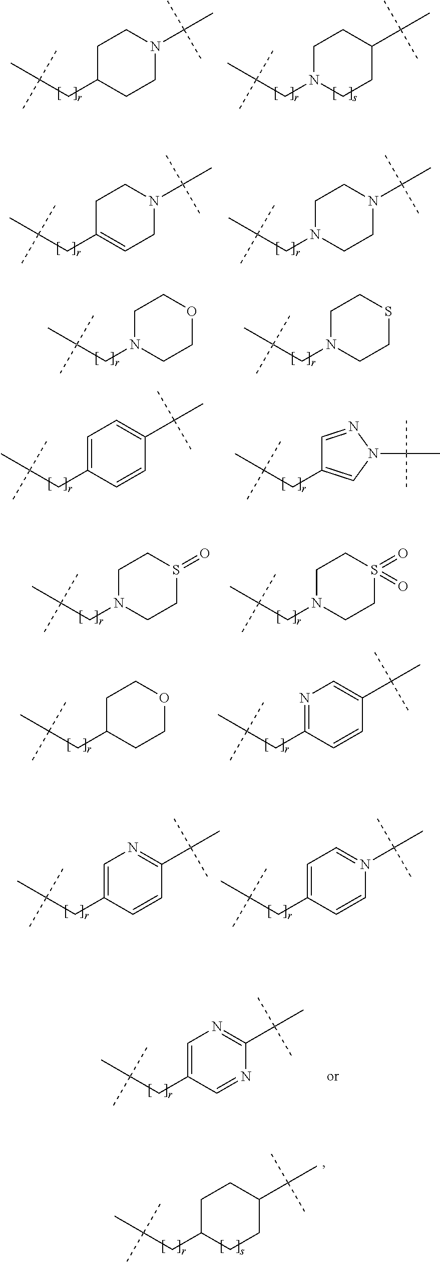

wherein r is 0, 1, or 2; and
wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or

R⁷ is:

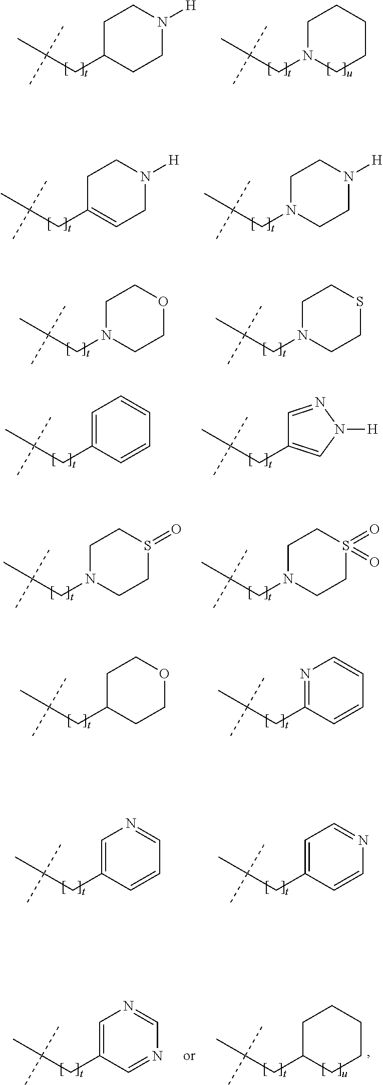

wherein t is 0, 1, or 2; and
wherein u is 0, 1, −1.

In some embodiments of Formula II, R⁶ is C₁₋₃ alkyl, hydroxy, hydroxy-C₁₋₃ alkylene, halo-C₁₋₃ alkylene, —C₁₋₃ alkylene-C(═O)—, —N(R^{f})C(═O)—, -alkylene-N(R^{f})C(═O)—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from:

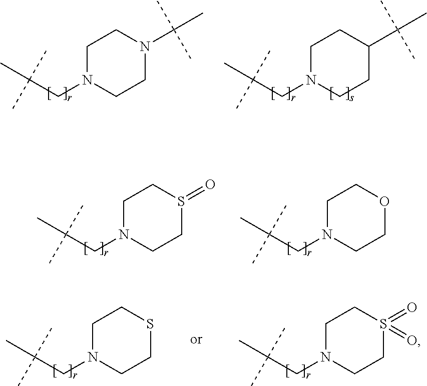

wherein r is 0, 1, or 2; and
wherein s is 0, 1, −1. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is:

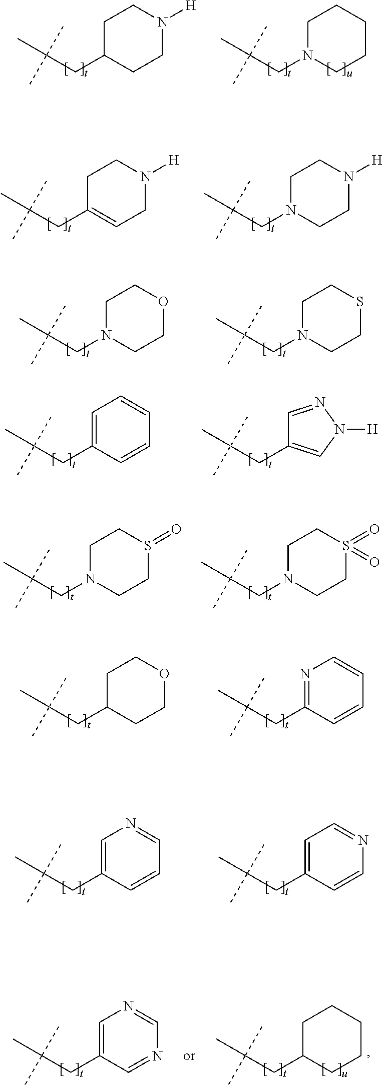

wherein t is 0, 1, or 2; and
wherein u is 0, 1, −1.

In some embodiments of Formula II, R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —C(═O)N(R^{f})R^{f}—, -alkylene-C(═O)N(R^{f})R^{g}—, —S(═O)₂N(R^{f})R^{g}—, -alkylene-S(═O)₂N(R^{f})R^{g}—, —S(═O)₂—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷. In some of these embodiments, R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, or amino-C₁₋₃ alkylene.

In some embodiments of Formula II, R⁶ is C₁₋₃ alkyl, hydroxy, hydroxy-C₁₋₃ alkylene, or halo-C₁₋₃ alkylene. In some embodiments of these embodiments, R⁷ is not present, is hydro, or is —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is:

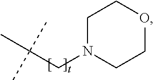

wherein t is 0, 1, or 2. In some of these embodiments of Formula Ib, t is 2.

In some embodiments of Formula II, R² and R⁴, together with the carbon atoms to which they are attached, form the following ring structure:

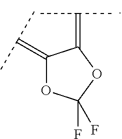

In some embodiments of Formula II, L¹ is direct bond or a linker chosen from: —O—, —S—, —S(═O)—, —S(═O)₂—, —N(R^{a})—, —CH(R^{a})—, —(CH₂)ₙ— wherein n is 1, 2 or 3, —C(═O)—, —C(═O)N(R^{a})—, wherein R^{a} is hydro or C₁₋₆ alkyl (e.g., methyl).

In some embodiments of Formula II, L¹ is —N(H)—.

In some embodiments of Formula II, L¹ is direct bond, —N(R^{a})— wherein R^{a} is hydro or C₁₋₃ alkyl (e.g., methyl or ethyl), —(CH₂)ₙ— wherein n is 1, 2 or 3, or —C(═O)—.

In some embodiments of Formula II, L² is direct bond, or a linker chosen from: —O—, —O-alkylene-, —C(═O)—, —C(═O)N(R^{a})— wherein R^{a} is hydro or C₁₋₃ alkyl (e.g., methyl or ethyl), alkylene, alkynylene.

In some embodiments of Formula II, L² is direct bond, or a linker chosen from: —O—, —O—(CH₂)ₙ— wherein n is 1, 2 or 3, —C(═O)—, —C(═O)N(R^{a})— wherein R^{a} is hydro or C₁₋₃ alkyl (e.g., methyl or ethyl), —(CH₂)ₙ— wherein n is 1, 2 or 3, —(CH₂)ₚ—C≡C—(CH₂)_{q}— wherein p and q are each independently 0, 1, 2 or 3.

In some embodiments of Formula II, L² is alkynylene, aryl, arylalkyl, heteraryl, heteroarylalkyl, or

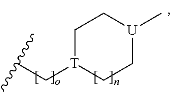

wherein T is carbon or nitrogen, U is carbon, nitrogen, sulfur, or oxygen, n is 0, 1, or −1, o is 0, 1, or 2, and there is optionally at least one ring carbon-ring carbon double bond.

In some embodiments of Formula II, L² is alkynylene or

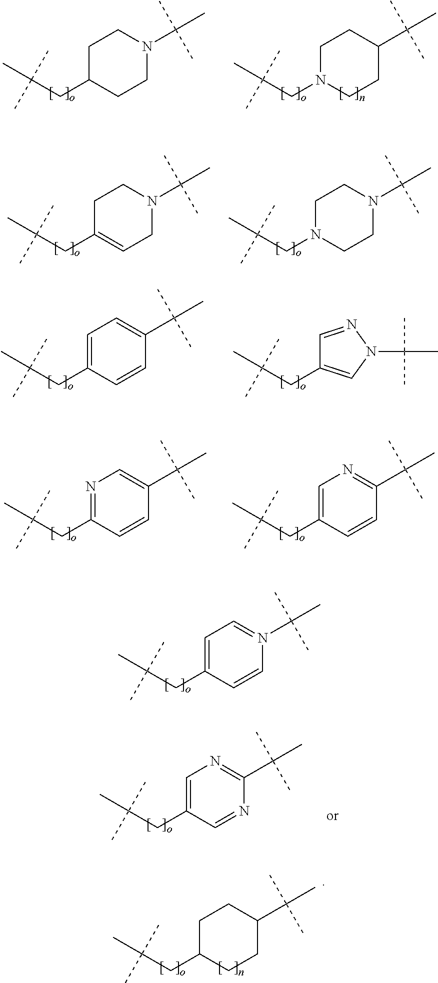

In some embodiments of Formula II, L¹ is —N(H)R¹ is cyclohexyl; R² is halo, methyl optionally substituted with halo, ethyl optionally substituted with halo, methylthio, ethylthio, methoxy, or ethoxy; R⁴ and R⁵ are not present or are Hydrogen; L² is alkylene, alkynylene, carbonyl, or:

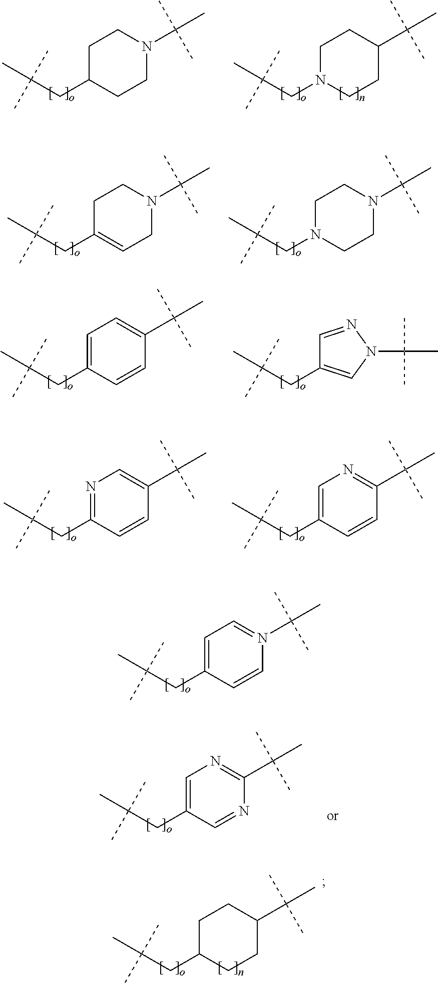

and R³ is —R⁶—R⁷, wherein R⁶ is —S(═O)₂—C₁₋₃ alkyl or is one of:

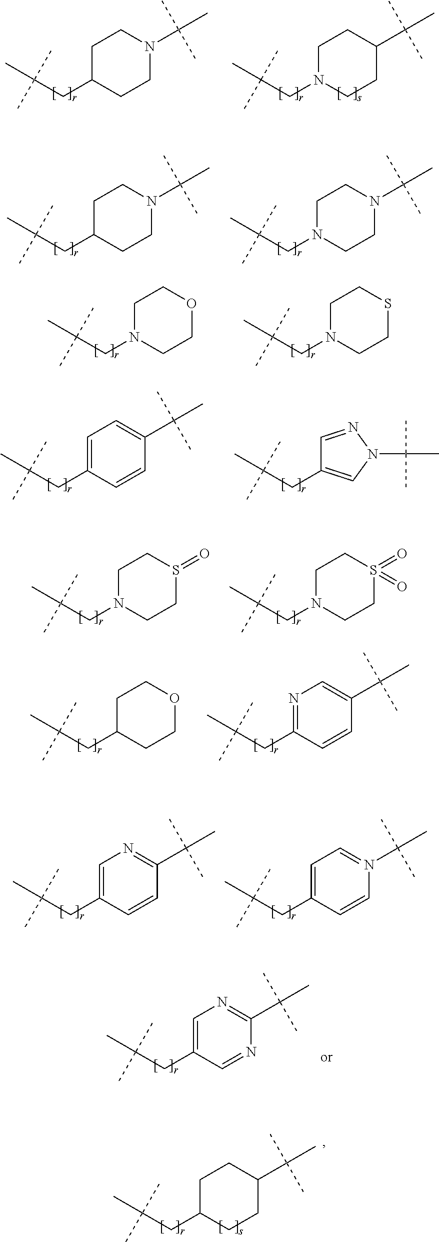

wherein r is 0 or 1 and s is 1; and, wherein R⁷ is not present, or is hydro.

In preferred embodiments, compounds are provided according to the above Formula I having an IC₅₀ of less than about 2.5 μM, 500 nM, 300 nM, or 200 nM, preferably less than about 100 nM, and most preferably less than about 80 nM, as determined in the HCT116 assay in Example 2.

A pharmaceutically acceptable salt of the compound of the present invention is exemplified by a salt with an inorganic acid and/or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

Additionally, the compounds of the present invention can contain asymmetric carbon atoms and can therefore exist in racemic and optically active forms. Thus, optical isomers or enantiomers, racemates, and diastereomers are also encompassed, so long as the stereochemistry of the core structure of the compounds is equivalent to that of Formula I. The methods of the present invention include the use of all such isomers and mixtures thereof. The present invention encompasses any isolated racemic or optically active form of compounds described above, or any mixture thereof, which possesses anti-cancer activity.

Unless specifically stated otherwise or indicated by direct bond symbol (dash or double dash), the connecting point to a recited group will be on the right-most stated group. Thus, for example, a hydroxyalkyl group is connected to the main structure through the alkyl and the hydroxyl is a substituent on the alkyl.

### 2. Definitions

The term “bioisostere”, as used herein, generally refers to compounds or moieties that have chemical and physical properties producing broadly similar biological properties. Examples of carboxylic acid bioisosteres include, but are not limited to, carboxyalkyl, carboxylic acid ester, tetrazole, oxadiazole, isoxazole, hydroxythiadiazole, thiazolidinedione, oxazolidinedione, sulfonamide, aminosulfonyl, sulfonamidecarbonyl, C-amido, sulfonylcarboxamide, phosphonic acid, phosphonamide, phosphinic acid, sulfonic acid, alkanoylaminosulfonyl, mercaptoazole, trifluoromethylcarbonyl, and cyanamide.

In the definitions below, ranges of carbon atoms are meant to imply all possible intergers inclusive in the range, including, for example, 1 carbon, 2 carbons, 3 carbons, and 4 carbons when a range such as C₁₋₄, C₁-C₄, or C₁ to C₄, is specified.

The term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1, 2 or 3 carbon atoms, or up to 20 carbon atoms). An alkyl group may be in unsubstituted form or substituted form with one or more substituents (generally one to three substitutents except in the case of halogen substituents, e.g., perchloro). For example, a C₁₋₆ alkyl group refers to a straight or branched aliphatic group containing 1, 2, 3, 4, 5, or 6 carbon atoms (e.g., including methyl, ethyl, propyl, isopropyl, butyl, sec-butyl, tert-butyl, 3-pentyl, hexyl, etc.), which may be optionally substituted. In most embodiments of the present invention, alkyl groups have between 1 and 6 carbons, unless otherwise specified. In such embodiments the carbons may optionally be substituted.

The term “alkylene” as used herein means a saturated aliphatic hydrocarbon straight chain or branched chain group having 1 to 20 carbon atoms having two connecting points. For example, “ethylene” represents the group —CH₂—CH₂— or —CH₂(CH₃)—. Alkylene groups may also be in unsubstituted form or substituted form with one or more substituents.

The term “alkenyl” as employed herein by itself or as part of another group means a straight or branched chain radical of 2 to 10 carbon atoms, unless the chain length is limited thereto, including at least one double bond between two of the carbon atoms in the chain. The alkenyl group may be in unsubstituted form or substituted form with one or more substituents (generally one to three substitutents except in the case of halogen substituents, e.g., perchloro or perfluoroalkyls). For example, a C₁₋₆ alkenyl group refers to a straight or branched chain radical containing 1 to 6 carbon atoms and having at least one double bond between two of the carbon atoms in the chain (e.g., ethenyl, 1-propenyl, 2-propenyl, 2-methyl-1-propenyl, 1-butenyl and 2-butenyl), which may be optionally substituted. The term “alkenylene” as used herein means an alkenyl group having two connecting points. For example, “ethenylene” represents the group —CH═CH— or —(C═CH₂)—. Alkenylene groups may also be in unsubstituted form or substituted form with one or more substituents.

The term “alkynyl” as used herein by itself or as part of another group means a straight or branched chain radical of 2 to 10 carbon atoms, unless the chain length is specifically limited, wherein there is at least one triple bond between two of the carbon atoms in the chain. The alkynyl group may be in unsubstituted form or substituted form with one or more substituents (generally one to three substitutents except in the case of halogen substituents, e.g., perchloro or perfluoroalkyls). For example, a C₁₋₆ alkynyl group refers to a straight or branched chain radical containing 1 to 6 carbon atoms and having at least one triple bond between two of the carbon atoms in the chain (e.g., ethynyl, 1-propynyl, 1-methyl-2-propynyl, 2-propynyl, 1-butynyl and 2-butynyl), which may be optionally substituted.

The term “alkynylene” as used herein means an alkynyl having two connecting points. For example, “ethynylene” represents the group —C≡C—. Alkynylene groups may also be in unsubstituted form or substituted form with one or more substituents.

The term “carbocycle” as used herein by itself or as part of another group means cycloalkyl and non-aromatic partially saturated carbocyclic groups such as cycloalkenyl and cycloalkynyl. A carbocycle may be in unsubstituted form or substituted form with one or more substituents so long as the resulting compound is sufficiently stable and suitable for the treatment method of the present invention.

The term “cycloalkyl” as used herein by itself or as part of another group refers to a fully saturated 3- to 8-membered (i.e., 3, 4, 5, 6, 7, or 8-membered) cyclic hydrocarbon ring (i.e., a cyclic form of an unsubstituted alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, or a spiral ring. When a cycloalkyl is referred to as a C_{X} cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl group may be unsubstituted or substituted with one or more substitutents so long as the resulting compound is sufficiently stable and suitable for the treatment method of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered (i.e., 3, 4, 5, 6, 7, or 8-membered) cyclic hydrocarbon ring having a double bond therein (i.e., a cyclic form of an unsubstituted alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is referred to as a C_{X} cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group may be in unsubstituted form or substituted form with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3 to 7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of carbon atoms with such other rings) (“polycyclic heterocycle”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be in unsubstituted form or substituted form with one or more substituents so long as the resulting compound is sufficiently stable and suitable for the treatment method of the present invention. Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In specific embodiments, aryl rings include 4, 5, 6, or 7 carbons. In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a C_{X} aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be in unsubstituted form or substituted form with one or more substituents so long as the resulting compound is sufficiently stable and suitable for the treatment method of the present invention.

The term “heteroaryl” as employed herein refers to a stable aromatic ring having up to 7 ring atoms (i.e., 3, 4, 5, 6, or 7 atoms) with 1, 2, 3 or 4 hetero ring atoms in the ring which are oxygen, nitrogen or sulfur or a combination thereof (“monocylic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be in unsubstituted form or substituted form with one or more substituents so long as the resulting compound is sufficiently stable and suitable for the treatment method of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-c]pyrimidin-4-one, pyrazolo[1,5-c]pyrimidinyl, including without limitation pyrazolo[1,5-c]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, or iodo.

As used herein, the term “hydro” refers to a bound hydrogen atom (—H group).

As used herein, the term “hydroxyl” refers to an —OH group.

As used herein, the term “alkoxy” refers to an —O—(C₁₋₁₂ alkyl). Lower alkoxy refers to —O—(lower [i.e., C₁-C₄] alkyl) groups.

As used herein, the term “alkynyloxy” refers to an —O—(C₁₋₁₂ alkynyl).

As used herein, the term “cycloalkyloxy” refers to an —O-cycloakyl group.

As used herein, the term “heterocycloxy” refers to an —O-heterocycle group.

As used herein, the term “aryloxy” refers to an —O-aryl group.

The term “heteroaryloxy” refers to an —O-heteroaryl group.

The terms “arylalkoxy” and “heteroarylalkoxy” are used herein to mean an alkoxy group substituted with an aryl group and a heteroaryl group, respectively.

As used herein, the term “mercapto” group refers to an —SH group.

The term “alkylthio” group refers to an —S-alkyl group.

The term “arylthio” group refers to an —S-aryl group.

The term “arylalkyl” is used herein to mean an alkyl group substituted with an aryl group. Examples of arylalkyl include benzyl, phenethyl or naphthylmethyl.

The term “heteroarylalkyl” is used herein to mean an alkyl group substituted with a heteroaryl group.

The term “arylalkenyl” is used herein to mean an alkenyl group substituted with an aryl group.

“Heteroarylalkenyl” means an alkenyl group substituted with a heteroaryl group.

“Arylalkynyl” means an alkynyl having a substituent that is an aryl group.

The term “heteroarylalkynyl” is used herein to mean an alkynyl group substituted with a heteroaryl group.

“Haloalkyl” means an alkyl group that is substituted with one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

As used herein, the term “carbonyl” group refers to a —C(═O)— group.

The term “thiocarbonyl” group refers to a —C(═S)— group.

“Alkanoyl” refers to an alkyl-C(═O)— group.

The term “acetyl” group refers to a —C(═O)CH₃ group.

“Alkylthiocamonyl” refers to an alkyl-C(═S)— group.

The term “cycloketone” refers to a carbocycle or heterocycle group in which one of the carbon atoms which form the ring has an oxygen double-bonded to it—i.e., one of the ring carbon atoms is a —C(═O)— group.

The term “O-carboxy” group refers to a R″C(═O)O— group, where R″ is as defined herein below.

The term “C-carboxy” group refers to a —C(═O)OR″ groups where R″ is as defined herein below.

The term “carboxylic acid” refers to —COOH.

The term “ester” is a C-carboxy group, as defined herein, wherein R″ is any of the listed groups other than hydro.

The term “C-carboxy salt” refers to a —C(═O)O⁻M⁺ group wherein M⁺ is selected from the group consisting of lithium, sodium, magnesium, calcium, potassium, barium, iron, zinc, copper, and ammonium.

The term “carboxyalkyl” refers to —C₁₋₆ alkylene-C(═O)OR″ (that is, a C₁₋₆ alkyl group connected to the main structure wherein the alkyl group is substituted with —C(═O)OR″ with R″ being defined herein below). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein below.

The term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

The term “carboxyalkoxy” refers to —O—(CH₂)ᵣC(═O)OR″ wherein r is 1-6, and R″ is as defined herein below.

“Cₓ carboxyalkanoyl” means a carbonyl group (—(O═)C—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—(O═)C—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein below.

“Amino” refers to an —NR^{x}R^{y} group, with R^{x} and R^{y} as defined herein.

“Alkylamino” means an amino group with a substituent being a C₁₋₆ alkyl.

“Aminoalkyl” means an alkyl group connected to the main structure of a molecule where the alkyl group has a substituent being amino.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

The term “O-carbamyl” refers to a —OC(═O)N(R^{x})(R^{y}) group with R^{x} and R^{y} as defined herein.

The term “N-carbamyl” refers to a R^{y}OC(═O)N(R^{x})— group, with R^{x} and R^{y} as defined herein.

The term “O-thiocarbamyl” refers to a —OC(═S)N(R^{x})(R^{y}) group with R^{x} and R^{y} as defined herein.

The term “N-thiocarbamyl” refers to a R^{X}OC(═S)NR^{y}— group, with R^{x} and R^{y} as defined herein.

“C-amido” refers to a —C(═O)N(R^{x})(R^{y}) group with R^{x} and R^{y} as defined herein.

“N-amido” refers to a R^{x}C(═O)N(R^{y})— group with R^{x} and R^{y} as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The terms “cyano” and “cyanyl” refer to a —C≡N group.

The term “cyanato” refers to a —CNO group.

The term “isocyanato” refers to a —NCO group.

The term “thiocyanato” refers to a —CNS group.

The term “isothiocyanato” refers to a —NCS group.

The term “sulfinyl” refers to a —S(═O)R″ group, where R″ is as defined herein below.

The term “sulfonyl” refers to a —S(═O)₂R″ group, where R″ is as defined herein below.

The term “sulfonamide” refers to a —(R^{x})N—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂— group with R^{x} and R^{y} as defined herein.

“Sulfonamidecarbonyl” means R″—S(═O)₂—N(R^{x})—C(═O)— with R″ and R^{x} as defined herein below.

“Alkanoylaminosulfonyl” refers to an alkyl-C(═O)—N(R^{x})—S(═O)₂— group with R^{x} as defined herein below.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is selected from the group consisting of hydro, alkyl, cycloalkyl, aryl, heteroaryl and heterocycle, each being optionally substituted.

R^{x}, R^{y}, and R^{z} are independently selected from the group consisting of hydro and optionally substituted alkyl.

The term “methylenedioxy” refers to a —OCH2O— group wherein the oxygen atoms are bonded to adjacent ring carbon atoms.

The term “ethylenedioxy” refers to a —OCH2CH2O— group wherein the oxygen atoms are bonded to adjacent ring carbon atoms.

The present invention provides methods for treating cancer, by treating a patient (either a human or another animal) in need of the treatment, with a compound of the present invention.

As used herein, the phrase “treating . . . with . . . a compound” means either administering the compound to cells or an animal, or causing the presence or formation of the compound inside the cells or the animal. Preferably, the methods of the present invention comprise administering to cells in vitro or to a warm-blood animal, particularly mammal, more particularly a human, a pharmaceutical composition comprising an effective amount of a compound according to the present invention.

### 3. Selectivity

Given their role in the cell-cycle, many kinases have become targets for anti-cancer treatments. Promising potential cancer targets include serine-threonine protein kinases such as the Aurora proteins (mostly Aurora A and B) or Polo-like kinases (PLK1), which are the subject of intense investigation. de Cárcer et al., Targeting cell cycle kinases for cancer therapy, Curr. Med. Chem. 14:969-985 (2007). One less studied mitotic kinase is TTK (also known as MPS1), whose role in mitotic progression and the spindle checkpoint suggests it might be a new target of interest in cancer therapy. Id. Although targeting cell cycle kinases is generally an efficient way to arrest aberrant cell proliferation, there is a need to find compounds that inhibit kinases such as TTK and thereby specifically kill cancer cells.

It has been discovered that compounds of the present invention are selectively active against the dual specificity protein kinase TTK (encoded by the TTK gene, i.e., GeneID No. 7272; see Example 3) while showing little or no activity against Aurora kinase (e.g., inhibiting TTK with an IC₅₀ at least 1000-fold lower than the IC₅₀ for Aurora kinase A inhibition, and at least 500-fold lower than the IC₅₀ for Aurora kinase B inhibition). Compounds of the invention show further promise by killing cancer cells (see Example 2) and tumors. This selectivity is further shown by the activity of the compounds in the G₂/M escape assay, which is selective for TTK inhibition by virtue of TTK's role in G₂/M escape (see Example 4). The selectivity of the compounds of the present invention for TTK over Aurora kinases may provide anti-cancer benefits while avoiding the side-effects, drawbacks and/or limitations of Aurora kinase inhibitors. For example, Aurora kinase inhibitors are known to cause polyploidy in cells while compounds of the invention do not (see Example 5).

In some embodiments, it is believed that compounds according to Formulas Ia2 and Ib1-Ib9 are selectively active against the protein kinase TTK while showing little or no activity against Aurora kinase. Without wishing to be bound by theory, at least a partial explanation for the selectivity of these compounds is the discovery that a group other than Hydrogen at the R² position (such as, for example, methyl, ethyl, methoxy, ethoxy, halo, and trifluoromethyl) results in selectivity for TTK kinase over Aurora A and B kinase (see Example 7).

Furthermore, without wishing to be bound by theory, it is believed that there are two possible binding modes for compounds according to Formulas Ia2 and Ib1-Ib9 with TTK. Molecular docking to X-ray crystal structures of TTK (Protein Data Bank ID 3GFW and 3H9F) revealed two possible binding modes of R². Formula Ib1 is shown below in two different binding modes. It should be understood that the two different representations of Formula Ib1 do not involve stereochemical differences, but are the result of free rotation around a single bond. It should also be understood that the discussion below regarding Formula Ib1 applies equally to Formulas Ia2 and 1b2-1b9.

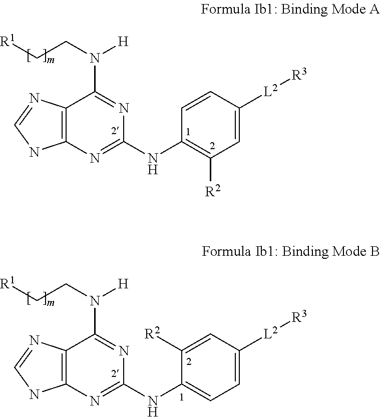

In binding mode A, the R² group is syn-coplanar to amide NH and directed towards kinase hinge loop of TTK. In binding mode B, the R² group is anti-coplanar to amide NH and directed into ribose binding pocket of TTK.

It is believed that binding mode A is favorable. The R² group makes van der Waals contacts with side chain of hinge residue Cys604 (residue i+2, where i is Gatekeeper residue) and with backbone of hinge residues Asn606 and Ile607 (residues i+4 and i+5). In Aurora A, respective positions of hinge loop are occupied by residues Tyr211, Pro213 and Leu214. Side chain of Tyr211, which is significantly larger than Cys605 in TTK, causes unfavorable contacts with the R² group in binding mode A. In addition, presence of proline in position i+4 significantly changes backbone conformation of hinge residues i+4 and i+5 in Aurora A. As a result, ATP binding site in Aurora A does not contain sufficient cavity in hinge loop area to accommodate any heavy (non-hydrogen) atom attached at the R² group. Thus, at least partially, explaining selectivity for TTK over Aurora A.

In binding mode B, the R² group makes van der Waals contacts with the side chain of Leu654 located in ribose binding pocket. Respective residue in Aurora, Leu262, assumes side chain conformation different from that of Leu654 in TTK because of van der Waals overlap with C_{β} methyl of hinge i+3 residue Ala212 (Gly605 in TTK). A C_{δ}-methyl group of Leu262 in Aurora A enters a cavity available in TTK for the R² group, which results in an unfavorable binding mode for compounds according to Formula Ia2 and Ib1-Ib9 in the ATP binding site of Aurora A. Thus, at least partially, explaining selectivity for TTK over Aurora A.

In general, compounds according to Formula Ia2 and Ib1-Ib9 are expected to show significant selectivity for TTK against kinases containing large side chain in the hinge loop position i+3 (Tyr, Phe), and moderately selective against kinases containing medium-size side chains (Leu, Ile, Met) in that position.

The R² group and amide NH assume syn-coplanar or anti-coplanar conformation with the torsion angle between C2-C1-NH-C2′ equal to 180±45° or 0±45°, respectively.

### 4. Therapeutic Methods

As used herein, the term “neoplastic” has its conventional meaning in the art. As used herein, neoplastic disease encompasses cancer. As used herein, the term “cancer” has its conventional meaning in the art. Cancer includes any condition of the animal or human body characterized by abnormal cellular proliferation. Compounds of the invention have been shown to be effective in standard cancer models, including an HCT116 colon cancer cell line cytotoxicity assay and mouse xenograft studies. Due to the fundamental role of TTK in cell-cycle progression, compounds of the invention should be active against most types of cancer. Thus, treating cancer will encompass the treatment of a person who has any type of cancer. That is, “treating cancer” should be understood as treating a patient who is at any one of the several stages of cancer, including diagnosed but as yet asymptomatic cancer.

A patient having cancer can be identified by conventional diagnostic techniques known in the art, and the identified patient can be treated with a compound of the present invention, preferably in a pharmaceutical composition having a pharmaceutically acceptable carrier.

In one aspect, the present invention provides methods for combination therapy for treating cancer by treating a patient (either a human or another animal) in need of the treatment with a compound of the present invention together with one or more other anti-cancer therapies. Such other anti-cancer therapies include traditional chemotherapy agents, targeted agents, radiation therapy, surgery, hormone therapy, etc. In the combination therapy, the compound of the present invention can be administered separately from, or together with the one or more other anti-cancer therapies.

### 5. Pharmaceutical Compositions and Formulations

In another aspect, the present invention further provides a medicament or a pharmaceutical composition having a therapeutically or prophylactically effective amount of a compound or a pharmaceutically acceptable salt thereof according to the present invention.

The active ingredient may be administered at once, or may be divided into a number of smaller doses to be administered at predetermined intervals of time. The suitable dosage unit for each administration can be determined based on the effective daily amount and the pharmacokinetics of the compounds. In the case of combination therapy, a therapeutically effective amount of one or more other anti-cancer compounds can be administered in a separate pharmaceutical composition, or alternatively included in the pharmaceutical composition according to the present invention which contains a compound according to the present invention. The pharmacology and toxicology of many of such other anti-cancer compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage range set forth above is exemplary only and is not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

The active compounds can also be administered parenterally in the form of solution or suspension, which can be prepared from a lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. See, e.g., Phillips et al., J. Pharmaceut. Sci., 73:1718-1720 (1984).

The active compounds can also be incorporated into a prodrug, e.g., conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Another typical prodrug form is an ester of the parent compound, as is generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated, so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

### 6. Methods of Determining Therapeutic Efficacy of Compounds

The present invention also generally relates to novel methods of determining the therapeutic efficacy of the compounds of the present invention, and other TTK-inhibiting compounds.

By way of background, it has been discovered that inhibition of TTK causes defects in chromosome alignment, resulting in lagging chromosomes during anaphase, micronuclei formation, aneuploidy, and/or tetraploidy. Furthermore, anaphase bridges may form, resulting in double-stranded DNA breaks. Additionally, inhibitors of TTK are known to abrogate the spindle assembly checkpoint during an unperturbed mitosis.

This aspect of the invention is based upon the unexpected discovery that inhibition of TTK leads to the stabilization and transcriptional activation of p53. p53 is important in multicellular organisms, where it regulates the cell cycle and thus functions as a tumor suppressor that is involved in preventing cancer. Importantly, p53 is a transcription factor that is activated in response to genotoxic stress including DNA double strand breaks, and in response to tetraploidy. p53 transcriptionally induces the expression of proteins involved in DNA repair, cell cycle arrest and/or apoptosis. For example, the cyclin-dependent kinase inhibitor p21 (also known as CDKN1A) is upregulated by p53, whereas the anti-apoptotic protein survivin is transcriptionally repressed by p53.

This discovery that p53 is stabilized and transcriptionally activated in response to TTK inhibition indicates that p53 has the potential to be used as a biomarker to monitor the effects of TTK inhibition in both cells in culture and animal models, and potentially in human cancers and human patients. One advantage to this biomarker “readout” for TTK activity is that it is a positive signal which is observed as an increase in signal intensity above background. Since TTK is active primarily in mitotic cells and mitotic cells represent only 5-10% of cells in an asynchronous cell population, having a positive readout can greatly enhance the ability to monitor TTK-inhibitory activity.

The observed p53 activation following TTK inhibition likely results from chromosome segregation defects, which may cause subsequent DNA damage and/or tetraploidy. However, it has been discovered that TTK inhibitor-induced cell death is not dependent upon p53 or caspase activity. Inhibitors of TTK-induced death in cells with either wild-type or mutant p53, indicating that p53 is not required for cell death. Additionally, it has been discovered that TTK inhibitor-induced phosphorylation of p53 is ATR-dependent but caspase-independent.

Further, it is known that TTK can phosphorylate proteins on serine, threonine, and tyrosine residues. However, the number of known TTK-protein substrates is limited. Identification of TTK-protein substrates would be valuable to drug development studies. TTK phosphorylation of its protein substrates could provide a biomarker for examination of enzyme activity in both cell-based assays as well as animal and human studies. It was discovered that TTK phosphorylates Hsp90. Hsp90 has several important cellular functions including the protein's chaperoning and trafficking activities.

In view of these unexpected discoveries, there are at least the following embodiments of the present invention:

A first embodiment of this aspect of the invention provides a method of monitoring TTK inhibition by the compounds of the present invention, or any other TTK inhibiting compounds, comprising determining a level of p53 activation in a first biological sample contacted with the TTK inhibitor and comparing said level of p53 activation with a baseline level of p53 activation from a second biological sample not contacted with said TTK inhibitor. If the level of p53 activation is greater in the first biological sample than the baseline level of p53 activation in the second biological sample, then TTK has been at least partially inhibited.

A second embodiment of this aspect of the present invention provides a method of monitoring TTK inhibition by the compounds of the present invention, or any other TTK inhibiting compounds, comprising determining a level of ATR activation in a first biological sample that has been contacted with the TTK inhibitor and comparing said level of ATR activation with a baseline level of ATR activation from a second biological sample that has not been contacted with said TTK inhibitor. If the level of ATR activation is greater in the first biological sample than the baseline level of ATR activation in the second biological sample, then TTK has been at least partially inhibited.

A third embodiment of this aspect of the present invention provides a method of monitoring TTK inhibition by the compounds of the present invention, or any other TTK inhibiting compounds, comprising determining a level of Hsp90 phosphorylation in a first biological sample that has been contacted with the TTK inhibitor and comparing said level of Hsp90 phosphorylation with a baseline level of Hsp90 phosphorylation from a second biological sample that has not been contacted with said TTK inhibitor. If the level of Hsp90 phosphorylation in the first biological sample is greater than the baseline level of Hsp90 phosphorylation in the second biological sample, then TTK has been at least partially inhibited.

In some of each of the foregoing embodiments, the level of p53 activation, ATR activation, or Hsp90 phosphorylation is quantified. The quantified level of p53 activation, ATR activation, or Hsp90 phosphorylation is then correlated with a percent TTK inhibition, in order to determine the percent TTK inhibition.

In some of each of the foregoing embodiments, the first and second biological samples are tissue samples.

In some of each of the foregoing embodiments, the first and second biological samples are tumor tissue samples.

In some of each of the foregoing embodiments, the first and second biological samples are cells from a cell culture.

In some of each of the foregoing embodiments, the first and second biological samples are obtained from animals administered the TTK inhibitor, or administered an appropriate control substance (e.g., a pharmaceutical formulation lacking the TTK inhibitor).

In some sub-embodiments of the first embodiment above, the genes encoding p53 in the first and second biological samples are wild-type p53 genes. In other sub-embodiments of the first embodiment above, the genes encoding p53 in the first and second biological samples are mutant p53 genes.

In some sub-embodiments of the second embodiment above, the method further comprises monitoring ATR activation, wherein ATR activation indicates TTK inhibition.

In some sub-embodiments of the first embodiment above, the method further comprises monitoring p53 activation, wherein p53 activation indicates TTK inhibition.

Any method of determining known in the art for determining p53 activation, ATR activation, or Hsp90 phosphorylation may be used in the corresponding embodiments of the present invention listed above.

Baseline levels may be determined by testing the respective level of p53 activation, ATR activation, or Hsp90 phosphorylation in a biological sample that has not been contacted with a TTK inhibitor, or has been contacted with a control substance, such as a carrier or pharmaceutical composition lacking the TTK inhibitor that is to be tested.

Non-limiting examples of TTK inhibitors that may be monitored with such embodiments of the present invention include essentially any TTK inhibitor, including the compounds of the present invention, as well as those disclosed in WO/2009024824, published Feb. 26, 2009; U.S. Provisional Application No. 61/162,974, filed Mar. 24, 2009; and U.S. Provisional Application No. 61/220,489, filed Jun. 25, 2009. Of course, it should be understood that any means of inhibiting TTK may be monitored using these three embodiments of this aspect of the present invention.

### 7. Methods of Making the Compounds of the Present Invention

Generally speaking, the compounds of the present invention can be synthesized using methods known in the art combined with the disclosure herein. In general, compounds of the invention can be synthesized according to Scheme 1 below. For example, C-6 substituted carbon analogs such as (v) below were prepared from 2,6-dichloropurine (i) in either four or two steps. The method may start with a commercially available 2,6-disubstituted purine compound (i). The substituent (e.g., —Cl in compound (i)) at C-6 is then displaced by an R¹ group through a linker by a nucleophilic aromatic substitution reaction (a) to form compound (ii) using thermal conditions (e.g., at temperatures 60-90° C.) in alcoholic solvents (e.g., ethanol, isopropanol, etc.). The hydrogen at N-9 of compound (ii) is then replaced with any suitable protecting group (PG; e.g., dihydropyran, MEM, p-toluene sulfonyl group, benzyl, etc.) by substitution reaction (b) to form compound (iii). The substituent (e.g., —Cl in compound (iii)) at C-2 is replaced by an R² group through a hetero atom linker (e.g., amino linker) to form compound (iv). This C-2 coupling reaction (c) may be performed thermally (e.g., 100-150° C.) using Buchwald coupling conditions with transition metal catalysts (e.g., palladium) in the presence of ligand (e.g., BINAP, Xanphos, s-Phos, etc.) and base (e.g., Cs₂CO₃, etc.) in organic solvents (e.g., toluene, etc.) with an appropriate aniline derivative (e.g., as shown in Scheme 12 below). Finally, removal of the protecting group at N-9 of compound (iv) employing either hydrolytic or hydrogenolysis conditions (e) yields compound (v). Alternatively, after step (a), one may skip the protecting group step (b) and replace the C-2 substituent (e.g., —Cl) with an appropriate aniline derivative to yield compound (v), experimental details of which are described in Scheme 2. This may be done, for example, employing acid catalyzed (e.g., p-toluene sulfonic acid, Camphor sulfonic acid, HCl, etc.) in solvents (e.g., CHCl₃, Dioxane, etc.) using either microwave or thermal conditions (e.g., between 100-150° C.).

Therefore, the present invention also provides methods for making compounds according to the present invention. One of the methods comprises reacting 2,6-disubstituted purine with -L¹-R¹ in a nucleophilic aromatic substitution reaction under suitable conditions and with suitable reactants to form a first intermediate substituted with -L¹-R¹ at the six position. This method also comprises reacting the first intermediate with a desired anilino derivative or analog to form a compound according to the present invention. In some embodiments of this method, the 2,6-disubstituted purine is 2,6-dichloropurine.

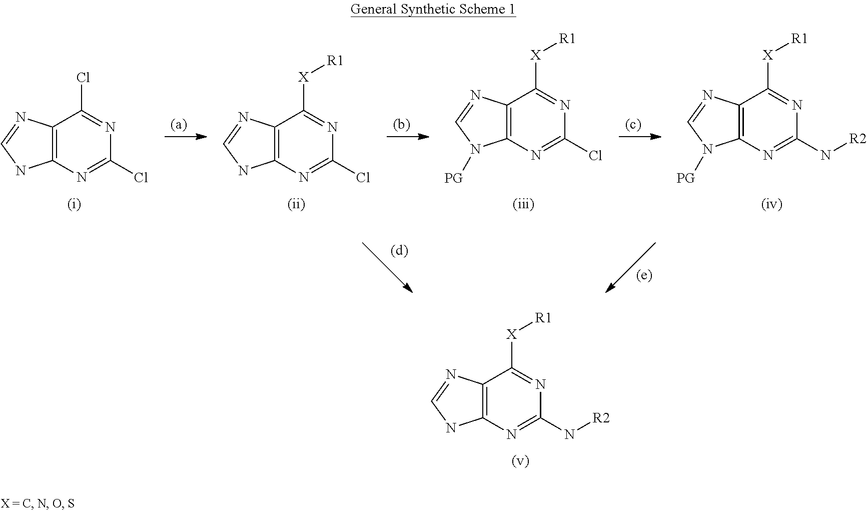

### EXAMPLES

Synthesis of compounds of the present invention can be accomplished according to the above general synthetic route, with reactants comprising most R-groups being commercially available and added to the general scaffold according to conventional techniques. See Table 1 for representative structures and relevant characterization data. Examples are given below to illustrate representative specific compounds.

### Example 1

### Synthesis of Compounds

### Synthesis of N*6*-Cyclohexyl-N*2*-(2-methyl-4-morpholin-4-yl-phenyl)-9H-purine-2,6-diamine (Table 1, C9)

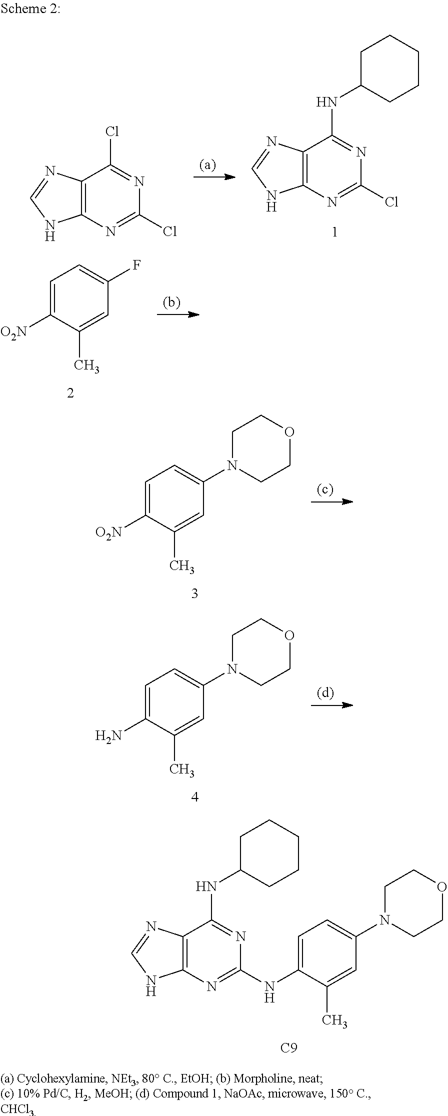

(2-Chloro-9H-purin-6-yl)-cyclohexyl-amine (1): A stirred mixture of 2,6-dichloropurine (7 g, 37.23 mmol), cyclohexylamine (4.7 mL, 41 mmol) and NEt₃ (10.4 mL, 75 mmol) in ethanol (185 mL) was heated to reflux overnight. Upon completion, the white precipitate was filtered and washed with ethyl ether. The precipitate (7 g, 75% yield) was dried for several hours before it was used in the next step without further purification. The product 2-chloro-N-cyclohexyl-9H-purin-6-amine was confirmed by LCMS and NMR analysis.

4-(3-Methyl-4-nitro-phenyl)-morpholine (3): To a solution of 4-fluoro-2-methyl-1-nitrobenzene (10 g, 64.52 mmol) in diethyl ether (20 mL,) was added morpholine in a drop wise fashion (11.3 mL, 130 mmol) at room temperature. The reaction was completed in 5 minutes as determined by LCMS and the mixture was stirred for 30 minutes with an HCl in ethyl ether solution (1M) until all product precipitated out of solution as a white powder. The product was isolated as the HCl salt of 4-(3-methyl-4-nitrophenyl)morpholine, whose structure was established based on its LCMS and ¹H NMR analysis.

2-Methyl-4-morpholin-4-yl-phenylamine (4): To a solution of 4-(3-methyl-4-nitrophenyl)morpholine (12.81 g, 57.7 mmol) in MeOH (350 mL, 0.16M) at room temperature was added 10% Pd/C (0.9 g) and hydrogenated overnight at 40 psi pressure in Parr hydrogenator. Upon completion, the Pd/C was filtered over a pad of Celite and the solvent was rotovaped until ˜100 mL of solvent was left. Diethyl ether (25 mL) was added to the solution and a solution of 1M HCl/ether was added until the purple color of the solution disappeared. The product 4 as a hydrochloride salt precipitated out of solution as a white solid. The white powder was filtered off and dried on lyophilizer overnight to yield the HCl salt of 2-methyl-4-(morpholin-4-yl)aniline (13 g, 99% yield).

N*6*-Cyclohexyl-N*2*-(2-methyl-4-morpholin-4-yl-phenyl)-9H-purine-2,6-diamine (Table 1, C9): Compound 1 (2.00 g, 7.91 mmol) was combined in an 80 mL microwave vial with compound 4 (2.53 g, 9.56 mmol), sodium acetate (980 mg, 11.95 mmol) and chloroform (40 mL) creating a suspension. The mixture was then heated to 150° C. in the microwave for 30 minutes. The solvents were then removed in vacuo and the crude solid purified via reverse phase MPLC to give the product as a white solid in yields ranging from 12-30%.

In Table 1, compounds C6, C40, C45, C48, C49, C51, C52, C76, C84, C85 and C88 were prepared employing similar reaction conditions described above, starting from appropriately substituted amine and aniline derivative.

In the Table 2, compounds C262-264 were prepared according to the procedure described above, with appropriately substituted starting materials.

In the Table 3, compounds 277-287 were prepared according to the procedure described above, with appropriately substituted starting materials.

### Synthesis of N*6*-Cyclohexyl-N*2*-[4-(1,1-dioxo-1lambda*6*-thiomorpholin-4-yl)-2-methyl-phenyl]-9H-purine-2,6-diamine (Table 1, C72) and N*6*-Cyclohexyl-N*2*-[2-methyl-4-(1-oxo-1lambda*4*-thiomorpholin-4-yl)-phenyl]-9H-purine-2,6-diamine (Table 1, C59)

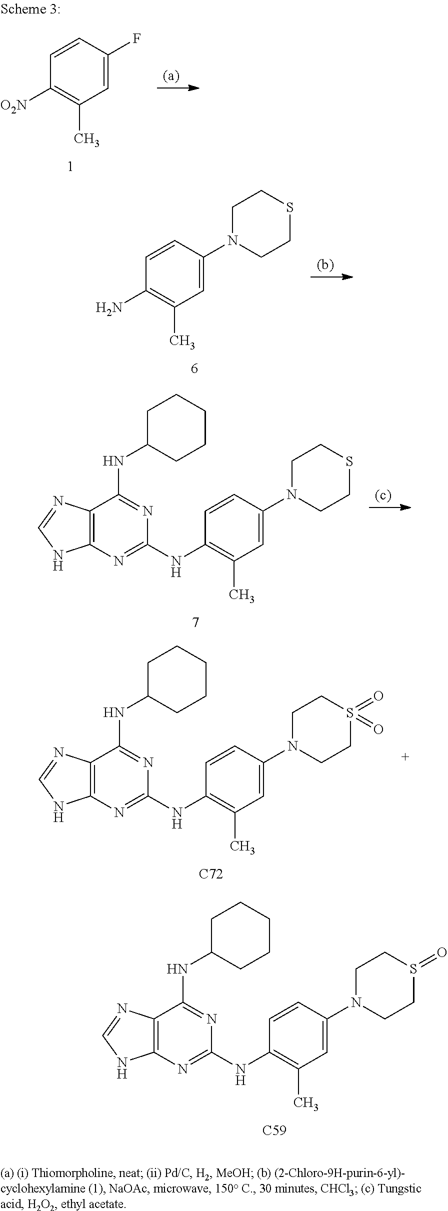

N*6*-Cyclohexyl-N*2*-(2-methyl-4-thiomorpholin-4-yl-phenyl)-9H-purine-2,6-diamine (7): Compound 6 was prepared in the manner previously described for compound 4 (Scheme 2) using thiomorpholine instead of morpholine. Then 6 was coupled to (2-Chloro-9H-purin-6-yl)-cyclohexylamine 1 to get compound 7, employing similar experimental conditions as described in Scheme 2.

N*6*-Cyclohexyl-N*2*-[4-(1,1-dioxo-1lambda*6*-thiomorpholin-4-yl)-2-methyl-phenyl]-9H-purine-2,6-diamine (Table 1, C72) and N*6*-Cyclohexyl-N*2*-[2-methyl-4-(1-oxo-1lambda*4*-thiomorpholin-4-yl)-phenyl]-9H-purine-2,6-diamine (Table 1, C59): Compound 7 (0.15 g, 0.354 mmol) and Na₂WO₄.2H₂O (4.7 mg, 0.014 mmol) were combined in ethyl acetate (2.8 mL) and chilled to 0° C. Hydrogen peroxide (35% aqueous, 0.779 mmol) was added. The reaction was stirred at 70° C. overnight. The solvent was removed in vacuo and the residue purified by HPLC giving compounds (Table 1, C72) and (Table 1, C59).

In the Table 2, C092 and C093 were prepared according to the similar procedure described above starting from appropriately substituted starting materials.

### Synthesis of N*2*-(2-Methyl-4-morpholin-4-yl-phenyl)-N*6*-(4-morpholin-4-yl-cyclohexyl)-9H-purine-2,6-diamine (Table 1, C68)

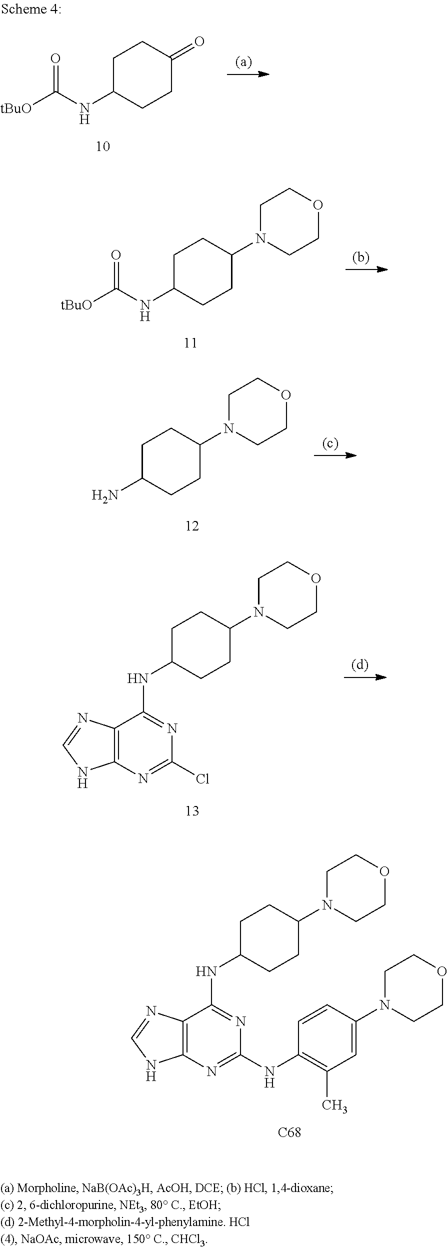

4-Morpholin-4-yl-cyclohexylamine (12): To a solution of N-Boc-4-aminocyclohexanone (10) (0.6 g, 2.82 mmol) in dichloroethane (10 mL) was added sodium triacetoxyborohydride (890 mg, 4.2 mmol), acetic acid (100 μL, 1.69 mmol) and morpholine (366 μL, 4.2 mmol). The reaction mixture was stirred overnight at room temperature. Upon completion it was extracted with ethyl acetate and washed the organic layer with saturated sodium bicarbonate solution. The organic layer was evaporated and the residue was dried in vacuo giving the product as a crude oil (11). The crude oil (11) was taken up in 4 N HCl in 1,4-dioxane and stirred at room temperature for 2 hours after which diethyl ether was added causing the product to precipitate from solution. The precipitate was collected by filtration to give the product as an off white solid (12).

N*2*-(2-Methyl-4-morpholin-4-yl-phenyl)-N*6*-(4-morpholin-4-yl-cyclohexyl)-9H-purine-2,6-diamine (C68): Compound (12) was coupled with 2,6-dichloropurine employing thermal conditions using triethylamine and ethanol, as described in Scheme 2, to get compound (13), which by typical acid catalyzed microwave conditions, as described in Scheme 2, was converted to the final compound (Table 1, C68).

### Synthesis of 4-[(2-{[2-chloro-4-(propan-2-yloxy)phenyl]amino}-9H-purin-6 yl)amino]cyclohexanol (Table 2, C265)

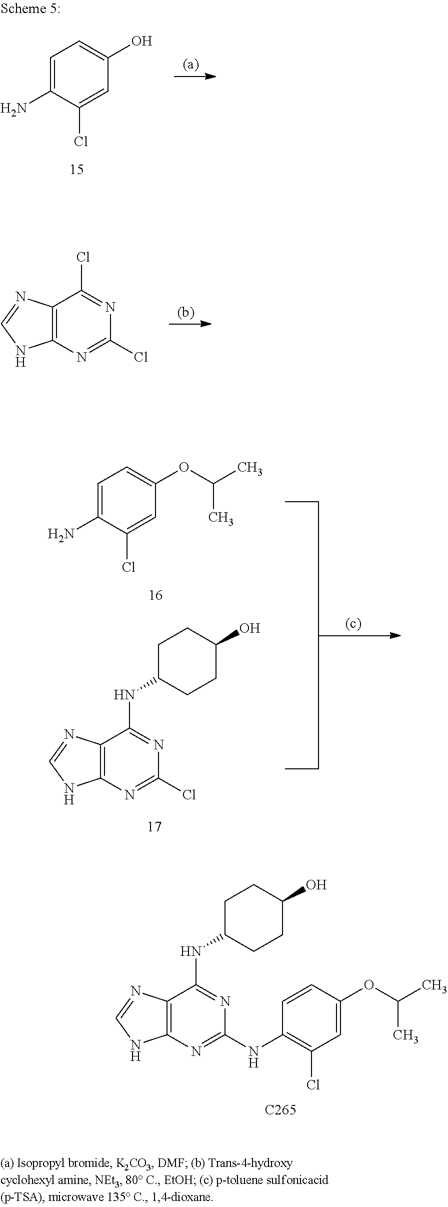

2-Chloro-4-isopropoxy-phenylamine (16): To a magnetically stirred solution of 15 (2 g, 11.17 mmol) in anhydrous DMF (12 mL) was added K₂CO₃ (3.24 g, 23.46 mmol) at room temperature, followed by isopropyl bromide (1.05 mL, 11.17 mmol) and the mixture was allowed to stir overnight at 80° C. under nitrogen atmosphere. Upon completion, the precipitate was filtered and the solvent was evaporated. The residue thus obtained was further purified by silica gel flash chromatography (30-100% ethyl acetate/hexane) to yield an oil 16 (1.5 g, 73% yield) for which the structure was established based on proton NMR and mass spectroscopy.

4-[(2-chloro-9H-purin-6-yl)amino]cyclohexanol (17): 4-[(2-chloro-9H-purin-6-yl)amino]cyclohexanol 17 was prepared using the procedure described for compound 1 in Scheme 2.

4-[(2-{[2-chloro-4-(propan-2-yloxy)phenyl]amino}-9H-purin-6 yl)amino]cyclohexanol (C265): To a magnetically stirred solution of 16 (148 mg, 0.98 mmol) in anhydrous 1,4-dioxane (4 mL) was added 4-[(2-chloro-9H-purin-6-yl)amino]cyclohexanol 17 (200 mg, 0.75 mmol) and p-TSA (57 mg, 0.3 mmol) and the mixture was microwaved for 1 hour at 135° C. Upon completion, the solvent was evaporated and the precipitate purified by reversed phase HPLC (ACN/H₂O/TFA) to yield a solid (Table 2, C265) (100 mg, 32% yield) for which the structure was established based on proton NMR and mass spectroscopy.

In Table 1, compounds C1, C3, C4, C5, C7, C8, C10, C11, C12, C16, C17, C23, C27, C28, C37, C41, C44, C50, C55, C62, C64, C65, C71, and C77 were prepared starting from appropriately substituted amine and aniline derivative using conditions described above.

### Synthesis of N*6*-(4-Ethoxy-cyclohexyl)-N*2*-(2-methyl-4-morpholin-4-yl-phenyl)-9H-purine-2,6-diamine (Table 1, C58)

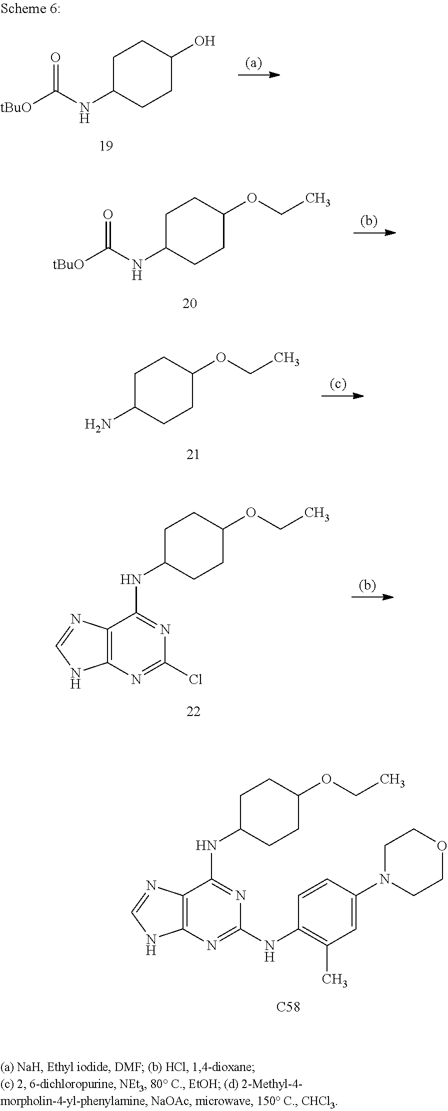

4-Ethoxy-cyclohexylamine (21): To a solution of N-boc-4-aminocyclohexanol 19 (0.610 g, 2.84 mmol) in DMF (10 mL) was added sodium hydride (148 mg, 3.69 mmol), and ethyl iodide (459 μL, 5.69 mmol). The reaction mixture was stirred overnight at room temperature followed by extraction with ethyl acetate and saturated sodium bicarbonate solution. The solvent was then removed in vacuo giving the product as a crude oil 20. The crude oil 20 from was taken up in 4 N HCl in 1,4-dioxane and stirred at room temperature for 2 hours after which diethyl ether was added causing the product to precipitate from solution. The precipitate was collected by filtration to give the product 21 as an off-white solid.

(2-Chloro-9H-purin-6-yl)-(4-ethoxy-cyclohexyl)-amine (22): Compound 21 was coupled with 2,6-dichloropurine to get 22 employing conditions as described for compound 1 in Scheme 2.

N*6*-(4-Ethoxy-cyclohexyl)-N*2*-(2-methyl-4-morpholin-4-yl-phenyl)-9H-purine-2,6-diamine (C58): The compound 22 and hydrochloride salt of 2-Methyl-4-morpholin-4-yl-phenylamine were coupled to get compound (Table 1, C58), using sodium acetate and microwave method as described in Scheme 2.

### Synthesis of N*6*-(4,4-Difluoro-cyclohexyl)-N*2*-(2-methyl-4-morpholin-4-yl-phenyl)-9H-purine-2,6-diamine (Table 1, C56) and N*6*-(4-Fluoro-cyclohex-3-enyl)-N*2*-(2-methyl-4-morpholin-4-yl-phenyl)-9H-purine-2,6-diamine (Table 1, C57)

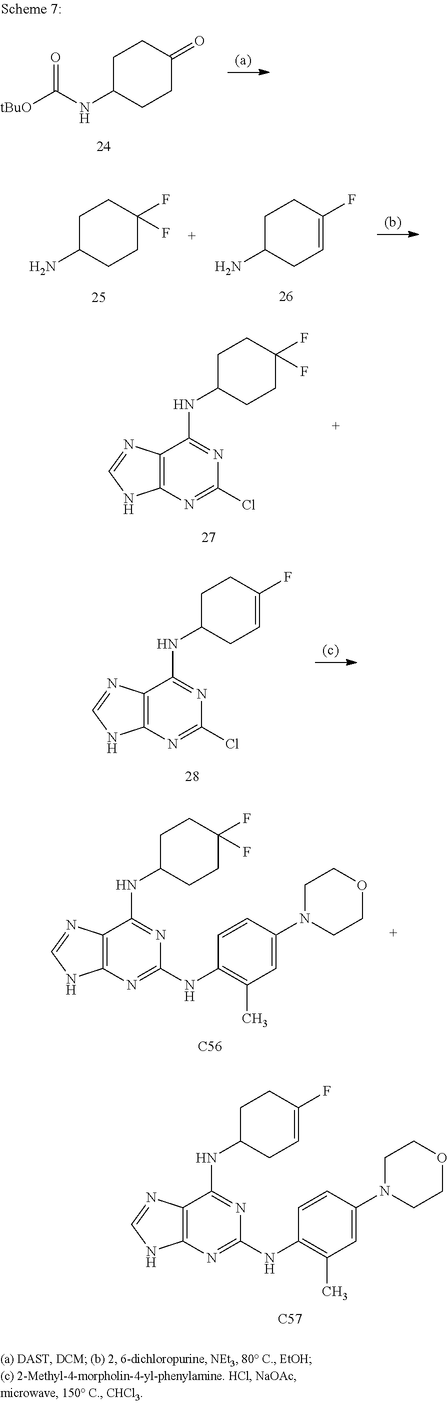

4,4-Difluoro-cyclohexylamine (25) and 4-Fluoro-cyclohex-3-enylamine (26): In a 100 mL Teflon round bottom flask, a solution of N-Boc-4-aminocyclohexanone 24 (5.0 g, 23.4 mmol) in dichloromethane (25 mL) was combined with diethylaminosulfur trifluoride (DAST) (5.5 mL, 39.9 mmol) under an inert nitrogen atmosphere. The reaction mixture was stirred overnight at room temperature followed by quenching with saturated sodium bicarbonate. The product was extracted into dichloromethane then precipitated from solution by the addition of HCl in ethyl ether. The product consisted of the two compounds 25 and 26 shown as compound mixture.

N*6*-(4,4-Difluoro-cyclohexyl)-N*2*-(2-methyl-4-morpholin-4-yl-phenyl)-9H-purine-2,6-diamine (C56) and N*6*-(4-Fluoro-cyclohex-3-enyl)-N*2*-(2-methyl-4-morpholin-4-yl-phenyl)-9H-purine-2,6-diamine (C57): Employing conditions described in Scheme 2, compound mixture 25 and 26 was coupled with 2,6-dichloropurine to get compound mixture 27 and 28, and were subsequently, coupled with 2-Methyl-4-morpholin-4-yl-phenylamine.HCl affording compound (Table 1, C56) and (Table 1, C57), which were separated by semi-prep HPLC.

### Synthesis of N*6*-Cyclohexyl-N*2*-(2-methyl-4-[1,2,4]triazol-1-yl-phenyl)-9H-purine-2,6-diamine (Table 1, C53)

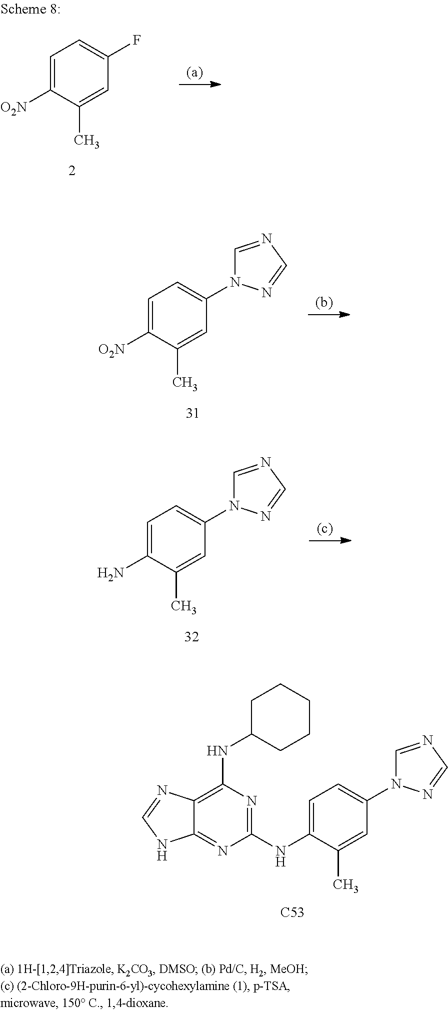

2-Methyl-4-[1,2,4]triazol-1-yl-phenylamine (32): To a round bottom flask containing DMSO (75 mL) was added 2 (5 g, 0.032 mol), 1H-[1,2,4]Triazole (2.45 g, 0.354 mol), and K₂CO₃ (8.9 g, 0.064 mol). The reaction was heated to 90° C. for 18 hours. The reaction was cooled to room temperature and then water was added to precipitate out product 31. The precipitate was filtered and dried to give 5.4 grams (83% yield) of product. Compound 31 (1.014 g, 0.0049 mol) was reduced using Pd/C (0.2028 g, 20%), H₂ and dry methanol (60 mL) to give compound 32 (0.8174 g, 94% yield).

N*6*-Cyclohexyl-N*2*-(2-methyl-4-[1,2,4]triazol-1-yl-phenyl)-9H-purine-2,6-diamine (C53): Compound 32 was coupled with (2-Chloro-9H-purin-6-yl)-cyclohexylamine 1 employing p-TSA and microwave conditions described before in Scheme 5 to get compound (Table 1, C53)

### Synthesis of N*6*-Cyclohexyl-N*2*-[4-(1-ethyl-pyrrolidin-3-yloxy)-2-methyl-phenyl]-9H-purine-2,6-diamine (Table 1, C39)

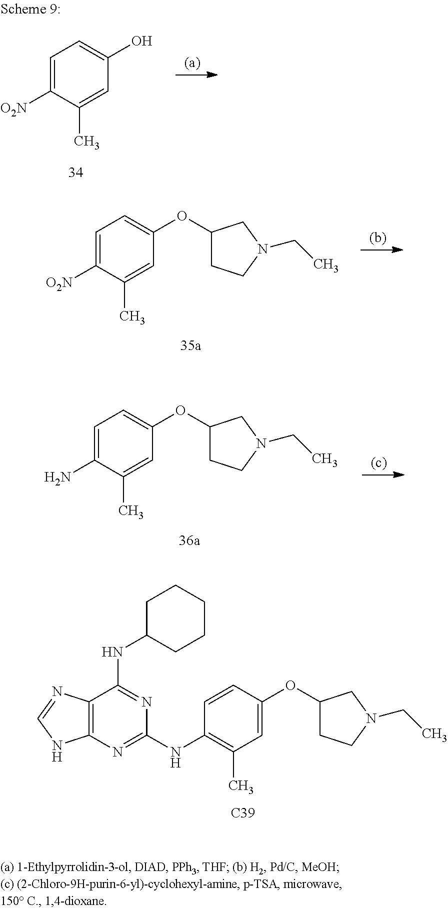

1-Ethyl-3-(3-methyl-4-nitro-phenoxy)-pyrrolidine (35a): To a magnetically stirred solution of 3-methyl-4-nitrophenol 34a (0.8 g, 5.09 mmol) in anhydrous THF (17 mL) was added DIAD (1.51 mL, 7.64 mmol), PPh₃ (2 g, 7.64 mmol) and 1-ethylpyrrolidin-3-ol (645 mg, 5.6 mmol) at room temperature and was allowed to stir overnight under nitrogen atmosphere. Upon completion, the solvent was evaporated under vacuum and the residue purified by silica gel flash chromatography (0-100% MeOH/DCM) to yield 1-ethyl-3-(3-methyl-4-nitrophenoxy)pyrrolidine 35a for which the structure was established based on proton NMR and mass spectroscopy.

4-(1-Ethyl-pyrrolidin-3-yloxy)-2-methyl-phenylamine (36a): To a magnetically stirred solution of 1-ethyl-3-(3-methyl-4-nitrophenoxy)pyrrolidine 35a (0.156 g, 0.624 mmol) in MeOH (13 mL) was added Pd/C (15 mg) and the mixture was stirred overnight in a Parr Hydrogenator under 40 psi. Upon completion, the precipitate was filtered and the solvent was concentrated in vacuo. 2-methyl-4-[2-(morpholin-4-yl)ethoxy]aniline 36a (136 mg, 99% yield) was used in the next step without further purification.

N*6*-Cyclohexyl-N*2*-[4-(1-ethyl-pyrrolidin-3-yloxy)-2-methyl-phenyl]-9H-purine-2,6-diamine (C39): Compound 36a was coupled to (2-Chloro-9H-purin-6-yl)-cyclohexyl-amine employing p-toluene sulfonic and microwave conditions described before in Scheme 5, to get compound (Table 1, C39) which was purified by reversed phase HPLC (ACN/H₂O/TFA) and the structure was established based on proton NMR and mass spectroscopy.

In Table 1, compound C70 was prepared in an analogous manner as described above starting from appropriate starting material 8-Methyl-8-aza-bicyclo[3.2.1]octan-3-ol. In Table 1, compound C83 was prepared using above procedure starting from 1-Ethyl-pyrrolidin-3-ol and 2-Ethoxy-4-fluoro-1-nitro-benzene.

### Synthesis of N*6*-Cyclohexyl-N*2*-[2-methyl-4-(2-morpholin-4-yl-ethoxy)-phenyl]-9H-purine-2,6-diamine (Table 1, C47)

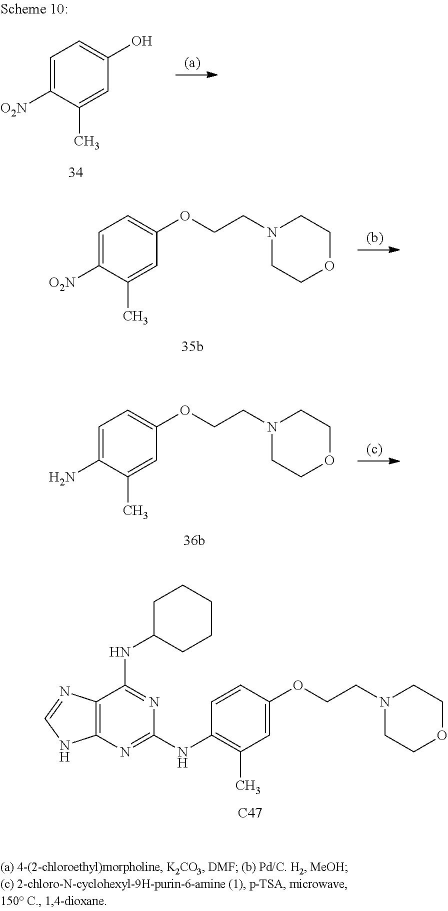

4-[2-(3-Methyl-4-nitro-phenoxy)-ethyl]-morpholine (35b): To a magnetically stirred solution of 3-methyl-4-nitrophenol 34 (0.2 g, 1.29 mmol) in anhydrous DMF (3 mL) was added K₂CO₃ (540 mg, 3.87 mmol) at room temperature, followed by the addition of 4-(2-chloroethyl)morpholine (288 mg, 1.55 mmol) and the mixture was allowed to stir overnight at 80° C. under nitrogen atmosphere. Upon completion, water was added and the aqueous layer extracted with ethyl acetate several times. The combined organic phases were dried over anhydrous sodium sulfate and concentrated under vacuum. The residue was purified by silica gel flash chromatography (0-30% ethyl acetate/hexane) to yield an orange oil 4-[2-(3-methyl-4-nitrophenoxy)ethyl]morpholine 35b (282 mg, 82% yield) for which the structure was established based on proton NMR and mass spectroscopy

2-Methyl-4-(2-morpholin-4-yl-ethoxy)-phenylamine (36b): To a magnetically stirred solution of 4-[2-(3-methyl-4-nitrophenoxy)ethyl]morpholine 35b (0.2 g, 0.88 mmol) in MeOH (18 mL) was added Pd/C (30 mg) and the mixture was stirred overnight in a Parr Hydrogenator under 60 psi. Upon completion, the precipitate was filtered and the solvent concentrated in vacuo. 2-methyl-4-[2-(morpholin-4-yl)ethoxy]aniline 36b was used in the next step without further purification.

N*6*-Cyclohexyl-N*2*-[2-methyl-4-(2-morpholin-4-yl-ethoxy)-phenyl]-9H-purine-2,6-diamine (C47): The compound 36b was coupled to 2-chloro-N-cyclohexyl-9H-purin-6-amine employing acid catalyzed microwave coupling conditions described before in Scheme 5 and the product (Table 1, C47) purified by reversed phase HPLC (ACN/H₂O/TFA) and the structure was established based on proton NMR and mass spectroscopy.

In Table 1, compound C78 was prepared using similar conditions described above in Scheme 10 starting from 4-amino-3-chloro-phenol.

### Synthesis of N*6*-Cyclohexyl-N*2*-[4-(1-ethyl-piperidin-4-ylmethoxy)-2-methyl-phenyl]-9H-purine-2,6-diamine (Table 1, C46)

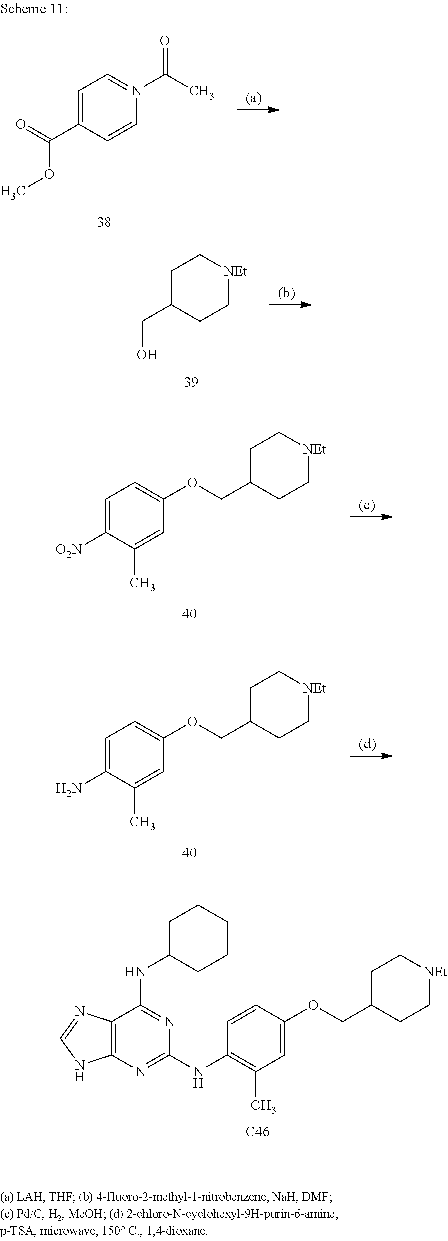

(1-Ethyl-piperidin-4-yl)-methanol (39): To a magnetically stirred solution of methyl isonipecotate 38 (4 g, 23.4 mmol) in anhydrous THF (350 mL) was added LAH (1 g) in portions at 0° C. Upon completion, 20 mL water was added, followed by 20 mL NaOH (15%) and 20 mL water. The solid was filtered off and solvent rotovaped. The residue (1-ethylpiperidin-4-yl)methanol 39 was purified by silica gel flash chromatography (0-100% MeOH/DCM).

4-(1-Ethyl-piperidin-4-ylmethoxy)-2-methyl-phenylamine (41): To a magnetically stirred solution of (1-ethylpiperidin-4-yl)methanol 39 (0.305 g, 2.13 mmol) in DMF (4 mL) was added NaH (85 mg, 2.13 mmol). The mixture was stirred 30 minutes at room temperature before adding 4-fluoro-2-methyl-1-nitrobenzene (0.3 g, 1.94 mmol). Upon completion, the reaction was quenched with water and extracted with DCM several times. The organic phases were combined and the solvent concentrated in vacuo. The residue 1-ethyl-4-[(3-methyl-4-nitrophenoxy)methyl]piperidine 40 was purified by silica gel flash chromatography (0-100% MeOH/DCM). It was converted to 41, using conditions as described for compound 36b (Scheme 10).

Synthesis of N*6*-Cyclohexyl-N*2*-[4-(1-ethyl-piperidin-4-ylmethoxy)-2-methyl-phenyl]-9H-purine-2,6-diamine (C46): Compound 41 was coupled with 2-chloro-N-cyclohexyl-9H-purin-6-amine (1) employing acid catalyzed microwave conditions described before in Scheme 5, to yield compound (Table 1, C46).

### Synthesis of N*6*-Cyclohexyl-N*2*-[4-(1-ethyl-piperidin-4-ylmethoxy)-2-methyl-phenyl]-9H-purine-2,6-diamine (Table 1, C9)

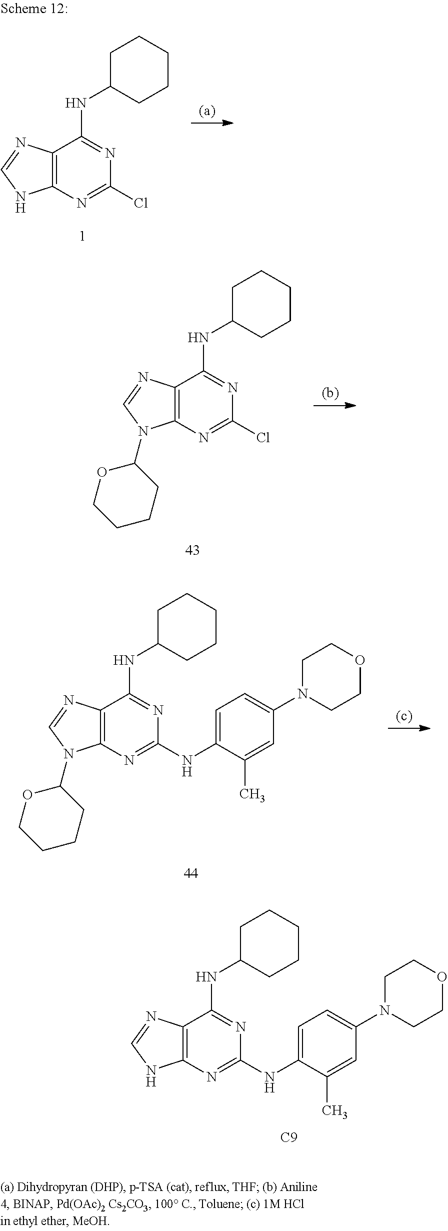

[2-Chloro-9-(tetrahydro-pyran-2-yl)-9H-purin-6-yl]-cyclohexyl-amine (43): To a mixture of 2-chloro-N-cyclohexyl-9H-purin-6-amine 1 (7 g, 84 mmol) in THF (300 mL, 0.3 mL) was added DHP (7.6 mL, 84 mmol) and p-TSA (1.6 g, 10 mmol) and the solution was heated to reflux over the weekend. Upon completion, the solvent was evaporated and the residue purified by silica gel flash chromatography with 30% ethyl acetate/hexanes as eluant to yield 2-chloro-N-cyclohexyl-9-(tetrahydro-2H-pyran-2-yl)-9H-purin-6-amine 43 as a white solid (8.4 g, 90% yield).

N*6*-Cyclohexyl-N*2*-(2-methyl-4-morpholin-4-yl-phenyl)-9-(tetrahydropyran-2-yl)-9H-purine-2,6-diamine (44): To a previously degassed stirred solution of Pd(OAc)₂ (0.999 g, 4.45 mmol) and BINAP (4.15 g, 6.67 mmol) in anhydrous toluene (300 mL) under a nitrogen atmosphere was added 2-chloro-N-cyclohexyl-9-(tetrahydro-2H-pyran-2-yl)-9H-purin-6-amine 43 (9.932 g, 29.65 mmol), Cs₂CO₃ (38.6 g, 118 mmol) and aniline 4 (Scheme 1) (7.43 g, 32.6 mmol). The mixture was heated to 100° C. overnight under nitrogen atmosphere. The reaction was monitored by LCMS, and water was added upon completion. The aqueous layer was extracted with ethyl acetate several times and the combined organic layers were dried over anhydrous sodium sulfate. Evaporation of the solvent yielded a residue that was purified by silica gel flash chromatography using MeOH/DCM as eluent (0-5%). N˜6˜-cyclohexyl-N˜2˜-[2-methyl-4-(morpholin-4-yl)phenyl]-9-(tetrahydro-2H-pyran-2-yl)-9H-purine-2,6-diamine was obtained as a white solid (8 g, 55% yield).

N*6*-Cyclohexyl-N*2*-(2-methyl-4-morpholin-4-yl-phenyl)-9H-purine-2,6-diamine (C9): To a solution of N˜6˜-cyclohexyl-N˜2˜-[2-methyl-4-(morpholin-4-yl)phenyl]-9-(tetrahydro-2H-pyran-2-yl)-9H-purine-2,6-diamine 44 (8 g, 16.3 mmol) in anhydrous MeOH (100 mL) was added 10 mL of a (1M) HCl/diethyl ether solution and the solution was stirred at room temperature. The reaction was monitored by LCMS for completion and the solvents were rotovaped. The residue was purified by C-18 reverse phase flash chromatography with MeOH/H₂O/0.1% TFA as eluant to provide N˜-6˜-cyclohexyl-N˜2˜-[2-methyl-4-(morpholin-4-yl)phenyl]-9H-purine-2,6-diamine (Table 1, C9) as a white powder (5.5 g, 83% yield, 99% purity).

In Table 1, compound C38 was prepared according the procedure described above using 2-Methyl-4-(4-methyl-piperazin-1-yl)-phenylamine instead of 2-Methyl-4-morpholin-4-yl-phenylamine.

In the Table 2, compound C259 was prepared according the procedure described above with appropriately substituted starting materials.

### Synthesis of [4-(6-Cyclohexylamino-9H-purin-2-ylamino)-3-methyl-phenyl]-(4-methyl-piperazin-1-yl)-methanone (Table 1, C60)

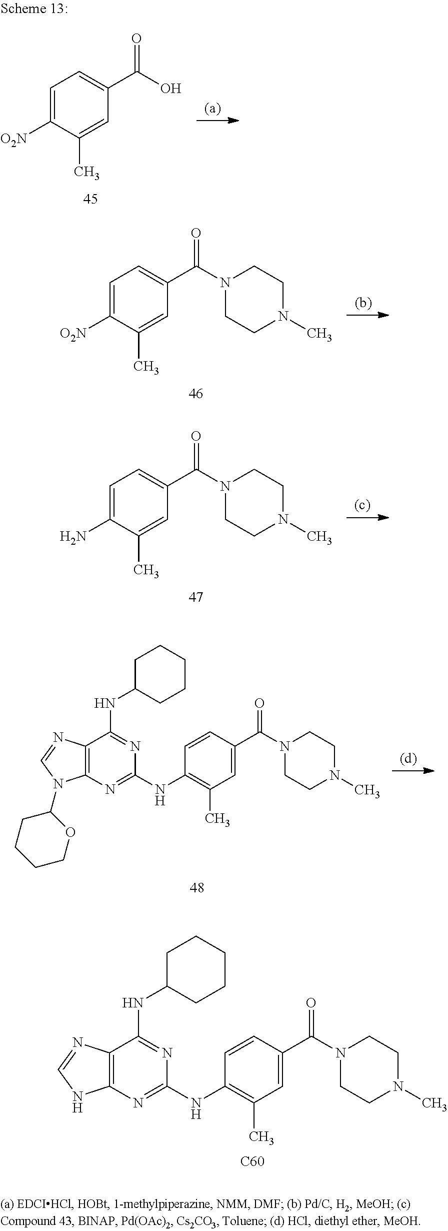

(3-Methyl-4-nitro-phenyl)-(4-methyl-piperazin-1-yl)-methanone (46): To a magnetically stirred solution of 3-methyl-4-nitrobenzoic acid 45 (0.73 g, 4.04 mmol, 1 equiv.) in anhydrous DMF (5 mL) was added N-methyl morpholine (NMM) (1.56 mL, 14.14 mmol), EDCI.HCl (930 mg, 4.85 mmol), HOBt (273 mg, 2.02 mmol) and 1-methylpiperazine (485 mg, 4.85 mmol). The mixture was allowed to stir overnight at room temperature under nitrogen atmosphere. Upon completion, solvent were concentrated in vacuo and the residue purified by silica gel flash chromatography (0-10% MeOH/DCM.) to yield (3-methyl-4-nitrophenyl)(4-methylpiperazin-1-yl)methanone 46 for which the structure was established based on proton NMR and mass spectroscopy.

(4-Amino-3-methyl-phenyl)-(4-methyl-piperazin-1-yl)-methanone (47): To a magnetically stirred solution of (3-methyl-4-nitrophenyl)(4-methylpiperazin-1-yl)methanone 46 (0.308 g, 1.17 mmol) in MeOH (5 mL) was added Pd/C (30 mg) and the mixture was stirred 4 hours in a Parr Hydrogenator under 40 psi. Upon completion, solvent were removed the product was precipitated as HCl salt by slow addition of HCl in ethyl ether. The precipitate was filtered to yield (4-amino-3-methylphenyl)(4-methylpiperazin-1-yl)methanone 47 which was used in the next step without further purification.

{-4-[6-Cyclohexylamino-9-(tetrahydro-pyran-2-yl)-9H-purin-2-ylamino]-3-methyl-phenyl}-(4-methyl-piperazin-1-yl)-methanone (48): A magnetically stirred solution Pd(OAc)₂ (3 mg, 0.013 mmol) and BINAP (12.1 mg, 0.02 mmol) was stirred at room temperature in anhydrous toluene (1 mL) under nitrogen atmosphere. After 30 minutes, 2-chloro-N-cyclohexyl-9-(tetrahydro-2H-pyran-2-yl)-9H-purin-6-amine 43 (43 mg, 0.129 mmol), (4-amino-3-methylphenyl)(4-methylpiperazin-1-yl)methanone 47 (69 mg, 0.258 mmol) and Cs₂CO₃ (254 mg, 0.78 mmol) were added and the mixture was heated overnight at 50° C. Upon completion, the solvent was evaporated and the residue purified by silica gel flash chromatography (0-100% ethyl acetate/hexane) to get 48.

[4-(6-Cyclohexylamino-9H-purin-2-ylamino)-3-methyl-phenyl]-(4-methyl-piperazin-1-yl)-methanone (C60): To a magnetically stirred solution of (4-{[6-(cyclohexylamino)-9-(tetrahydro-2H-pyran-2-yl)-9H-purin-2-yl]amino}-3-methylphenyl)(4-methylpiperazin-1-yl)methanone 48 (28 mg, 0.052 mmol) in MeOH (2 mL) was added HCl (0.2 mL, 1 M HCl in ether) and the mixture was stirred 4 hours. Upon completion, solvent was removed and residue purified by reversed phase HPLC (0-100% MeOH/H₂O/TFA) to yield (Table 1, C60) for which the structure was established based on proton NMR and mass spectroscopy.

In Table 1, compound C61 was prepared according to the similar procedure described above using morpholine instead of piperazine in the first step.

### Synthesis of N*6*-Cyclohexyl-N*2*-[1-methyl-4-(4-methyl-piperazin-1-ylmethyl)-phenyl]-9H-purine-2,6-diamine (Table 1, C69)

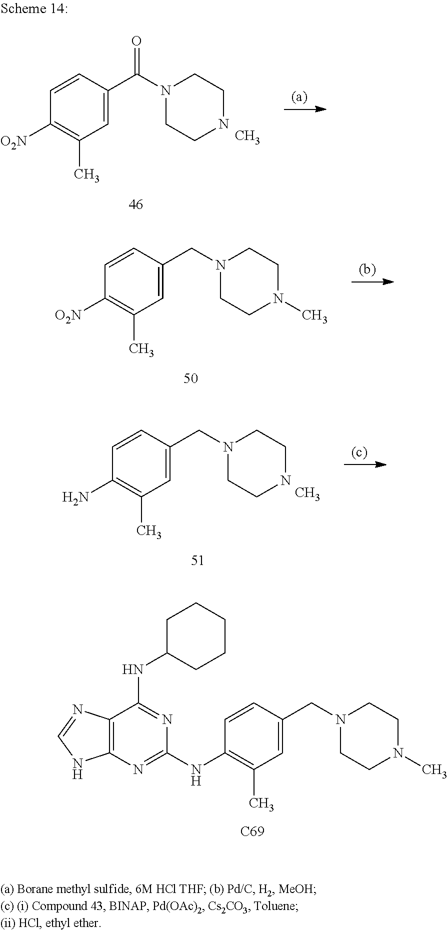

1-Methyl-4-(3-methyl-4-nitro-benzyl)-piperazine (50): To a magnetically stirred solution of (3-methyl-4-nitrophenyl)(4-methylpiperazin-1-yl)methanone 46 (0.77 g, 2.93 mmol) in THF (0.8 mL) heated to reflux was added drop wise borane methyl sulfide (9 mL, 17.6 mmol, 2M in THF solution) over a period of 15 minutes, 0.4 mL 6M HCl was added and the reaction heated to 100° C. After 30 minutes, a clear solution was obtained which was cooled to room temperature before adding NaOH (4N). The aqueous layer was then saturated with K₂CO₃ and was extracted with diethyl ether. Solvent was concentrated in vacuo and the residue was purified by silica gel flash chromatography (0-5% MeOH/DCM) to get compound 50.

N*6*-Cyclohexyl-N*2*-[2-methyl-4-(4-methyl-piperazin-1-ylmethyl)-phenyl]-9H-purine-2,6-diamine (C69): The compound 50 was hydrogenated employing similar conditions described for compound 47 in Scheme 13. Finally, compound 51 was converted to compound (Table 1, C69) using palladium catalyzed Buchwald coupling and hydrolysis procedures as described in Scheme 12.

In Table 1, Compound C63 was prepared similar to the procedure described above, using (3-Methyl-4-nitro-phenyl)-morpholin-4-yl-methanone in the first step.

In the Table 2, compounds C094, C095, C096, C097 and C098 were prepared according the procedure described above, with appropriately substituted starting materials.

### N*6*-Cyclohexyl-N*2*-(2-ethoxy-4-morpholin-4-ylmethyl-phenyl)-9H-purine-2,6-diamine (Table 1, C82)

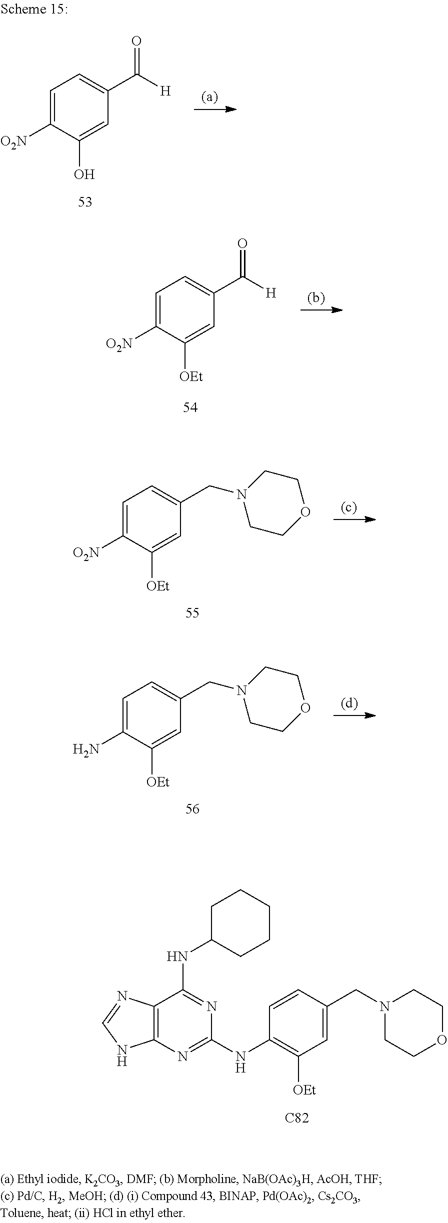

3-Ethoxy-4-nitro-benzaldehyde (54): To a magnetically stirred solution of 3-hydroxy-4-nitrobenzaldehyde 53 (0.65 g, 3.89 mmol) in anhydrous DMF (3 mL) was added K₂CO₃ (1.5 g, 10.5 mmol) at room temperature, followed by ethyl iodide (410 μL, 5.1 mmol) and the mixture was allowed to stir overnight at 80° C. under nitrogen atmosphere. Upon completion, water was added and the aqueous layer extracted with ethyl acetate several times. The combined organic phases were dried with sodium sulfate and concentrated in vacuo. The residue thus obtained was further purified by silica gel flash chromatography (0-20% ethyl acetate/hexane) to yield 3-ethoxy-4-nitrobenzaldehyde 54 for which the structure was established based on proton NMR and mass spectroscopy.

N*6*-Cyclohexyl-N*2*-(2-ethoxy-4-morpholin-4-ylmethyl-phenyl)-9H-purine-2,6-diamine (C82): Sodium triacetoxyborohydride (5.12 mmol, 2 equiv.) was added to a mixture of morpholine (223 μL, 2.56 mmol, 1 equiv.), 3-ethoxy-4-nitrobenzaldehyde 54 (0.5 g, 2.56 mmol), acetic acid (20 μL), and THF (25 mL, 0.1 M). After 18 hours, the reaction was quenched with water. The solution was diluted with ethyl acetate, washed with 1 M NaOH or 1 M K₂CO₃, washed with brine, dried (anhydrous Na₂SO₄), and concentrated. Purification by flash silica gel chromatography yielded 4-(3-ethoxy-4-nitrobenzyl)morpholine 55. Employing similar experimental conditions described above, hydrogenation of 55, followed by coupling of the aniline 56 obtained with compound 43 and hydrolysis of the protecting group, as described in Scheme 12, gave compound (Table 1, C82).

### N*6*-Cyclohexyl-N*2*-[4-(1-ethyl-piperidin-4-yl)-2-methyl-phenyl]-9H-purine-2,6-diamine (Table 1, C79)

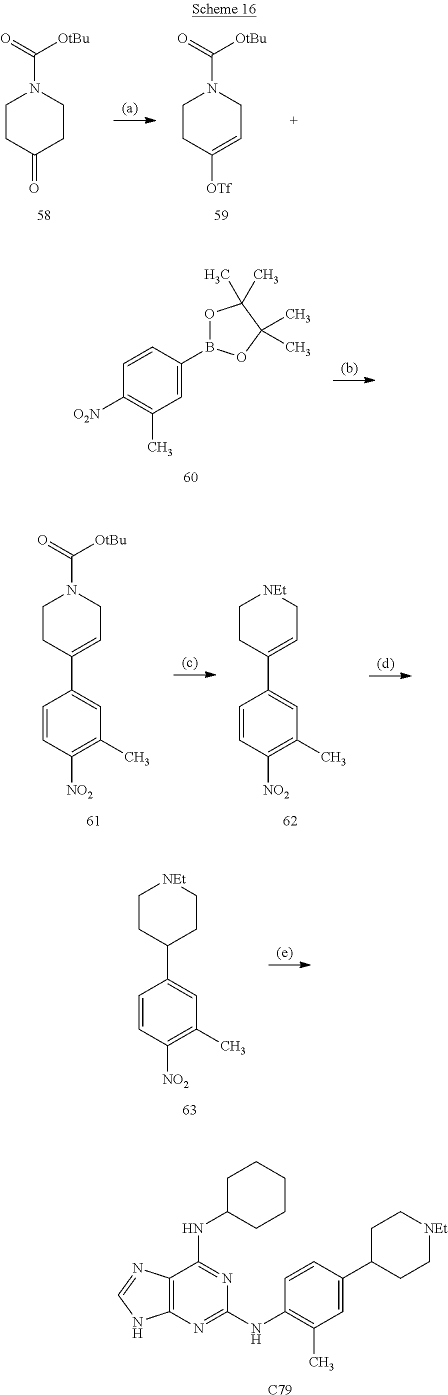

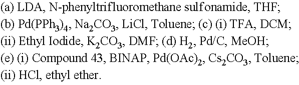

4-Trifluoromethanesulfonyloxy-3,6-dihydro-2H-pyridine-1-carboxylic acid tert-butyl ester (59): To a magnetically stirred solution of LDA (18 mmol, prepared from nBuLi and diisopropylamine in THF) at −78° C. was added drop wise over 25 minutes tert-butyl 4-oxopiperidine-1-carboxylate 58 (3.25 g, 16.33 mmol) in THF (25 mL). After 20 minutes at that temperature, a solution of N-phenyltrifluoromethane sulfonimide in 25 mL THF was added and the mixture was stirred an additional 4 hours at 0° C. Upon completion, the solvent was concentrated in vacuo and filtered over a pad of alumina using hexanes:ethyl acetate (9:1) as eluant to afford the product tert-butyl 4-{[(trifluoromethyl)sulfonyl]oxy}-3,6-dihydropyridine-1(2H)-carboxylate 59.

4-(3-Methyl-4-nitro-phenyl)-3,6-dihydro-2H-pyridine-1-carboxylic acid tert-butyl ester (61): A magnetically stirred solution of tert-butyl 4-{[(trifluoromethyl)sulfonyl]oxy}-3,6-dihydropyridine-1(2H)-carboxylate 59 (1.51 g, 5.74 mmol), Pd(PPh₃)₄ (671 mg, 0.58 mmol), 3,3,4,4-tetramethyl-1-(3-methyl-4-nitrophenyl)borolane 60 (1.9 g, 5.74 mmol), LiCl (730 mg, 17.22 mmol) and Na₂CO₃ (8.2 mL) in toluene was heated to reflux at 80° C. under N₂ atm. Upon completion, the reaction was cooled to room temperature, the solvent was concentrated in vacuo. The residue thus obtained, was suspended in water, and extracted several times with ethyl acetate. The organic phases were washed with water and brine and the residue obtained after evaporation of the solvent was purified by flash chromatography on silica gel (elution with 15% ethyl acetate/hexanes) to give tert-butyl 4-(3-methyl-4-nitrophenyl)-3,6-dihydropyridine-1(2H)-carboxylate 61 as a red solid in 54% yield.

1-Ethyl-4-(3-methyl-4-nitro-phenyl)-1,2,3,6-tetrahydro-pyridine (62): To a magnetically stirred solution of tert-butyl 4-{[(trifluoromethyl)sulfonyl]oxy}-3,6-dihydropyridine-1(2H)-carboxylate 61 (0.5 g, 1.57 mmol) in DCM (1 mL) was added TFA (3 mL) and the mixture was allowed to stir overnight under nitrogen atmosphere. Upon completion, the solvents were removed by evaporation and the residue dried overnight. The structure was established based on proton NMR and not further purified. The TFA salt (0.24 g, 0.723 mmol) was dissolved in anhydrous DMF (1 mL) and K₂CO₃ (210 mg, 1.52 mmol) was added at room temperature, followed by the addition of ethyl iodide (65 μL, 0.79 mmol) and the mixture was allowed to stir overnight at 80° C. under nitrogen atmosphere. Upon completion, water was added and the aqueous layer extracted with ethyl acetate several times. The combined organic phases were dried with anhydrous sodium sulfate and concentrated in vacuo. The residue thus obtained was further purified by silica gel flash chromatography (0-10% ethyl acetate/hexane.) to yield 1-ethyl-4-(3-methyl-4-nitrophenyl)piperidine 62 for which the structure was established based on proton NMR and mass spectroscopy.

N*6*-Cyclohexyl-N*2*-[4-(1-ethyl-piperidin-4-yl)-2-methyl-phenyl]-9H-purine-2,6-diamine (C791: The compound 62 was hydrogenated and the resulting aniline 63 was coupled with compound 43 employing Buchwald coupling condition and hydrolyzed the N-9 protecting group to get compound (Table 1, C79), using similar experimental conditions described earlier in Scheme 12.

In the Table 2, compounds C99 and C100 were prepared according to the procedure described above, with appropriately substituted starting materials.

### Synthesis of (6-Cyclohexyloxy-9H-purin-2-yl)-(2-methyl-4-morpholin-4-yl-phenyl)-amine (Table 1, C54)

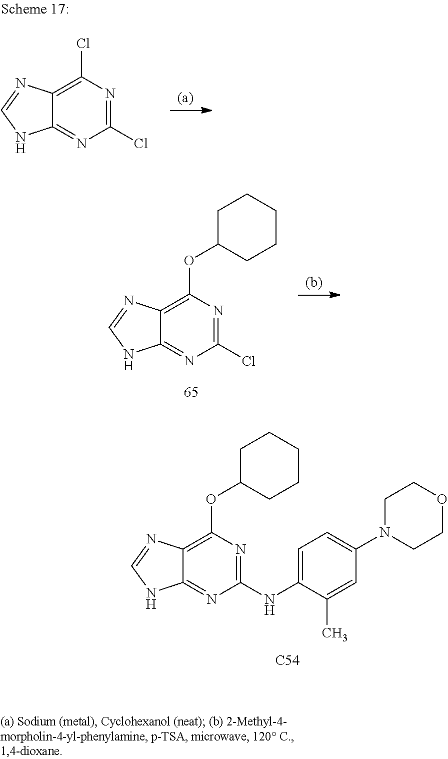

2-Chloro-6-cyclohexyloxy-9H-purine (65): Cyclohexanol (20 mL, 0.2 mol) was added to a round bottom flask and heated to 90° C. Sodium metal (1.45 g, 0.063 mol) was added slowly over 3 hours. The 2,6-dichloropurine (2.0 g, 0.01 mol) was then added and the reaction mixture was allowed to stir at 90° C. for 18 hours. The reaction mixture was then cooled to room temperature and neutralized with acetic acid. The solution was then extracted with CH₂Cl₂. The organic layer was concentrated and residue was triturated with ethanol to form a solid which was filtered to give 1.4 grams (53%) of product 65.

(6-Cyclohexyloxy-9H-purin-2-yl)-(2-methyl-4-morpholin-4-yl-phenyl)-amine (C54): 2-Methyl-4-morpholin-4-yl-phenylamine coupled to 65 employing acid catalyzed microwave conditions as described in Scheme 5, to get compound (Table 1, C54).

### Synthesis of 1H-Imidazole-2-carboxylic acid {3-[4-(6-cyclohexylamino-9H-purin-2-ylamino)-3-methyl-phenoxy]-propyl}-amide (Table 1, C14)

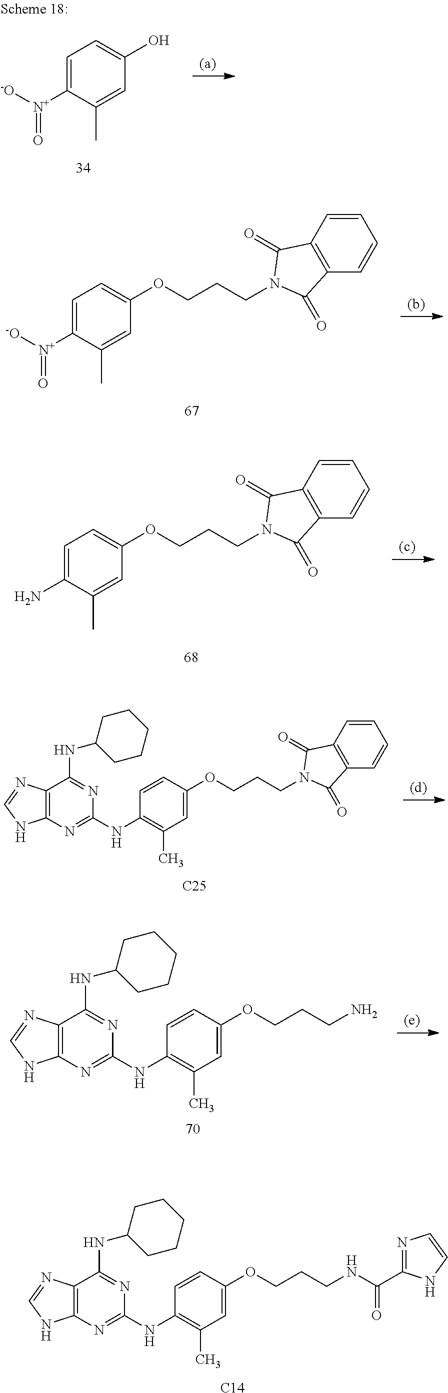

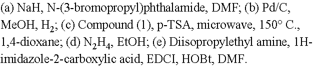

N-(3-(-3-Methyl-4-nitrophenoxy)-propyl)-phthalamide (67): Sodium hydride (60%, 1.25 g, 31.2 mmol) was slowly added to a stirring solution of 4-nitro-m-cresol (3.98 g, 26.0 mmol) in dry DMF (65 mL). After 20 minutes, N-(3-bromopropyl)phthalamide (7.65 g, 28.5 mmol) was added and the reaction was stirred over night at ambient temperature. The compound was precipitated with water collected by vacuum filtration, washed with water followed by diethyl ether. The material was carried on without further purification.

2-[3-(4-{[6-(cyclohexylamino)-9H-purin-2-yl]amino}-3-methylphenoxy)propyl]-1H-isoindole-1,3(2H)-dione (C25): A 1-L flask was charged with N-(3-(-3-Methyl-4-nitrophenoxy)-propyl)-phthalamide (7.22 g, 21.1 mmol) and methanol (400 mL). The flask was purged with nitrogen, charged with palladium on carbon (10%, 0.036 g), equipped with a balloon of H₂, and stirred for 60 hours or until the reaction was determined to be complete. The content of the flask was passed through a pad of Celite™. The solvent was removed in vacuo to afford 68 as an orange solid, which was used without further purification. This compound was coupled to (2-Chloro-9H-purin-6-yl)-cyclohexyl-amine 1 employing acid catalyzed microwave conditions as described in Scheme 5, to afford compound (Table 1, C25).

N2-(4-(3-Aminopropoxy)-2-methylphenyl)-N6-cyclohexyl-9H-purine-2,6-diamine (70): (3-(4-(6-cyclohexylamino-9H-purin-2-yl-amino)-3-methylphenoxy)-propyl-N-phthalamide (0.136 g, 0.259 mmol) was treated with excess hydrazine monohydrate (64%, N₂H₄), either neat or as a solution in ethanol, and stirred overnight from ambient temperature to 80° C. After 12 hours, the reaction appeared complete by LCMS. The hydrazine was removed in vacuo and the crude product was purified by reverse-phase HPLC.

1H-Imidazole-2-carboxylic acid {3-[4-(6-cyclohexylamino-9H-purin-2-ylamino)-3-methylphenoxy]propyl}amide (C14): Diisopropylethyl amine (0.10 g, 0.81 mmol) was added to a solution of 1H-imidazole-2-carboxylic acid (0.025 g, 0.22 mmol), N-2-[4-(3-aminopropoxy)-2-methyl-phenyl]-N-6-cyclohexyl-9H-purine-2,6-diamine (0.08 g, 0.20 mmol), EDCI (0.046 g, 0.24 mmol), and HOBt (0.037 g, 0.24 mmol) in DMF (1.0 mL), and the resulting mixture was stirred overnight at ambient temperature. The solvent was removed and the crude material was purified by reverse-phase HPLC to provide compound (Table 1, C14).

In Table 1, C2, C15, C18, C19, C20, C29, C30, C31, C32, C33, C34, C66, C67, C80, and C81 were prepared employing similar reaction conditions described above using a starting amine such as compound 70 or appropriately substituted derivative thereof, and acid or acid chloride derivative.

### Synthesis of 1-Ethyl-3-{4-[2-(2-methyl-4-morpholin-4-yl-phenylamino)-9H-purin-6-ylamino]-cyclohexylmethyl}-urea (Table 1, C24)

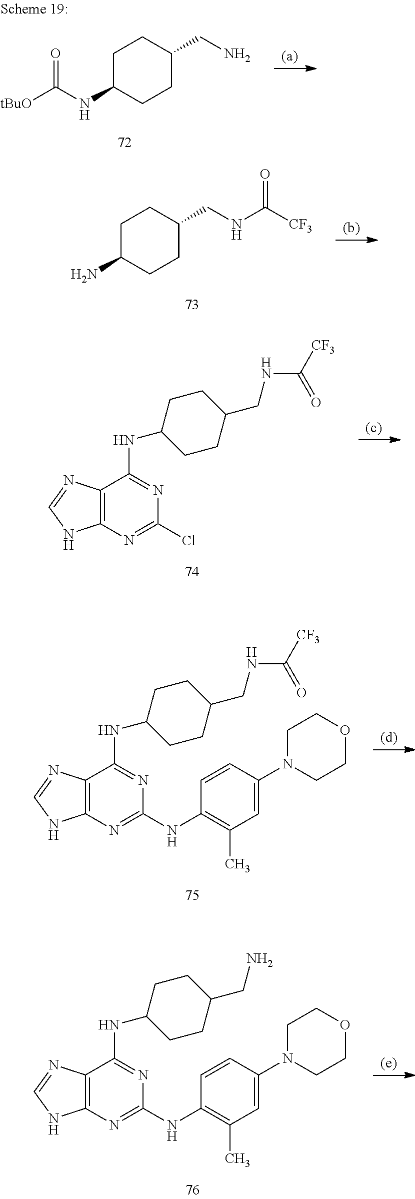

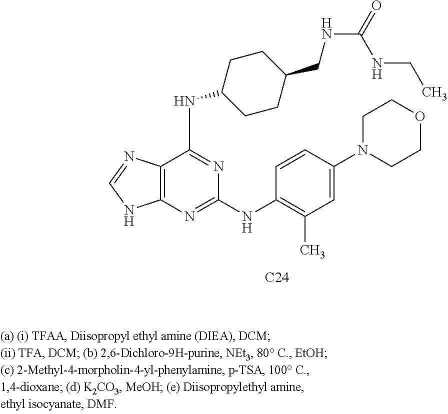

N-(4-Amino-cyclohexylmethyl)-2,2,2-trifluoro-acetamide (73): (4-Aminomethyl-cyclohexyl)-carbamic acid tert-butyl ester (Albany Molecular Research, 1.0 g, 4.3 mmol) and Hunig's base (1.5 mL, 8.6 mmol) were dissolved in dichloromethane (21 mL). Trifluoroacetic anhydride (0.73 mL, 5.3 mmol) was added drop wise and stirred at room temperature overnight. The reaction mixture was diluted into 100 mL dichloromethane, washed with sat. Na₂CO₃, water and brine, dried with Na₂SO₄ and concentrated. The BOC group was removed from the residue by dissolving in trifluoroacetic acid (3 mL) and removing solvent under reduced pressure yielding the product as the TFA salt.

N-[4-(2-Chloro-9H-purin-6-ylamino)-cyclohexylmethyl]-2,2,2-trifluoro-acetamide (74): 2,6-Dichloro-9H-purine (40 mg, 0.21 mmol), N-(4-Amino-cyclohexylmethyl)-2,2,2-trifluoro-acetamide (80 mg, 0.23 mmol) and triethyl amine (0.38 mL, 0.42 mmol) were combined in 1.0 mL ethanol and heated with magnetic stirring overnight at 80° C. Product was isolated by MPLC: silica (hexane/ethyl acetate).

N*6*-(4-Aminomethyl-cyclohexyl)-N*2*-(2-methyl-4-morpholin-4-yl-phenyl)-9H-purine-2,6-diamine (76): N-[4-(2-Chloro-9H-purin-6-ylamino)-cyclohexylmethyl]-2,2,2-trifluoro-acetamide (75) (1.2 g, 3.2 mmol), 2-Methyl-4-morpholin-4-yl-phenylamine (0.92 g, 4.8 mmol) and p-TSA (0.49 g, 2.56 mmol) were combined in 1,4-dioxane (16 mL) and heated at 100° C. overnight. The reaction mixture was evaporated to dryness under reduced pressure. The trifluoroacetamide group was removed by dissolving reaction residue in methanol (16 mL) and stirring with K₂CO₃ (2.2 g, 16 mmol) overnight. The residue obtained after evaporation of the solvent was purified by reverse phase MPLC (ACN/H₂O, 0.1% TFA).

1-Ethyl-3-{4-[2-(2-methyl-4-morpholin-4-yl-phenylamino)-9H-purin-6-ylamino]-cyclohexylmethyl}-urea (C24): N*6*-(4-Aminomethyl-cyclohexyl)-N*2*-(2-methyl-4-morpholin-4-yl-phenyl)-9H-purine-2,6-diamine (75 mg, 0.14 mmol) was dissolved in DMF (0.700 mL). Diisopropylethyl amine (0.0275 mL, 0.41 mmol) and ethyl isocyanate (0.013 mL, 0.17 mmol) were added and the reaction stirred overnight at ambient temperature. The reaction mixture was purified by reverse phase HPLC to get compound (Table 1, C24).

In Table 1, compounds C22, C35, and C36 were prepared from compound 76 and appropriate acid or acid chloride derivative. Compound C21 (Table 1) N*6*-(4-Amino-cyclohexyl)-N*2*-(2-methyl-4-morpholin-4-yl-phenyl)-9H-purine-2,6-diamine was prepared employing similar conditions described above and was coupled with trifluoracetic acid to get C21.

### Synthesis of N-{4-[2-(2,4-Dimethoxy-phenylamino)-9H-purin-6-ylamino]-cyclohexylmethyl}-methanesulfonamide (C13)

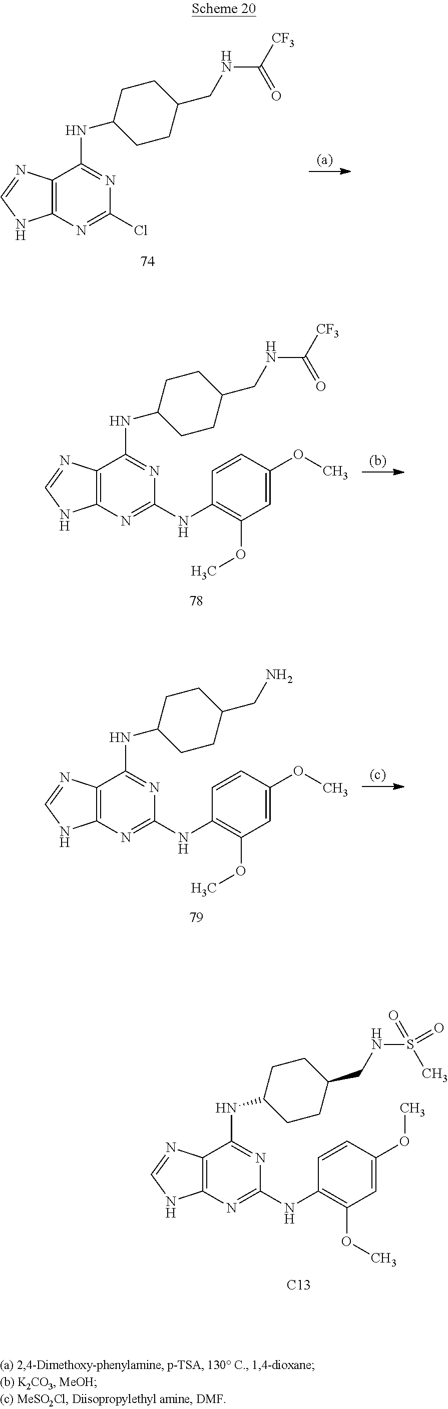

N*6*-(4-Aminomethyl-cyclohexyl)-N*2*-(2,4-dimethoxy-phenyl)-9H-purine-2,6-diamine (79): The compound 74 was converted to 78 using 2,4-Dimethoxy-phenylamine (commercially available from Aldrich) employing similar conditions as described in Scheme 19.

N-{4-[2-(2,4-Dimethoxy-phenylamino)-9H-purin-6-ylamino]-cyclohexylmethyl}-methanesulfonamide (C13): N*6*-(4-Aminomethyl-cyclohexyl)-N*2*-(2,4-dimethoxy-phenyl)-9H-purine-2,6-diamine 79 (28 mg, 0.055 mmol) was dissolved in DMF (0.27 mL). Diisopropylethyl amine (0.020 mL, 0.11 mmol) and methanesulfonyl chloride (6.87 mg, 0.060 mmol) were added and the reaction stirred overnight at ambient temperature to get compound (Table 1, C13), which was purified by reverse phase HPLC.

### Synthesis of 4-(6-Cyclohexylamino-9H-purin-2-ylamino)-3-methyl-N-(2-pyridin-3-yl-ethyl)-benzamide (Table 1, C86)

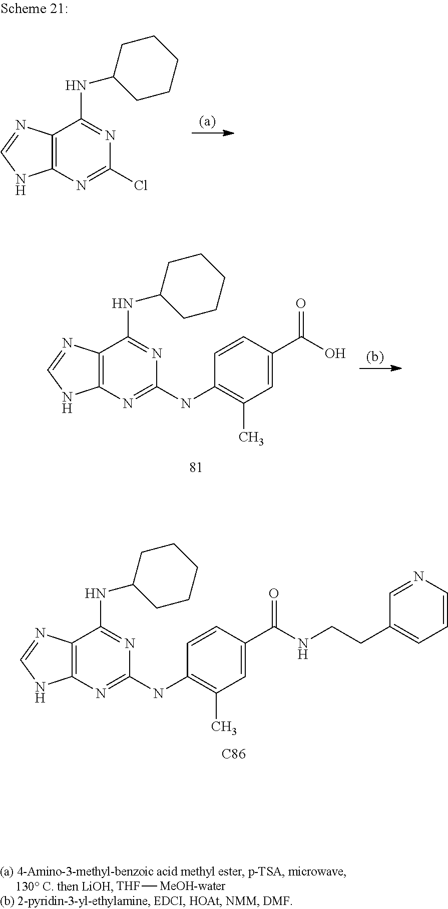

4-(6-Cyclohexylamino-9H-purin-2-ylamino)-3-methyl-benzoic acid (81): A suspension of (2-chloro-9H-purin-6-yl)cyclohexylamine 1 (0.20 g, 0.80 mmol), 4-amino-3-methylbenzoic acid methyl ester (0.20 g, 1.20 mmol), and p-TSA (0.15 g, 0.80 mmol) in of 1,4-dioxane (1.0 mL) was irradiated at 130° C. for 3 hours in a microwave and then was concentrated and purified by silica gel chromatography to provide intermediate ester (0.11 g, 36%). The above ester was hydrolyzed in a solution of lithium hydroxide (10 equiv.) in THF:MeOH:water (2:1:1) The solution was stirred for 16 hours at room temperature, then cooled in an ice bath and then 2 N HCl solution was slowly added to afford a precipitate. The precipitate 81 was collected, dried, and then used in the next reaction without purification.

4-(6-Cyclohexylamino-9H-purin-2-ylamino)-3-methyl-N-(2-pyridin-3-yl-ethyl)benzamide (C86): A mixture of 4-(6-cyclohexylamino-9H-purin-2-ylamino)-3-methylbenzoic acid 81 (50 mg, 0.14 mmol), 2-pyridin-3-yl-ethylamine (20 mg, 1.2 equiv.), N-(3-dimethylaminopropyl)-N′-ethylcarbodiimide hydrochloride (EDCI.HCl) (32 mg, 1.2 equiv.), and 1-hydroxy-7-azabenzotriazole (HOBt) (11 mg, 0.6 equiv.) was dissolved in 2.0 mL of DMF and then N-methylmorpholine (43 mg, 3 equiv.) was added at room temperature. The mixture was stirred for 15 hours at room temperature then diluted with 5 mL of water to provide a precipitate, which was collected by filtration and purified by silica gel chromatography to give the title compound (Table 1, C86) (51 mg, 80%).

In Table 1, compounds C26, C42, C43, C73, C74, C75, and C87 were prepared according to the procedure described above starting from compound 81 and appropriate amine derivative.

### Synthesis of (6-Cyclohexylmethyl-9H-purin-2-yl)-(2-methoxy-4-morpholin-4-yl-phenyl)-amine ((Table 1, C89)

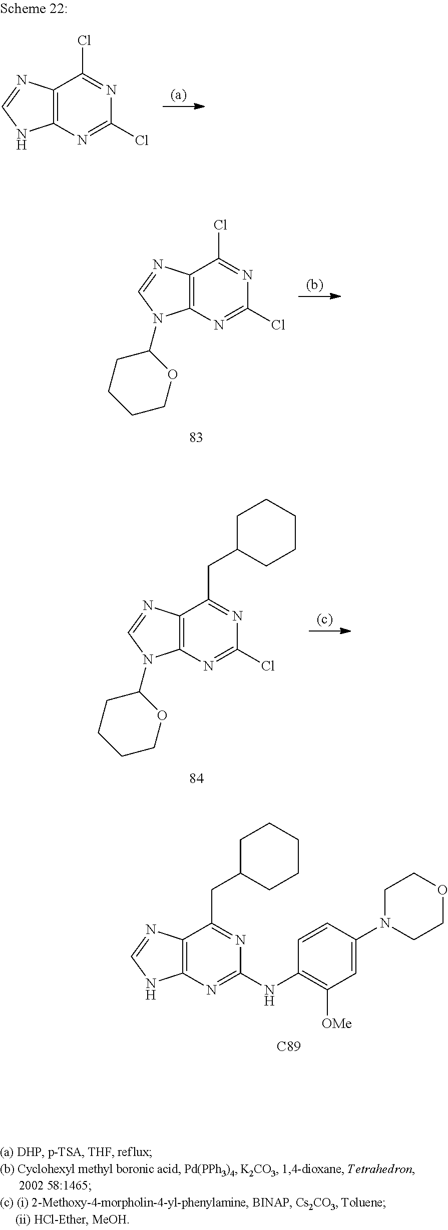

C-6 substituted carbon analogs such as compound C89 can be prepared from 2,6-dichloropurine in two steps via protection of N-7 of purine derivative with protecting groups such as dihydropyran, MEM, p-Toluene sulfonyl group etc, followed by transistion metal (such as palladium) catalyzed Buchwald coupling with appropriate aniline derivative as shown in Scheme 12.

2,6-Dichloro-9-(tetrahydro-pyran-2-yl)-purine (83): To a solution of 2,6-dichloropurine (1 g, 5 mmol) in THF (50 mL) was added p-TSA (0.2 g, 1 mmol) and 3,4 dihydro-2H-pyran (1.4 mL, 16 mmol). The reaction was heated to reflux and allowed to stir for 18 hours. The reaction was concentrated and partitioned between ethyl acetate (100 mL) and a solution of saturated citric acid (100 mL). The organic layer was collected, washed with a saturated solution of NaHCO₃ (100 mL) then brine (100 mL). The organic layer was dried (Na₂SO₄), concentrated in vacuo, and purified via silica gel column chromatography (Hexane:Ethyl acetate) to afford the desired product 83 (1.1 g) as a white solid.

2-Chloro-6-cyclohexylmethyl-9-(tetrahydro-pyran-2-yl)-purine (84): Compound 84 was synthesized using Suzuki cross-coupling reaction conditions as described in Tetrahedron, 2002, 58:1465. To a suspension of 83 (75 mg, 0.28 mmol), the boronic acid (39 mg, 0.28 mmol), K₂CO₃ (110 mg, 0.82 mmol) in 1,4-dioxane (3 mL) was added Pd(PPh₃)₄ under nitrogen. The reaction was heated to reflux for 24 hours. The reaction was concentrated and purified via silica gel column chromatorgraphy (Hex: ethyl acetate) to afford the desired product 84 (49 mg) as a light yellowish oil.

(6-Cyclohexylmethyl-9H-purin-2-yl)-(2-methoxy-4-morpholin-4-yl-phenyl)-amine (C89): To a suspension BINAP (11 mg, 0.23 mmol) and Palladium acetate (3 mg, 0.15 mmol) in toluene (1 mL) under nitrogen was added the 2-methoxy-4-morpholin-4-yl-phenylamine (25 mg, 0.9 mmol), Cs₂CO₃ (120 mg, 0.37 mmol) and a solution of 84 (25 mg, 0.8 mmol) in toluene (1 mL). The suspension was degassed with nitrogen for 5 minutes then heated to 100° C. for 16 hours. The reaction was concentrated then partitioned between ethyl acetate (20 mL) and water (10 mL). The organic layer was collected, washed with water (10 mL) then concentrated to affored the desired N-protected intermediate used without further purification. To the N-protected intermediate was added MeOH (5 mL) then 2N HCl in ethyl ether (0.5 mL). The reaction was stirred for 4 hours at which point no starting material remained. The reaction was concentrated and purified via preparative HPLC to afford the desired product (Table 1, C89) (16 mg) as light brown solid.

### Synthesis of N*6*-Cyclohexyl-N*2*-[2-methyl-4-(3-morpholin-4-yl-prop-1-ynyl)-phenyl]-9H-purine-2,6-diamine (Table 1, C91)

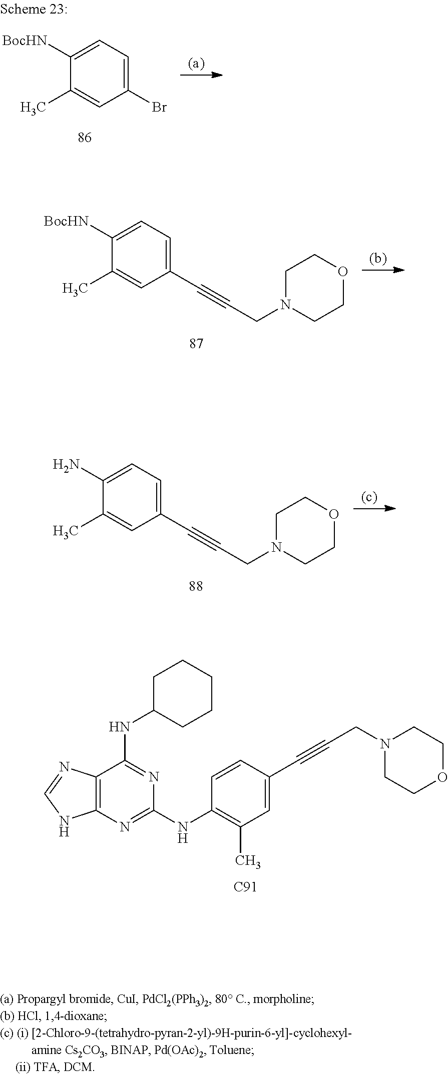

4-[3-(3,4-Dimethyl-phenyl)-prop-2-ynyl]-morpholine (87): Compound 87 was synthesized using Sonagashira cross-coupling reaction conditions as described in Tetrahedron 2007, 63, 10671. To a solution of the Aryl bromide 86 (0.5 g, 1.74 mmol), CuI (0.03 g, 0.174 mmol), PdCl₂(PPh₃)₂ (0.06 g, 0.087 mmol) in morpholine (15 mL) cooled to 0° C. was added propargyl bromide (0.32 mL, 2.1 mmol) semi-drop wise. The reaction was allowed to warm to room temperature, then heated to 80° C. for 16 hours. The resulting suspension was concentrated in vacuo and purified via silica column chromatography (Hexane:Ethyl acetate) to afford the desired product as an orange oil and it was used without further purification.

2-Methyl-4-(3-morpholin-4-yl-prop-1-ynyl)-phenylamine (88): To a solution of 87 (130 mg, 0.39 mmol) in 1,4-dioxane (10 mL) was added HCl in 1,4-dioxane (1.0 mL, 3.9 mmol) semi-drop wise. The reaction was allowed to stir for 6 hours then concentrated concentrated in vacuo to afford the desired product 88 and used without further purification.

N*6*-Cyclohexyl-N*2*-[2-methyl-4-(3-morpholin-4-yl-prop-1-ynyl)-phenyl]-9H-purine-2,6-diamine (C91): To a suspension BINAP (48 mg, 0.77 mmol) and palladium acetate (3 mg, 0.5 mmol) in toluene (1 mL) under nitrogen was added a suspension of the aniline 88 (135 mg, 0.44 mmol), Cs₂CO₃ (560 mg, 1.72 mmol) and the [2-Chloro-9-(tetrahydro-pyran-2-yl)-9H-purin-6-yl]-cyclohexyl-amine 43 (115 mg, 0.34 mmol) in toluene (1 mL). The suspension was degassed with nitrogen for 5 minutes then heated to 100° C. for 16 hours. The reaction was concentrated in vacuo. The resulting residue was taken up in MeOH (10 mL) and then filtered over Celite and concentrated. The resulting residue was taken up in DCM (10 mL) and cooled to 0° C. followed by addition of TFA (1 mL). After stirring for 1 hour at room temperature, the reaction was concentrated and purified via preparative HPLC to afford the desired product (Table 1, C91). In the Table 2, compounds C101-C143 were prepared according to the similar procedure described above, with appropriately substituted starting materials.

### Synthesis of [2-(2-Methoxy-4-morpholin-4-yl-phenylamino)-9H-purin-6-yl]-morpholin-4-yl-methanone (Table 1, C90)

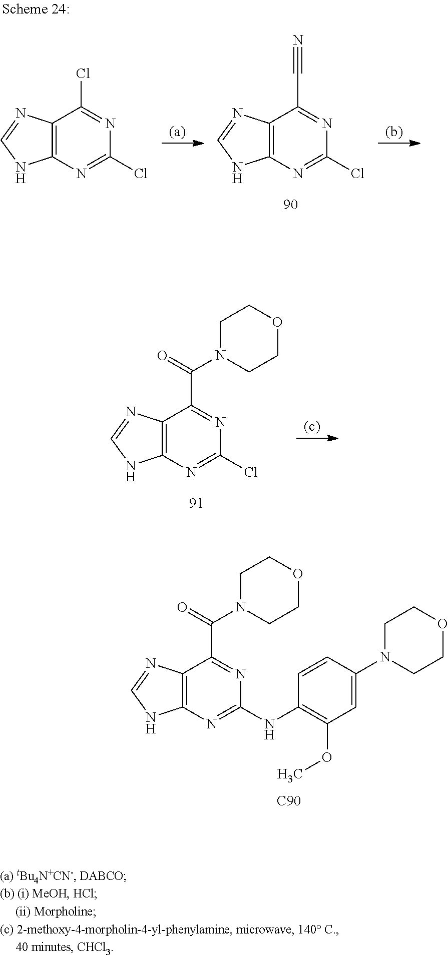

2-Chloro-9H-purine-6-carbonitrile (90): To a solution of 2,6-dichloropurine (0.5 g, 2.7 mmol) in acetonitrile (15 mL) was added tetrabutylammonium cyanide (1.1 g, 4.0 mmol) and DABCO (446 mg, 4.0 mmol). The reaction was stirred overnight after which it was extracted with chloroform and washed with water. Hexanes was added to the organic partition was causing the product to precipitate from solution as a brownish yellow solid. The solid was isolated by filtration giving compound 90.

(2-Chloro-9H-purin-6-yl)-morpholin-4-yl-methanone (91): Compound 90 was dissolved in concentrated anhydrous HCl in methanol and stirred for 1 hour after which the solvent was removed in vacuo and ˜2 mL of morpholine was added. The reaction was stirred for ˜2 hours and the excess morpholine was removed in vacuo to give compound 91 as the crude product.

[2-(2-Methoxy-4-morpholin-4-yl-phenylamino)-9H-purin-6-yl]-morpholin-4-yl-methanone (C90): Compound 91 and the 2-methoxy-4-morpholin-4-yl-phenylamine were combined with sodium acetate and chloroform and exposed to microwave irradiation in the previously described manner (Scheme 2) to give compound (Table 1, C90), which was then purified by reverse phase semi-prep HPLC.

### Synthesis of (6-Cyclohexylsulfanyl-9H-purin-2-yl)-(2-methoxy-4-morpholin-4-yl-phenyl)-amine (96) and 6-cyclohexylsulfonyl-N-(2-methoxy-4-morpholino-phenyl)-9H-purin-2-amine (97)

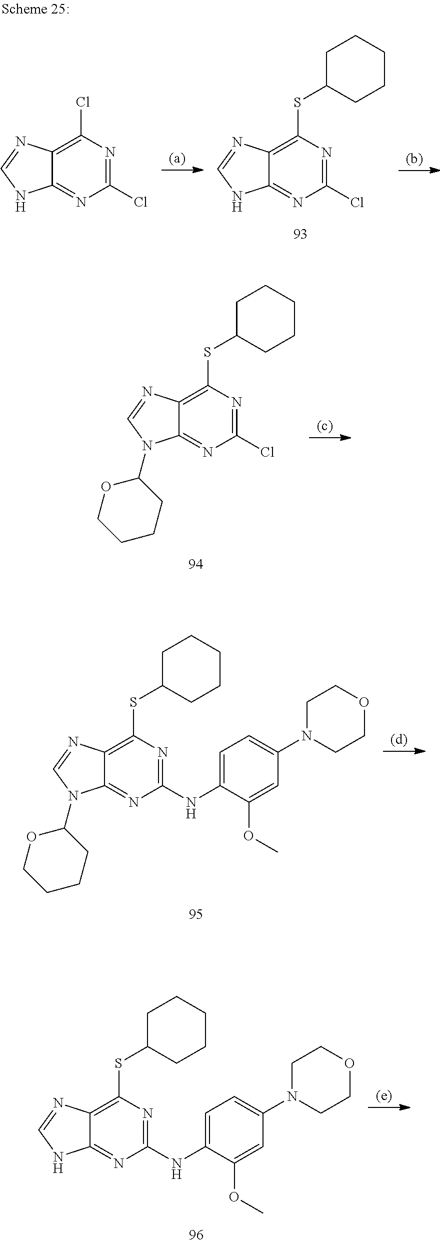

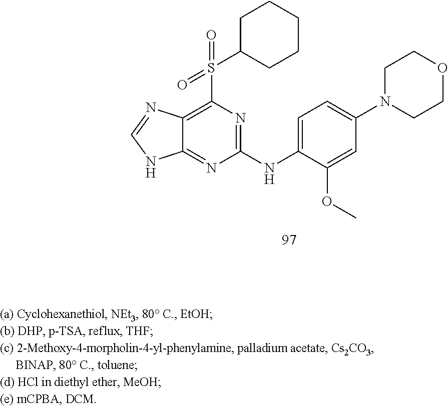

2-Chloro-6-cyclohexylsulfanyl-9H-purine (93): 2,6-Dichloropurine (3.9054 g, 0.02 mol) was added to a round bottom flask containing ethanol (70 mL). To this was added compound cyclohexanethiol (2.8 mL, 0.22 mol), and triethylamine (5.8 mL, 0.413 mol). The reaction mixture was allowed to stir at 90° C. for 18 hours. The reaction was cooled and the precipitate was filtered and washed with ethanol to give 3.7721 g of 93 (0.014 mol, 70% yield). Mass Spec (m/z): 268.9 (M+1).

[6-Cyclohexylsulfanyl-9-(tetrahydro-pyran-2-yl)-9H-purin-2-yl]-(2-methoxy-4-morpholin-4-yl-phenyl)-amine (94): Compound 93 (0.8135 g, 0.003 mol) was added to a round bottom flask containing THF (92 mL), followed by the addition of DHP (1.53 g, 0.018 mol) and p-TSA (0.086 g, 0.45 mmol) and heated to 75° C. for 18 hours. The reaction was extracted with ethyl acetate and water. The crude mixture was purified on silica gel to give 0.96 grams of product 94 (0.0027 mol, 90% yield). Mass Spec (m/z): 353.2 (M+1).

(6-Cyclohexylsulfanyl-9H-purin-2-yl)-(2-methoxy-4-morpholin-4-yl-phenyl)-amine (96): Dry toluene (5 mL) was added to a flame dried vial, followed by Pd(OAC)₂, (0.026 g, 0.115 mmol) and BINAP (0.105 g, 0.16 mmol). Nitrogen was bubbled through the solvent for a minimum of 30 minutes. Compound 94 (0.271 g, 0.76 mmol), compound 2-Methoxy-4-morpholin-4-yl-phenylamine (0.1761 g, 0.8 mmol, 1.1 eq), and Cs₂CO₃ (1.0 g, 0.003 mol) were added to the reaction mixture and heated to 100° C. for 18 hours. The reaction mixture was concentrated and then purified on a silica gel column eluting with hexanes and ethyl acetate to give of product 95 (0.122 g) (0.2 mmol, 30% yield). Mass Spec (m/z): 525.2 (M+1). Compound 95 (0.122 g, 0.2 mmol) was stirred in HCl in ethyl ether (1 mL), and dry methanol (6 mL) for 3 hours to give compound 96 in quantitative yield. Mass Spec (m/z): 441.2 (M+1).

(6-Cyclohexanesulfonyl-9H-purin-2-yl)-(2-methoxy-4-morpholin-4-yl-phenyl)-amine (97): Compound 96 was oxidized employing typical oxidative conditions with mCPBA in dichloromethane at room temperature to get compound 97.

### Synthesis of 1-[1-[4-[[6-(cyclohexylamino)-9H-purin-2-yl]amino]-3-methoxy-phenyl]-4-piperidyl]piperidin-3-ol (C144)

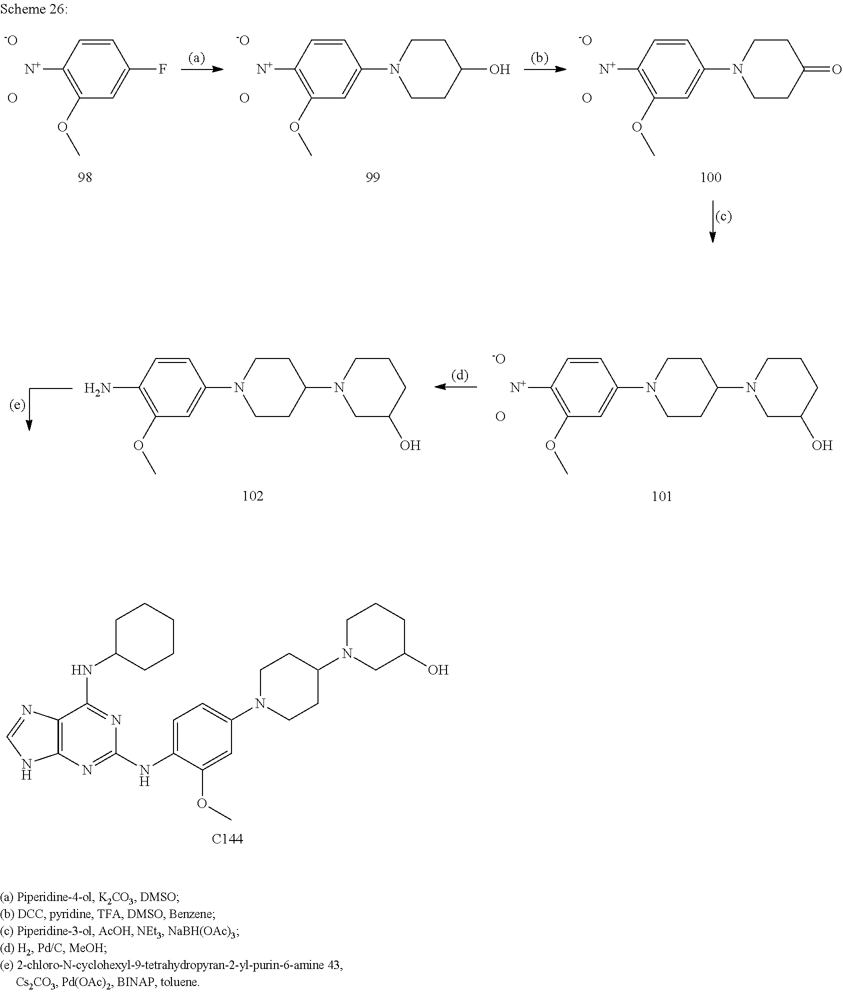

1-(3-methoxy-4-nitro-phenyl)piperidin-4-ol (99): To a solution of compound 98 (4.8 g, 0.028 mol, 1 eq) in 60 mL of DMSO was added piperidine-4-ol (2.84 g, 0.028 mol, 1 eq) and potassium carbonate (7.7 g, 0.056 mol, 2 eq). The reaction mixture was stirred at room temperature for 18 hours. Water was added to the reaction mixture until a precipitate formed. The precipitate was then filtered and washed several times with water to give an off white solid product 99 (5.3 g, 75% yield).

1-(3-methoxy-4-nitro-phenyl)piperidin-4-one (100): To a solution of DMSO (1.75 mL), benzene (3.25 mL), and 1 M dicyclohexylcarbodiimide (3.57 mL, 0.00357 mol, 3 eq) in a round bottom flask was added compound 99 (0.30 g, 0.0019 mol, 1 eq). The reaction mixture was cooled to 0° C., at which time pyridine (0.1 mL), and trifluoroacetic acid (0.05 mL) was added. The reaction stirred from 0° C. to room temperature for 18 hours. Ethyl acetate was added to the reaction mixture to precipitate out the dicyclohexylurea. The dicyclohexylurea was filtered off and the filtrate was washed 3 times with water, dried over magnesium sulfate, and then the organic solvent was concentrated. The final product was purified by reverse phase column chromatography to give compound 100 (0.29 g, 97% yield).

1-[1-(3-methoxy-4-nitro-phenyl)-4-piperidyl]piperidin-3-ol (101): To a round bottom flask was added compound 100 (0.50 g, 0.002 mol, 1 eq), dichloromethane (10 mL), piperidine-3-ol (0.55 g, 0.004 mol, 2 eq), acetic acid (0.17 mL, 0.003 mol, 1.5 eq), and triethylamine (0.42 mL, 0.003 mol, 1.5 eq) and stirred at room temperature for a minimum of 0.5 hours. Triacetoxyborohydride (0.51 g, 0.0024 mol, 1.2 eq) was added and continued stirring at room temperature for 18 hours. The reaction mixture was then neutralized with saturated sodium bicarbonate, and then extracted with dichloromethane to give the final product 101 was without further purification (0.4880 g, 0.0145 mol, 75% yield).

1-[1-(4-amino-3-methoxy-phenyl)-4-piperidyl]piperidin-3-ol (102): To a Parr shaker flask was added palladium on carbon (0.10 g) followed by methanol (30 mL), and compound 101 (0.4880 g, 0.0014 mol, 1 eq). The flask was placed on the Parr shaker under hydrogen atmosphere at 45 psi for 18 hours. The reaction mixture was filtered through Celite and the organic layer was concentrated to give the final product 102 (0.295 g, 69% yield).

1-[1-[4-[[6-(cyclohexylamino)-9H-purin-2-yl]amino]-3-methoxy-phenyl]-4-piperidyl]piperidin-3-ol (C144): 1-[1-(4-amino-3-methoxy-phenyl)-4-piperidyl]piperidin-3-ol 102 and 2-chloro-N-cyclohexyl-9-tetrahydropyran-2-yl-purin-6-amine 43 were coupled according the procedure described in Scheme 12 to get C144.

Compounds C145, C146, C147, C148, C154 and C155 in the Table 2, were prepared according to the procedure described above with appropriately substituted starting materials.

### Synthesis of N6-cyclohexyl-N2-[2-methoxy-4-(1-piperidyl)phenyl]-9H-purine-2,6-diamine (C156)

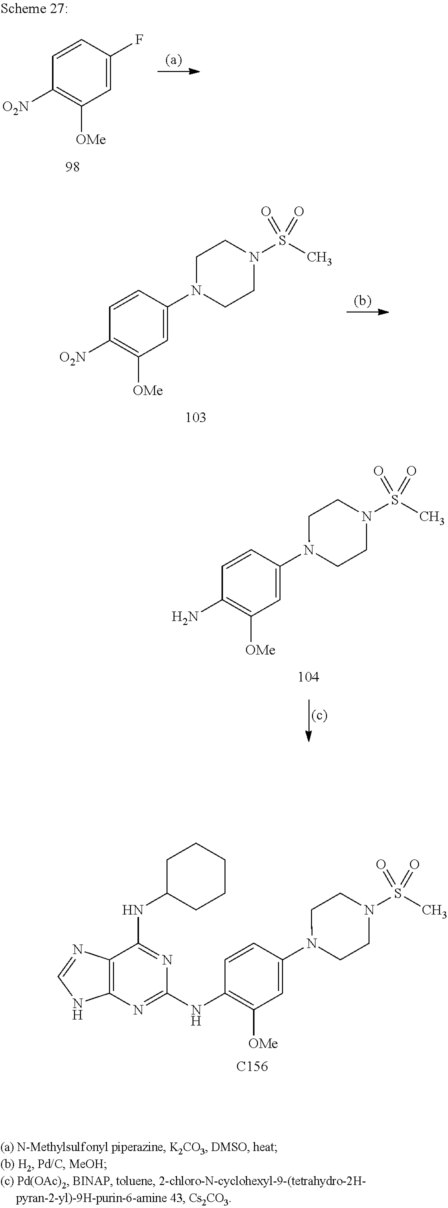

1-(3-methoxy-4-nitro-phenyl)-4-methylsulfonyl-piperazine (103): To a solution of 4-fluoro-2-methyl-1-nitrobenzene 98 (2.2 g, 12.7 mmol) and N-methylsulfonyl piperazine (2.5 g, 15.2 mmol) in DMSO (35 mL) was added potassium carbonate (2.1 g, 15.2 mmol). The reaction was heated to 80° C. for 21 hours. Upon cooling, water (250 mL) was added to the reaction followed by extraction with diethyl ether (2×250 mL). The combined organics were dried (Na₂SO₄) and concentrated in vacuo to afford 103 (5.37 g, >100% yield) used without further purification.

2-methoxy-4-(4-methylsulfonylpiperazin-1-yl)aniline (104): To a suspension of 103 (4.5 g, 14.3 mmol) in MeOH (200 mL) was added 10% Pd/C (0.5 g) and hydrogenated for 22 hours at 1 atmospheric pressure. The Pd/C was filtered over a pad of Celite and the solvent removed in vacuo. The residue was dissolved in minimal MeOH and a solution of 1M HCl/ether added to precipitate the compound. The solvents were removed in vacuo and the remaining solid triturated with diethyl ether to afford 104 as the HCl salt (4.5 g, 97% yield) used without further purification.

N6-cyclohexyl-N2-[2-methoxy-4-(1-piperidyl)phenyl]-9H-purine-2,6-diamine (C156): 2-methoxy-4-(4-methylsulfonylpiperazin-1-yl)aniline 104 and 2-chloro-N-cyclohexyl-9-tetrahydropyran-2-yl-purin-6-amine 43 were coupled according to the procedure described in Scheme 12 to get C156.

In the Table 2, C157 and C158 were prepared in a similar way as described above, using appropriately substituted starting material in the Buchwald coupling step.

### Synthesis of N6-cyclohexyl-N2-(2-methoxy-4-piperazin-1-yl-phenyl)-9H-purine-2,6-diamine (106)

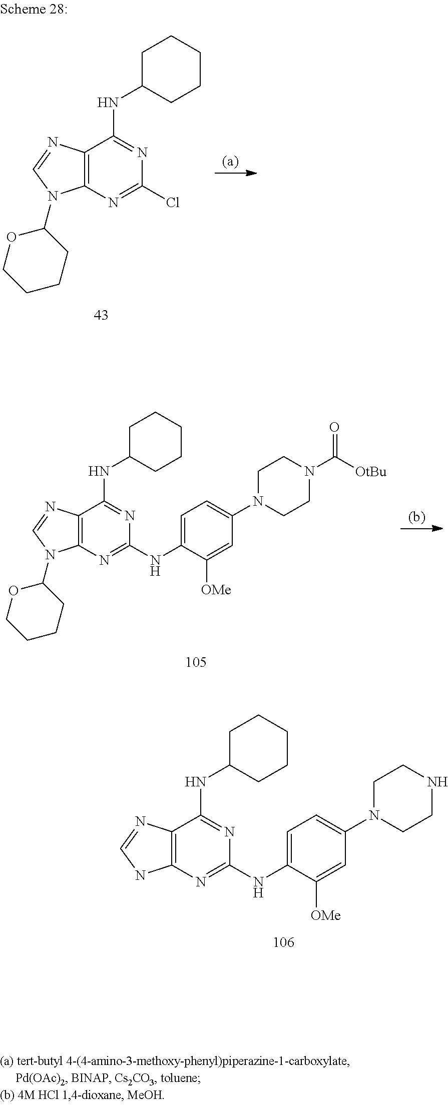

To a degassed stirred solution of Pd(OAc)₂ (0.23 g, 0.001 mol) and BINAP (1.06 g, 0.002 mol) in anhydrous toluene (70 mL) under a nitrogen atmosphere was added 2-chloro-N-cyclohexyl-9-(tetrahydro-2H-pyran-2-yl)-9H-purin-6-amine 43 (2.29 g, 0.07 mol), Cs₂CO₃ (6.66 g, 0.2 mmol) and tert-butyl 4-(4-amino-3-methoxy-phenyl)piperazine-1-carboxylate (2.3 g, 0.08 mol) (prepared from compound 48 and tert-butyl piperazine-1-carboxylate as described in Scheme 27). The mixture was heated to 90° C. for 22 hours under nitrogen atmosphere. The reaction was concentrated and suspended between 250 mL DCM and 250 mL water. The layers were separated and the aqueous layer was extracted with Ethyl acetate (2×200 mL). The combined organic layers were dried (sodium sulfate) and concentrated in vacuo. The resulting residue was purified by silica gel flash chromatography using MeOH/DCM as eluent (0-10%) to afford the desired 105 as a brown solid (3.9 g).

To a solution of 105 (3.9 g) in 1,4-dioxane (20 mL) was added 4M HCl in 1,4-dioxane solution (18 mL). The solution was stirred at room temperature and MeOH was added to keep the solids in solution. After stirring for 3 hours, the reaction was determined to be complete by LCMS analysis. The reaction was concentrated in vacuo and then suspended in ethyl acetate/diethyl ether solution (100 mL of a 3:1 mixture). The precipitate was collected and placed under vacuum to afford the desired product 106 as a tan solid (2.8 g).

C159 and C160 in Table 2 were also prepared according to the similar procedure described above starting from tert-butyl 3,6-diazabicyclo[2.2.1]heptane-3-carboxylate.

### Synthesis of N6-cyclohexyl-N2-[2-methoxy-4-[4-(2-pyrrolidin-1-ylethyl)piperazin-1-yl]phenyl]-9H-purine-2,6-diamine (C161)

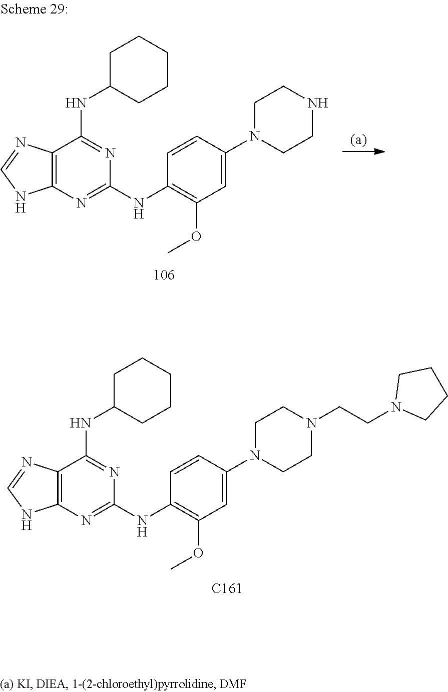

To a solution of N6-cyclohexyl-N2-(2-methoxy-4-piperazin-1-yl-phenyl)-9H-purine-2,6-diamine 106 (95 mg, 0.19 mmol) and 1-(2-chloroethyl)pyrrolidine (36 mg, 0.21 mmol) in DMF (2 mL) was added a catalytic amount of KI and DIEA (0.13 mL, 0.77 mmol). The reaction was heated to 100° C. and allowed to stir for 24 hours. The reaction was monitored by LCMS for completion, the solvents removed, and the residue was purified by reverse phase flash chromatography with MeOH/H₂O/0.1% TFA as eluent to provide the desired product C161.

In the Table 2, C136, C162, C163, C189 and C192 were prepared according to the similar procedure described above using appropriate alkyl halide.

### Synthesis of 4-[4-[[6-(cyclohexylamino)-9H-purin-2-yl]amino]-3-methoxy-phenyl]-N-cyclopentyl-piperazine-1-carboxamide (C164)

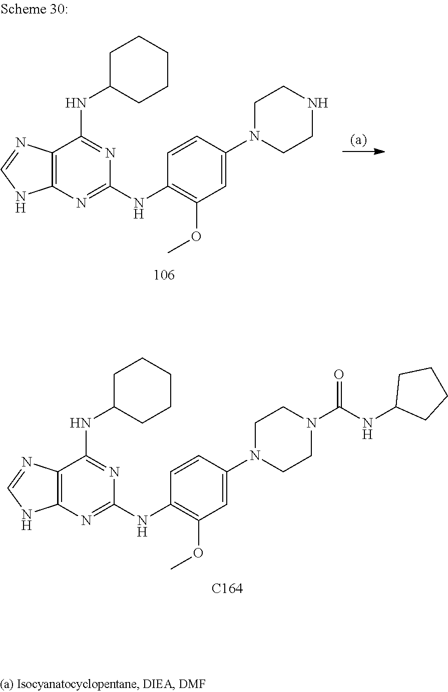

To a solution of 106 (80 mg, 0.16 mmol) in DMF (1 mL) was added DIEA (0.11 mL, 0.65 mmol). After stirring for 5 minutes the desired isocyanate was added to the reaction and stirred at room temperature. The reaction was monitored by LCMS for completion and after 2 hours the solvents removed and the residue purified by reverse phase flash chromatography with MeOH/H₂O/0.1% TFA as eluent to provide the desired product C164.

Compounds C167 and C170 in the Table 2 were prepared according the procedure described above using appropriately substituted isocyanate.

### Synthesis of 1-[4-[4-[[6-(cyclohexylamino)-9H-purin-2-yl]amino]-3-methoxy-phenyl]piperazin-1-yl]-2-(4-methylpiperazin-1-yl)ethane-1,2-dione (C171)

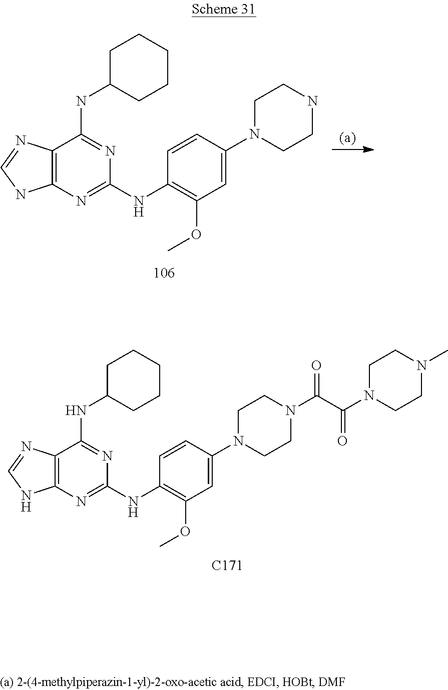

To 2-(4-methylpiperazin-1-yl)-2-oxo-acetic acid (44 mg, 0.25 mmol) in DMF (2 mL) was added DIEA (0.12 mL, 0.71 mmol) followed by HOBt-H₂O (39 mg, 0.25 mmol) and EDCI-HCl (48 mg, 0.25 mmol). After stirring for 5 minutes 106 (100 mg, 0.20 mmol) was added to the solution and allowed to stir at room temperature for 18 hr. The reaction was concentrated and the residue purified by reverse phase flash chromatography with MeOH/H₂O/0.1% TFA as eluent to provide the desired product C171.

### Synthesis of N6-cyclohexyl-N2-[2-methoxy-4-[4-[3-(4-methylpiperazin-1-yl)propylsulfonyl]piperazin-1-yl]phenyl]-9H-purine-2,6-diamine (C172) And N6-cyclohexyl-N2-[4-(4-ethylsulfonylpiperazin-1-yl)-2-methoxy-phenyl]-9H-purine-2,6-diamine (C177)

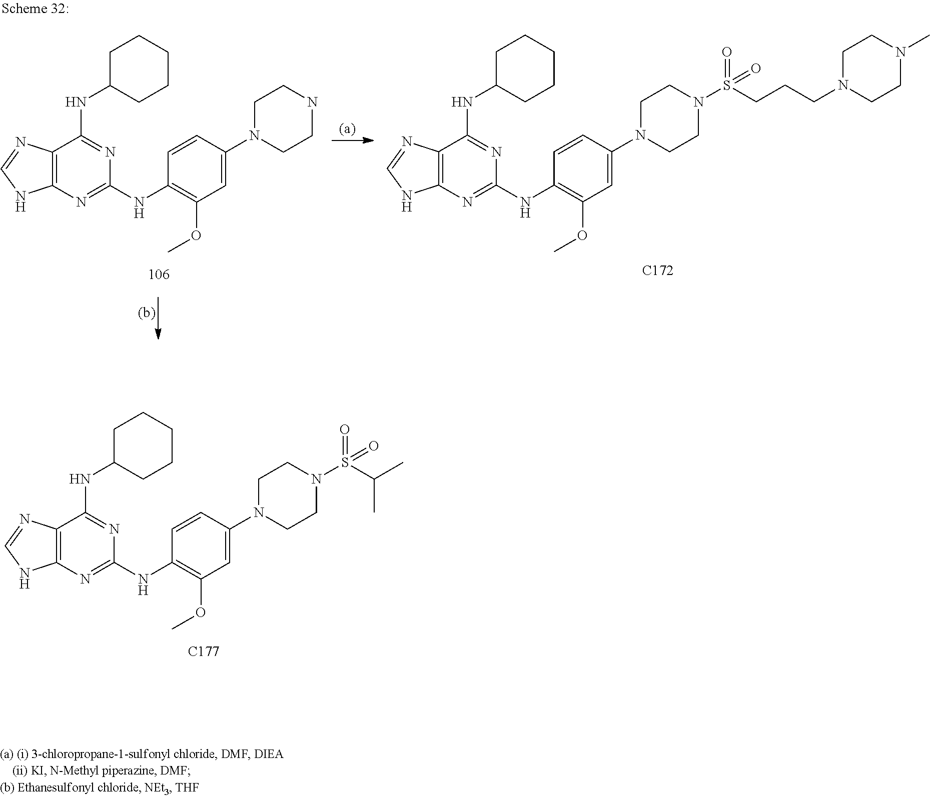

N6-cyclohexyl-N2-[2-methoxy-4-[4-[3-(4-methylpiperazin-1-yl)propylsulfonyl]piperazin-1-yl]phenyl]-9H-purine-2,6-diamine (C172): To a solution of 106 (100 mg, 0.20 mmol) in DMF (2 mL) was added DIEA (0.11 mL, 0.65 mmol). After stirring for 5 minutes the desired sulfonyl chloride (0.029 mL, 0.022 mmol) was added drop wise to the reaction and stirred at room temperature for 6 hours. To the reaction mixture was added a catalytic amount of KI and the N-Methyl piperazine (0.23 mL, 2.0 mmol). The reaction was heated to 80° C. and allowed to stir for 18 hours. The reaction was concentrated and the residue purified by reverse phase flash chromatography with MeOH/H₂O/0.1% TFA as eluent to provide the desired product C172.

N6-cyclohexyl-N2-[4-(4-isopropylsulfonylpiperazin-1-yl)-2-methoxy-phenyl]-9H-purine-2,6-diamine (C177): To a solution of 106 (bis HCl salt) (70 mg, 0.14 mmol) in DMF (1.5 mL) was added diisopropyl ethyl amine (93 μL, 0.57 mmol) stirred for 5 minutes. Isopropylsulfonyl chloride (22 μL, 0.16 mmol) was added and stirred at room temperature for 18 hours. The residue obtained after solvent evaporation was purified using reverse phase HPLC to get C177.

Compounds C172 to C181 in the Table 2, were prepared according to the procedure describe above using appropriate halo alkyl sulfonyl chloride or sulfonyl chloride and amine.

### Synthesis of 2-[4-[4-[[6-(cyclohexylamino)-9H-purin-2-yl]amino]-3-methoxy-phenyl]piperazin-1-yl]acetamide (C182)

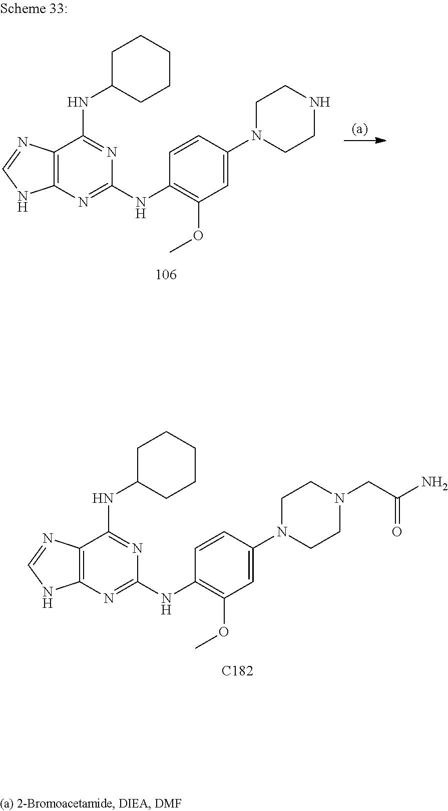

To a solution of 106 (75 mg, 0.15 mmol) in DMF (1 mL) was added DIEA (0.75 mL, 0.45 mmol). After stirring for 5 minutes bromoacetamide (23 mg, 0.17 mmol) was added to the reaction and stirred at room temperature. After stirring for 2 days, the reaction was concentrated and the residue purified by reverse phase flash chromatography with MeOH/H₂O/0.1% TFA as eluent to provide the desired product C182.

### Synthesis of [4-[4-[[6-(cyclohexylamino)-9H-purin-2-yl]amino]-3-methoxy-phenyl]piperazin-1-yl]-(2,2-dimethyltetrahydropyran-4-yl)methanone (C183)

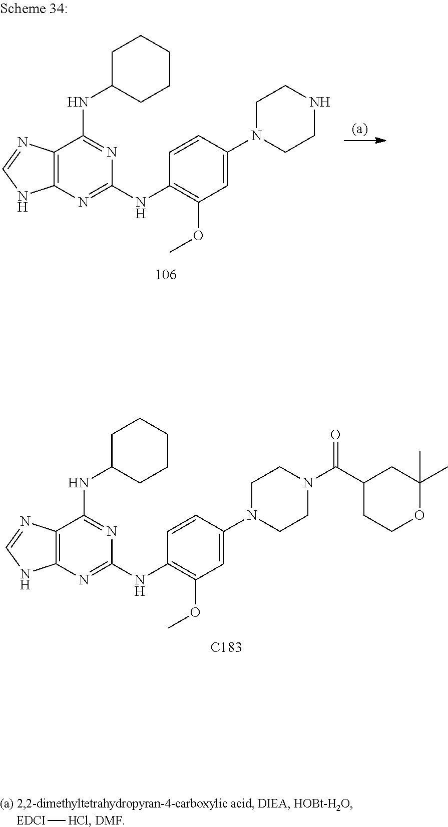

To the 2,2-dimethyltetrahydropyran-4-carboxylic acid (60 mg, 0.38 mmol) in DMF (2 mL) was added DIEA (0.22 mL, 1.29 mmol) followed by HOBt-H₂O (58 mg, 0.38 mmol) and EDCI-HCl (73 mg, 0.38 mmol). After stirring for 5 minutes 106 (150 mg, 0.30 mmol) was added to the solution and allowed to stir at room temperature for 6 hr. The reaction was concentrated and the residue purified by reverse phase flash chromatography with MeOH/H₂O/0.1% TFA as eluent to provide the desired product C183. Compounds C184 to C187 in the Table 2 were prepared according to the procedure described above using appropriate carboxylic acid.

### Synthesis of N6-cyclohexyl-N2-[2-methoxy-4-[4-(tetrahydrofuran-3-ylmethyl)piperazin-1-yl]phenyl]-9H-purine-2,6-diamine (C188)

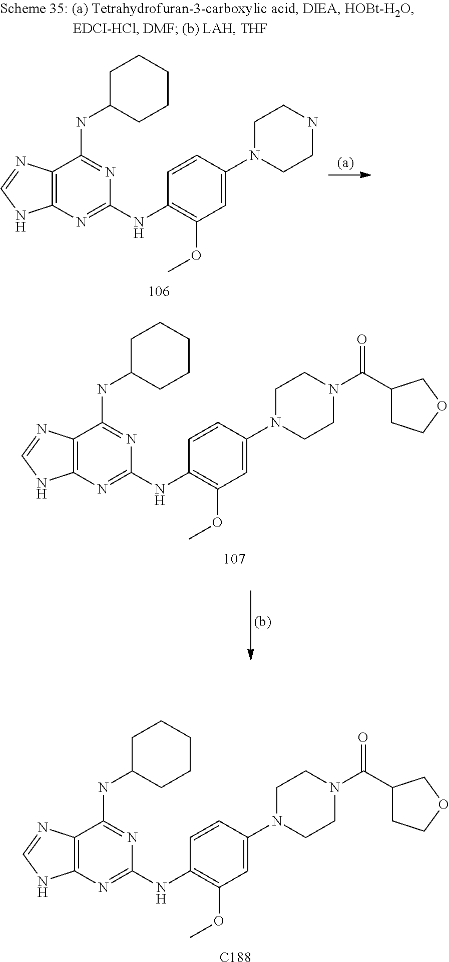

To tetrahydrofuran-3-carboxylic acid (19 μL, 0.19 mmol) in DMF (1 mL) was added DIEA (0.11 mL, 0.64 mmol) followed by HOBt-H₂O (29 mg, 0.19 mmol) and EDCI-HCl (36 mg, 0.19 mmol). After stirring for 5 minutes 106 (75 mg, 0.15 mmol) was added to the solution and allowed to stir at room temperature for 3 hr. The reaction was concentrated and the residue purified by reverse phase flash chromatography with MeOH/H₂O/0.1% TFA as eluent to provide the desired intermediate amide product 107. To the intermediate amide (40 mgs) in THF (1 mL) was added LAH (40 mgs). After stirring for 2 hour at room temperature, the reaction was quenched with addition of MeOH, filtered over Celite, and concentrated to afford a grey residue. The resulting residue was purified by reverse phase flash chromatography with MeOH/H₂O/0.1% TFA as eluent to provide the desired product C188.

### Synthesis of [4-[4-[[6-(cyclohexylamino)-9H-purin-2-yl]amino]-3-methoxy-phenyl]piperazin-1-yl]-(2-pyridyl)methanone (C190) and N6-cyclohexyl-N2-[2-methoxy-4-[4-(2-pyridylmethyl)piperazin-1-yl]phenyl]-9H-purine-2,6-diamine (C191)

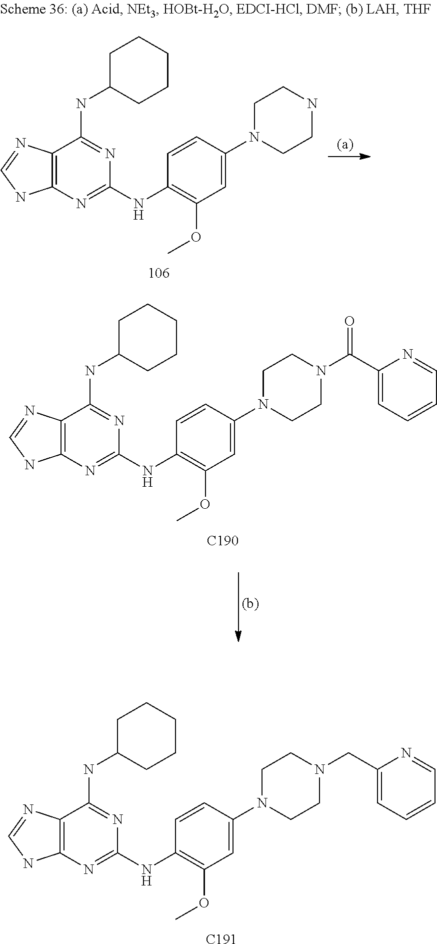

[4-[4-[[6-(cyclohexylamino)-9H-purin-2-yl]amino]-3-methoxy-phenyl]piperazin-1-yl]-(2-pyridyl)methanone (C190): To a solution of 106 (200 mg, 0.4 mmol) in DMF (2.5 mL) was added EDCI.HCl (97 mg, 0.5 mmol), HOBt.H₂O (31 mg, 0.2 mmol), triethylamine (0.239 mL, 1.7 mmol), and finally pyridine-2-carboxylic acid (74 mg, 0.6 mmol). The reaction was allowed to stir at room temperature for 4 hours. The reaction was monitored by LCMS for completion, the solvents removed, and the residue was purified by reverse phase flash chromatography with MeOH/H₂O/0.1% TFA as eluent to provide the desired product C190.

N6-cyclohexyl-N2-[2-methoxy-4-[4-(2-pyridylmethyl)piperazin-1-yl]phenyl]-9H-purine-2,6-diamine (C191: To a solution of C190 (200 mg, 0.38 mmol) in THF (1.0 mL) was added a slurry of LAH (144 mg, 3.8 mmol) in THF (2 mL). The reaction was allowed to stir at room temperature for 18 hours, methanol (5 mL) is added to the reaction mixture and filtered using another addition of methanol (10 mL). This is then concentrated in vacuo, was purified by reverse phase flash chromatography with MeOH/H₂O/0.1% TFA as eluent to provide the desired product C191.

### Synthesis of [4-[4-[[6-(cyclohexylamino)-9H-purin-2-yl]amino]-3-methoxy-phenyl]piperazin-1-yl]-morpholino-methanone (C193)

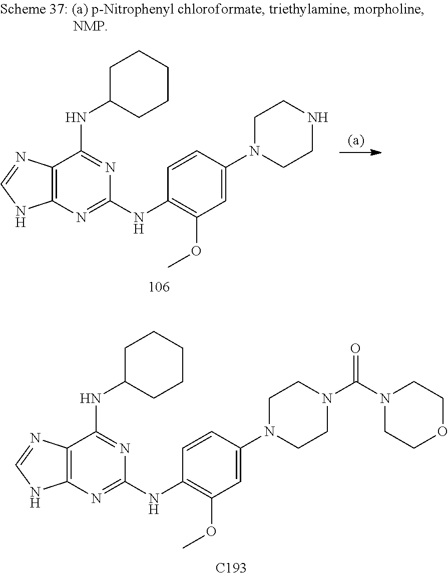

To a solution of 106 (200 mg, 0.4 mmol) in NMP (2.5 mL) was added triethylamine (0.239 mL, 1.7 mmol), p-nitrophenyl chloroformate (90 mg, 0.44 mmol) and finally morpholine (0.05 2 mL, 0.6 mmol). The reaction was allowed to stir at room temperature for 3 hours. The reaction was monitored by LCMS for completion, the solvents removed, and the residue was purified by reverse phase flash chromatography with MeOH/H₂O/0.1% TFA as eluent to provide the desired product C193.

C165, C166, C168 and C169 in the Table 2 were prepared according to the similar procedure described above from appropriately substituted starting materials.

### Synthesis of 2-[4-[4-[[6-(cyclohexylamino)-9H-purin-2-yl]amino]-3-methoxy-phenyl]piperazin-1-yl]-1-(4-methylpiperazin-1-yl)ethanone (C194)

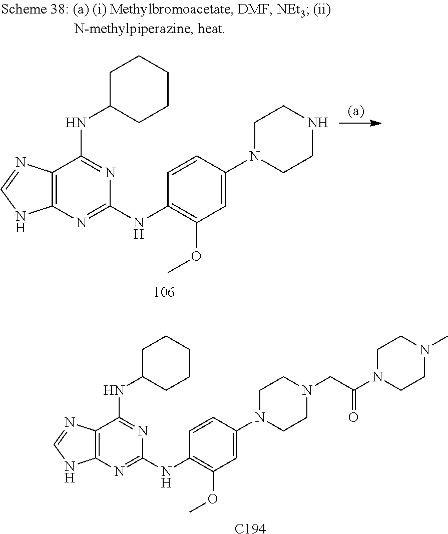

To a solution of 106 (200 mg, 0.4 mmol) in DMF (2.5 mL) was added triethylamine (0.239 mL, 1.7 mmol), and finally methylbromoacetate (0.04 mL, 0.42 mmol). The reaction was allowed to stir at room temperature for 4 hours. The reaction was monitored by LCMS for completion, the solvents removed, and the residue was purified by reverse phase flash chromatography with MeOH/H₂O/0.1% TFA as eluent to provide the desired ester intermediate.

A solution of ester intermediate (not shown) (200 mg, 0.35 mmol) in N-methylpiperazine (0.5 mL) was heated in the microwave at 140° C. for 30 minutes. The residue was purified by reverse phase flash chromatography with MeOH/H₂O/0.1% TFA as eluant to provide the desired product C194.

Compound C195 in Table 2 was prepared according the similar procedure described above using morpholine instead of methyl piperazine in the peptide coupling step.

### Synthesis of N6-cyclohexyl-N2-[2-methoxy-4-(4-morpholinosulfonylpiperazin-1-yl)phenyl]-9H-purine-2,6-diamine (C196)

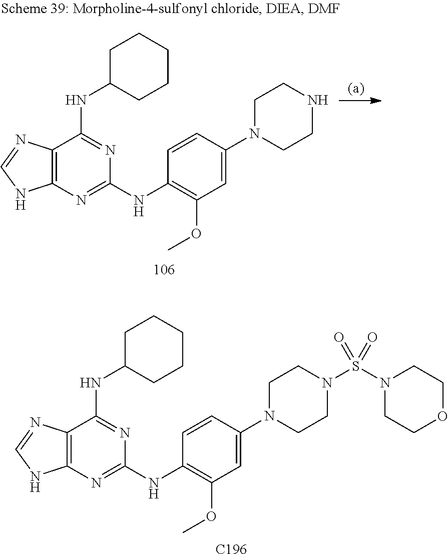

To a solution of the bis-HCl salt of N6-cyclohexyl-N2-(2-methoxy-4-piperazin-1-yl-phenyl)-9H-purine-2,6-diamine 106 (100 mg, 0.20 mmol) in DMF (2 mL) was added DIEA (0.1 mL, 0.67 mmol). After stirring for 5 minutes the desired sulfonyl chloride reagent (0.22 mmol) was added. The reaction was allowed to stir over the weekend, the solvents removed, and the residue was purified by reverse phase flash chromatography with MeOH/H₂O/0.1% TFA as eluant to provide the desired product C196. Compounds C197-C200, in the Table 2, were prepared according the procedure described above using appropriately substituted sulfonyl chlorides.

### Synthesis of N6-cyclohexyl-N2-[2-methoxy-4-[4-(1-methyl-4-piperidyl)piperazin-1-yl]phenyl]-9H-purine-2,6-diamine (C201)

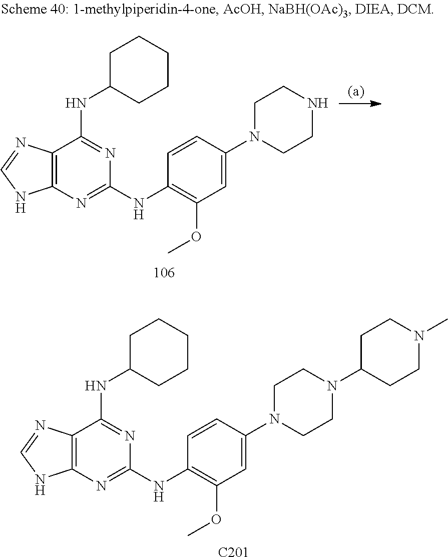

To a solution of the bis-HCl salt of N6-cyclohexyl-N2-(2-methoxy-4-piperazin-1-yl-phenyl)-9H-purine-2,6-diamine 106 (75 mg, 0.15 mmol) in DCM (2 mL) was added DIEA (0.8 mL, 0.45 mmol) followed by 1-methylpiperidin-4-one (0.3 mL, 0.23 mmol) and AcOH (0.05 mL). After stirring for 30 minutes NaBH(OAc)₃ (0.1 g, 0.45 mmol) was added to the reaction. The reaction was allowed to stir over the weekend, quenched with addition of MeOH, the solvents removed, and the residue purified by reverse phase flash chromatography with MeOH/H₂O/0.1% TFA as eluant to provide the desired product C201.

Compound C202 was prepared according to the similar procedure described above using cyclohexanone in the reductive amination step.

### Synthesis of N6-cyclohexyl-N2-[2-methoxy-4-(4-phenylpiperazin-1-yl)phenyl]-9H-purine-2,6-diamine (C203)

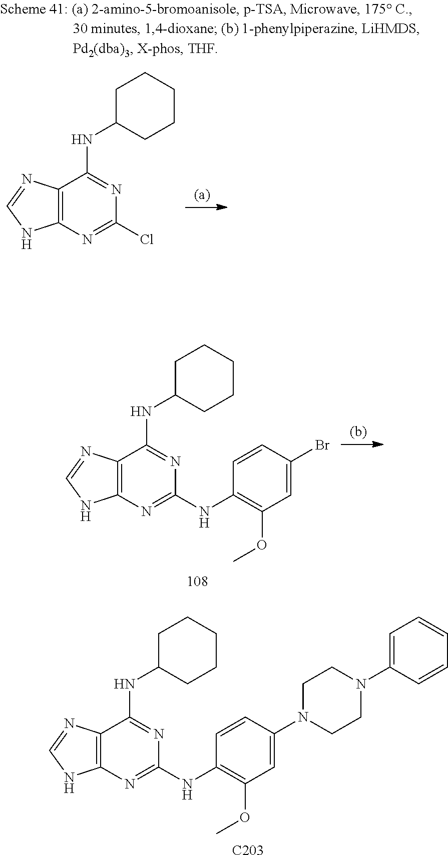

N2-(4-bromo-2-methoxy-phenyl)-N-6-cyclohexyl-9H-purine-2,6-diamine (108): To a solution of 2-chloro-6-aminocyclohexylpurine (500 mg, 2.0 mmol) in 1,4-dioxane (2.5 mL) was added p-TSA (264 mg, 1.4 mmol), and 2-amino-5-bromoanisole (482 mg, 2.4 mmol). The reaction was heated in the microwave at 175° C. for 30 minutes. The solvents were removed and the residue was purified by flash chromatography with dichloromethane/methanol as eluant to provide the desired product 108.

N6-cyclohexyl-N2-[2-methoxy-4-(4-phenylpiperazin-1-yl)phenyl]-9H-purine-2,6-diamine (C203): To compound 108 (1 equiv.) and desired amine (1.5 equiv.) was added LiHMDS in THF (1.5 mL, 1 M), followed by Pd₂(dba)₃ (0.02 equiv.) and X-phos (0.08 equiv.). The mixture was evacuated and purged with N₂ (3 cycles), then heated to 65° C. under N₂ overnight. The reaction was monitored by LCMS analysis. After the reaction was completed, it was cooled to room temperature, diluted with MeOH (2 mL) and concentrated HCl (0.5 mL) and concentrated. The residue was purified by reverse phase HPLC to provide C203.

Compounds C149-C153 and C204-C218 in the Table 2 were prepared according to the similar procedure described above using appropriately substituted starting materials.

### Synthesis of N2-(2-methoxy-4-morpholino-phenyl)-N-6-(1-methylsulfonyl-3-piperidyl)-9H-purine-2,6-diamine (C219)

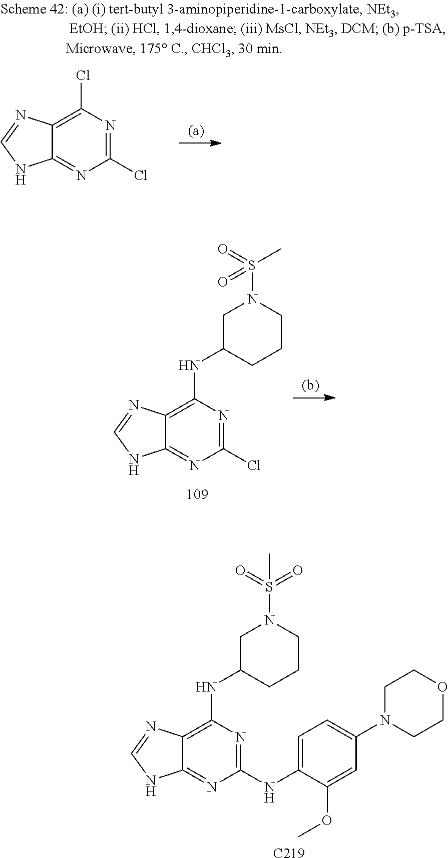

2-chloro-N-(3-piperidyl)-9H-purin-6-amine: Tert-butyl 3-aminopiperidine-1-carboxylate (1.46 g, 7.26 mmol), 2,6-dichloropurine (1.24 g, 6.6 mmol) and triethylamine (1.37 mL, 9.9 mmol) were combined in 33 mL ethanol and heated to 80° C. for 18 hours. The reaction was concentrated in vacuo and the residue purified by MPLC [silica: hexane/ethyl acetate]. Following purification the BOC group was removed by dissolving residue in 10 mL methanol and adding 4 mL 4N HCl in 1,4-dioxane. Product amine was isolated as the HCl salt (1.47 g, 5.1 mmol, 77%) and identified by LCMS [M+1]⁺=253. ¹H-NMR is consistent with the proposed structure.

2-chloro-N-(1-methylsulfonyl-3-piperidyl)-9H-purin-6-amine (109): 2-chloro-N-(3-piperidyl)-9H-purin-6-amine (1.47 g, 5.1 mmol) and triethylamine (1.77 mL, 12.8 mmol) were dissolved in 25 mL methylene chloride. Methanesulfonyl chloride (0.285 mL, 6.1 mmol) dissolved in 5 mL methylene chloride was added drop wise with magnetic stirring. The reaction was stirred for 1 hour at room temperature, at which time the entire reaction was diluted into 100 mL methylene chloride and washed with saturated sodium bicarbonate solution, water, and brine. The organic layer was dried over anhydrous sodium sulfate and concentrated to yield the desired compound 109 (1.32 g, 4.0 mmol, 78%) [M+1]⁺=331.

N2-(2-methoxy-4-morpholino-phenyl)-N-6-(1-methylsulfonyl-3-piperidyl)-9H-purine-2,6-diamine (219): 2-chloro-N-(1-methylsulfonyl-3-piperidyl)-9H-purin-6-amine 109 (100 mg, 0.30 mmol), 2-methoxy-4-morpholino-aniline (89 mg, 0.36 mmol) and p-TSA (46 mg, 0.24 mmol) were combined in 0.5 mL chloroform and heated in microwave reactor at 175° C. for 30 minutes. The reaction mixture was concentrated and the residue was purified by reverse HPLC [C-18: acetonitrile/water (0.01% trifluoroacetic acid) yielding product C219 as the TFA salt. The ¹H-NMR is consistent with proposed structure.

Compound C220, C221 and C222 in the Table 2, was prepared according to the similar procedure as described above, using either 3 or 4-amino-piperidine in the first step and appropriate aniline in the last step.

### Synthesis of N6-cyclohexyl-N2-[2-methoxy-4-[1-(2-morpholinoethyl)pyrazol-4-yl]phenyl]-9H-purine-2,6-diamine (C223)

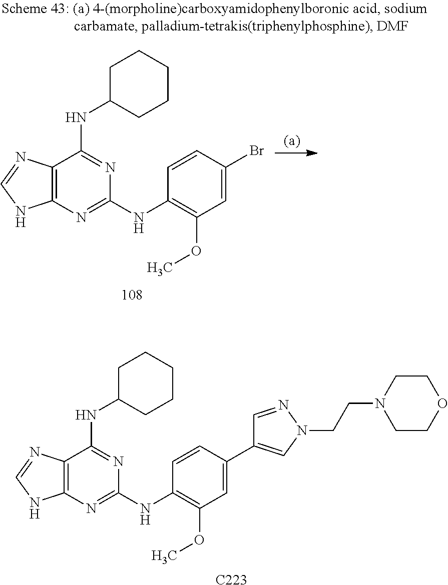

N2-(4-bromo-2-methoxy-phenyl)-N-6-cyclohexyl-9H-purine-2,6-diamine (108): It was prepared according to the procedure described earlier in Scheme 41.

N6-cyclohexyl-N2-[2-methoxy-4-[1-(2-morpholinoethyl)pyrazol-4-yl]phenyl]-9H-purine-2,6-diamine (C223): To a solution of 108 (200 mg, 0.4 mmol) in DMF (2.5 mL) was added sodium carbonate (106 mg, 1.0 mmol), 4-(morpholine)carboxamidophenylboronic acid (90 mg, 0.7 mmol) and finally palladium-tetrakis(triphenylphosphine) (0.1 mmol, 110 mg). The reaction was heated in the microwave at 150° C. for 40 minutes. The reaction mixture was concentrated and the residue was purified by reverse phase flash chromatography with MeOH/H₂O/0.1% TFA as eluant to provide the desired product C223.

Compounds C224 to C227 in the Table 2 were prepared according to the similar procedure described above using appropriately substituted starting materials.

### Synthesis of N6-cyclohexyl-N2-[2-methoxy-4-[1-(2-pyridyl)-3,6-dihydro-2H-pyridin-4-yl]phenyl]-9H-purine-2,6-diamine (C228)

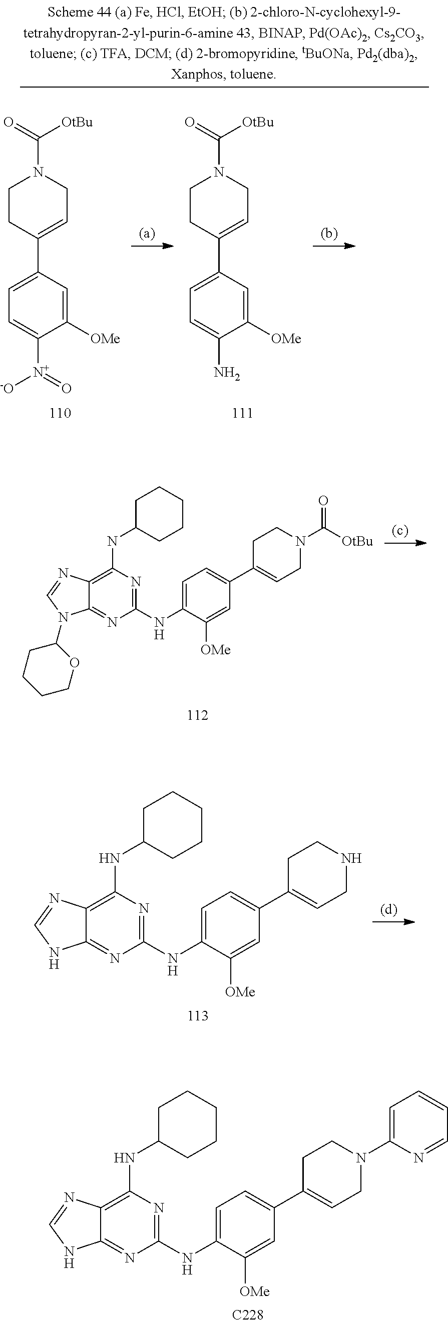

tert-butyl 4-(3-methoxy-4-nitro-phenyl)-3,6-dihydro-2H-pyridine-1-carboxylate (110): Compound 110 was prepared similar to the procedure described above in Scheme 16, using 2-(3-methoxy-4-nitro-phenyl)-4,4,5,5-tetramethyl-1,3,2-dioxaborolane in the first step.

tert-butyl 4-(4-amino-3-methoxy-phenyl)-3,6-dihydro-2H-pyridine-1-carboxylate (111): To a solution of tert-butyl 4-(3-methoxy-4-nitro-phenyl)-3,6-dihydro-2H-pyridine-1-carboxylate 110 from the previous step (1 g, 3 mmol) iron (powder, 420 mg) in 30 mL ethanol was added of 1N aqueous HCl (0.5 mL). Following this addition, the reaction was heated at reflux for 2 hours. The reaction mixture was cooled down and filtered through a pad of Celite. The reaction was concentrated in vacuo and the residue was purified by silica gel chromatography using MeOH/DCM as an eluant to provide 111.

tert-butyl-4-[4-[[6-(cyclohexylamino)-9-tetrahydropyran-2-yl-purin-2-yl]amino]-3-methoxy-phenyl]-3,6-dihydro-2H-pyridine-1-carboxylate (112): Compound 112 was prepared similar to the procedure described in Scheme 12, using tert-butyl 4-(4-amino-3-methoxy-phenyl)-3,6-dihydro-2H-pyridine-1-carboxylate 111 and 2-chloro-N-cyclohexyl-9-tetrahydropyran-2-yl-purin-6-amine 43.

N6-cyclohexyl-N2-[2-methoxy-4-(1,2,3,6-tetrahydropyridin-4-yl)phenyl]-9H-purine-2,6-diamine (113): Compound 112 (500 mg, 0.83 mmol) was dissolved in DCM/TFA (5 mL, 4:1) and the mixture stirred for 4 hours. The reaction was concentrated in vacuo and residue was purified by reverse phase chromatography using MeOH/H₂O/TFA as eluent to provide compound 113.

N6-cyclohexyl-N2-[2-methoxy-4-[1-(2-pyridyl)-3,6-dihydro-2H-pyridin-4-yl]phenyl]-9H-purine-2,6-diamine (C228): To 2-bromopyridine (9 μL, 0.1 mmol, 1.3 equiv.) and amine 113 (30 mg, 0.07 mmol, 1.0 equiv.) in dry toluene (1 mL) was added ^{t}BuONa (11 mg, 0.1 mmol, 1.5 equiv.), Pd₂(dba)₃ (1.3 mg, 1 μMol, 0.02 equiv.) and Xantphos (2.5 mg, 4 μMol, 0.06 equiv.). The mixture was evacuated and purged with N₂ (3 cycles), then heated to 95-100° C. under N₂ for 3 hours. The reaction was monitored by LCMS. After the reaction was completed, it was cooled to room temperature, diluted with ethyl acetate (2 mL), and washed with water (1 mL). The organic solution was concentrated in vacuo and the residue was purified by reverse phase HPLC to provide compound C228.

### Synthesis of [4-[4-[[6-(cyclohexylamino)-9H-purin-2-yl]amino]-3-methoxy-phenyl]-3,6-dihydro-2H-pyridin-1-yl]-(1-methylcyclopropyl)methanone (C229)

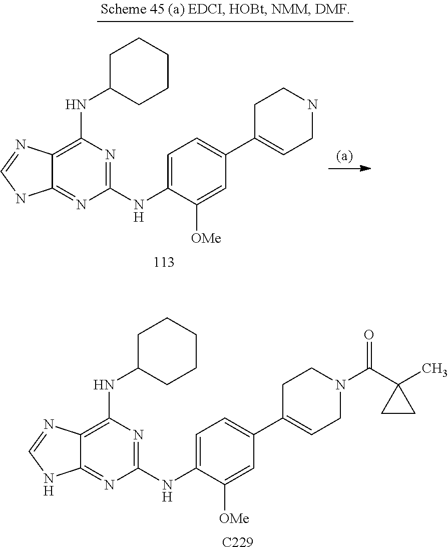

Amine 113 (100 mg, 0.24 mmol, 1.0 equiv.), 1-methylcyclopropanecarboxylic acid (24 mg, 0.24 mmol, 1.0 equiv.), EDCI (56 mg, 0.3 mmol, 1.2 equiv.), HOBt (14 mg, mmol, 0.5 equiv.), NMM (N-methyl morpholine) (120 μL, 0.131 mmol, 4.5 equiv.) were stirred at room temperature in DMF (2.4 mL) for 18 hours. The reaction was monitored by LCMS, the residue obtained after evaporation of the solvent was purified by reverse phase HPLC to provide C229.

### Synthesis of N6-cyclohexyl-N2-[2-methoxy-4-(1-methylsulfonyl-3,6-dihydro-2H-pyridin-4-yl)phenyl]-9H-purine-2,6-diamine (C230)

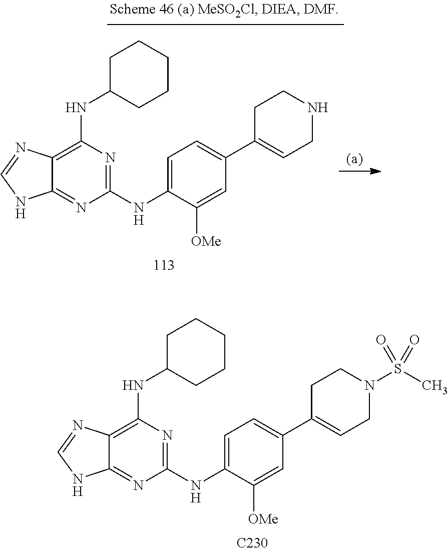

To amine 113 (12 mg, 0.023 mmol, 1.0 equiv.) in DMF (0.5 mL) at 0° C. was added N-ethyl-N-isopropyl-propan-2-amine (DIEA) (9.8 μL, 0.056 mmol, 2.5 equiv.) followed by MeSO₂Cl (2 μL, 0.025 mmol, 1.1 equiv.). The reaction was stirred at room temperature for 18 hours. The reaction was monitored by LCMS and the residue obtained after evaporation of the solvent was purified by reverse HPLC to provide C230.

### Synthesis of 1-[4-[4-[[6-(cyclohexylamino)-9H-purin-2-yl]amino]-3-methoxy-phenyl]-1-piperidyl]-2-(4-methylpiperazin-1-yl)ethane-1,2-dione (C231)

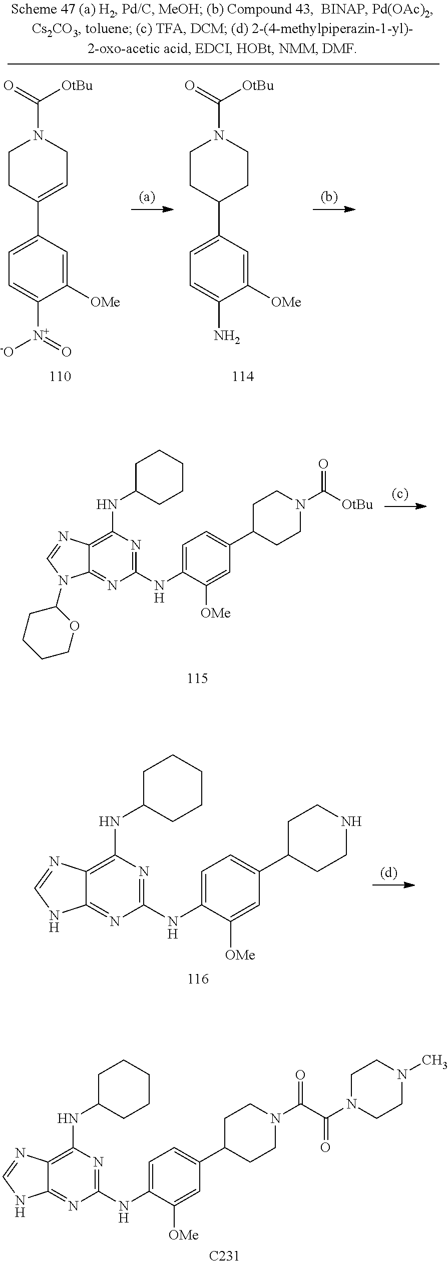

N6-cyclohexyl-N2-[2-methoxy-4-(4-piperidyl)phenyl]-9H-purine-2,6-diamine (116): The compound 110 was reduced employing standard hydrogenation conditions using Pd/C in methanol and the resulting aniline 114 was coupled with compound 43 employing Buchwald coupling condition followed by hydrolysis to get compound 116, as described earlier in Scheme 44.

1-[4-[4-[[6-(cyclohexylamino)-9H-purin-2-yl]amino]-3-methoxy-phenyl]-1-piperidyl]-2-(4-methylpiperazin-1-yl)ethane-1,2-dione (C231): Amine 116 (40 mg, 0.087 mmol, 1.0 equiv.), 2-(4-methylpiperazin-1-yl)-2-oxo-acetic acid (29 mg, 0.1 mmol, 1.2 equiv.), EDCI (20 mg, 0.1 mmol, 1.2 equiv.), HOBt (6 mg, 0.044 mmol, 0.5 equiv.), NMM (43 μL, 0.4 mmol, 4.5 equiv.) were stirred at room temperature in DMF (1 mL) for 18 hours. The reaction was monitored and purified by reverse HPLC to provide C231.

Compounds C232-233 in the Table 2, were prepared according to the similar procedure described above using appropriate starting materials.

### Synthesis of [4-[4-[[6-(cyclohexylamino)-9H-purin-2-yl]amino]-3-methoxy-phenyl]-1-piperidyl]-morpholino-methanone (C234) and N6-cyclohexyl-N2-[2-methoxy-4-(1-morpholinosulfonyl-4-piperidyl)phenyl]-9H-purine-2,6-diamine (C235)

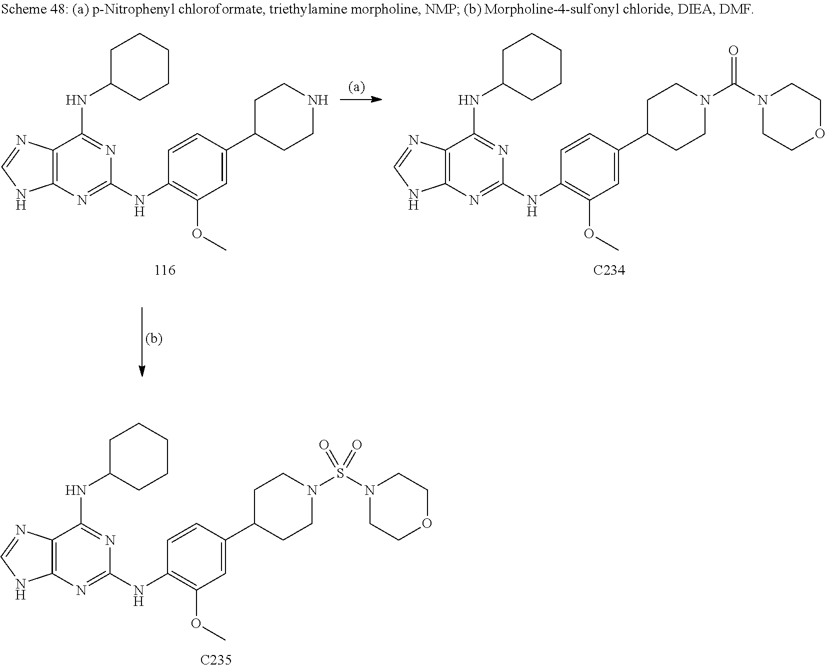

[4-[4-[[6-(cyclohexylamino)-9H-purin-2-yl]amino]-3-methoxy-phenyl]-1-piperidyl]-morpholino-methanone (C234): Prepared according to the procedure described earlier (Scheme 37) starting from N6-cyclohexyl-N2-[2-methoxy-4-(4-piperidyl)phenyl]-9H-purine-2,6-diamine 116 and appropriate amine and p-nitrophenyl chloroformate to get C234.

N6-cyclohexyl-N2-[2-methoxy-4-(1-morpholinosulfonyl-4-piperidyl)phenyl]-9H-purine-2,6-diamine (C235): Prepared according to the procedure described earlier (Scheme 39) starting from N6-cyclohexyl-N2-[2-methoxy-4-(4-piperidyl)phenyl]-9H-purine-2,6-diamine 116 and appropriate sulfonyl chloride to get C235.

### Synthesis of N6-cyclohexyl-N2-[2-methoxy-4-[1-(3-morpholinopropylsulfonyl)-4-piperidyl]phenyl]-9H-purine-2,6-diamine (C236), N6-cyclohexyl-N2-[2-methoxy-4-(1-methylsulfonyl-4-piperidyl)phenyl]-9H-purine-2,6-diamine (C238) and N6-cyclohexyl-N2-[2-methoxy-4-[1-(3-morpholinosulfonylpropyl)-4-piperidyl]phenyl]-9H-purine-2,6-diamine (C239)

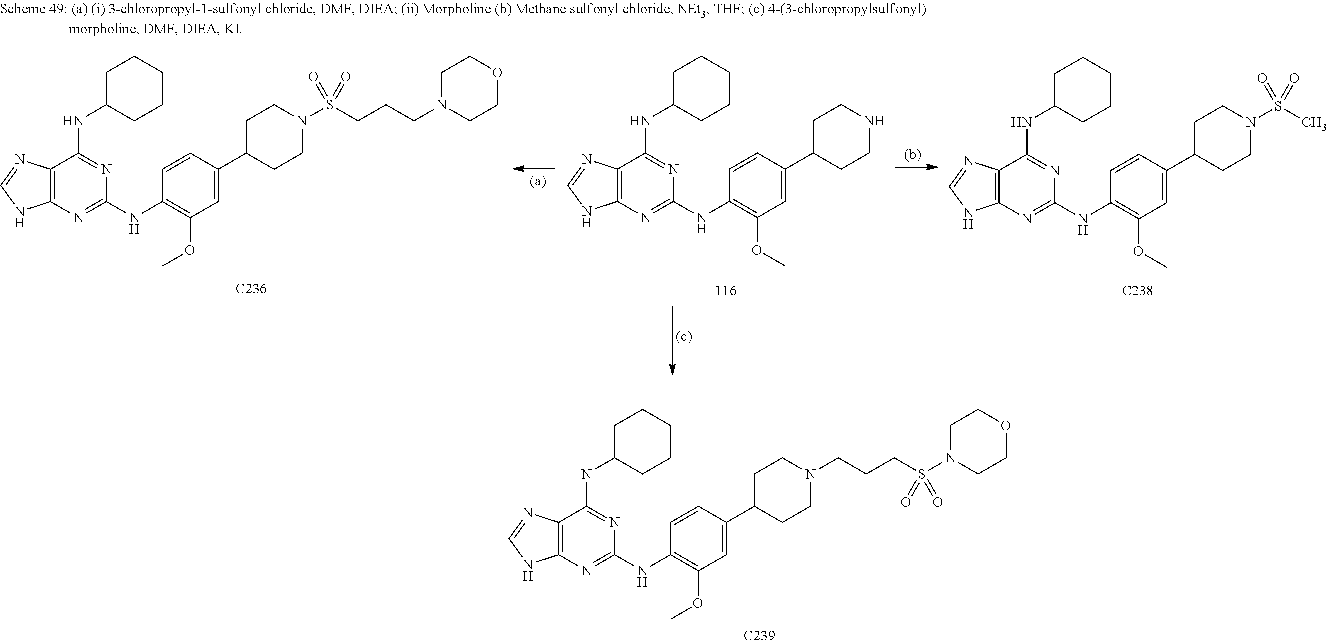

N6-cyclohexyl-N2-[2-methoxy-4-[1-(3-morpholinopropylsulfonyl)-4-piperidyl]phenyl]-9H-purine-2,6-diamine (C236): Prepared starting from compound 116 and appropriate sulfonyl chloride and amine according to the procedure as described in Scheme 32.

Compound C237 in the Table 2 was prepared in an analogous manner with appropriate starting materials.

N6-cyclohexyl-N2-[2-methoxy-4-(1-methylsulfonyl-4-piperidyl)phenyl]-9H-purine-2,6-diamine (C238) Prepared according to the procedure described earlier (Scheme 32) starting from N6-cyclohexyl-N2-[2-methoxy-4-(4-piperidyl)phenyl]-9H-purine-2,6-diamine 116 to get C238.

N6-cyclohexyl-N2-[2-methoxy-4-[1-(3-morpholinosulfonylpropyl)-4-piperidyl]phenyl]-9H-purine-2,6-diamine (C239): To the amine 116 (40 mg, 0.088 mmol, 1.0 equiv.) in DMF (0.5 mL) at 0° C. was added DIEA (230 μL, 1.32 mmol, 15 equiv.). 4-(3-chloropropyl-sulfonyl)morpholine (2 μL, 0.025 mmol, 1.1 equiv., prepared by treating 3-chloropropane-1-sulfonyl chloride (103 μL, 0.284 mmol), DIEA (222 μL, 0.426 mmol), morpholine (26 μL, 0.3 mmol) in DMF (1 mL)) was added after 5 minutes, followed with a catalytic amount of KI and the reaction was stirred at room temperature for 18 hours. The residue obtained after evaporation of the solvent was purified by reverse phase HPLC to get C239.

### Synthesis of [1-[4-[[6-(cyclohexylamino)-9H-purin-2-yl]amino]-3-methoxy-phenyl]-4-piperidyl]-morpholino-methanone (C240) And N6-cyclohexyl-N2-[2-methoxy-4-[4-(morpholinomethyl)-1-piperidyl]phenyl]-9H-purine-2,6-diamine (C241)

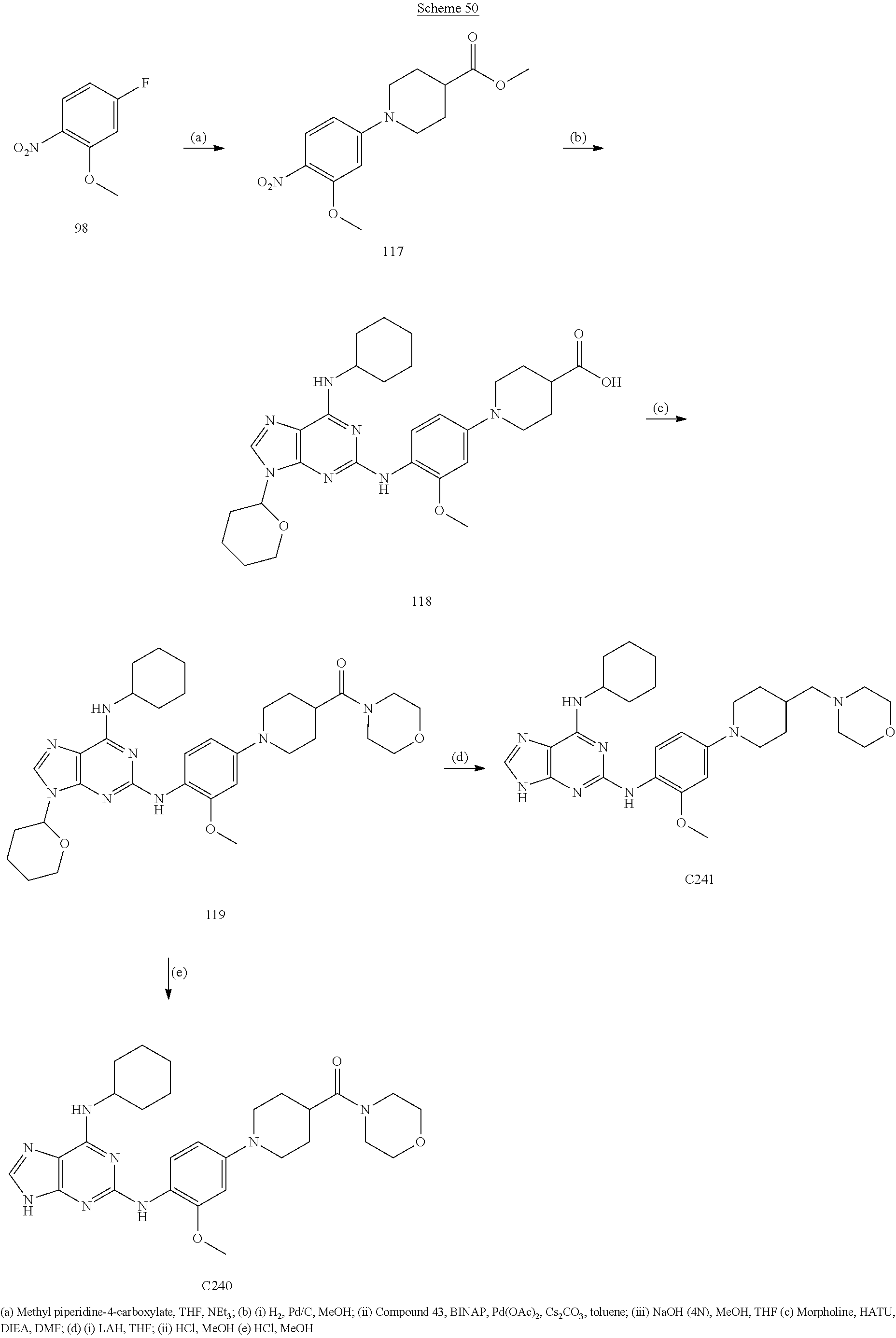

Methyl 1-(3-methoxy-4-nitro-phenyl)piperidine-4-carboxylate (117): To a solution of 4-fluoro-2-methyl-1-nitrobenzene (1 g, 5.77 mmol, 1 equiv.) in THF (30 mL) was added methyl piperidine-4-carboxylate HCl (895 mg, 6.93 mmol, 1.2 equiv.) and triethyl amine (NEt₃) (2.4 mL, 17 mmol, 3 equiv.). The reaction was stirred at reflux and monitored by LCMS for completion after 2 hours. The solvent was removed in vacuo and the residue diluted with ethyl acetate and washed with water. The organic layer was concentrated and the residue was purified by silica gel flash chromatography using MeOH/DCM as eluent, and its structure 117 was established based on LCMS and ¹H NMR analysis.

Methyl 1-[4-[[6-(cyclohexylamino)-9-tetrahydropyran-2-yl-purin-2-yl]amino]-3-methoxy-phenyl]piperidine-4-carboxylate (118): The compound 117 was hydrogenated employing similar conditions described for compound 110 in Scheme 47, and subsequently coupled (using palladium catalyzed Buchwald coupling procedure as described in Scheme 12) to compound 43.

To a solution of methyl 1-[4-[[6-(cyclohexylamino)-9-tetrahydropyran-2-yl-purin-2-yl]amino]-3-methoxy-phenyl]piperidine-4-carboxylate (2 g, 5.55 mmol, 1 equiv.) (not shown) obtained from the above step, in MeOH/THF mixture (100 mL, 1/1) was added aq. NaOH (20 mL, 4N). The reaction was stirred at room temperature and monitored by LCMS for completion after overnight. The reaction was concentrated in vacuo and the residue was diluted with ethyl acetate, washed with water followed by 4 N HCl (20 mL). The organic layer was separated and concentrated in vacuo to provide 118.

[1-[4-[[6-(cyclohexylamino)-9-tetrahydropyran-2-yl-purin-2-yl]amino]-3-methoxy-phenyl]-4-piperidyl]-morpholino-methanone (119): To the compound 118 (2.6 g, 4.74 mmol, 1 equiv.) in DMF (50 mL, 0.1M) was added HATU (2.18 g, 5.69 mmol, 1.2 equiv.), DIEA (1.7 mL, 9.5 mmol, 2 equiv.), morpholine (620 μL, 7.1 mmol, 1.5 equiv.) and the mixture was stirred for 18 hours at room temperature. Upon completion, solvent were concentrated in vacuo and residue purified by silica gel flash chromatography with MeOH/DCM as eluant to provide 119.

[1-[4-[[6-(cyclohexylamino)-9H-purin-2-yl]amino]-3-methoxy-phenyl]-4-piperidyl]-morpholino-methanone (C240): The compound 119 was hydrolyzed with 4 M HCl MeOH as described before (Scheme 12) to provide C240.

N6-cyclohexyl-N2-[2-methoxy-4-[4-(morpholinomethyl)-1-piperidyl]phenyl]-9H-purine-2,6-diamine (C241): To a suspension of LAH (2 g, 52.6 mmol, 10 equiv.) in THF (25 mL) at 0° C. was added (in a drop wise fashion) a solution of compound 119 (3.25 g, 5.26 mmol, 1 equiv.) in THF (25 mL). The reaction was stirred at room temperature for 4 hours and monitored by LCMS for completion. The reaction was then quenched with the successive addition at 0° C. of 2 mL water, 2 mL aq. NaOH (15%), 6 mL water and MgSO₄. The mixture was stirred at room temperature for several hours before filtration through a pad of Celite. The filtrate was concentrated in vacuo and the residue purified by reverse phase chromatography with MeOH/TFA/H₂O as eluant. Similar experimental conditions described earlier in Scheme 12 were used for the hydrolysis of the protecting group to provide compound C241.

Compound C242 in the Table 2, was prepared in an analogous manner with appropriately substituted starting materials. Compound C261 Table 2, was prepared n analogous manner except commercially available piperidine-4-carboxamide was used in the first step.

### Synthesis of 2-[1-[4-[[6-(cyclohexylamino)-9-tetrahydropyran-2-yl-purin-2-yl]amino]-3-methoxy-phenyl]-4-piperidyl]-1-pyrrolidin-1-yl-ethanone (C244)

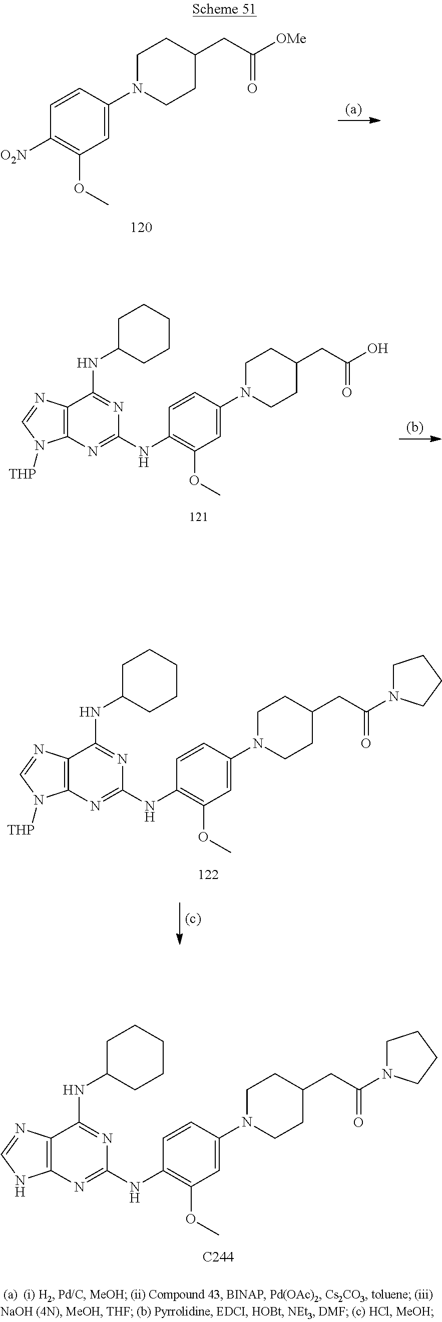

The compound 120 was obtained employing similar conditions described in Scheme 50 using methyl-2-(4-piperidyl)acetate and was hydrogenated employing standard conditions as described before, the intermediate aniline was converted to compound C244 via intermediates 121 and 122 as described in Scheme 50.

C243 in the Table 2, was made similarly employing procedure describe above, using morpholine in the coupling step.

### Synthesis of N6-cyclohexyl-N2-[4-(1-ethyl-3-piperidyl)-2-methoxy-phenyl]-9H-purine-2,6-diamine (C245)

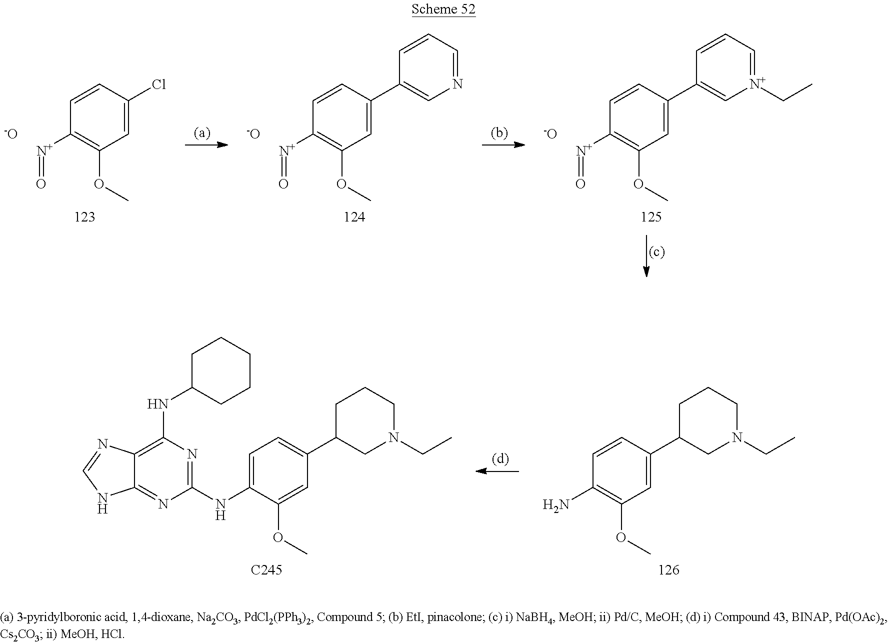

Compound 126 was prepared according to the procedure reported in WO 2009/020990.

3-(3-methoxy-4-nitro-phenyl)pyridine (124): Nitrogen was bubbled through 1,4-dioxane (70 mL) for 15 minutes prior to the addition of 4-chloro-2-methoxy-1-nitro-benzene 123 (4 g, 21.4 mmol, 1.0 equiv.), 3-pyridylboronic acid (3.15 g, 25.7 mmol, 1.2 equiv.), PdCl₂(PPh₃)₂ (0.75 g, 1 mmol, 0.05 equiv.) and degassed aqueous Na₂CO₃ solution (24 mL, 3N, 64.2 mmol). The reaction mixture was heated at 80° C. for 4 hours. Additional water (100 mL) was added to the reaction and it was cooled to room temperature. Crude product was extracted from the reaction mixture with ethyl acetate. The combined org. phases were dried (over anhydrous Na₂SO₄), concentrated, and purified by silica gel column chromatography to afford 3-(3-methoxy-4-nitro-phenyl)pyridine (124).

1-ethyl-3-(3-methoxy-4-nitro-phenyl)pyridin-1-ium (125): To the solution of pinacolone (43 mL, 0.1M) was added compound 124 (1 g, 4.35 mmol, 1 equiv.) and ethyl iodide (EtI) (1.46 mL, 18.1 mmol, 4.2 equiv.). The reaction mixture was heated at 102° C. for 12 hours. The resulting precipitate was collected by filtration, washed with MeOH and dried to afford 1-ethyl-3-(3-methoxy-4-nitro-phenyl)pyridin-1-ium (125).

4-(1-ethyl-3-piperidyl)-2-methoxy-aniline (126): To a stirred solution of 1-ethyl-3-(3-methoxy-4-nitro-phenyl)pyridin-1-ium 125 (1.68 g, 4.35 mmol, 1 equiv.) in MeOH (40 mL, 0.1 mL) at −10° C. was added NaBH₄ (500 mg, 13 mmol, 3 equiv.) slowly over 5 minutes. The reaction was continued to stir at that temperature for additional 1 hour. The mixture was concentrated and diluted with ethyl acetate before quenching with saturated aqueous NaHCO₃. The organic phase was washed with brine, concentrated, and dried. Purification by chromatography on silica gel afforded 1-ethyl-5-(3-methoxy-4-nitro-phenyl)-3,6-dihydro-2H-pyridine. To the residue (1 g, 7.63 mmol) dissolved in MeOH (80 mL) was added 10% Pd/C (150 mg) and the reaction was stirred at room temperature over the weekend under 40 psi H₂ atmosphere in the Parr hydrogenator. The suspension was filtered over a pad of Celite and the residue purified by chromatography on silica gel to afford 4-(1-ethyl-3-piperidyl)-2-methoxy-aniline (126).

N6-cyclohexyl-N2-[4-(1-ethyl-3-piperidyl)-2-methoxy-phenyl]-9H-purine-2,6-diamine (C245): Compound 126 was coupled to (2-Chloro-9H-purin-6-yl)-cyclohexylamine 43 and submitted to protecting group hydrolysis employing similar experimental conditions as described earlier (Scheme 12).

Compound C246 in the Table 2 was prepared in a similar manner as described above, using appropriate starting materials. C247 in the Table 2, was prepared in a similar manner employing conditions described in WO 2009/020990.

### Synthesis of N6-cyclohexyl-N2-[2-methoxy-4-[3-(morpholinomethyl)pyrrolidin-1-yl]phenyl]-9H-purine-2,6-diamine (C-248)

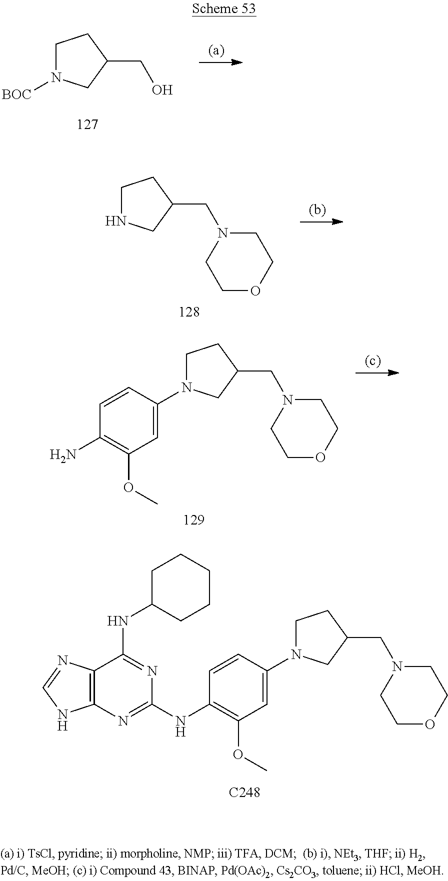

4-(pyrrolidin-3-ylmethyl)morpholine (128): p-toluenesulfonyl chloride (p-TSA) (1.2 g, 6.25 mmol, 1.25 equiv.) was added to a solution of alcohol 127 (1 g, 5 mmol, 1 equiv.) in dry pyridine (15 mL) at 0° C., and the mixture was stirred at this temperature for 24 h. After addition of water (10 mL) and extraction with DCM, the organic layer was washed with water, dried over Na₂SO₄ and concentrated to dryness, affording dark brown oil that upon column chromatography with 1:1 ethyl acetate/hexane as eluant gave pure tosylate. A solution of tosylate (500 mg, 1.4 mmol) and morpholine (350 μL, 4 mmol) was stirred in NMP (10 mL) for 16 hours at 85° C. The solvent was removed in vacuo, and the residue was dissolved in DCM. This solution was washed twice with water, dried (Na₂SO₄), and the solvent was removed in vacuo, affording oil following silica gel column chromatography with MeOH/DCM as eluant. The oil was dissolved in a 1:3 mixture of TFA/DCM and the reaction was stirred overnight at room temperature until completion. The solvent were then evaporated to provide 128 as a TFA salt.

4-[[1-(3-methoxy-4-nitro-phenyl)pyrrolidin-3-yl]methyl]morpholine (C248): Compound 129 was prepared in the manner previously described for compound 117 (Scheme 50) using 4-(pyrrolidin-3-ylmethyl)morpholine 128 instead of methyl piperidine-4-carboxylate HCl, followed by hydrogenation to get 129. Then 129 was coupled to (2-Chloro-9H-purin-6-yl)-cyclohexylamine and submitted to protecting group hydrolysis employing similar experimental conditions as described earlier (Scheme 12) to get compound C248.

### Synthesis of 1-[4-[[6-(cyclohexylamino)-9H-purin-2-yl]amino]-3-methoxy-phenyl]-4-(morpholinomethyl)piperidin-4-ol (C249)

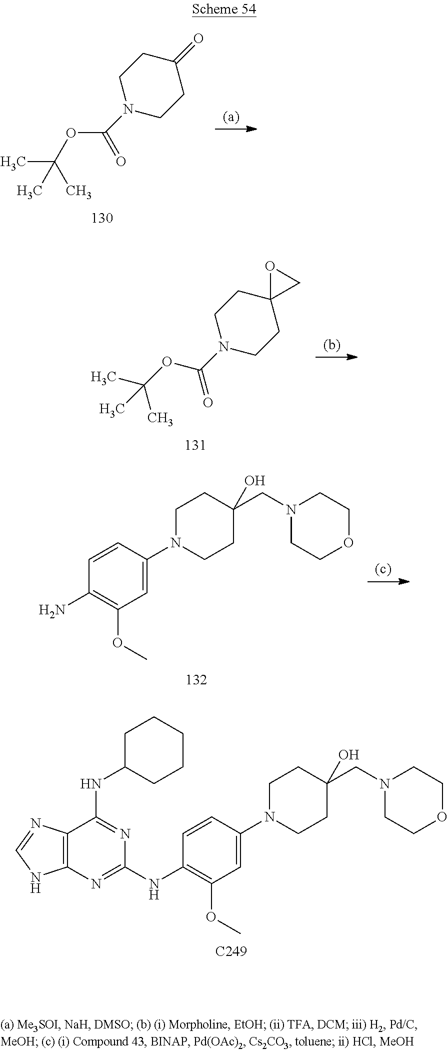

tert-butyl 2-oxa-6-azaspiro[2.5]octane-6-carboxylate (131) Trimethylsulfonium iodide (29 g, 132 mmol) was added to a suspension of NaH (5.3 g, 132 mmol, 60% in oil) in DMSO (250 mL) cooled to 0° C. The reaction mixture was then allowed to warm to room temperature and stirred for 40 minutes. tent-Butyl 4-oxopiperidine-1-carboxylate (25 g, 125 mmol) was added to the reaction mixture, followed by stirring at room temperature for 1 hour and then at 55° C. for 1.5 hours. The reaction mixture was then poured onto water and extracted with ethyl acetate. The combined organic layers were washed with water, dried (anhydrous Na₂SO₄) and concentrated in vacuo to provide 131.

1-(4-amino-3-methoxy-phenyl)-4-(morpholinomethyl)piperidin-4-ol (132): To a solution of compound 131 (200 mg, 0.94 mmol, 1 equiv.) in ethanol (2 mL) was added morpholine (115 μl, 1.1 mmol, 1.1 equiv.) and the reaction was heated to 50° C. for 2 hours. Solvent was removed in vacuo and the residue purified by silica gel flash chromatography with MeOH/DCM as eluent. The BOC group was removed from the resultant intermediate (not shown) by dissolving in a 1:3 mixture of TFA/DCM (4 mL) stirring overnight at room temperature. The solvent was removed in vacuo to provide desired intermediate 4-(morpholinomethyl)piperidin-4-ol (not shown) as the TFA salt. Compound 132 was prepared in the manner previously described for compound 117 (Scheme 50) using 4-(morpholinomethyl)piperidin-4-ol TFA, followed by hydrogenation to get 132.

1-[4-[[6-(cyclohexylamino)-9H-purin-2-yl]amino]-3-methoxy-phenyl]-4-(morpholinomethyl)piperidin-4-ol (C249): Compound 132 was coupled to (2-Chloro-9H-purin-6-yl)-cyclohexylamine and submitted to protecting group hydrolysis employing similar experimental conditions as described earlier (Scheme 12).

### Synthesis of 1-[4-[[6-(cyclohexylamino)-9H-purin-2-yl]amino]-3-methoxy-phenyl]-2-morpholino-ethanol (C250)

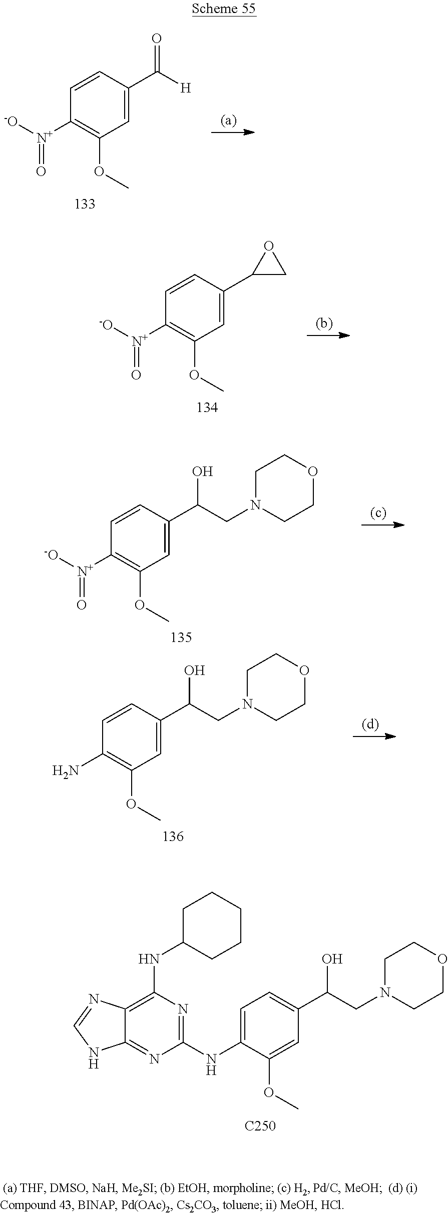

2-(3-methoxy-4-nitro-phenyl)oxirane (134): The suspension of NaH (438 mg, 11 mmol, 1 equiv.)(washed with hexanes) in DMSO (70 mL) in a dry 3-necked flask was heated at 60° C. for 1 hour, the resulting greenish solution was diluted with dry THF (70 mL) and was cooled to 0° C. Solution of Me₃SI (2.2 g, 11 mmol, 1 equiv.) in dry DMSO (44 mL) was added slowly over a 15 minute period. After a few minutes, a solution of the 3-methoxy-4-nitro-benzaldehyde 133 (2 g, 11 mmol, 1 equiv.) in dry THF (37 mL) was added drop wise to the sulfur glide. The dark purple mixture was stirred until the reaction was complete in ˜15 minutes as determined by TLC and then added to water (1 L). The mixture was extracted with ether (3×300 mL), and the combined organic layers were washed with water and dried over anhydrous MgSO4, and filtered. The solvent were evaporated and the resulting oil was quickly passed through a pad of silica gel with ethyl ether. The residue obtained after evaporation of the solvent was purified by flash chromatography using ethyl acetate/hexanes as eluents.

1-(4-amino-3-methoxy-phenyl)-2-morpholino-ethanol (136): To a solution of the 2-(3-methoxy-4-nitro-phenyl)oxirane 134 (213 mg, 1.1 mmol, 1 equiv.) in EtOH (15 mL, 0.07M) was added morpholine (4.2 mL, 48 mmol, 44 equiv.) and the reaction was heated at 80° C. for 8 hours. The solvent was removed and the residue purified by silica gel flash chromatography (MeOH/DCM as eluent). 1-(3-methoxy-4-nitro-phenyl)-2-morpholino-ethanol 135 was hydrogenated using PtO₂ at 40 psi in methanol for 12 hours to provide compound 136 .

1-[4-[[6-(cyclohexylamino)-9H-purin-2-yl]amino]-3-methoxy-phenyl]-2-morpholino-ethanol (C250): Compound 136 was coupled to (2-Chloro-9H-purin-6-yl)-cyclohexylamine and submitted to protecting group hydrolysis employing similar experimental conditions as described earlier (Scheme 12).

Compound C251 was prepared in an analogous manner with appropriate starting materials.

### Synthesis of N2-[2-methoxy-4-(1-morpholinoethyl)phenyl]-N-6-tetrahydropyran-4-yl-9H-purine-2,6-diamine (C252)

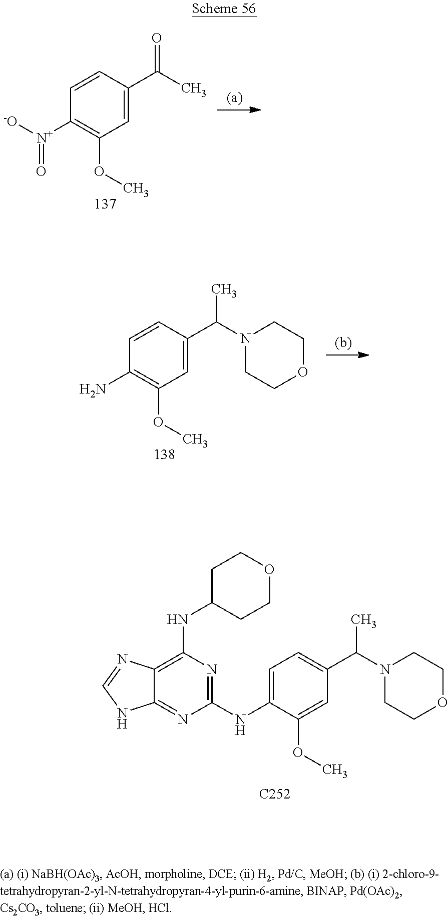

2-methoxy-4-(1-morpholinoethyl)aniline (138): To a solution of 1-(3-methoxy-4-nitro-phenyl)ethanone (137) (0.2 g, 1 mmol, 1 equiv.) in DCE (1 mL) was added AcOH (85 μL, 12 M) and morpholine (178 μL, 2 mmol, 2 equiv.). The reaction was stirred 30 minutes before the addition of NaBH(OAc)₃ (260 mg, 1.23 mmol, 1.2 equiv.) followed by additional 12 hours stirring at room temperature. Aqueous NaHCO₃ was added and the product extracted with DCM. The product was further purified by chromatography on silica gel. The hydrogenation procedure was similar to above to afford 2-methoxy-4-(1-morpholinoethyl)aniline (138).

N2-[2-methoxy-4-(1-morpholinoethyl)phenyl]-N-6-tetrahydropyran-4-yl-9H-purine-2,6-diamine (C252)Compound 138 was coupled to 2-chloro-9-tetrahydropyran-2-yl-N-tetrahydropyran-4-yl-purin-6-amine and submitted to protecting group hydrolysis employing similar experimental conditions as described earlier (Scheme 12).

### Synthesis of N2-[4-(1,1-dimethyl-2-morpholino-ethyl)-2-methoxy-phenyl]-N-6-tetrahydropyran-4-yl-9H-purine-2,6-diamine (C253)

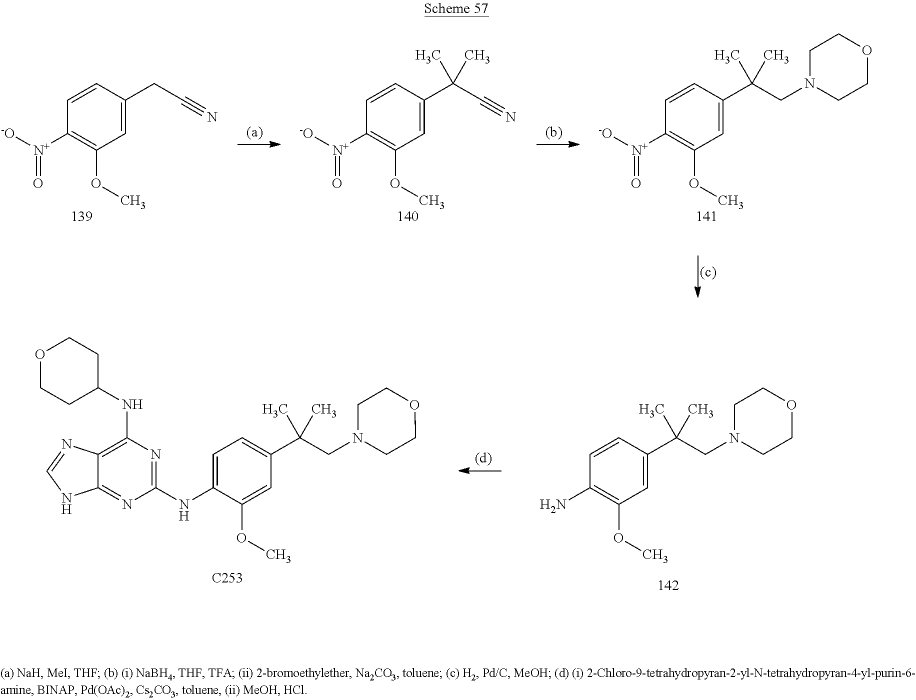

2-(3-methoxy-4-nitro-phenyl)-2-methyl-propanenitrile (140): To a suspension of NaH (125 mg, 3.13 mmol, 3 equiv.) in THF (4 mL) was added drop wise 2-(3-methoxy-4-nitro-phenyl)acetonitrile 139 (0.2 g, 1 mmol, 1 equiv.) and after 20 minutes MeI (156 μL, 2.5 mmol, 2.4 equiv.) was added. The solution was stirred for 15 hours at ambient temperature and quenched with i-PrOH followed by water. The resulting mixture was extracted with ether. The combined organic layer was washed with aq. NH₄Cl, water and evaporated to obtain the desired product, it was used directly in the next step.

4-[2-(3-methoxy-4-nitro-phenyl)-2-methyl-propyl]morpholine (141): To a stirred suspension of NaBH₄ (104 mg, 2.74 mmol, 4 equiv.) in THF (1 mL) a TFA solution in THF (204 μL, 2.74 mmol, 4 equiv., 0.4 mL of THF) was added over 5 minutes at room temperature. A THF solution of 2-(3-methoxy-4-nitro-phenyl)-2-methyl-propanenitrile (140) (151 mg, 0.7 mmol, 1 equiv.) was then added and the reaction was stirred at room temperature for 12 hours. After evaporation to dryness, ice water was added and the reaction basified with KOH before extracting the product with ether. The organic layer was extracted with dilute HCl. The aqueous acidic solution was basified and extracted again with ether. The solvents removed in vacuo to afford the primary amine (not shown).

To a round-bottomed flask containing a teflon-coated magnetic stirring bar was added the previously obtained amine (70 mg, 0.31 mmol, 1 equiv.), 2-bromoethyl ether (80 mg, 0.34 mmol, 1.1 equiv.), sodium bicarbonate (58 mg, 0.68 mmol, 2.2 equiv.) and toluene (1 mL, 1.4M). The reaction flask was heated to 115° C. for 22.5 hours. The reaction mixture was allowed to cool to room temperature, filtered, and the filtrate was washed with water (100 mL). The product was extracted from the organic solution into aqueous citric acid solution (30% by weight, 2×200 mL). The combined citric acid layers were cooled in an ice-water bath and aqueous sodium hydroxide solution (6 N) was added slowly to the cooled solution to an endpoint of pH 13. The resulting basic aqueous mixture was extracted with toluene (3×300 mL). The toluene layers were combined and the combined solution was washed sequentially with water (200 mL) and a brine solution (300 mL). The washed product solution was dried over sodium sulfate, concentrated in vacuo and the residue purified by silica gel flash chromatography to afford the 4-[2-(3-methoxy-4-nitro-phenyl)-2-methyl-propyl]morpholine (141).

N2-[4-(1,1-dimethyl-2-morpholino-ethyl)-2-methoxy-phenyl]-N-6-tetrahydro-pyran-4-yl-9H-purine-2,6-diamine (C253): Compound 141 was subjected to standard (Pd/C) hydrogenation conditions to afford 4-(1,1-dimethyl-2-morpholino-ethyl)-2-methoxy-aniline 142. Compound 142 was then coupled to 2-chloro-9-tetrahydropyran-2-yl-N-tetrahydropyran-4-yl-purin-6-amine 43 and submitted to protecting group hydrolysis employing similar experimental conditions as described earlier (Scheme 12).

### Synthesis of N-(2-chloro-4-isopropoxy-phenyl)-6-(2-morpholinoethyl)-9H-purin-2-amine (C254)

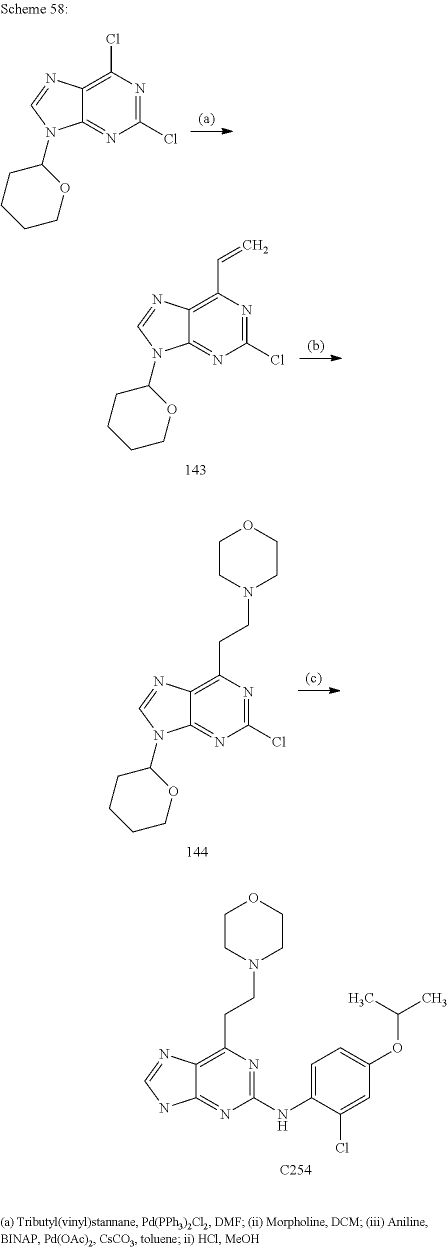

2-Chloro-9-tetrahydropyran-2-yl-6-vinyl-purine (143): To a solution of 2,6-dichloro-9-tetrahydropyran-2-yl-purine (1.0 g, 3.7 mmol) and tributyl(vinyl)stannane (1.1 mL, 3.7 mmol) in DMF (30 mL) was added Pd(PPh₃)₂Cl₂ (0.13 g, 0.18 mmol). The solution was degassed then heated to 55° C. for 22 hours under nitrogen atmosphere. The reaction was concentrated and the resulting residue purified by silica gel flash chromatography using Ethyl acetate/Hexanes as eluent to afford the desired intermediate 143 as an oil (0.65 g).

4-[2-(2-chloro-9-tetrahydropyran-2-yl-purin-6-yl)ethyl]morpholine (144): To a solution of 143 (0.38 g, 1.4 mmol) in DCM (15 mL) was added morpholine (0.23 mL, 2.3 mmol). The solution was allowed to stir for 22 hours under nitrogen atmosphere. The reaction was concentrated and desired product 144 precipitated from ether as the HCl salt.

N-(2-chloro-4-isopropoxy-phenyl)-6-(2-morpholinoethyl)-9H-purin-2-amine (C252): Buchwald reaction of intermediate 144 with 2-chloro-4-isopropoxy-aniline followed by protecting group removal gives C254, as described in Scheme 12. Compounds C255-C257 in the Table 2, were prepared in an analogous manner with appropriately substituted starting materials.

### Synthesis of N6-cyclohexyl-N2-[2-methyl-4-[(E)-3-morpholinoprop-1-enyl]phenyl]-9H-purine-2,6-diamine (C258)

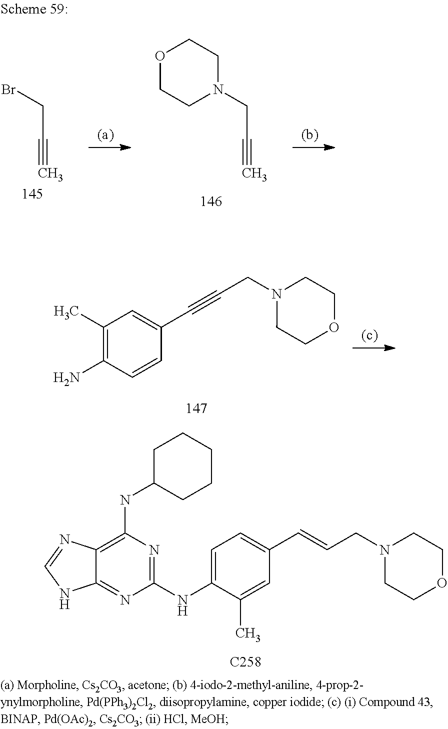

4-Prop-2-ynylmorpholine (146): To a solution of morpholine (14 mmol) in acetone (30 mL) was added Cs₂CO₃ (14 mmol). To the stirring suspension was added 3-bromoprop-1-yne 145 (14 mmol) drop wise. The reaction was allowed to stir over the weekend, filtered, and concentrated. The resulting residue was taken up in Ethyl acetate (100 mL), washed with a saturated NaHCO₃ solution (100 mL), dried (sodium sulfate) and concentrated in vacuo to afford 4-prop-2-ynylmorpholine 146 as viscous oil.

2-methyl-4-(3-morpholinoprop-1-ynyl)aniline (147): To a solution of 4-iodo-2-methyl-aniline (2.2 mmol), 4-prop-2-ynylmorpholine (2.2 mol) and Pd(PPh₃)₂Cl₂ (0.22 mmol) under nitrogen in diisopropylamine (5 mL) was added copper iodide (0.22 mmol). The solution was degassed then heated to 80° C. for 4 hours under nitrogen atmosphere. The reaction was concentrated, dissolved in methanol, filtered over Celite, and concentrated in vacuo to afford 2-methyl-4-(3-morpholinoprop-1-ynyl)aniline 147 used without further purification.

N6-cyclohexyl-N2-[2-methyl-4-[(E)-3-morpholinoprop-1-enyl]phenyl]-9H-purine-2,6-diamine (C258): Buchwald reaction of intermediate 147 with compound 43 followed by protecting group removal furnished C258 as a minor product, which was purified by reverse phase HPLC. Compound C91 from Table 1, was the major product.

### Synthesis of 2-[4-[[6-(cyclohexylamino)-9H-purin-2-yl]amino]-3-methoxy-phenyl]-1-morpholino-propan-1-one (C260)

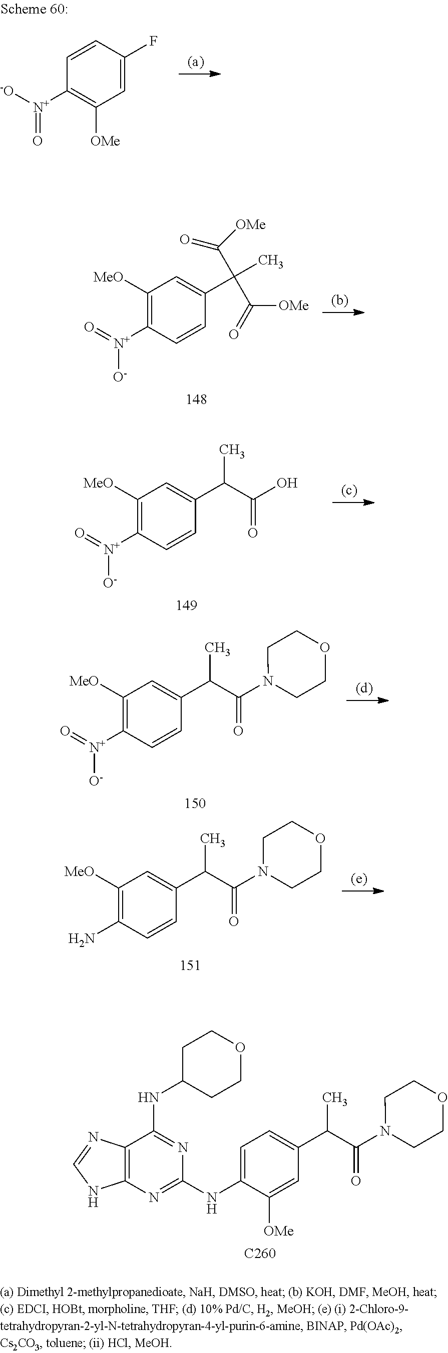

Dimethyl 2-(3-methoxy-4-nitro-phenyl)-2-methyl-propanedioate (148): Prepared according to the procedure reported in Synthesis, 1993, 51-53, starting from 4-fluoro-2-methoxy-1-nitro-benzene and dimethyl 2-methylpropanedioate.

2-(3-methoxy-4-nitro-phenyl)propanoic acid (149): The compound 148 was hydrolyzed to get 149, according to the procedure reported in J. Med. Chem., 26(2), 1983, 222-226.

2-(3-methoxy-4-nitro-phenyl)-1-morpholino-propan-1-one (150): Employing standing peptide coupling conditions reported earlier (Scheme 13), compound 149 was coupled to morpholine to provide 150.

2-(4-amino-3-methoxy-phenyl)-1-morpholino-propan-1-one (151): Compound 150 was hydrogenated using standard reduction condition described earlier with Pd/C in MeOH to provide 151.

2-[3-methoxy-4-[[6-(tetrahydropyran-4-ylamino)-9H-purin-2-yl]amino]phenyl]-1-morpholino-propan-1-one (C260): 2-Chloro-9-tetrahydropyran-2-yl-N-tetrahydropyran-4-yl-purin-6-amine was coupled with compound 151 using Buchwald conditions followed by protecting group hydrolysis described earlier in Scheme 12, to provide compound C260.

The structures and characterization of the exemplary compounds made and the synthetic scheme of each (appropriate, commercially available starting materials are known to those skilled in the art) are provided in Table 1 below.

TABLE 1
Exemplary Compounds of the Invention

| | | | | HPLC, Mass |
|---|---|---|---|---|
| | | | | Spec. and |
| | | | Syn- | ¹H-NMR |
| Comp. | | | thetic | Data (where |
| # | IUPAC Name | Structure | Scheme | applicable) |
| C1 | N~6~- cyclopentyl- N~2~-(2,4- dimethoxy- phenyl)- 9H-purine- 2,6- diamine | 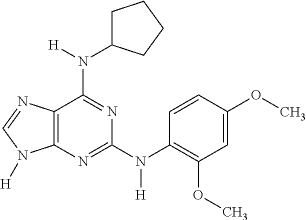 | 5 | Ret. Time (HPLC): 6.774 min; Mass Spec.: 355.18765 (M + 1) |
| C2 | 2-{3-[4-({6- [(trans-4- hydroxy- cyclohexyl) amino]-9H- purin- 2-yl} amino)-3- methyl- phenoxy] propyl}-1H- isoindole- 1,3(2H)- dione | 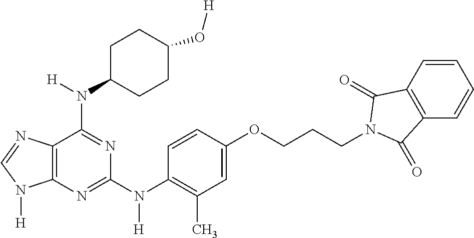 | 18 | Ret. Time (HPLC): 6.268 min; Mass Spec.: 542.25162 (M + l); 1H NMR Data: (400 MHz, DMSO-d6) d 8.17 (bs, 1H), 7.86 (m, 4H), 7.25 (d, 8.0 Hz, 1H), 6.71 (s, 1H), 6.66 (d, 8.8 Hz, 1H), 4.01 (t, 6.0 Hz, 2H), 3.78 (t, 6.4 Hz, 2H), 3.42 (m, 1H), 2.14 (s, 3H), 2.07 (p, 6.4 Hz, 2H), 1.87 (m, 4H), 1.36 (dd, 2H), 1.16 (dd, 2H) |
| C3 | trans-4- [(2-{[2- methyl-4- (trifluoro- methoxy) phenyl] amino}- 9H- purin-6- yl)amino] cyclo- hexanol | 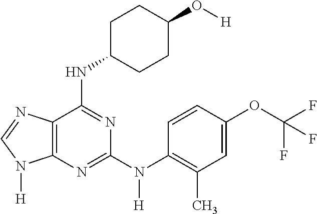 | 5 | Mass Spec.: 423.17509 (M + H); 1H NMR Data: (400 MHz, DMSO-d6) δ 8.30 (s, 1H), 7.64 (d, J = 8.8 Hz, 1H), 7.31 (s, 1H), 7.20 (d, J = 8.8 Hz, 1H), 3.80- 3.30 (m, 6H), 2.28 (s, 3H), 1.87 (m, 4H), 1.35 (m, 2H), 1.15 (m, 2H). |
| C4 | 4-[(2- {[4-(2- ethoxy- ethoxy)-2- methyl- phenyl] amino}-9H- purin-6- yl)amino] cyclo- hexanol | 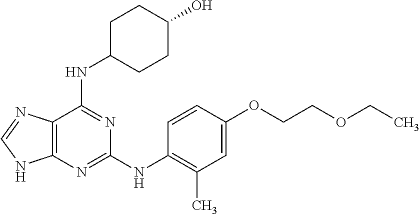 | 5 | Ret. Time (HPLC): 5.88 min; Mass Spec.: 427.24596 (M + 1) |
| C5 | 4-{[2- ({2-methyl- 4-[3-(1H- pyrrol-1- yl)propoxy] phenyl} amino)- 9H-purin- 6- yl]amino} cyclo- hexanol | 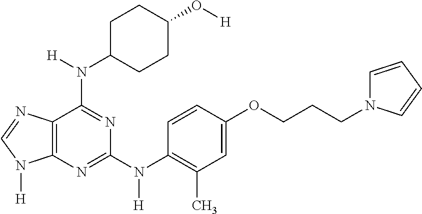 | 5 | Ret. Time (HPLC): 6.65 min; Mass Spec.: 462.2788 (M + 1) |
| C6 | 3-({2- [(2,2- difluoro- 1,3- benzo dioxol-4- yl)amino]- 9H- purin-6- yl}amino) cyclo- hexanol | 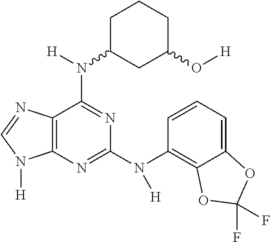 | 2 | Ret. Time (HPLC): 7.084 min; Mass Spec.: 405.14893 (M + l) |
| C7 | trans-4- [(2-{[2- methyl- 4-(3- phenyl- propoxy) phenyl] amino}-9H- purin- 6- yl)amino] cyclo- hexanol | 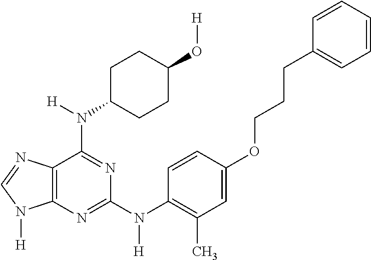 | 5 | Ret. Time (HPLC): 7.3 min; Mass Spec.: 473.2659 (M + l) |
| C8 | trans-4- [(2-{[2- methyl- 4-(2- phenyl- ethoxy) phenyl] amino}- 9H- purin-6- yl)amino] cyclo- hexanol | 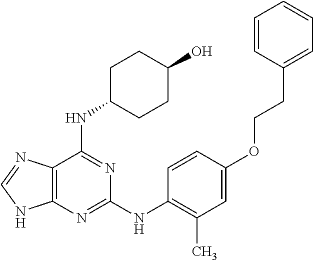 | 5 | Ret. Time (HPLC): 5.29 min; Mass Spec.: 459.2501 (M + l) |
| C9 | N~6~- cyclohexyl- N~2~- [2-methyl- 4-(morpholin- 4- yl)phenyl]- 9H- purine- 2,6-diamine | 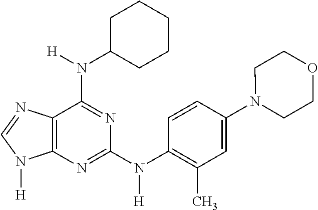 | 2 or 12 | Ret. Time (HPLC): 7.219 min; Mass Spec.: 408.25029 (M + H); 1H NMR Data: (400 MHz, DMSO-d6) δ 12.20 (bs, 1H), 7.68 (s, 1H), 7.58 (bs, 1H), 7.36 (d, J = 8 Hz, 1H), 6.94 (bs, 1H), 6.77 (d, J = 2.5 Hz, 1H), 6.71 (dd, J = 8, 2.5 Hz, 1H), 3.99 (bs, 1H), 3.74 (m, 4H), 3.04 (m, 4H), 2.17 (s, 3H), 1.88 (d, J = 11 Hz, 2H), 1.73 (d, J = 13 Hz, 2H), 1.61 (d, J = 13 Hz, 1H), 1.31 (m, 4H), 1.14 (m, 1H). |
| C10 | N~6~- cyclohexyl- N~2~- [2-methyl- 4-(piperidin- 1- yl)phenyl]- 9H- purine- 2,6-diamine | 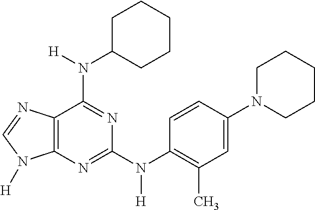 | 5 | Ret. Time (HPLC): 5.989 min; Mass Spec.: 406.26860 (M + l) |
| C11 | N~6~- cyclohexyl- N~2~- (2,2- difluoro-1,3- benzodioxol- 4-yl)- 9H-purine- 2,6- diamine | 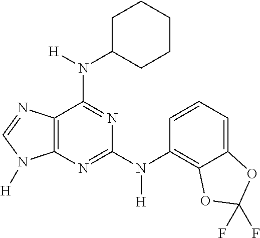 | 5 | Ret. Time (HPLC): 8.03 min; Mass Spec.: 389.1532 (M + l) |
| C12 | ethyl 4- [(2-{[2- methyl-4- (morpholin-4- yl)phenyl] amino}- 9H- purin-6- yl)amino] piperidine- 1-carboxylate | 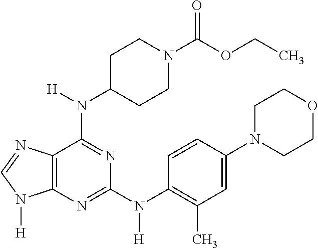 | 5 | Ret. Time (HPLC): 6.244; Mass Spec.: 481.2755 (M + H)+; 1H NMR Data: (400 MHz, DMSO-d6) δ 9.20 (bs, 1H), 8.56 (bs, 1H), 8.24 (s, 1H), 7.23 (d, 8.91 Hz, 1H), 6.89 (d, 2.80 Hz, 1H), 6.83 (dd, 2.80, 8.91 Hz, 1H), 4.05 (q, 6.98 Hz, 2H), 3.97 (m, 3H), 3.73- 3.76 (m, 4H), 3.10-3.13 (m, 4H), 2.76-2.89 (m, 2H), 2.17 (s, 3H), 1.91 (d, 11.0 hz, 2H), 1.42 (q, 11.0 Hz, 2H), 1.19 (t, 3H). |
| C13 | N-{[trans- 4-({2- [(2,4- dimethoxy- phenyl) amino]-9H- purin-6- yl}amino) cyclohexyl] methyl} methane- sulfonamide | 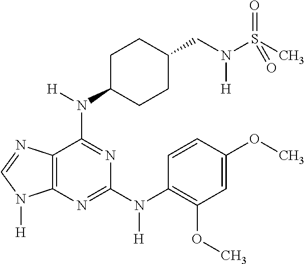 | 20 | Ret. Time (HPLC): 7.04 min; Mass Spec.: 476.2263 (M + H)+ |
| C14 | N-[3- (4-{[6- (cyclohexyl- amino)- 9H-purin- 2- yl]amino}- 3- methyl- phenoxy) propyl]-1H- imidazole-2- carboxamide | 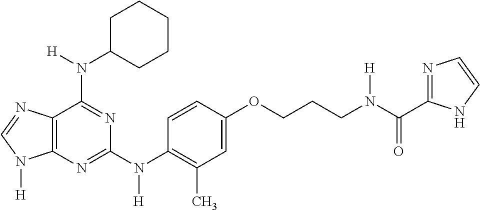 | 18 | Ret. Time (HPLC): 6.31 min; Mass Spec.: 490.26791 (M +1); 1H NMR Data: (400 MHz DMSO-d6) δ 8.56 (t, J = 6.0 Hz, 1H), 8.21 (br s, 1H), 7.28 (d, J = 8.8 Hz, 1H), 7.18 (s, 2H), 6.88 (d, J = 2.4 Hz, 1H), 6.80 (dd, J = 8.4, 2.0 Hz, 1H), 4.02 (t, J = 6.0 Hz, 2H), 3.42 (q, J = 6.0 Hz, 2H), 2.17 (s, 3H), 1.99 (quint, J = 6.8 Hz, 2H), 2.01-1.98 (m, 2H), 1.78- 1.62 (m, 2H), 1.31-1.18 (m, 5H). |
| C15 | N-[3- (4-{[6- (cyclohexyl- amino)- 9H-purin-2- yl]amino}-3- methyl- phenoxy) propyl]-3- methoxy- propanamide | 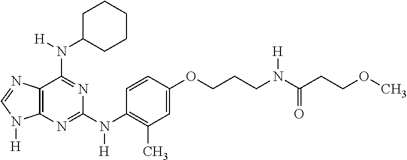 | 18 | Ret. Time (HPLC): 6.39 min; Mass Spec.: 482.2913; 1H NMR Data: (400 MHz, CD3OD) δ 8.02 (s, 1H), 7.26 (d, J = 8.4 Hz, 1H), 6.92 (d, J = 2.8 Hz, 1H), 6.85 (dd, J = 8.4, 3.2 Hz, 1H), 4.04 (t, J = 6.4 Hz, 2H), 3.63 (t, J = 6.4 Hz, 2H), 3.38 (t, J = 6.8 Hz, 2H), 3.31-3.29 (m, 10 H), 2.43 (t, J = 6.4 Hz, 2H), 2.26 (s, 3H), 2.02-1.95 (m, 4H), 1.81 (2H), 1.67 (d, J = 10.8 Hz, 1H), 1.37-1.25 (m, 5H) |
| C16 | N~6~- cyclohexyl- N~2~- (2,4- dimethoxy- phenyl)- 9H-purine- 2,6- diamine | 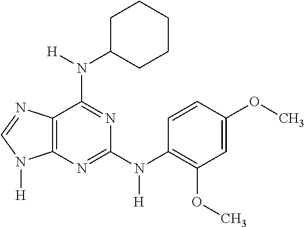 | 5 | Ret. Time (HPLC): 7.49 min; Mass Spec.: 369.2068 (M + l) |
| C17 | N~2~- (2,4- dimethoxy- phenyl)- N~6~- (tetrahydro- 2H-pyran- 4-yl)- 9H-purine- 2,6- diamine | 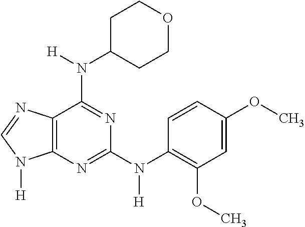 | 5 | Ret. Time (HPLC): 6.069 min; Mass Spec.: 371.18229 (M + H)+ |
| C18 | N-[3- (4-{[6- (cyclohexyl- amino)- 9H-purin-2- yl]amino}-3- methyl- phenoxy) propyl] pyridine-4- carboxamide | 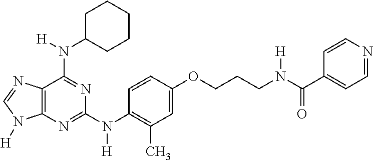 | 18 | Ret. Time (HPLC): 6.24 min; Mass Spec.: 501.2721; 1H NMR Data: (400 MHz, d6-DMSO) δ 9.28 (br s, 1H), 8.91 (t, J = 5.2 Hz, 1H), 8.75 (d, J = 5.2 Hz, 2H), 8.54 (br s, 1H), 8.25 (s, 1H), 7.81 (dd, J = 4.4, 1.6 Hz, 2H), 7.29 (d, J = 9.2 Hz, 1H), 6.88 (d, J = 2.4 Hz, 1H), 6.80 (dd, J = 8.4, 2.0 Hz, 1H), 4.05 (t, J = 6.4 Hz, 2H), 3.46 (q, J = 5.6 Hz, 2H), 2.18 (s, 3H), 2.01 (quint, J = 6.4 Hz, 2H), 1.90-1.87 (m, 2H), 1.71- 1.69 (m, 2H) 1.56 (1H), 1.31-1.13 (m, 5H) |
| C19 | N-[3- (4-{[6- (cyclohexyl- amino)- 9H-purin-2- yl]amino}-3- methyl- phenoxy) propyl] benzamide | 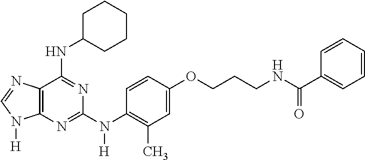 | 18 | Ret. Time (HPLC): 7.10 min; Mass Spec.: 500.27864 (M + 1) |
| C20 | 2-{5-[4-({6- [(trans-4- hydroxy- cyclohexyl) amino]-9H- purin- 2-yl} amino)-3- methyl- phenoxy] pentyl}-1H- isoindole- 1,3(2H)- dione | 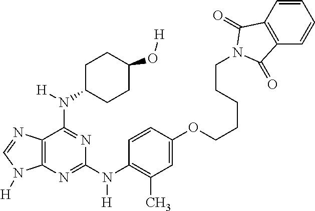 | 18 | Ret. Time (HPLC): 7.0 min; Mass Spec.: 570.2817 (M + l) |
| C21 | 2,2,2- trifluoro-N- {4-[(2- {[2-methyl- 4-(morpholin-4- yl)phenyl] amino}- 9H-purin-6- yl)amino] cyclohexyl} acetamide | 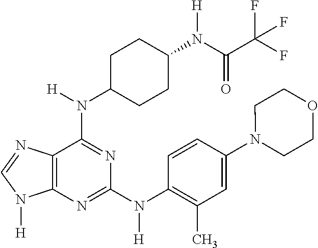 | 19 | Ret. Time (HPLC): 7.11 min; Mass Spec.: 519.24244 (M + 1); 1H NMR Data: (400 MHz, d6-DMSO) δ 9.32 (d, J = 8 Hz, 1H), 8.17 (br s, 1H), 7.23 (d, J = 8.4 Hz, 1H), 6.87-6.80 (m, 1H), 3.74 (t, J = 4.0 Hz, 4H), 3.10 (t, J = 4.4 Hz, 4H), 2.17 (s, 3H), 2.14-1.89 (m, 2H), 1.84-1.82 (m, 2H), 1.53-1.32 (m, 4H) |
| C22 | 2,2,2- trifluoro-N- ({trans-4- [(2-{[2- methyl-4- (morpholin-4- yl)phenyl] amino}- 9H- purin-6- yl)amino] cyclohexyl} methyl) acetamide | 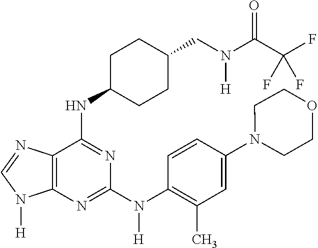 | 19 | Ret. Time (HPLC): 6.366; Mass Spec.: 533.26124 (M + H)+ |
| C23 | N~6~- cyclohexyl- N~2~- (2-methyl- 4-{[2- (1H-pyrrol- 1- yl)ethyl] amino} phenyl)-9H- purine- 2,6-diamine | 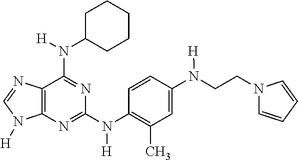 | 5 | Ret. Time (HPLC): 7.13 min; Mass Spec.: 431.2666 (M + l) |
| C24 | 1-ethyl- 3-({trans- 4-[(2- {[2-methyl- 4- (morpholin-4- yl)phenyl] amino}- 9H- purin-6- yl)amino] cyclohexyl} methyl)urea | 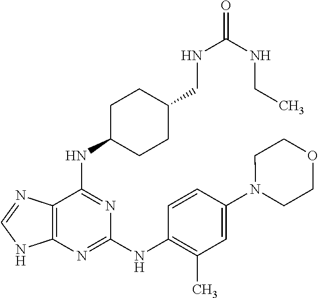 | 19 | Ret. Time (HPLC): 6.205 min; Mass Spec.: 508.28938 (M + H); 1H NMR Data: (400 MHz, d6-DMSO) δ 9.15 (bs, 1H), 8.49 (bs, 1H), 8.21 (bs, 1H), 7.23 (d, 8.92 Hz, 1H), 7.14 (t, 6.17 Hz, 1H), 6.88 (s, 1H), 6.80 (d, 8.92 Hz, 1H), 3.97 (q, 6.75 Hz, 2H), 3.73-3.77 (m, 5H), 3.08-3.14 (m, 4H), 2.83 (t, 5.90 Hz, 2H), 2.16 (s, 3H), 1.96 (d, 10.6 Hz, 2H), 1.75 (d, 10.6 Hz, 2H), 1.21-1.5 (m, 3H), 1.15 (t, 5.90 Hz, 3H), 0.81-0.94 (m, 2H). |
| C25 | 2-[3- (4-{[6- (cyclohexyl- amino)- 9H-purin-2- yl]amino}-3- methyl- phenoxy) propyl]-1H- isoindole- 1,3(2H)- dione | 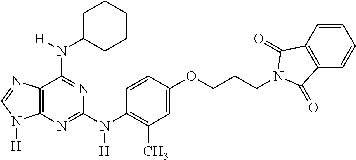 | 18 | Ret. Time (HPLC): 7.328 min MPI-1; Mass Spec.: 526.25625 (M + l) |
| C26 | 4-{[6- (cyclohexyl- amino)- 9H-purin-2- yl]amino}-3- methyl- N-[2- (pyridin-2- yl)ethyl] benzamide | 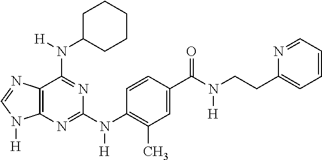 | 21 | Ret. Time (HPLC): 5.99; Mass Spec.: 471 (M + H); 1H NMR Data: (400 MHz, d6- DMSO) δ 8.52 (d, J = 5.2 Hz, 1H), 8.40 (t, J = 5.6 Hz, 1H), 8.11 (d, J = 8.0 Hz, 1H), 7.81 (s, 1H), 7.70 (t, J = 7.6 Hz, 1H), 7.65 (s, 1H), 7.59 (d, J = 8.4 Hz, 1H), 7.27 (d, J = 8.0 Hz, 1H), 7.23 (t, J = 7.2 Hz, 1H), 4.01 (s, 1H), 3.61 (m, 3H), 3.00 (t, J = 7.6 Hz, 2H), 2.30 (s, 3H), 1.95-1.15 (m, 10H); |
| C27 | 4-({2-[(2- chloro-4- ethoxy- phenyl) amino]- 9H-purin-6- yl}amino) cyclohexanol | 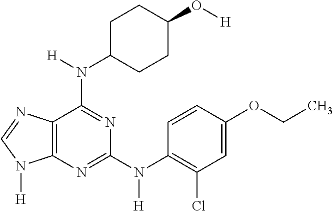 | 5 | Ret. Time (HPLC): 6.67 min; Mass Spec.: 425.16438 (M + H).; 1H NMR Data: (400 MHz, CDCl3) δ 8.3 (s, 1H), 7.57 (s, 1H), 7.15 (s, 1H), 6.96 (d, J = 9.2 Hz, 1H), 4.08 (qt, J = 6.8 Hz, 1H), 3.92 (qt, J = 7.2 Hz, 1H), 3.75 (s, 1H), 3.43 (s, 1H), 2.0-1.6 (m, 4H), 2.0- 1.1 (m, 8H); |
| C28 | trans-4- ({2-[(2,2- difluoro-1,3- benzo- dioxol-4- yl)amino]-9H- purin-6- yl}amino) cyclohexanol | 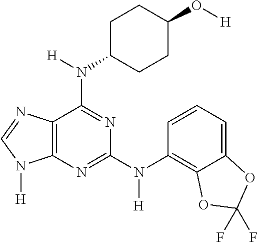 | 5 | Ret. Time (HPLC): 7.4 min; Mass Spec.: 341.2212 (M + l) |
| C29 | N-[3- (4-{[6- (cyclohexyl- amino)- 9H-purin-2- yl]amino}-3- methyl- phenoxy) propyl]-2- methyl- propanamide | 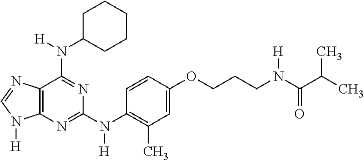 | 18 | Ret. Time (HPLC): 6.60 min; Mass Spec.: 466.29351 (M + 1) |
| C30 | N-[4- (4-{[6- (cyclohexyl- amino)- 9H-purin-2- yl]amino}-3- methyl- phenoxy) butyl]ethane- sulfonamide | 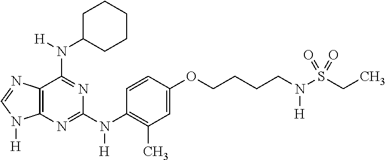 | 18 | Ret. Time (HPLC): 7.10 min; Mass Spec.: 502.25383 (M + 1); 1H NMR Data: (400 MHz, CD3OD) δ 8.03 (s, 1H), 7.70 (dd, J = 1.6, 6.4 Hz, 1H), 7.26-7.22 (m, 1H), 6.92 (d, J = 2.8 Hz, 1H), 6.85 (dd, J = 3.2, 8.8 Hz, 1H), 4.04 (t, J = 6.4 Hz, 2H), 3.22 (q, J = 7.2 Hz, 1H), 3.13 (t, J = 2.8 Hz, 2H), 3.08 (q, J = 7.6 Hz, 2H), 2.26 (s, 3H), 2.32-1.98 (m, 2H), 1.91-1.66 (m, 7H), 1.37-1.25 (m, 7H) |
| C31 | N-[4- (4-{[6- (cyclohexyl- amino)- 9H-purin-2- yl]amino}-3- methyl- phenoxy) butyl]-2- methyl- propanamide | 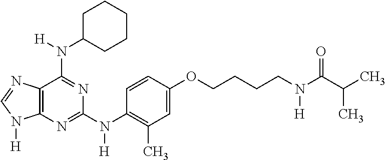 | 18 | Ret. Time (HPLC): 7.08 min; Mass Spec.: 480.30770 (M + 1) |
| C32 | N-[4- (4-{[6- (cyclohexyl- amino)- 9H-purin-2- yl]amino}-3- methyl- phenoxy) butyl] cyclopropane- carboxamide | 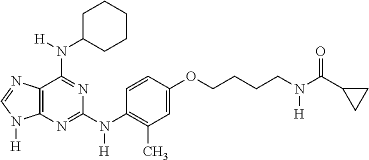 | 18 | Ret. Time (HPLC): 6.99 min ; Mass Spec.: 478.29253; 1H NMR Data: (400 MHz, DMSO-d6) δ 8.21 (br s, 1H), 7.23 (d, J = 6.4 Hz, 1H), 6.89 (d, J = 2.8 Hz, 1H), 4.01 (t, J = 6.5 Hz, 2H), 3.16 (t, J = 6.4 Hz, 2H), 2.18 (s, 3H), 1.92 (m, 2H), 1.85-1.73 (m, 4H), 1.66-1.56 (m, 4H), 1.15- 1.38 (m, 6H) |
| C33 | N-[4- (4-{[6- (cyclohexyl- amino)- 9H-purin-2- yl]amino}-3- methyl- phenoxy) butyl]-3- methoxy- propanamide | 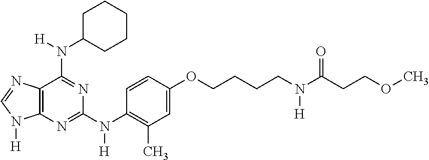 | 18 | Ret. Time (HPLC): 6.72 min; Mass Spec.: 496.30243 (M + 1) |
| C34 | N-[4- (4-{[6- (cyclohexyl- amino)- 9H-purin-2- yl]amino}-3- methyl- phenoxy) butyl] pyridine-4- carboxamide | 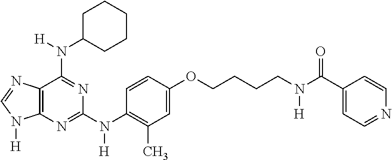 | 18 | Ret. Time (HPLC): 6.74 min; Mass Spec.: 515.28716 (M + 1) |
| C35 | N-({trans- 4-[(2- {[2-methyl-4- (morpholin-4- yl)phenyl] amino}- 9H-purin-6- yl)amino] cyclohexyl} methyl) benzene sulfonamide | 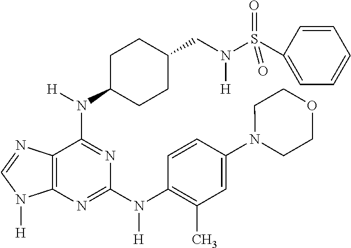 | 19 | Ret. Time (HPLC): 7.014; Mass Spec.: 577.26823 (M + H)+ |
| C36 | 5-methyl-N- ({trans-4- [(2-{[2- methyl-4- (morpholin-4- yl)phenyl] amino}- 9H-purin-6- yl)amino] cyclohexyl} methyl) thiophene- 2- sulfonamide | 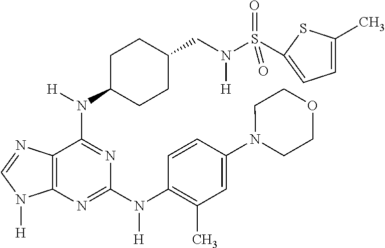 | 19 | Ret. Time (HPLC): 7.185; Mass Spec.: 597.23998 (M + H)+; 1H NMR Data: (400 MHz, CD3OD) δ 8.04 (s, 1H), 7.38 (s, 1H), 7.26- 7.31 (m, 1H), 7.01 (s, 1H), 6.92-6.98 (m, 1H), 6.83 (s, 1H), 3.84-3.88 (m, 5H), 3.19-3.24 (m, 4H), 2.72- 2.77 (m, 2H), 2.53 (s, 3H), 2.27 (s, 3H), 2.07 (d, 11.8 Hz, 2H), 1.86 (d, 11.8 Hz, 2H), 1.46 (bs, 1H), 1.33 (q, 11.8 Hz, 2H), 0.98 (q, 11.8 Hz, 2H). |
| C37 | N~6~- (cyclopropyl- methyl)- N~2~-[2- methyl-4- (morpholin-4- yl)phenyl]- 9H- purine- 2,6-diamine | 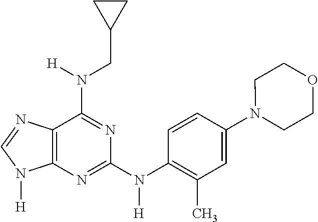 | 5 | Ret. Time (HPLC): 6.63 min; Mass Spec.: 380.2193 (M + l) |
| C38 | N~6~- cyclohexyl- N~2~- [2-methyl- 4-(4- methyl- piperazin-1- yl)phenyl]- 9H- purine- 2,6-diamine | 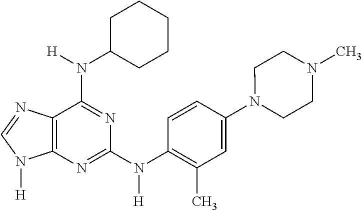 | 12 | Ret. Time (HPLC): 5.96 min; Mass Spec.: 421.2823 (M + l) |
| C39 | N~6~- cyclohexyl- N~2~-{4-[(1- ethyl pyrrolidin-3- yl)oxy]-2- methyl phenyl}- 9H- purine-2,6- diamine | 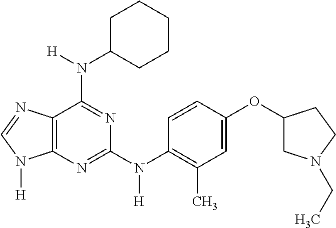 | 9 | Ret. Time (HPLC): 6.08 min; Mass Spec.: 436.28194 (M + H); 1H NMR Data: (400 MHz, DMSO-d6) δ 10.10-9.90 (s, 1H), 8.21 (s, 1H), 7.37 (d, J = 8.8 Hz, 1H), 6.89 (m, 1H), 6.82 (m, 1H), 5.05- 5.20 (m, 1H), 3.90-3.60 (m, 3H), 3.10- 3.50 (m, 4H), 2.20 (s, 3H), 2.10-1.10 (m, 15H); |
| C40 | 4-[(2- {[2-methyl- 4-(morpholin- 4- yl)phenyl] amino}- 9H-purin-6- yl)amino] cyclohexanol | 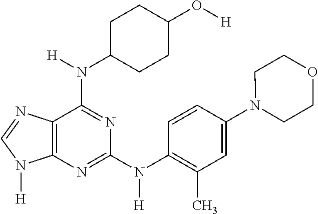 | 2 | Ret. Time (HPLC): 5.813 min; Mass Spec.: 424.24449 (M + H); 1H NMR Data: (400 MHz, d6-DMSO): δ 8.24 (bs, 1H), 7.23 (d, J = 8.5 Hz, 1H), 6.89 (s, 1H), 6.82 (d, J = 8.5 Hz, 1H), 3.75 (m, 4H), 3.11 (m, 4H), 2.17 (s, 3H), 1.89 (m, 4H), 1.35 (m, 1H), 1.17 (m, 1H); |
| C41 | 3-({2- [(2,4- dimethoxy- phenyl) amino]-9H- purin-6- yl}amino) cyclohexanol | 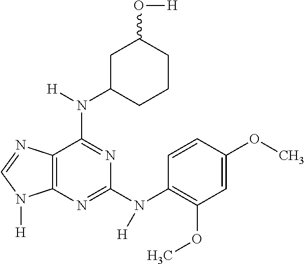 | 5 | Ret. Time (HPLC): 5.8 min; Mass Spec.: 385.1982 (M + l) |
| C42 | 4-{[6- (cyclohexyl- amino)- 9H-purin-2- yl]amino}-3- methyl-N-[2- (pyridin-4- yl)ethyl] benzamide | 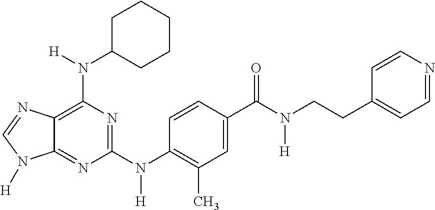 | 21 | Ret. Time (HPLC): 5.98; Mass Spec.: 471 (M + H).; 1H NMR Data: (400 MHz, d4-MeOH) δ 8.43 (d, J = 6.4 Hz, 2H), 8.38 (d, J = 8.8 Hz, 1H), 7.81 (s, 1H), 7.62 (m, 2H), 7.36 (d, J = 6.4 Hz, 2H), 4.09 (s, 1H), 3.66 (t, J = 6.8 Hz, 2H), 3.26 (m, 1H), 2.99 (t, J = 6.8 Hz, 2H), 2.37 (s, 3H), 2.11-1.28 (m, 10H); |
| C43 | 4-{[6- (cyclohexyl- amino)- 9H-purin-2- yl]amino}- 3- methyl-N- [3-(2-oxo- pyrrolidin- 1-yl)propyl] benzamide | 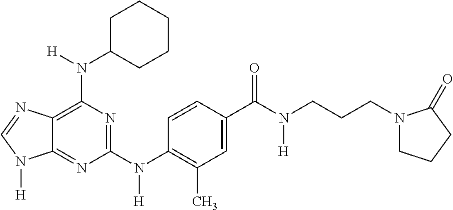 | 21 | Ret. Time (HPLC): 6.65; Mass Spec.: 491 (M + H); 1H NMR Data: (400 MHz, d4- MeOH) δ 8.40 (d, J = 8.8 Hz, 1H), 7.81 (s, 1H), 7.70 (s, 1H), 7.69 (d, J = 8.4 Hz, 1H), 4.10 (m, 1H), 3.51 (t, J = 7.2 Hz, 2H), 3.38 (m, 4H), 2.40 (m, 5H), 2.09 (m, 4H), 1.90-1.20 (m, 10H). |
| C44 | N~2~- (2,4- dimethoxy- phenyl)- N~6~- (2,2,6,6- tetramethyl- tetrahydro- 2H-pyran-4- yl)-9H- purine-2,6- diamine | 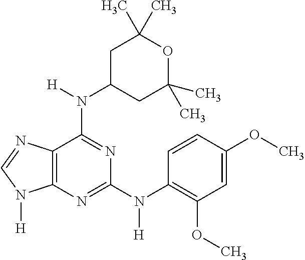 | 5 | Ret. Time (HPLC): 6.61 min; Mass Spec.: 427.24695 (M + 1) |
| C45 | N~6~- cyclohexyl- N~2~-{4- [(2R,6S)- 2,6- dimethyl- morpholin- 4-yl]-2- methyl- phenyl}- 9H-purine- 2,6- diamine | 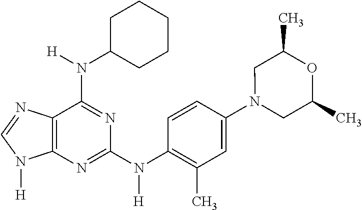 | 2 | Ret. Time (HPLC): 8.010 min; Mass Spec.: 436.28348 (M + l) |
| C46 | N~6~- cyclohexyl- N~2~- {4-[(1- ethyl- piperidin-4- yl)methoxy]- 2- methyl- phenyl}- 9H-purine- 2,6- diamine | 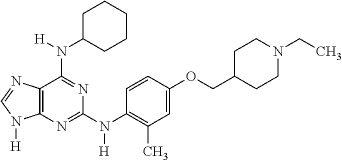 | 11 | Ret. Time (HPLC): 6.37 min; Mass Spec.: 464.313224 (M + H); 1H NMR Data: (400 MHz, CDCl3) δ 9.00 (s, 1H), 8.20 (s, 1H), 7.32 (d, J = 8.8 Hz, 1H), 6.86 (s, 1H), 6.79 (m, 1H), 3.86 (d, J = 5.2 Hz, 2H), 3.53 (m, 2H), 3.11 (t, J = 5.2 Hz, 2H), 2.92 (qt, J = 11.6 Hz, 2H),2.02 (s, 3H), 2.0-1.1 (m, 21H); |
| C47 | N~6~- cyclohexyl- N~2~- {2-methyl- 4-[2- (morpholin-4- yl)ethoxy] phenyl}- 9H-purine- 2,6- diamine | 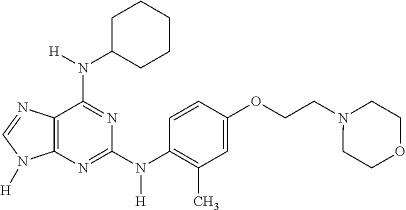 | 10 | Ret. Time (HPLC): 6.06 min; Mass Spec.: 452.2769 (M + 1) |
| C48 | N~6~- cyclohexyl- N~2~-[4- (morpholin- 4-yl)- 2- (trifluoro- methyl) phenyl]-9H- purine- 2,6-diamine | 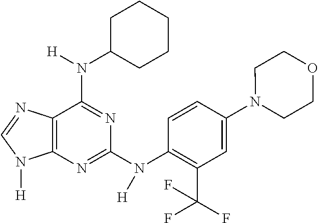 | 2 | Ret. Time (HPLC): 7.829 min; Mass Spec.: 462.22179 (M + l); 1H NMR Data: (400 MHz, DMSO-d6): δ 8.24 (bs, 1H), 7.46 (d, J = 9 Hz, 1H), 7.26 (d, J = 9 Hz, 1H), 7.21 (s, 1H), 3.77 (m, 4H), 3.21 (m, 4H), 1.87 (m, 2H), 1.70 (m, 2H), 1.58 (bs, 1H), 1.22 (m, 5H). |
| C49 | N~6~- cyclohexyl- N~2~-[2- ethoxy-4- (morpholin- 4- yl)phenyl]- 9H- purine-2,6- diamine | 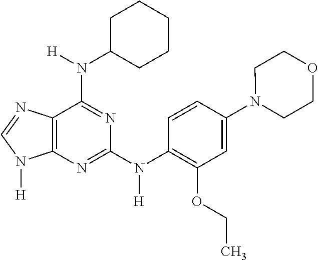 | 2 | Ret. Time (HPLC): 7.542 min; Mass Spec.: 438.25936 (M + H); 1H NMR Data: (400 MHz, DMSO-6) δ 8.86 (bs, 1H), 8.77 (bs, 1H), 8.25 (s, 1H), 7.72 (d, J = 9 Hz, 1H), 6.69 (d, J = 2 Hz, 1H), 6.54 (dd, J = 9, 2 Hz, 1H), 4.11 (q, J = 7 Hz, 2H), 3.94 (bs, 1H), 3.76 (m, 4H), 3.14 (m, 4H), 1.97 (m, 2H), 1.78 (m, 2H), 1.64 (d, J = 12 Hz, 1H), 1.32 (m, 8H); |
| C50 | N~6~- (cyclohexyl- methyl)- N~2~- [2-methyl- 4-(morpholin- 4- yl)phenyl]- 9H- purine-2,6- diamine | 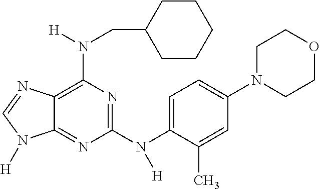 | 5 | Ret. Time (HPLC): 7.639 min; Mass Spec.: 422.2633 (M + H); 1H NMR Data: (400 MHz, DMSO-d6): δ 7.67 (1H, br s), 7.36 (1H, d, J = 8.8 Hz), 6.77 (1H, d, J = 2.8 Hz), 6.71 (1H, dd, J = 8.8 Hz, 2.8 Hz), 3.74 (4H, m), 3.03 (4H, m), 3.4-3.15 (2H, m), 2.16 (3H, s), 1.75-1.55 (7H, m), 1.4-1.1 (5H, m). |
| C51 | 3-[(2- {[2-methyl- 4- (trifluoro- methoxy) phenyl] amino}- 9H-purin- 6- yl)amino] cyclohexanol | 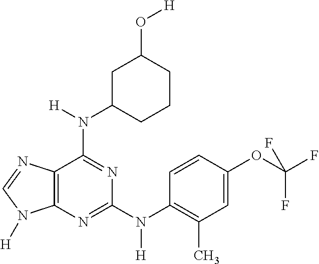 | 2 | Ret. Time (HPLC): 2.920 min; Mass Spec.: 423.17533 (M + l) |
| C52 | N~2~- [2-chloro-4- (morpholin-4- yl)phenyl]- N~6~- cyclohexyl- 9H- purine-2,6- diamine | 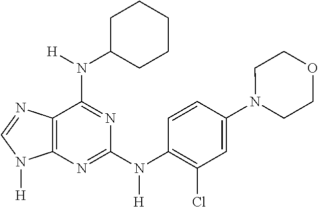 | 2 | Ret. Time (HPLC): 7.61; Mass Spec.: 428 (M + H).; 1H NMR Data: (400 MHz, DMSO-d6) d 7.50 (s, 1H), 7.24 (s, 1H), 7.07 (d, J = 9.2 Hz, 1H), 6.98 (s, 1H), 6.96 (d, J = 9.2 Hz, 1H), 3.74 (m, 4H), 3.14 (m, 4H), 1.95-1.15 (m, 10H); |
| C53 | N~6~- cyclohexyl- N~2~- [2-methyl- 4-(1H- 1,2,4- triazol-1- yl)phenyl]- 9H- purine-2,6- diamine | 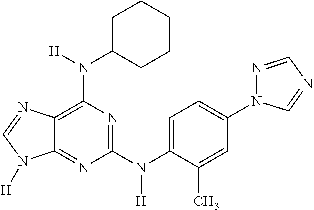 | 8 | Ret. Time (HPLC): 6.84 min; Mass Spec.: 390.21502; 1H NMR Data: (400 MHz, DMSO-d6) δ 9.268 (s, 1H), 8.238 (s, 1H), 7.790 (bs, 1H), 7.692 (s, 1H), 2.333 (s, 3H), 1.909- 1.238 (m, 16H, CH); |
| C54 | 6-(cyclo- hexyloxy)- N-[2- methyl-4- (morpholin-4- yl)phenyl]- 9H- purin-2- amine | 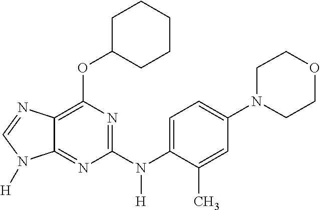 | 17 | Ret. Time (HPLC): 7.6 min; Mass Spec.: 409.23541 (M + H).; 1H NMR Data: (400 MHz, DMSO-d6) d 8.732 (s, 1H), 8.591 (s, 1H), 7.237 (d, J = 8.4 Hz, 1H,), 6.866 (d, J = 2.4 Hz, 1H), 6.813 (dd, J = 8.4 Hz, J = 2.4 Hz, J = 2.8 Hz, 3H), 3.756 (t, J = 9.2 Hz, J = 4.4 Hz, J = 4.8 Hz, 4H), 3.098 (t, J = 9.6 Hz, J = 4.8 Hz, J = 4.8 Hz, 4H), 2.169 (s, 3H), 2.027 (bs, 2H), 1.777 (bs, 2H), 1.573 (bs, 3H), 1.327 (bs, 3H); |
| C55 | N~6~- cyclohexyl- N~2~- [2-methoxy- 4-(morpholin- 4- yl)phenyl]- 9H- purine-2,6- diamine | 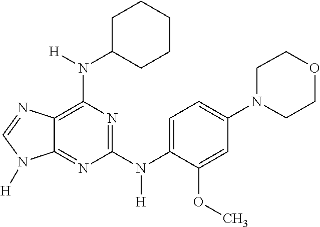 | 5 | Ret. Time (HPLC): 7.26 min; Mass Spec.: 424.2449 (M + l) |
| C56 | N~6~- (4,4- difluoro- cyclohexyl)- N~2~-[2- methyl-4- (morpholin- 4- yl)phenyl]- 9H- purine-2,6- diamine | 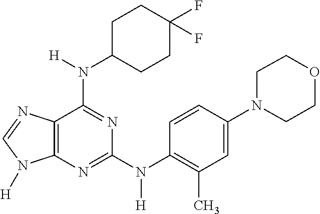 | 7 | Ret. Time (HPLC): 7.204 min; Mass Spec.: 424.22592 (M + H); 1H NMR Data: (400 MHz, DMSO-d6): δ 8.23 (bs, 1H), 7.24 (m, 1H), 6.87 (d, J = 2 Hz, 1H), 6.82 (dd, J = 9, 2 Hz, 1H), 3.75 (m, 4H), 3.11 (m, 4H), 2.18 (s, 3H), 2.09 (m, 2H), 1.99 (m, 2H), 1.63 (m, 1H), 1.24 (m, 2H); |
| C57 | N~6~- (4- fluoro- cyclohex-3- en-1-yl)- N~2~-[2- methyl-4- (morpholin- 4- yl)phenyl]- 9H- purine-2,6- diamine | 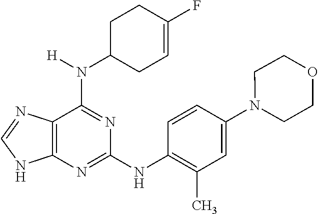 | 7 | Ret. Time (HPLC): 7.131 min; Mass Spec.: 444.22723 (M + l); 1H NMR Data; 1H NMR (400 MHz, d6- DMSO) δ 8.54 (bs, 1H), 8.23 (bs, 1H), 7.22 (d, J = 8.5 Hz, 1H), 6.88 (s, 1H), 6.82 (d, J = 8.5 Hz, 1H), 5.22 (d, J = 17 Hz, 1H), 3.75 (m, 4H), 3.11 (m, 4H), 2.19 (m, 7H), 1.97 (m, 1H), 1.79 (m, 1H), 1.21 (m, 1H); |
| C58 | N~6~- (4- ethoxycyclo- hexyl)- N~2~-[2- methyl- 4-(morpholin- 4- yl)phenyl]- 9H- purine-2,6- diamine | 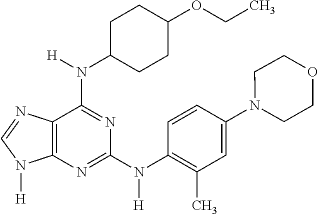 | 6 | Ret. Time (HPLC): 6.869 min; Mass Spec.: 452.27681(M + H); 1H NMR Data: (400 MHz, DMSO- d6) d 9.50 (s, 1H), 9.35 (m, 1H), 8.38 (s, 1H), 7.61 (d, J = 8.5 Hz, 1H), 7.35 (s, 1H), 7.28 (d, J = 8.5 Hz, 1H), 3.93 (m, 4H), 3.45 (q, J = 7 Hz, 2H), 3.36 (m, 4H), 3.25 (m, 1H), 2.29 (s, 1H), 1.99 (m, 4H), 1.41 (m, 2H), 1.19 (m, 2H), 1.10 (t, J = 7 Hz, 3H); |
| C59 | N~6~- cyclohexyl- N~2~- [2-methyl- 4-(1- oxidothio- morpholin- 4-yl)phenyl]- 9H- purine-2,6- diamine | 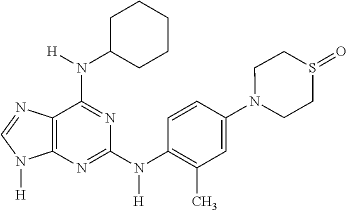 | 3 | Ret. Time (HPLC): 6.451 min; Mass Spec.: 456.21768 (M + 1) |
| C60 | (4-{[6- (cyclohexyl- amino)- 9H-purin-2- yl]amino}-3- methyl- phenyl)(4- methyl- piperazin-1- yl)methanone | 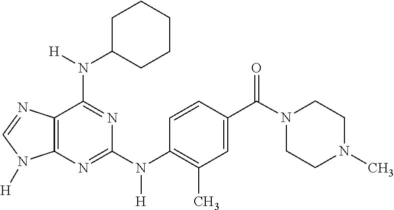 | 13 | Ret. Time (HPLC): 5.70 min; Mass Spec.: 449.27679 (M + H); 1H NMR Data: (400 MHz, DMSO-d6) δ 7.87 (s, 1H), 7.36 (m, 1H), 7.31 (m, 1H), 3.57 (s, 3H), 3.30-3.50 (m, 4H), 3.10 (s, 1H), 2.80 (s, 3H), 2.33 (s, 4H), 1.93 (s, 2H), 1.76 (s, 2H), 1.65-1.50 (m, 2H), 1.50-1.10 (m, 8H, CH); |
| C61 | (4-{[6- (cyclo- hexylamino)- 9H-purin- 2- yl]amino}- 3- methyl- phenyl) (morpholin- 4-yl) methanone | 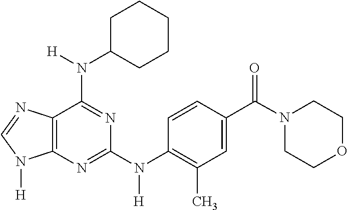 | 13 | Ret. Time (HPLC): 6.74 min; Mass Spec.: 436.24555 (M + H)+; 1H NMR Data: (400 MHz, CDCl3) d 8.37 (s, 1H), 7.76 (s, 1H), 7.33 (s, 1H), 7.25 (d, J = 7.6 Hz, 1H), 4.00-3.50 (m, 12H), 2.31 (s, 3H), 1.92 (m, 2H), 1.77 (m, 2H), 1.61 (m, 1H), 1.40-1.10 (m, 5H); |
| C62 | N~2~- [2-methyl- 4-(morpholin- 4- yl)phenyl]- N~6~- (2,2,6,6- tetramethyl- tetrahydro- 2H-pyran-4- yl)-9H- purine-2,6- diamine | 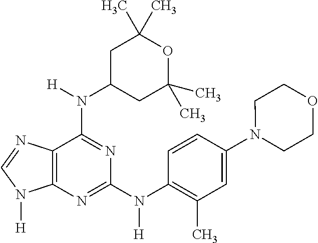 | 5 | Ret. Time (HPLC): 6.25 min; Mass Spec.: 466.29136 (M + 1) |
| C63 | N~6~- cyclohexyl- N~2~- [2-methyl- 4-(morpholin- 4- ylmethyl) phenyl]- 9H-purine- 2,6- diamine | 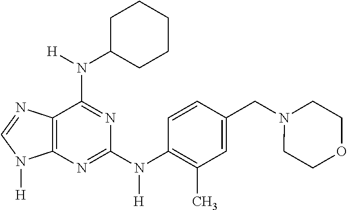 | 14 | Ret. Time (HPLC): 5.87 min; Mass Spec.: 422.26629 (M + H); 1H NMR Data: (400 MHz, DMSO-d6) δ 10.91 (s, 1H), 7.81 (d, J = 8.8 Hz, 1H), 7.48 (s, 1H), 7.44 (d, J = 8.8 Hz, 1H), 4.3 (m, 2H), 3.95 (d, J = 12 Hz , 2H), 3.77 (t, J = 12 Hz, 2H), 3.24 (d, J = 12 Hz, 2H), 3.08 (m, 2H), 2.5 (m, 4H), 2.32 (s, 3H), 2.00-1.10 (m, 10H, CH); |
| C64 | N~6~- cyclohexyl- N~6~- methyl- N~2~- [2-methyl- 4-(morpholin- 4-yl)phenyl]- 9H- purine-2,6- diamine | 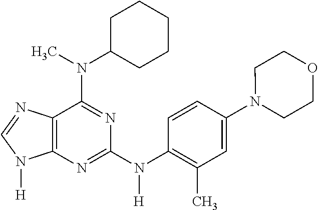 | 5 | Ret. Time (HPLC): 6.71 min; Mass Spec.: 422.24859 (M + H) |
| C65 | N~6~- cyclohexyl- N~2~- (2,4- dimethoxy- phenyl)- N~6~- methyl-9H- purine-2,6- diamine | 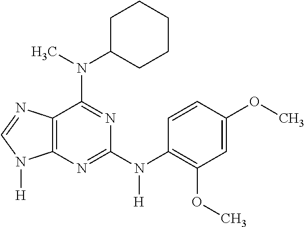 | 5 | Ret. Time (HPLC): 7.10 min; Mass Spec.: 383.22059; 1H NMR Data: (400 MHz, DMSO-d6): δ 7.98 (bs, 1H), 6.68 (d, J = 2.4 Hz, 1H), 6.54 (dd, J = 2.4 Hz, 8.5 Hz, 1H), 3.84 (s, 3H), 3.77 (s, 3H), 1.78-1.85 (m, 2H), 1.53-1.74 (m, 5H), 1.34 (bs, 2H), 1.08-1.22 (m, 1H) |
| C66 | N-[3- (4-{[6- (cyclohexyl- amino)- 9H-purin- 2- yl]amino}- 3-methyl- phenoxy) propyl]-2- (3,5- dimethyl- 1H- pyrazol-1- yl)-3- methyl- butanamide | 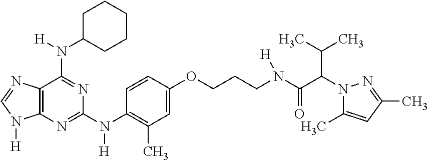 | 18 | Ret. Time (HPLC): 8.225 min; Mass Spec.: 574.36035; 1H NMR Data: (400 MHz, DMSO-d6): δ 9.32 (s, 1H), 8.60 (s, 1H), 8.26 (s, 1H), 8.15 (s, 0.5H), 8.07 (t, J = 5.9 Hz, 1H), 7.96 (s, 0.3H), 7.30 (d, J = 8.3 Hz, 1H), 6.84 (s, 1H), 6.76 (d, J = 8.3 Hz, 1H), 5.84 (s, 1H, 4.21 (d, J = 12 Hz, 1H), 3.93 (t, J = 6.0 Hz, 2H), 3.81 (bs, 1H), 3.24 (q, J = 6.0 Hz, 2H), 2.54-2.64 (m, 1H), 2.22 (s, 3H), 2.19 (s, 3H), 2.19 (s, 3H), 2.10 (s, 3H), 1.80-1.94 (m, 5H), 1.66-1.78 (m, 3H), 1.52-1.65 (m, 2H), 1.08- 1.41 (m, 8H), 0.94 (d, J = 6.7 Hz, 3H), 0.81-0.89 (m, 1H), 0.65 (d, J = 6.7 Hz, 3H) |
| C67 | N-[3- (4-{[6- (cyclohexyl- amino)- 9H-purin-2- yl]amino}- 3-methyl- phenoxy) propyl]-L- valinamide trifluoro- acetate | 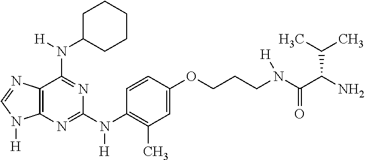 | 18 | Ret. Time (HPLC): 6.293 min; Mass Spec.: 495.31764 (M + 1) |
| C68 | N~2~- [2-methyl- 4-(morpholin- 4-yl)phenyl]- N~6~- [4-(morpholin- 4- yl)cyclohexyl]- 9H- purine-2,6- diamine | 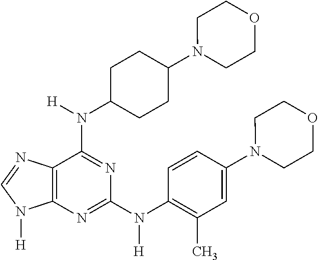 | 4 | Ret. Time (HPLC): 5.387 min 5.507 min; Mass Spec.: 493.30250 (M + 1) |
| C69 | N~6~- cyclohexyl- N~2~- {2-methyl- 4-[(4- methyl- piperazin-1- yl)methyl] phenyl}- 9H-purine- 2,6- diamine | 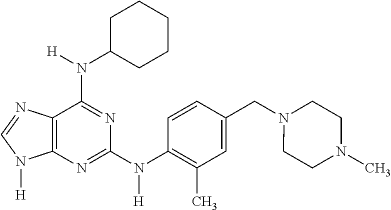 | 14 | Ret. Time (HPLC): 5.75 min; Mass Spec.: 435.2979 (M + 1); 1H NMR Data: (400 MHz, DMSO- d6) δ 7.81 (s, 1H), 7.48 (m, 2H), 3.89 (s, 1H), 3.07 (m, 2H), 2.8 (m, 4H), 2.3 (m, 4H), 2.00-1.10 (m, 17H) |
| C70 | N~6~- cyclohexyl- N~2~- {2-methyl- 4-[(8- methyl-8- azabicyclo [3.2.1]oct-3- yl)oxy] phenyl}- 9H-purine- 2,6- diamine | 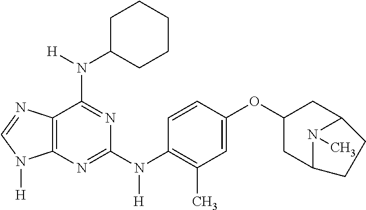 | 9 | Ret. Time (HPLC): 6.09 min; Mass Spec.: 462.2983 (M + 1) |
| C71 | N~6~- (bicyclo [2.2.1]hept- 2-yl)-N~2~-[2- methyl-4- (morpholin-4- yl)phenyl]-9H- purine-2,6- diamine | 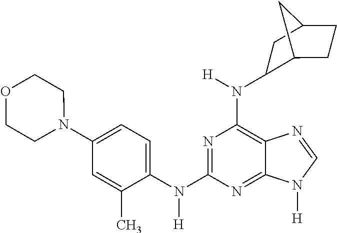 | 5 | Ret. Time (HPLC): 7.39 min; Mass Spec.: 420.25014 (M + 1) |
| C72 | N~6~- cyclohexyl- N~2~-[4-(1,1- dioxidothio- morpholin- 4-yl)-2- methylphenyl]- 9H- purine-2,6- diamine | 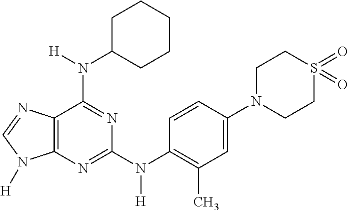 | 3 | Ret. Time (HPLC): 7.137 min; Mass Spec.: 456.21867 (M + 1) |
| C73 | (4-{[6- (cyclohexyl amino)- 9H-purin- 2-yl]amino}- 3- methylphenyl) [4-(pyrrolidin- 1-yl)piperidin- 1-yl] methanone | 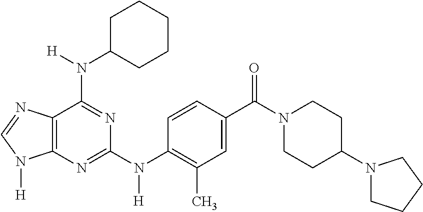 | 21 | Ret. Time (HPLC): 5.9; Mass Spec.: 503 (M + H).; 1H NMR Data: (400 MHz, d6-DMSO) δ 7.90 (d, J = 8.0 Hz, 1H), 7.76 (s, 1H), 7.19 (s, 1H), 7.11 (d, J = 8.0 Hz, 1H), 3.45-3.20 (m, 10H), 2.67 (m, 2H), 2.33 (m, 1H), 2.29 (s, 3H), 1.95- 1.15 (m, 15H); |
| C74 | (4-{[6- (cyclohexyl- amino)- 9H-purin-2- yl]amino}- 3-methyl- phenyl)[4- (propan-2-yl) piperazin-1- yl]methanone | 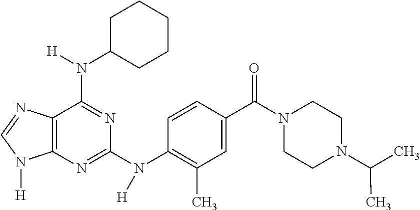 | 21 | Ret. Time (HPLC): 5.96 min |
| C75 | 4-{[6- (cyclohexyl- amino)- 9H-purin- 2-yl]amino}- N-ethyl-3- methyl- benzamide | 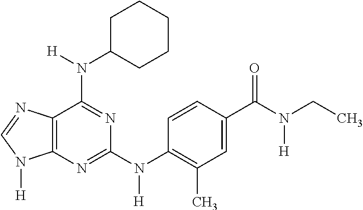 | 21 | Ret. Time (HPLC): 6.99; Mass Spec.: 394 (M + H).; 1H NMR Data: (400 MHz, d6-DMSO) δ 8.12 (d, J = 8.0 Hz, 1H), 7.81 (s, 1H), 7.67 (s, 1H), 7.61 (d, J = 8.0 Hz, 1H), 3.10 (m, 3H), 2.31 (s, 3H), 1.95- 1.15 (m, 10H), 1.12 (t, J = 7.6 Hz, 3H); |
| C76 | N~6~-(2,3- dihydro- 1H-inden- 1-yl)-N~2~- [2-ethoxy-4- (morpholin- 4-yl)phenyl]- 9H- purine-2,6- diamine | 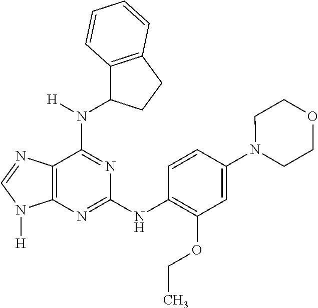 | 2 | Ret. Time (HPLC): 7.692 min; Mass Spec.: 472.24471 (M + 1) |
| C77 | N~6~- (bicyclo [2.2.1]hept- 2-yl)-N~ 2~-[2- methyl-4- (morpholin- 4-yl)phenyl] (8-~2~H)-9H- purine-2,6- diamine | 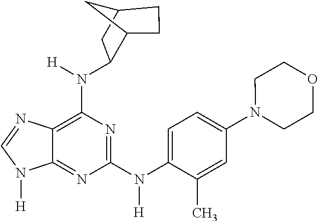 | 5 | Ret. Time (HPLC): 7.38 min; Mass Spec.: 420.25064 (M + 1) |
| C78 | N~2~- {2-chloro-4- [2-(morpholin- 4-yl)ethoxy] phenyl}- N~6~- cyclohexyl- 9H-purine- 2,6- diamine | 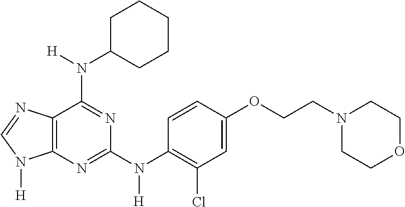 | 10 | Ret. Time (HPLC): 6.07 min; Mass Spec.: 472.2222 (M + 1); 1H NMR Data: (400 MHz, DMSO-d6) δ 7.21 (s, 1H), 7.00 (d, J = 8.8 Hz, 1H), 6.58 (s, 1H), 4.37 (m, 2H), 3.99 (m, 1H), 3.70 (m, 2H), 3.6-3.4 (m, 4H), 3.20 (m, 1H), 2.00-1.15 (m, 14H). |
| C79 | N~6~- cyclohexyl- N~2~-[4-(1- ethyl- piperidin-4- yl)-2-methyl- phenyl]-9H- purine-2,6- diamine | 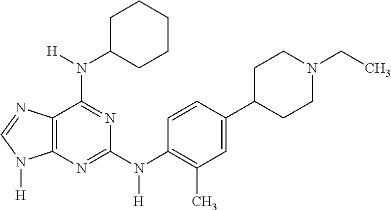 | 16 | Ret. Time (HPLC): 6.18 min; Mass Spec.: 434.3027 (M + 1); 1H NMR Data: (400 MHz, DMSO-d6) δ 9.11 (s, 0.5H), 8.22 (s, 1H), 7.51 (d, J = 8.0 Hz, 1H), 7.12 (s, 1H), 7.06 (d, J = 8.0 Hz, 1H), 3.81 (s, 0.5H), 3.58 (m, 2H), 3.15 (m, 2H), 3.00 (qt, J = 9.6 Hz, 2H), 2.80 (m, 1H), 2.23 (s, 3H), 2.1-1.5 (m, 9H), 1.40-1.10 (m, 10H). |
| C80 | 1-amino- N-[3-(4- {[6- (cyclohexyl- amino)- 9H-purin- 2-yl]amino}- 3-methyl- phenoxy) propyl]cyclo- propane carboxamide trifluoro- acetate | 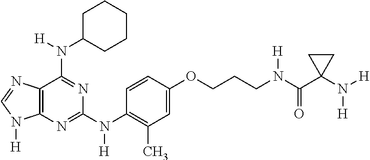 | 18 | Ret. Time (HPLC): 6.138 min; Mass Spec.: 479.28715 (M + H) |
| C81 | N-[3- (4-{[6- (cyclohexyl- amino)- 9H-purin-2- yl]amino}-3- methyl- phenoxy) propyl]-3- methyl-5- phenyl-1,2- oxazole-4- carboxamide | 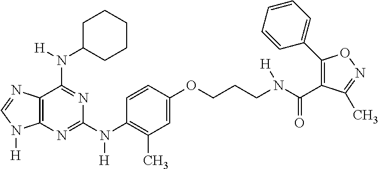 | 18 | Ret. Time (HPLC): 7.2 min; Mass Spec.: 581.3001 (M + 1) |
| C82 | N~6~- cyclohexyl- N~2~- [2-ethoxy-4- (morpholin-4- ylmethyl) phenyl]- 9H-purine- 2,6- diamine | 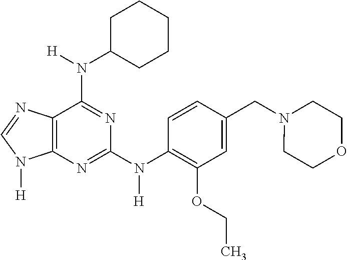 | 15 | Ret. Time (HPLC): 6.21 min; Mass Spec.: 452.2769 (M + 1); 1H NMR Data: (400 MHz, DMSO) δ 10.06 (s, 1H), 8.43 (s, 1H), 8.25 (s, 1H), 7.21 (s, 1H), 7.09 (d, J = 7.6 Hz, 1H), 4.31 (s, 2H), 4.18 (qt, J = 7.2 Hz, 2H), 3.98 (m, 2H), 3.64 (t, J = 12 Hz, 2H), 2.28 (m, 2H), 3.11 (s, 2H), 2.00 (m, 2H), 1.82 (m, 2H), 1.66 (m, 1H), 1.50-1.10 (m, 10H). |
| C83 | N~6~- cyclohexyl- N~2~- {2-ethoxy- 4-[(1-ethyl- pyrrolidin-3- yl)oxy] phenyl}- 9H-purine- 2,6- diamine | 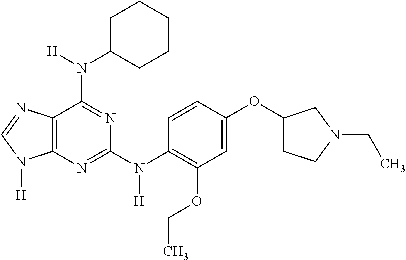 | 9 | Ret. Time (HPLC): 6.25 min; Mass Spec.: 466.2925 (M + 1) |
| C84 | N~6~- cyclohexyl- N~2~- [2-ethoxy-4- (morpholin-4- yl)phenyl]- N~6~- methyl-9H- purine- 2,6-diamine | 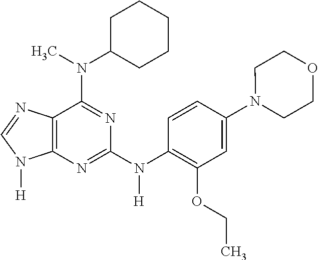 | 2 | Ret. Time (HPLC): 7.768 min; Mass Spec.: 452.27688 (M + 1); 1H NMR Data: (400 MHz, DMSO- d6) δ 8.58 (bs, 1H), 8.12 (bs, 1H), 7.72 (bs, 1H), 6.69 (d, J = 2 Hz, 1H), 6.54 (dd, J = 9, 2 Hz, 1H), 4.11 (q, J = 7 Hz, 2H), 3.94 (bs, 1H), 3.76 (m, 4H), 3.12 (m, 4H), 1.81 (m, 2H), 1.64 (m, 5H), 1.44-1.05 (m, 6H) |
| C85 | N~2~- [2-methyl- 4-(morpholin- 4- yl)phenyl]- N~6~- (tetrahydro- 2H-thiopyran- 4-yl)- 9H-purine- 2,6- diamine | 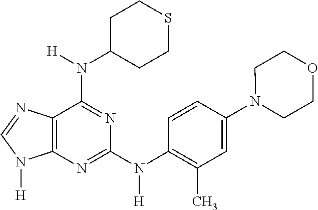 | 2 | Ret. Time (HPLC): 6.793 min; Mass Spec.: 426.20803 (M + 1) |
| C86 | 4-{[6- (cyclohexyl- amino)- 9H-purin-2- yl]amino}-3- methyl-N-[2- (pyridin-3- yl)ethyl] benzamide | 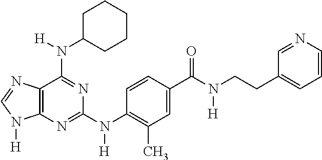 | 21 | Ret. Time (HPLC): 5.97; Mass Spec.: 471 (M + H); 1H NMR Data: (400 MHz, DMSO-d6) δ 8.45 (s, 1H), 8.41 (m, 2H), 8.10 (d, J = 8.4 Hz, 1H), 7.65 (m, 3H), 7.59 (d, J = 8.4 Hz, 1H), 7.31 (dd, J = 8.0, 4.8 Hz, 1H), 4.02 (s, 1H), 3.61 (m, 1H), 3.50 (dd, J = 12.8, 7.2 Hz, 2H), 2.87 (t, J = 6.8 Hz, 2H), 2.30 (s, 3H), 1.95-1.15 (m, 10H); |
| C87 | (4-{[6- (cyclohexyl- amino)- 9H-purin-2- yl]amino}-3- methyl- phenyl)[4- (2-hydroxy- ethyl) piperidin-1- yl]methanone | 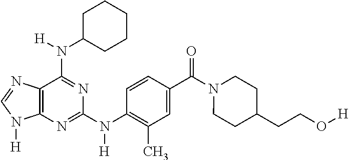 | 21 | Ret. Time (HPLC): 6.61; Mass Spec.: 478 (M + H); 1H NMR Data: (400 MHz, DMSO-d6) δ 7.91 (d, J = 8.0 Hz, 1H), 7.77 (s, 1H), 7.17 (s, 1H), 7.12 (d, J = 8.0 Hz, 1H), 4.40 (m, 2H), 3.45 (m, 5H), 2.67 (m, 2H), 2.33 (m, 1H), 2.27 (s, 3H), 1.95- 1.15 (m, 14H); |
| C88 | N~2~- [2-methoxy-4- (morpholin-4- yl)phenyl]- N~6~- (tetrahydro- 2H-pyran-4- yl)-9H- purine-2,6- diamine | 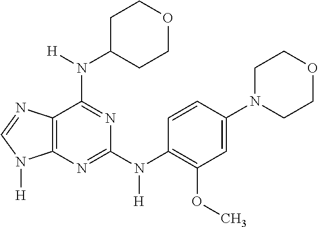 | 2 | Ret. Time (HPLC): 6.165 min; Mass Spec.: 426.3167 (M + 1) |
| C89 | 6- (cyclohexyl- methyl)- N-[2- methoxy-4- (morpholin-4- yl)phenyl]- 9H- purin-2- amine | 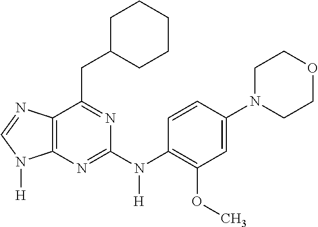 | 22 | Ret. Time (HPLC): 7.48 min; Mass Spec.: 423.25030 (M + 1); 1H NMR Data: (400 MHz, DMSO-d6) δ 8.51 (s,1H), 8.35 (bs, 1H), 7.83 (d, J = 8.8 Hz, 1H), 6.71 (s, 1H), 6.56 (d, J = 8.8 Hz, 1H), 3.81 (s, 3H), 3.77 (m, 4H), 3.14 (m, 4H), 2.86 (d, J = 7.2 Hz, 1H), 1.92 (m, 1H), 1.64 (m, 5H), 1.23-1.00 (m,5H). |
| C90 | (2-{[2- methoxy-4- (morpholin-4- yl)phenyl] amino}- 9H-purin-6- yl)(morpholin- 4- yl)methanone | 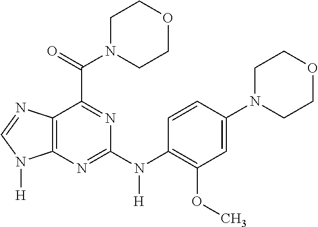 | 24 | Ret. Time (HPLC): 8.08 min; Mass Spec.: 440.20413 (M +1 ) |
| C91 | N~6~- cyclohexyl- N~2~-{2-methyl- 4-[3- (morpholin-4- yl)prop-1-yn-1- yl]phenyl}- 9H- purine-2,6- diamine | 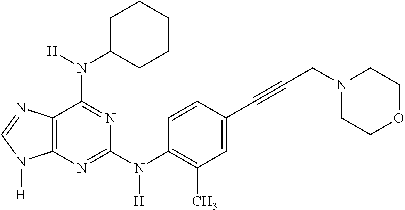 | 23 | 1H NMR Data: (400 MHz, CD3OD) dppm 8.09 (s, 1H), 7.76 (bd, J = 8 Hz, 1H), 7.48 (bs, 1H), 7.41 (dd, J = 8 Hz, 2 Hz, 1H), 4.32 (s, 2H), 3.95 (m, 5H), 3.39 (m, 4H), 2.33 (s, 3H), 2.00 (m, 2H), 1.80 (m, 2H), 1.66 (m, 1H), 1.39-1.28 (m, 5H). |

The structures and characterization of additional exemplary compounds made and the synthetic scheme of each (appropriate, commercially available starting materials are known to those skilled in the art) are provided in Table 2 below.

TABLE 2
Additional Exemplary Compounds of the Invention

| Com- | | | | |
|---|---|---|---|---|
| pound | | | | |
| # | IUPAC Name | Structure | Synthesis | Analytical Data |
| C092 | N~2~- [4-(1,1- dioxidothio- morpholin- 4-yl)-2- methoxy- phenyl]- N~6~- (tetrahydro- 2H- pyran-4- yl)-9H- purine- 2,6-diamine | 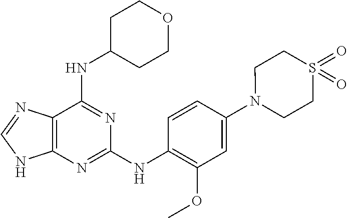 | Scheme 3 | Ret. Time (HPLC): 5.799 min; Mass Spec.: 474.19241 (M + 1) |
| C093 | N~6~- cyclohexyl- N~2~- [4-(1,1- dioxidothio- morpholin- 4-yl)-2- methoxy- phenyl]- 9H-purine- 2,6-diamine | 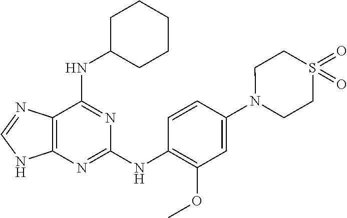 | Scheme 3 | Ret. Time (HPLC): 7.534 min; Mass Spec.: 472.21233 (M + 1) |
| C094 | N~6~- Cyclohexyl- N~2~- {2-methyl- 4-[(2- methyl- piperazin- 1- yl)methyl] phenyl}- 3H-purine- 2,6-diamine | 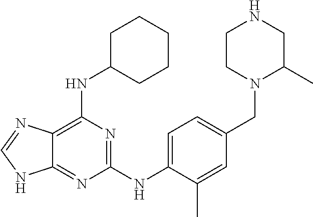 | Scheme 14 | Ret. Time (HPLC): 5.955 min; Mass Spec.: 435.3004 (M + 1) |
| C095 | N~6~- Cyclohexyl- N~2~- [2-methyl- 4-(piperidin- 1- ylmethyl) phenyl]- 3H-purine- 2,6-diamine | 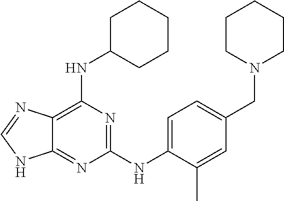 | Scheme 14 | Ret. Time (HPLC): 7.250 min; Mass Spec.: 420.3232 (M + 1) |
| C096 | N~6~- Cyclohexyl- N~2~- {4-[(4- ethyl- piperazin-1- yl)methyl]-2- methyl- phenyl}- 3H-purine- 2,6-diamine | 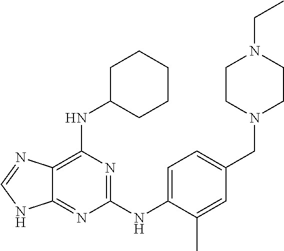 | Scheme 14 | Ret. Time (HPLC): 7.112 min; Mass Spec.: 449.3117 (M + 1) |
| C097 | N~6~- Cyclohexyl- N~2~- {4-[(2,6- dimethyl- morpholin- 4-yl) methyl]- 2- methyl phenyl}- 3H-purine- 2,6-diamine | 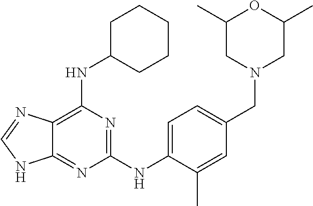 | Scheme 14 | Ret. Time (HPLC): 5.439 min; Mass Spec.: 450.2868 (M + 1) |
| C098 | N~2~- [2-Chloro- 4- (morpholin-4- ylmethyl) phenyl]- N~6~- cyclohexyl- 3H- purine- 2,6-diamine | 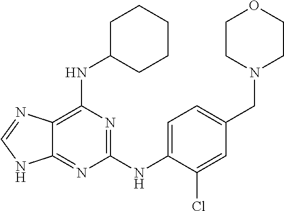 | Scheme 14 | Ret. Time (HPLC): 9.193 min; Mass Spec.: 442.2137 (M + 1) |
| C099 | N~2~- [4-(1- ethyl- piperidin-4- yl)-2- methoxy- phenyl]- N~6~- (tetrahydro- 2H- pyran-4- yl)-9H- purine- 2,6-diamine | 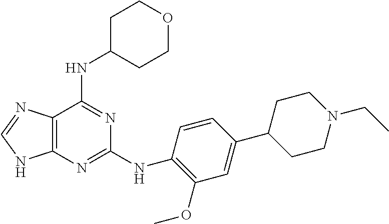 | Scheme 16 | Ret. Time (HPLC): 4.705 min; Mass Spec.: 452.27823 (M + 1) |
| C100 | N~6~- cyclo- hexyl- N~2~- [4-(1-ethyl- piperidin-4- yl)-2- methoxy- phenyl] 9H-purine- 2,6-diamine | 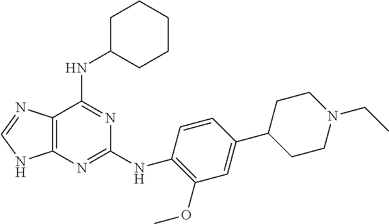 | Scheme 16 | Ret. Time (HPLC): 5.67 min; Mass Spec.: 450.29613 (M + 1) |
| C101 | rel-N~6~- Cyclohexyl- N~2~- (4-{3- [(2R,6S)- 2,6- dimethyl- morpholin- 4-yl] prop-1- yn-1- yl}-2- methyl- phenyl)- 3H-purine- 2,6-diamine | 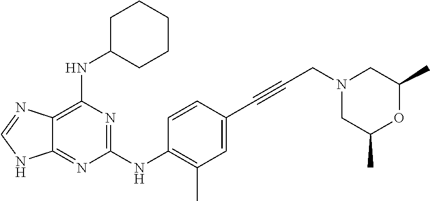 | Scheme 23 | Ret. Time (HPLC): 7.724 min; Mass Spec.: 474.29744 (M + 1) |
| C102 | 2-{4- [3-(3- Methyl- 4-{[6- (tetrahydro- 2H-pyran- 4-ylamino)- 3H-purin-2- yl]amino} phenyl) prop-2- yn-1-yl] piperazin- 1-yl} ethanol | 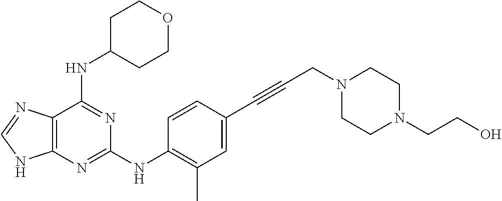 | Scheme 23 | Ret. Time (HPLC): 4.804 min; Mass Spec.: 491.28720 (M + 1) |
| C103 | N-[(4- {[2-({2- Methyl- 4-[3- (morpholin- 4- yl)prop-1- yn-1-yl] phenyl} amino)- 3H-purin- 6- yl]amino} cyclohexyl) methyl] acetamide | 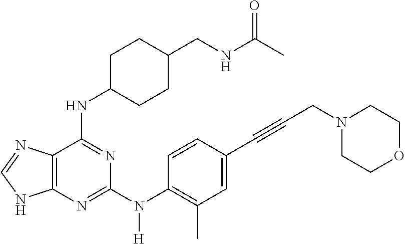 | Scheme 23 | Ret. Time (HPLC): 6.229 min; Mass Spec.: 517.30291 (M + 1) |
| C104 | N~2~-[4- (Cyclo- hexyl- ethynyl)- 2- methyl- phenyl]- N~6~- (tetrahydro- 2H- pyran-4- yl)-3H- purine- 2,6-diamine | 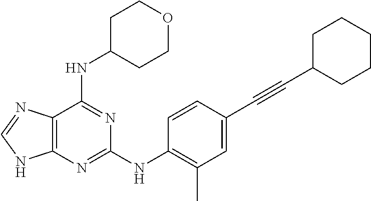 | Scheme 23 | Ret. Time (HPLC): 7.940 min; Mass Spec.: 431.25729 (M + 1) |
| C105 | rel-N~2~-(2- Chloro- 4-{3- [(2R,6S)- 2,6- dimethyl- morpholin- 4-yl] prop-1- yn-1-yl} phenyl)- N~6~- cyclohexyl- 3H- purine- 2,6-diamine | 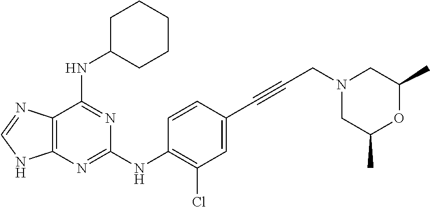 | Scheme 23 | Ret. Time (HPLC): 6.472 min; Mass Spec.: 494.24296 (M + 1) |
| C106 | N~2~- {4-[3-(1,1- Dioxidothio- morpholin- 4-yl)prop- 1-yn- 1-yl]-2- methyl- phenyl}- N~6~- (tetrahydro- 2H- pyran-4- yl)-3H- purine- 2,6-diamine | 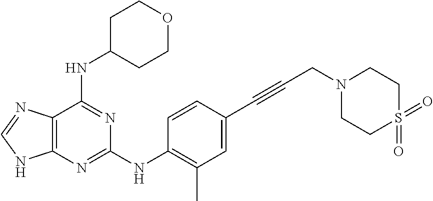 | Scheme 23 | Ret. Time (HPLC): 5.720 min; Mass Spec.: 496.21192 (M + 1) |
| C107 | N~2~-{2- Methyl- 4-[3- (piperazin-1- yl)prop- 1-yn-1- yl]phenyl}- N~6~- (tetrahydro- 2H- pyran-4- yl)-3H- purine- 2,6-diamine | 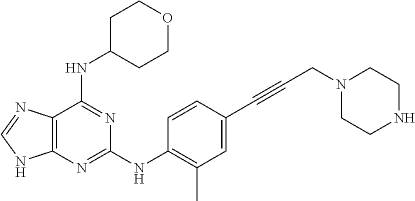 | Scheme 23 | Ret. Time (HPLC): 4.795 min; Mass Spec.: 447.26140 (M + 1) |
| C108 | rel-N~6~- Cyclohexyl- N~2~- (4-{3- [(2R,6S)- 2,6- dimethyl- morpholin- 4-yl] prop-1- yn-1-yl}-2- methoxy- phenyl)- 3H-purine- 2,6-diamine | 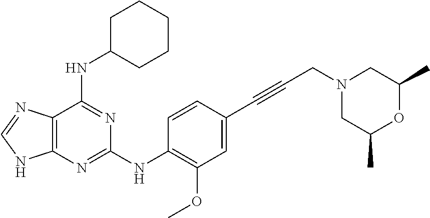 | Scheme 23 | Ret. Time (HPLC): 6.010 min; Mass Spec.: 490.29191 (M + 1) |
| C109 | rel-N~2~- (2-Chloro- 4-{3- [(2R,6S)- 2,6- dimethyl- morpholin- 4-yl]prop-1- yn-1-yl} phenyl)- N~6~- (tetrahydro- 2H- pyran-4- yl)-3H- purine- 2,6-diamine | 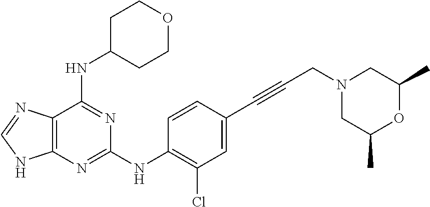 | Scheme 23 | Ret. Time (HPLC): 5.638 min; Mass Spec.: 496.22082 (M + 1) |
| C110 | N~6~- Cyclohexyl- N~2~- {2-methyl- 4-[3- (piperidin- 1-yl)prop- 1-yn-1-yl] phenyl}- 3H- purine- 2,6-diamine | 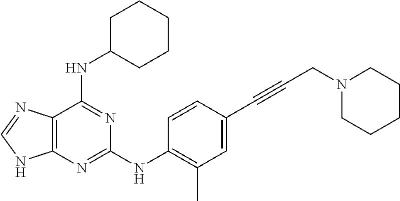 | Scheme 23 | Ret. Time (HPLC): 5.593 min; Mass Spec.: 444.28634 (M + 1) |
| C111 | N~2~- {2-Chloro- 4-[3-(1,1- dioxidothio- morpholin- 4-yl)prop- 1-yn-1-yl] phenyl}- N~6~- (tetrahydro- 2H-pyran-4- yl)-3H- purine- 2,6-diamine | 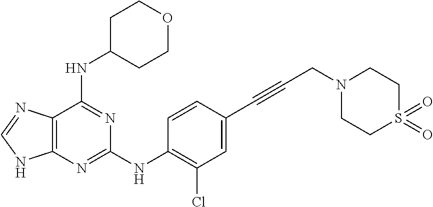 | Scheme 23 | Ret. Time (HPLC): 6.261 min; Mass Spec.: 516.15791 (M + 1) |
| C112 | N~2~- {2-Chloro- 4-[3- (morpholin- 4-yl)prop- 1-yn-1-yl] phenyl}- N~6~- cyclohexyl- 3H- purine- 2,6-diamine | 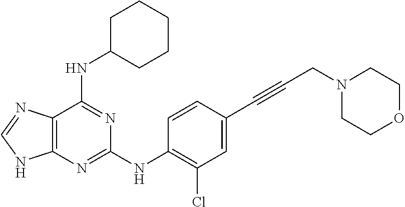 | Scheme 23 | Ret. Time (HPLC): 6.042 min; Mass Spec.: 466.21234 (M + 1) |
| C113 | N~6~- Cyclohexyl- N~2~- [2-methyl- 4-(pyridin- 3- ylethynyl) phenyl]- 3H-purine- 2,6-diamine | 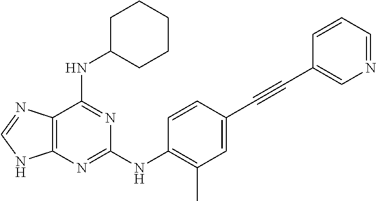 | Scheme 23 | Ret. Time (HPLC): 7.961 min; Mass Spec.: 424.22519 (M + 1) |
| C114 | 1-[(3- Methyl-4- {[6-(tetra- hydro- 2H-pyran- 4- ylamino)- 3H- purin-2- yl]amino} phenyl) ethynyl] cyclopentanol | 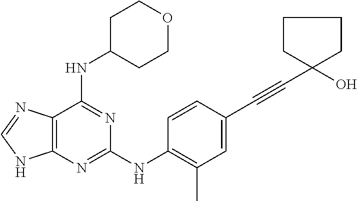 | Scheme 23 | Ret. Time (HPLC): 9.635 min; Mass Spec.: 433.23093 (M + 1) |
| C115 | N~6~- Cyclohexyl- N~2~- {2-methyl- 4-[3- methyl-3- (morpholin-4- yl)but- 1-yn-1- yl]phenyl}- 3H- purine- 2,6-diamine | 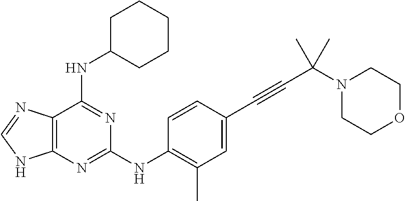 | Scheme 23 | Ret. Time (HPLC): 5.667 min; Mass Spec.: 474.29829 (M + 1) |
| C116 | N~2~- [2-Methyl- 4-(pyridin- 2- ylethynyl) phenyl]- N~6~- (tetrahydro- 2H- pyran-4- yl)-3H- purine- 2,6-diamine | 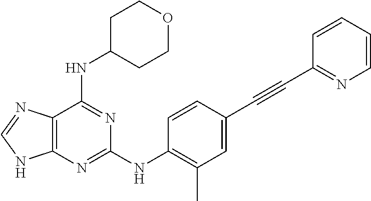 | Scheme 23 | Ret. Time (HPLC): 6.223 min; Mass Spec.: 426.20225 (M + 1) |
| C117 | N~6~- Cyclohexyl- N~2~-{2- methoxy-4-[3- methyl-3- (morpholin-4- yl)but- 1-yn-1-yl] phenyl}- 3H-purine- 2,6-diamine | 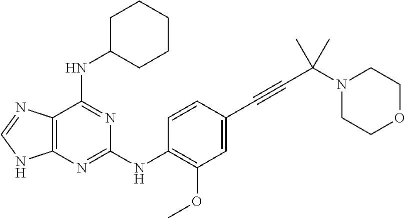 | Scheme 23 | Ret. Time (HPLC): 6.753 min; Mass Spec.: 490.29673 (M + 1) |
| C118 | N~6~- Cyclohexyl- N~2~-{2- methoxy- 4-[3- (piperazin- 1-yl)prop- 1-yn-1- yl]phenyl}- 3H- purine- 2,6-diamine | 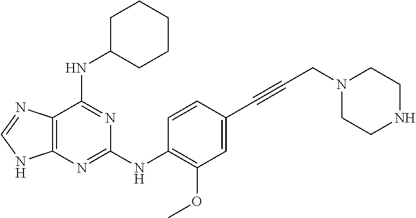 | Scheme 23 | Ret. Time (HPLC): 5.560 min; Mass Spec.: 461.27799 (M + 1) |
| C119 | N~2~-{2- Methyl- 4-[3- methyl-3- (morpholin- 4-yl)but-1- yn-1-yl] phenyl}- N~6~- (tetrahydro- 2H- pyran-4- yl)-3H- purine- 2,6-diamine | 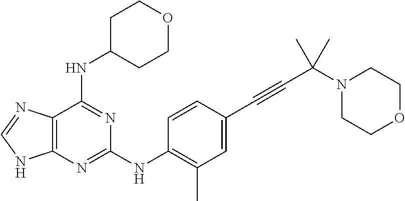 | Scheme 23 | Ret. Time (HPLC): 6.311 min; Mass Spec.: 476.27590 (M + 1) |
| C120 | N~2~- {2-Chloro- 4-[3- (morpholin- 4-yl)prop- 1-yn-1-yl] phenyl}- N~6~- (tetrahydro- 2H- pyran-4- yl)-3H- purine- 2,6-diamine | 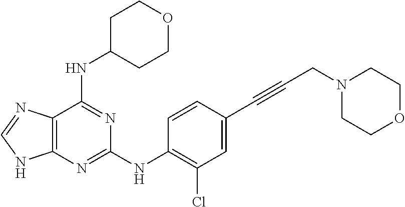 | Scheme 23 | Ret. Time (HPLC): 5.262 min; Mass Spec.: 468.19042 (M + 1) |
| C121 | N~2~-{2- Methyl- 4-[3- (morpholin-4- yl)prop- 1-yn-1-yl] phenyl}- N~6~- (tetrahydro- 2H- pyran-4- yl)-3H- purine- 2,6-diamine | 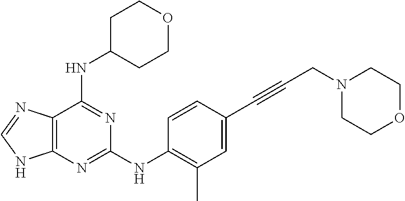 | Scheme 23 | Ret. Time (HPLC): 4.785 min; Mass Spec.: 448.24541 (M + 1) |
| C122 | N~6~- Cyclohexyl- N~2~- {2-methyl- 4-[3- (morpholin- 4-yl)prop- 1-yn-1-yl] phenyl}-3H- purine- 2,6-diamine | 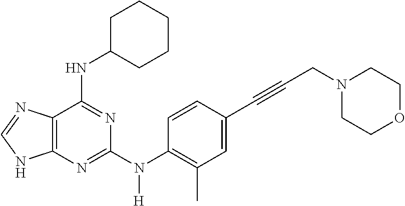 | Scheme 23 | Ret. Time (HPLC): 7.505 min; Mass Spec.: 446.26647 (M + 1) |
| C123 | N~2~-{2- Methyl- 4-[3-(4- methyl- piperazin- 1-yl)prop- 1-yn-1-yl] phenyl}- N~6~- (tetrahydro- 2H-pyran-4- yl)-3H- purine- 2,6-diamine | 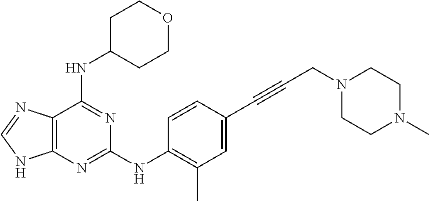 | Scheme 23 | Ret. Time (HPLC): 4.868 min; Mass Spec.: 461.27803 (M + 1) |
| C124 | N~6~- Cyclohexyl- N~2~- {2-methyl- 4-[3-(4- methyl- piperazin- 1-yl)prop- 1-yn-1-yl] phenyl}- 3H-purine- 2,6-diamine | 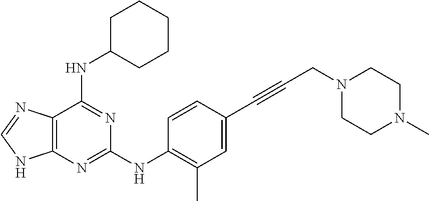 | Scheme 23 | Ret. Time (HPLC): 7.192 min; Mass Spec.: 459.29805 (M + 1) |
| C125 | N~6~- Cyclohexyl- N~2~- {2-methyl- 4-[3- (piperazin- 1-yl)prop- 1-yn-1-yl] phenyl}- 3H-purine- 2,6-diamine | 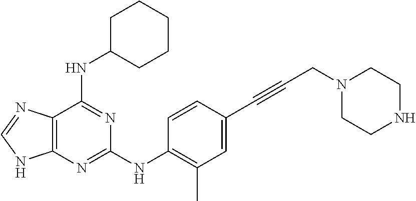 | Scheme 23 | Ret. Time (HPLC): 7.223 min; Mass Spec.: 445.28386 (M + 1) |
| C126 | N~6~- Cyclohexyl- N~2~- {4-[3- (diethyl- amino) prop-1- yn-1-yl]-2- methyl- phenyl}- 3H-purine- 2,6-diamine | 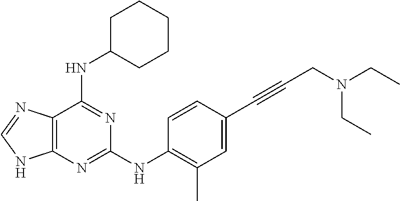 | Scheme 23 | Ret. Time (HPLC): 5.757 min; Mass Spec.: 432.28760 (M + 1 |
| C127 | 4-{[2-({2- Chloro-4- [3- (morpholin- 4-yl)prop- 1-yn-1- yl]phenyl} amino)- 3H-purin- 6-yl] amino} cyclohexanol | 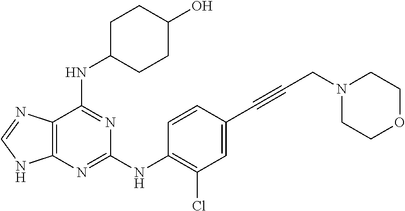 | Scheme 23 | Ret. Time (HPLC): 5.037 min; Mass Spec.: 482.20658 (M + 1) |
| C128 | N~6~- Cyclohexyl- N~2~- {4-[3- (diethyl- amino) prop-1-yn- 1-yl]-2- methoxy- phenyl}- 3H-purine- 2,6-diamine | 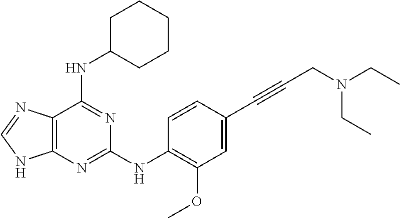 | Scheme 23 | Ret. Time (HPLC): 5.897 min; Mass Spec.: 448.28257 (M + 1) |
| C129 | 2-{4-[3- (4-{[6- (Cyclohexyl- amino)- 3H-purin-2- yl]amino}-3- methyl- phenyl) prop-2- yn-1-yl] piperazin-1- yl}ethanol | 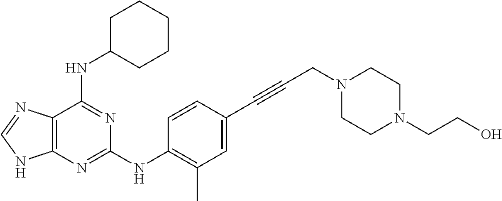 | Scheme 23 | Ret. Time (HPLC): 5.407 min; Mass Spec.: 489.30725 (M + 1) |
| C130 | N~2~- [2-Methyl- 4-(pyridin- 3-ylethynyl) phenyl]- N~6~- (tetrahydro- 2H-pyran-4- yl)-3H- purine- 2,6-diamine | 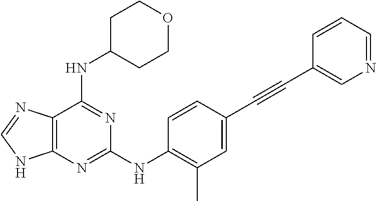 | Scheme 23 | Ret. Time (HPLC): 7.023 min; Mass Spec.: 426.20442 (M + 1) |
| C131 | N~6~- Cyclohexyl- N~2~-{4- [3-(1,1- dioxidothio- morpholin- 4-yl)prop- 1-yn-1- yl]-2- methoxy- phenyl}- 3H-purine- 2,6-diamine | 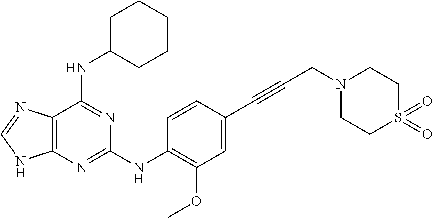 | Scheme 23 | Ret. Time (HPLC): 6.756 min; Mass Spec.: 510.22875 (M + 1) |
| C132 | N~2~- {2-Chloro- 4-[3- (1,1- dioxidothio- morpholin- 4-yl)prop- 1-yn-1- yl]phenyl}- N~6~- cyclohexyl- 3H-purine- 2,6-diamine | 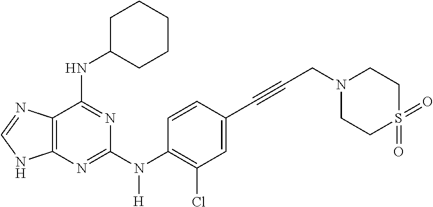 | Scheme 23 | Ret. Time (HPLC): 8.079 min; Mass Spec.: 514.18041 (M + 1) |
| C133 | N~6~- Cyclohexyl- N~2~- {4-[3-(1,1- dioxidothio- morpholin- 4-yl)prop- 1-yn-1- yl]-2- methyl- phenyl}- 3H-purine- 2,6-diamine | 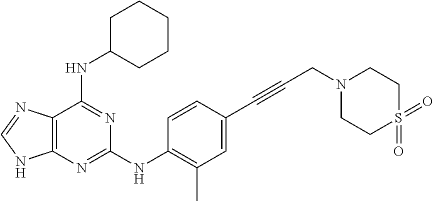 | Scheme 23 | Ret. Time (HPLC): 6.521 min; Mass Spec.: 494.23188 (M + 1) |
| C134 | N~2~-{2- Methoxy-4- [3- (morpholin- 4-yl)prop-1- yn-1- yl]phenyl}- N~6~- (tetrahydro- 2H- pyran-4-yl)- 3H-purine- 2,6-diamine | 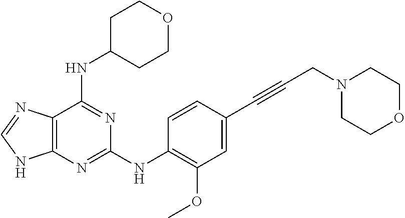 | Scheme 23 | Ret. Time (HPLC): 4.981 min; Mass Spec.: 464.24014 (M + 1) |
| C135 | rel-N~2~- (4-{3- [(2R,6S)- 2,6- Dimethyl- morpholin- 4-yl]prop-1- yn-1-yl}-2- methyl- phenyl)- N~6~- (tetrahydro- 2H- pyran-4- yl)-3H- purine- 2,6-diamine | 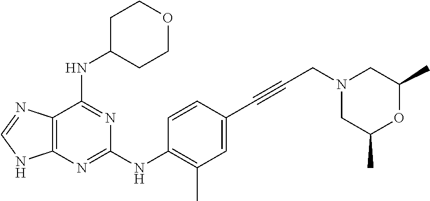 | Scheme 23 | Ret. Time (HPLC): 5.120 min; Mass Spec.: 476.27685 (M + 1) |
| C136 | 2-Methyl- 4-(3- methyl- 4-{[6- (tetrahydro- 2H- pyran-4- ylamino)- 3H- purin-2- yl]amino} phenyl) but-3- yn-2-ol | 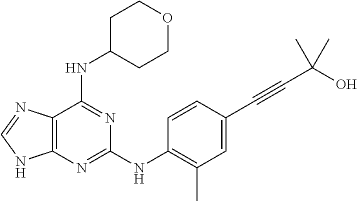 | Scheme 23 | Ret. Time (HPLC): 6.578 min; Mass Spec.: 407.22055 (M + 1) |
| C137 | 4-(4-{[6- (Cyclohexyl- amino)- 3H- purin-2- yl]amino}- 3- methyl- phenyl) but-3- yn-2-ol | 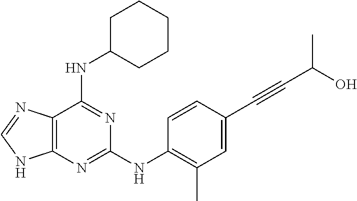 | Scheme 23 | Ret. Time (HPLC): 6.570 min; Mass Spec.: 391.22159 (M + 1) |
| C138 | 4-(4- {[6- (Cyclohexyl- amino)- 3H- purin-2- yl]amino}-3- methyl- phenyl)-2- methylbut- 3-yn-2-ol | 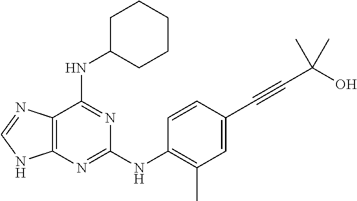 | Scheme 23 | Ret. Time (HPLC): 6.827 min; Mass Spec.: 405.23651 (M + 1) |
| C139 | 4-(3- Methyl-4- {[6-(tetra- hydro-2H- pyran-4- ylamino)- 3H- purin-2- yl]amino} phenyl) but-3- yn-2-ol | 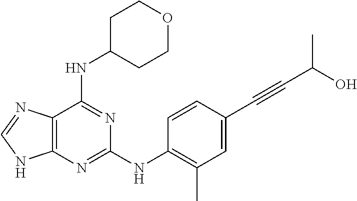 | Scheme 23 | Ret. Time (HPLC): 6.570 min; Mass Spec.: 393.20577 (M + 1) |
| C140 | N~6~- Cyclohexyl- N~2~- [2-methyl- 4-(pyridin-2- ylethynyl) phenyl]- 3H-purine- 2,6-diamine | 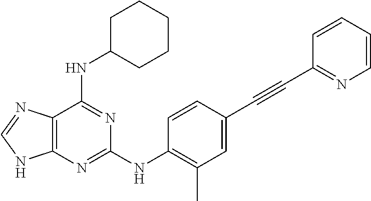 | Scheme 23 | Ret. Time (HPLC): 7.107 min; Mass Spec.: 424.22299 (M + 1) |
| C141 | 4-{[2-({2- Methyl-4-[3- (morpholin-4- yl)prop- 1-yn-1-yl] phenyl} amino)- 3H-purin- 6- yl]amino} cyclohexanol | 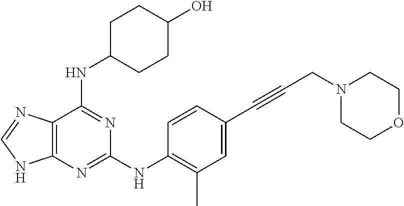 | Scheme 23 | Ret. Time (HPLC): 4.967 min; Mass Spec.: 462.26173 (M + 1) |
| C142 | 3-(4- {[6- (Cyclohexyl- amino)- 3H-purin-2- yl]amino}-3- methyl- phenyl) prop-2- yn-1-ol | 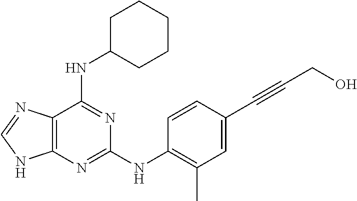 | Scheme 23 | Ret. Time (HPLC): 6.342 min; Mass Spec.: 377.21070 (M + 1) |
| C143 | N~6~- Cyclohexyl- N~2~-{2- methoxy- 4-[3- (morpholin-4- yl)prop- 1-yn-1- yl]phenyl}- 3H-purine- 2,6-diamine | 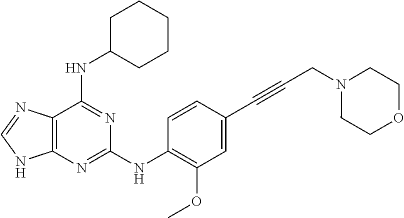 | Scheme 23 | Ret. Time (HPLC): 5.320 min; Mass Spec.: 447.26153 (M + l) |
| C144 | 1′-(4-{[6- (cyclohexyl- amino)- 9H-purin-2- yl]amino}-3- methoxy- phenyl)- 1,4′- bipiperidin- 3-ol | 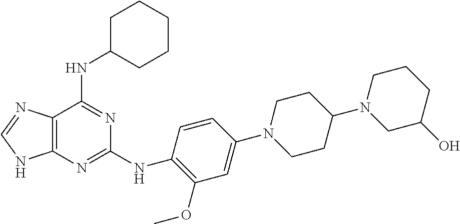 | Scheme 26 | MS (m/z): 521.3353 (M + 1). Retention time: 6.180 minutes Method info: Column 1, XTerra, MS18, 4.6 X 150 mm, 3.5 um, 0.01% TFA |
| C145 | 1-[1- (4-{[6- (Cyclohexyl- amino)- 3H-purin-2- yl]amino}-3- methoxy- phenyl) piperidin-4- yl]azetidin- 3-ol | 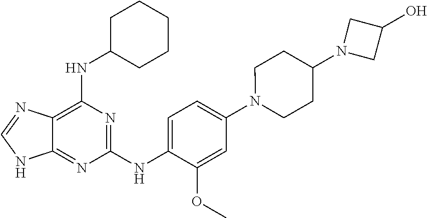 | Scheme 26 | Ret. Time (HPLC): 6.429 min; Mass Spec.: 493.3040 (M + 1) |
| C146 | N~6~- cyclohexyl- N~2~- {2-methyl- 4-[4- (morpholin- 4-yl) piperidin-1- yl]phenyl}- 9H-purine- 2,6-diamine | 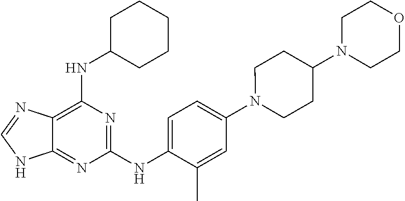 | Scheme 26 | Ret. Time (HPLC): 5.319 min; Mass Spec: 491.3310 (M + 1) |
| C147 | N~6~- Cyclohexyl- N~2~- {2-methoxy- 4-[4-(4- methyl- piperazin- 1-yl) piperidin-1- yl]phenyl}- 3H-purine- 2,6-diamine | 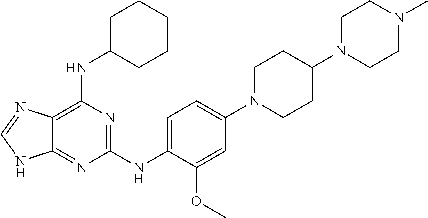 | Scheme 26 | Ret. Time (HPLC): 4.967 min; Mass Spec.: 520.3588 (M + 1) |
| C148 | N~2~- [4-(1,4′- Bipiperidin- 1′-yl)-2- methoxy- phenyl]- N~6~- cyclohexyl- 3H- purine- 2,6-diamine | 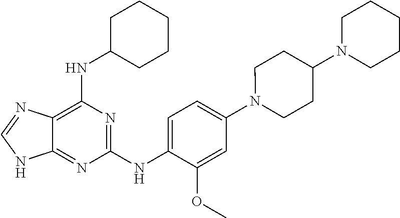 | Scheme 26 | Ret. Time (HPLC): 6.328 min; Mass Spec.: 505.3399 (M + 1) |
| C149 | N~6~- cyclohexyl- N~2~-{2- methoxy- 4-[3- (morpholin-4- yl) pyrrolidin-1- yl]phenyl}- 9H-purine- 2,6-diamine | 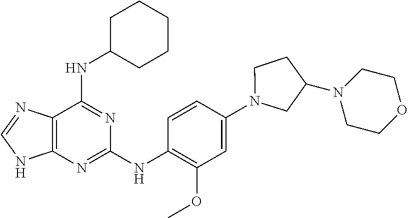 | Scheme 41 | Ret. Time (HPLC): 5.444 min; Mass Spec.: 493.30259 (M + 1) |
| C150 | N~6~- cyclohexyl- N~2~-{2- methoxy- 4-[3-(4- methyl- piperazin- 1-yl) pyrrolidin- 1-yl] phenyl}- 9H-purine- 2,6-diamine | 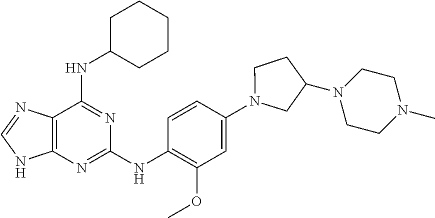 | Scheme 41 | Ret. Time (HPLC): 5.484 min; Mass Spec.: 506.33406 (M + 1) |
| C151 | N~2~- [4-(1,3′- bipyrrolidin- 1′-yl)-2- methoxy- phenyl]- N~6~- cyclohexyl- 9H-purine- 2,6-diamine | 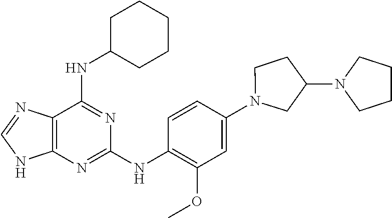 | Scheme 41 | Ret. Time (HPLC): 5.519 min; Mass Spec.: 477.30714 (M + 1) |
| C152 | N~6~- cyclohexyl- N~2~-{2- methoxy- 4-[3- (piperidin- 1-yl) pyrrolidin-1- yl]phenyl}- 9H-purine- 2,6-diamine | 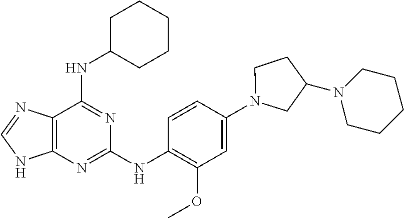 | Scheme 41 | Ret. Time (HPLC): 5.622 min; Mass Spec.: 491.32228 (M + 1) |
| C153 | N~2~-{2- Methoxy- 4-[3- (morpholin- 4-yl) pyrrolidin-1- yl]phenyl}- N~6~- (tetrahydro- 2H- pyran-4- yl)-3H- purine- 2,6-diamine | 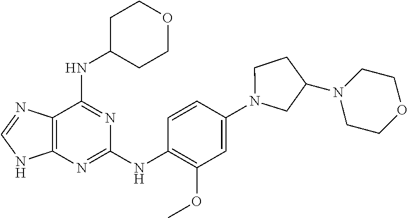 | Scheme 41 | Ret. Time (HPLC): 5.784 min; Mass Spec.: 495.28221 (M + 1 |
| C154 | N~6~- cyclohexyl- N~2~-{2- methoxy- 4-[4- (morpholin-4- yl)piperidin-1- yl]phenyl}- 9H-purine- 2,6-diamine | 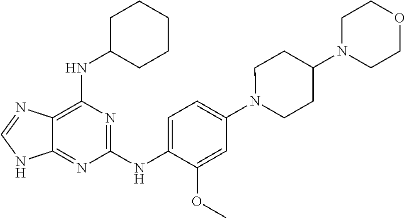 | Scheme 26 | MS (m/z): 507.31833 (M + 1). Retention time: 5.419 minutes Method info: XBridge C18, 4.6 X 150 mm, 3.5 um, ACN/Water 0.01% TFA |
| C155 | N~6~- cyclohexyl- N~2~- [4-(3,3- difluoro- 1,4′- bipiperidin- 1′-yl)-2- methoxy- phenyl]- 9H-purine- 2,6-diamine | 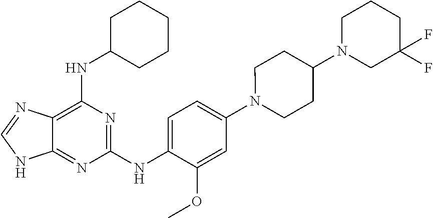 | Scheme 26 | Ret. Time (HPLC): 5.503 min; Mass Spec.: 541.3214 (M + 1) |
| C156 | N~6~- Cyclohexyl- N~2~-{2- methoxy- 4-[4- (methyl- sulfonyl) piperazin-1- yl]phenyl}- 3H-purine- 2,6-diamine | 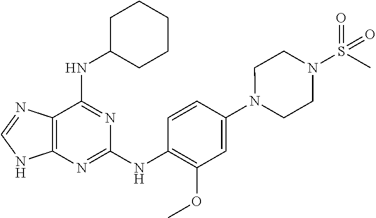 | Scheme 27 | Ret. Time (HPLC): 6.440 min; Mass Spec.: 501.2471 (M + 1) |
| C157 | N~2~-{2- Methoxy- 4-[4- (methyl- sulfonyl) piperazin-1- yl]phenyl}- N~6~- (tetrahydro- 2H-pyran- 4-yl)-3H- purine- 2,6-diamine | 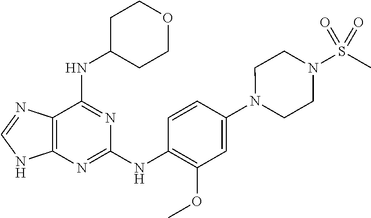 | Scheme 27 | Ret. Time (HPLC): 6.763 min; Mass Spec.: 503.21783 |
| C158 | N~2~-{2- methoxy- 4-[4- (methyl- sulfonyl) piperazin-1- yl]phenyl}- N~6~- (tetrahydro- 2H-pyran-3- yl)-9H- purine- 2,6-diamine | 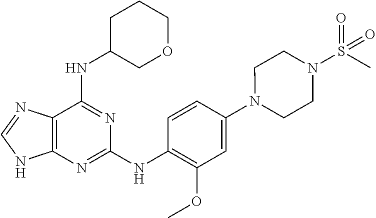 | Scheme 27 | Ret. Time (HPLC): 6.212 min; Mass Spec.: 503.21855 (M + 1) |
| C159 | N~2~-[2- Methoxy-4- (piperazin-1- yl)phenyl]- N~6~- (tetrahydro-2H- pyran-4- yl)-3H- purine-2,6- diamine | 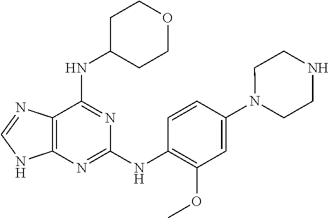 | Scheme 28 | Ret. Time (HPLC): 5.580 min; Mass Spec.: 425.23945 (M + 1) |
| C160 | N~6~- cyclohexyl- N~2~- [4-(2,5- diazabicyclo [2.2.1] hept-2-yl)-2- methoxy- phenyl]- 9H-purine- 2,6-diamine | 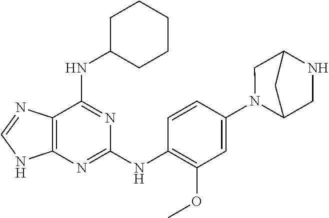 | Scheme 28 | Ret. Time (HPLC): 5.143 min; Mass Spec.: 435.26089 (M + 1) |
| C161 | N~6~- cyclohexyl- N~2~-(2- methoxy- 4-{4-[2- (pyrrolidin-1- yl)ethyl] piperazin- 1-yl}phenyl)- 9H-purine- 2,6-diamine | 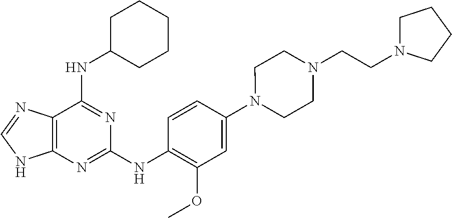 | Scheme 29 | Ret. Time (HPLC): 6.073 min; Mass Spec.: 520.3350 (M + 1)] |
| C162 | N-{2-[4- (4-{[6- (cyclohexyl- amino)- 9H-purin-2- yl]amino}-3- methoxy- phenyl) piperazin-1- yl]ethyl} acetamide | 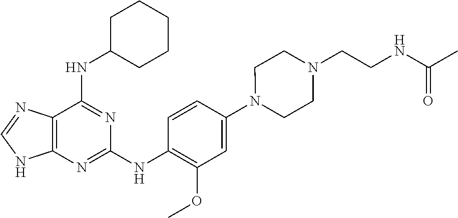 | Scheme 29 | Ret. Time (HPLC): 5.073 min; Mass Spec.: 508.31698 (M + 1) |
| C163 | N~2~- {4-[4-(2- Aminoethyl) piperazin- 1-yl]-2- methoxy- phenyl}- N~6~- cyclohexyl- 3H-purine- 2,6-diamine | 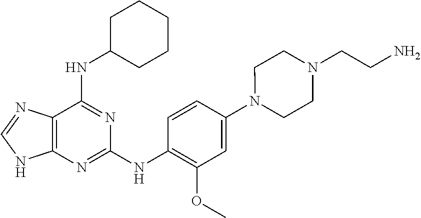 | Scheme 29 | Ret. Time (HPLC): 6.771 min; Mass Spec.: 466.30173 (M + 1) |
| C164 | 4-(4-{[6- (cyclohexyl- amino)- 9H-purin-2- yl]amino}-3- methoxy- phenyl)-N- cyclopentyl- piperazine- 1-carboxamide | 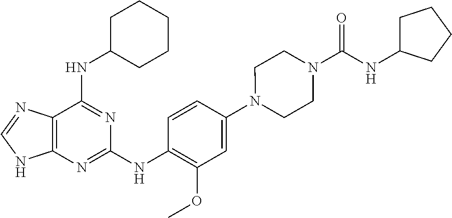 | Scheme 30 | Ret. Time (HPLC): 7.269 min; Mass Spec.: 534.3294 (M + 1) |
| C165 | 4-(4-{[6- (cyclohexyl- amino)- 9H-purin-2- yl]amino}-3- methoxy- phenyl)- N,N- dimethyl- piperazine- 1-carboxamide | 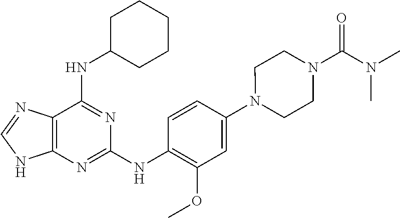 | Scheme 37 | Ret. Time (HPLC): 8.197 min; Mass Spec.: 494.29729 |
| C166 | [4-(4-{[6- (cyclohexyl- amino)- 9H-purin-2- yl]amino}-3- methoxy- phenyl) piperazin- 1-yl](4- hydroxy- piperidin-1- yl)methanone | 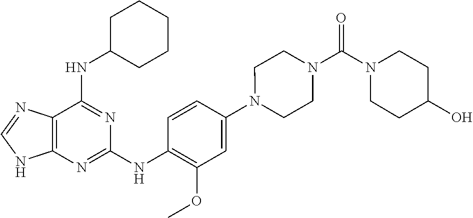 | Scheme 37 | Ret. Time (HPLC): 6.171 min; Mass Spec.: 550.32434 |
| C167 | 4-(4-{[6- (Cyclohexyl- amino)- 3H-purin-2- yl]amino}-3- methoxy- phenyl)- N-[2- (dimethyl- amino)ethyl] piperazine- 1-carboxamide | 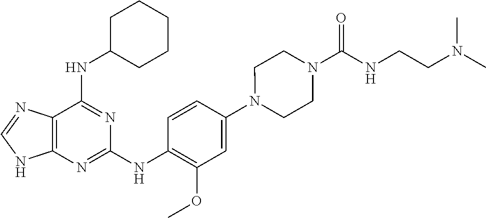 | Scheme 30 | Ret. Time (HPLC): 5.743 min; Mass Spec.: 537.33908 (M + 1) |
| C168 | [4-(4-{[6- (cyclohexyl- amino)- 9H-purin-2- yl]amino}-3- methoxy- phenyl) piperazin- 1-yl][3- (dimethyl- amino) pyrrolidin-1- yl]methanone | 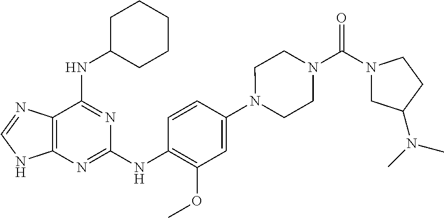 | Scheme 37 | Ret. Time (HPLC): 5.463 min; Mass Spec.: 563.35603 (M + 1) |
| C169 | 4-(4-{[6- (cyclohexyl- amino)- 9H-purin-2- yl]amino}-3- methoxy- phenyl)- N-[2- (dimethyl- amino) ethyl]-N- methyl- piperazine-1- carboxamide | 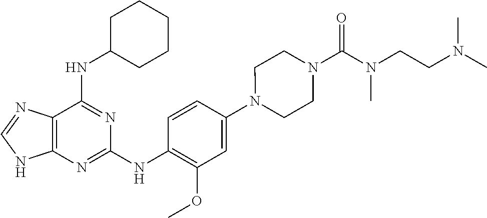 | Scheme 37 | Ret. Time (HPLC): 5.561 min; Mass Spec.: 551.35579 (M + 1) |
| C170 | 4-(4-{[6- (Cyclohexyl- amino)- 3H-purin-2- yl]amino}-3- methoxy- phenyl)- N-ethyl- piperazine- 1-carboxamide | 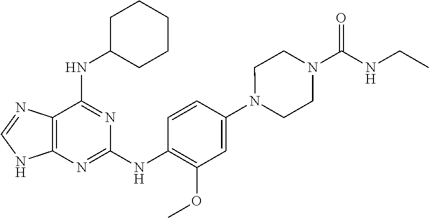 | Scheme 30 | Ret. Time (HPLC): 6.854 min; Mass Spec.: 494.29973 (M + 1) |
| C171 | 1-[4- (4-{[6- (cyclohexyl- amino)- 9H-purin-2- yl]amino}-3- methoxy- phenyl) piperazin- 1-yl]-2-(4- methyl- piperazin- 1-yl) ethane-l,2- dione | 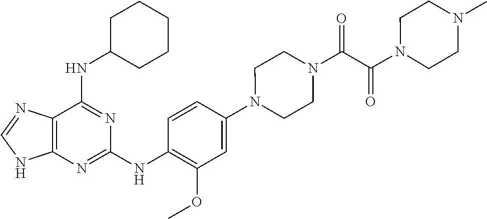 | Scheme 31 | Ret. Time (HPLC): 5.481 min; Mass Spec.: 577.3318 (M + 1)] |
| C172 | N~6~- cyclohexyl- N~2~-[2- methoxy- 4-(4-{[3-(4- methyl- piperazin-1- yl)propyl] sulfonyl} piperazin-1- yl)phenyl]- 9H-purine- 2,6-diamine | 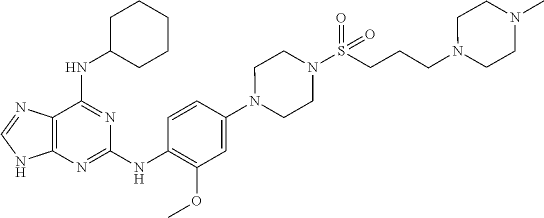 | Scheme 32 | Ret. Time (HPLC): 5.630 min; Mass Spec.: 627.3547 (M + 1) |
| C173 | N~6~- cyclohexyl- N~2~-[2- methoxy- 4-(4-{[3- (morpholin- 4- yl)propyl] sulfonyl} piperazin-1- yl)phenyl]- 9H-purine- 2,6-diamine | 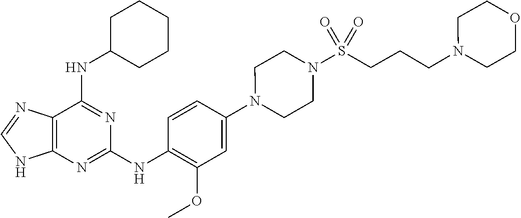 | Scheme 32 | Ret. Time (HPLC): 5.932 min; Mass Spec.: 614.32281 |
| C174 | N~6~- cyclohexyl- N~2~-(2- methoxy- 4-{4-[(2- methylpropyl) sulfonyl] piperazin- 1-yl}phenyl)- 9H- purine- 2,6-diamine | 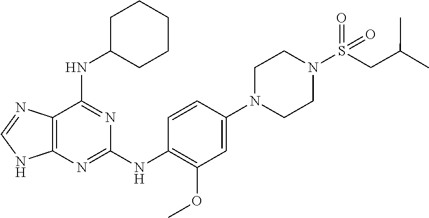 | Scheme 32 | Ret. Time (HPLC): 7.480 min; Mass Spec.: 543.28539 |
| C175 | 1-(3-{[4- (4-{[6- (cyclohexyl- amino)- 9H-purin-2- yl]amino}-3- methoxy- phenyl) piperazin-1- yl]sulfonyl} propyl) piperidin-4-ol | 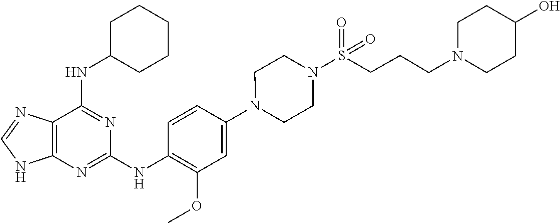 | Scheme 32 | Ret. Time (HPLC): 5.654 min; Mass Spec.: 628.33905 |
| C176 | N~2~- (4-{4-[(2- aminoethyl) sulfonyl] piperazin-1- yl}-2- methoxy- phenyl)- N~6~- cyclohexyl- 9H- purine-2,6- diamine | 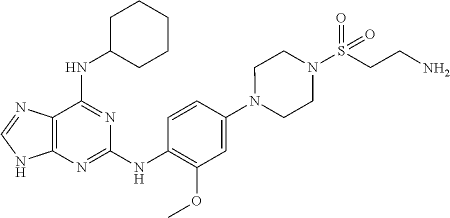 | Scheme 32 | Ret. Time (HPLC): 5.931 min; Mass Spec.: 530.26358 |
| C177 | N6-cyclohexyl- N2-[4-(4- isopropyl- sulfonyl- piperazin-1- yl)-2- methoxy- phenyl]- 9H-purine- 2,6-diamine | 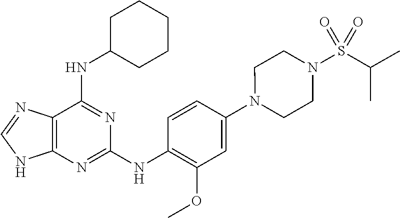 | Scheme 32 | Ret. Time (HPLC): 7.115 min; Mass Spec.: 529.26661 (M + 1) |
| C178 | N~6~- cyclohexyl- N~2~- {4-[4-({3- [3- (dimethyl- amino) pyrrolidin-1- yl]propyl} sulfonyl) piperazin-1- yl]-2- methoxy- phenyl}- 9H-purine- 2,6-diamine | 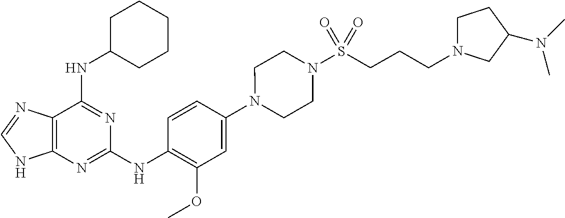 | Scheme 32 | Ret. Time (HPLC): 5.435 min; Mass Spec.: 641.37063 (M + 1) |
| C179 | 1-(3- {[4-(4-{[6- (cyclohexyl- amino)- 9H-purin-2- yl]amino}-3- methoxy- phenyl) piperazin-1- yl]sulfonyl} propyl) pyrrolidin- 3-ol | 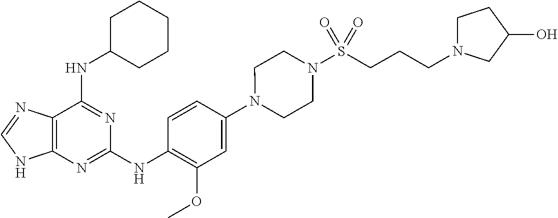 | Scheme 32 | Ret. Time (HPLC): 5.771 min; Mass Spec.: 614.32302 (M + 1) |
| C180 | 2-[(3- {[4-(4-{[6- (cyclohexyl- amino)- 9H-purin-2- yl]amino}-3- methoxy- phenyl) piperazin-1- yl]sulfonyl} propyl) (methyl) amino]ethanol | 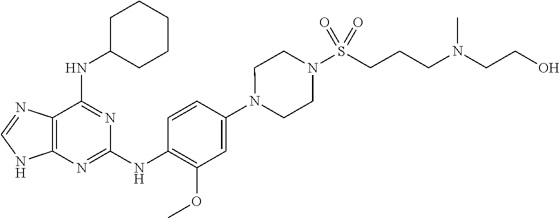 | Scheme 32 | Ret. Time (HPLC): 5.619 min; Mass Spec.: 602.32336 (M + 1) |
| C181 | N~6~- cyclohexyl- N~2~-{2- methoxy- 4-[5- (methyl- sulfonyl)- 2,5- diazabicyclo [2.2.1] hept-2- yl]phenyl}- 9H-purine- 2,6-diamine | 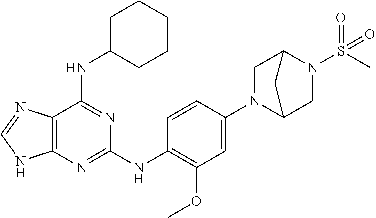 | Scheme 32 | Ret. Time (HPLC): 6.624 min; Mass Spec.: 513.23963 (M + 1) |
| C182 | 2-[4- (4-{[6- (cyclohexyl- amino)- 9H-purin-2- yl]amino}-3- methoxy- phenyl) piperazin-1- yl]acetamide | 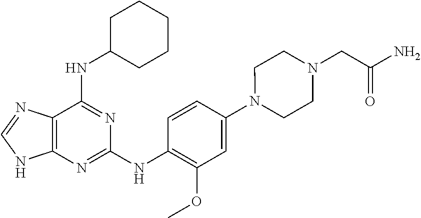 | Scheme 33 | Ret. Time (HPLC): 6.337 min; Mass Spec.: 480.2822 (M + 1) |
| C183 | [4-(4-{[6- (cyclohexyl- amino)- 9H-purin-2- yl]amino}-3- methoxy- phenyl) piperazin-1- yl](2,2- dimethyl- tetrahydro- 2H-pyran- 4-yl) methanone | 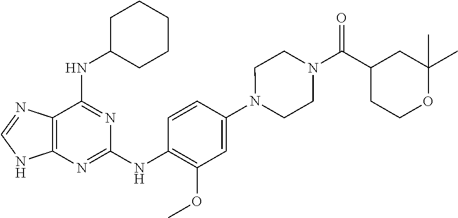 | Scheme 34 | Ret. Time (HPLC): 6.868 min; Mass Spec.: 563.3435 (M + 1) |
| C184 | [4-(4-{[6- (cyclohexyl- amino)- 9H-purin-2- yl]amino}-3- methoxy- phenyl) piperazin-1- yl](piperidin-3- yl)methanone | 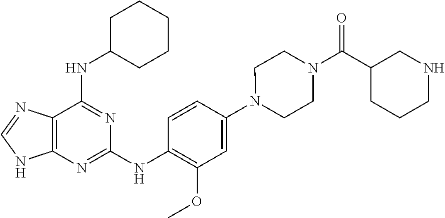 | Scheme 34 | Ret. Time (HPLC): 5.493 min; Mass Spec.: 534.32962 (M + 1) |
| C185 | 2-Amino- 1-[4-(4-{[6- (cyclohexyl- amino)- 3H-purin-2- yl]amino}-3- methoxy- phenyl) piperazin-1- yl]ethanone | 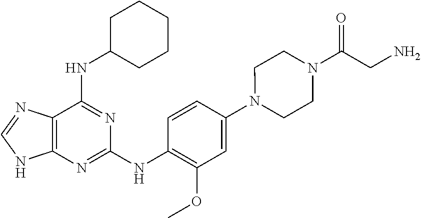 | Scheme 34 | Ret. Time (HPLC): 5.820 min; Mass Spec.: 480.28484 (M + 1) |
| C186 | [4-(4-{[6- (cyclohexyl- amino)- 9H-purin-2- yl]amino}-3- methoxy- phenyl) piperazin-1- yl](tetrahydro- 2H-pyran-4- yl)methanone | 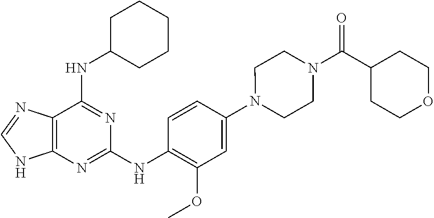 | Scheme 34 | Ret. Time (HPLC): 6.542 min; Mass Spec.: 535.31553 (M + 1) |
| C187 | 2-Amino- 1-[4-(4-{[6- (cyclohexyl- amino)- 3H-purin-2- yl]amino}-3- methoxy- phenyl) piperazin- 1-yl]-3- hydroxy- propan-1-one | 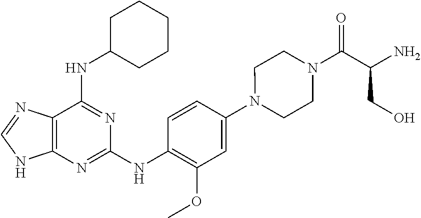 | Scheme 34 | Ret. Time (HPLC): 5.601 min; Mass Spec.: 510.29514 (M + 1) |
| C188 | N~6~- cyclohexyl- N~2~-{2- methoxy- 4-[4- (tetrahydro- furan-3- ylmethyl) piperazin- 1-yl]phenyl}- 9H-purine- 2,6-diamine | 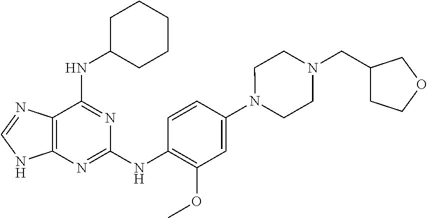 | Scheme 35 | Ret. Time (HPLC): 6.591 min; Mass Spec.: 507.3261 (M + 1) |
| C189 | N~2~- {4-[4-(2- Aminoethyl) piperazin-1- yl]-2- methoxy- phenyl}- N~6~- (tetrahydro- 2H-pyran-4- yl)-3H- purine- 2,6-diamine | 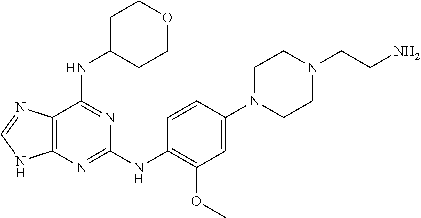 | Scheme 29 | Ret. Time (HPLC): 6.298 min; Mass Spec.: 468.28083 (M + 1) |
| C190 | [4-(4-{[6- (cyclohexyl- amino)- 9H-purin-2- yl]amino}-3- methoxy- phenyl) piperazin-1- yl](pyridin-2- yl)methanone | 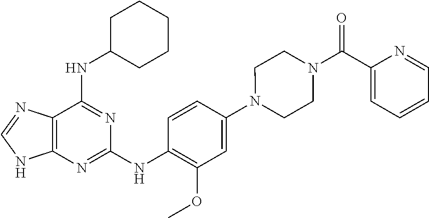 | Scheme 36 | Ret. Time (HPLC): 6.557 min; Mass Spec.: 528.28288 (M + 1) |
| C191 | N~6~- cyclohexyl- N~2~-{2- methoxy- 4-[4- (pyridin-2- ylmethyl) piperazin- 1-yl]phenyl}- 9H-purine- 2,6-diamine | 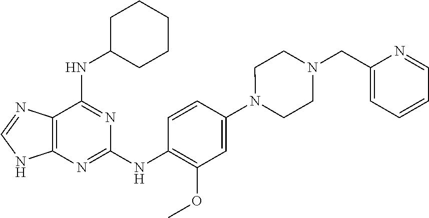 | Scheme 36 | Ret. Time (HPLC): 5.603 min; Mass Spec.: 514.2973 (M + 1) |
| C192 | N~6~- cyclohexyl- N~2~-(2- methoxy- 4-{4-[2- (morpholin-4- yl)ethyl] piperazin- 1-yl} phenyl)- 9H-purine- 2,6-diamine | 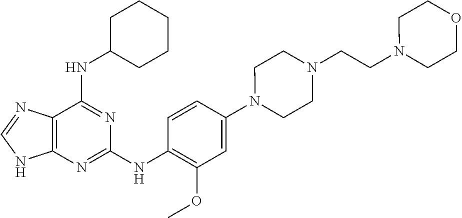 | Scheme 29 | Ret. Time (HPLC): 5.002 min; Mass Spec.: 536.34545 |
| C193 | [4-(4-{[6- (cyclohexyl- amino)- 9H-purin-2- yl]amino}-3- methoxy- phenyl) piperazin- 1-yl] (morpholin- 4-yl) methanone | 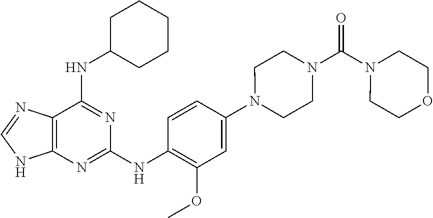 | Scheme 37 | Ret. Time (HPLC): 6.569 min; Mass Spec.: 536.3078 (M + 1) |
| C194 | 2-[4- (4-{[6- (cyclohexyl- amino)- 9H-purin-2- yl]amino}-3- methoxy- phenyl) piperazin- 1-yl]-1-(4- methyl- piperazin- 1-yl)ethanone | 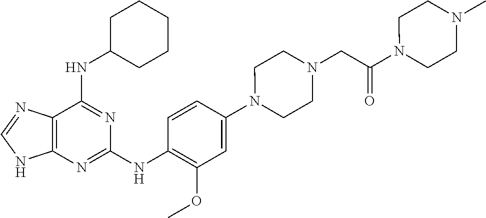 | Scheme 38 | Ret. Time (HPLC): 4.895 min; Mass Spec.: 563.3663 (M + 1) |
| C195 | 2-[4-(4-{[6- (cyclohexyl- amino)- 9H-purin-2- yl]amino}-3- methoxy- phenyl) piperazin- 1-yl]-1- (morpholin-4- yl)ethanone | 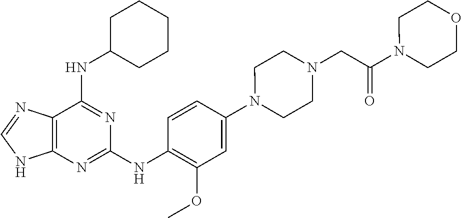 | Scheme 38 | Ret. Time (HPLC): 5.154 min; Mass Spec.: 550.32458 (M + 1) |
| C196 | N~6~- Cyclohexyl- N~2~-{2- methoxy-4-[4- (morpholin-4- ylsulfonyl) piperazin- 1-yl]phenyl}- 3H-purine- 2,6-diamine | 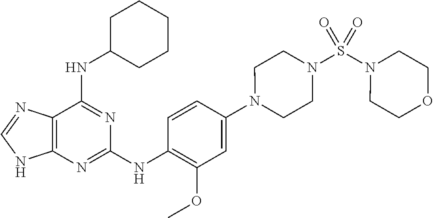 | Scheme 39 | Ret. Time (HPLC): 7.934 min; Mass Spec.: 572.27619 (M + 1) |
| C197 | 4-(4-{[6- (Cyclohexyl- amino)- 3H-purin-2- yl]amino}-3- methoxy- phenyl)- N,N- dimethyl- piperazine- 1-sulfonamide | 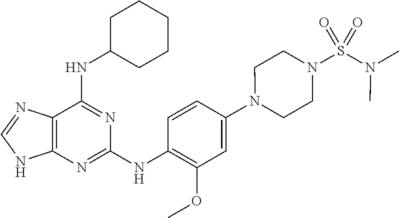 | Scheme 39 | Ret. Time (HPLC): 8.801 min; Mass Spec.: 530.26637 (M + 1) |
| C198 | N~6~- cyclohexyl- N~2~-(2- methoxy- 4-{4-[(4- methyl- piperazin-1- yl)sulfonyl] piperazin-1- yl}phenyl)- 9H-purine- 2,6-diamine | 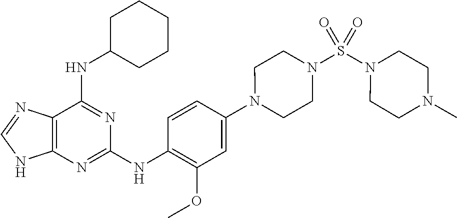 | Scheme 39 | Ret. Time (HPLC): 5.694 min; Mass Spec.: 585.30749 (M + 1) |
| C199 | N~6~- cyclohexyl- N~2~-{2- methoxy- 4-[4- (piperazin-1- ylsulfonyl) piperazin- 1-yl]phenyl}- 9H-purine- 2,6-diamine | 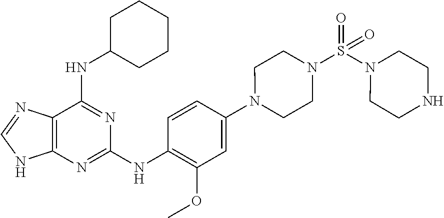 | Scheme 39 | Ret. Time (HPLC): 6.053 min; Mass Spec.: 571.29162 (M + 1) |
| C200 | N~6~- Cyclohexyl- N~2~- {4-[4-(1H- imidazol-4- ylsulfonyl) piperazin- 1-yl]-2- methoxy- phenyl}- 3H-purine- 2,6-diamine | 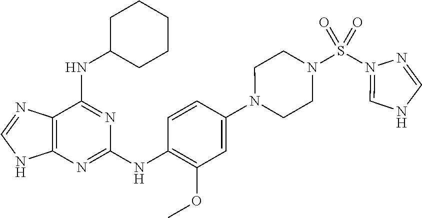 | Scheme 39 | Ret. Time (HPLC): 8.083 min; Mass Spec.: 553.24384 |
| C201 | N~6~- cyclohexyl- N~2~-{2- methoxy- 4-[4-(1- methyl- piperidin- 4-yl) piperazin-1- yl]phenyl}- 9H-purine- 2,6-diamine | 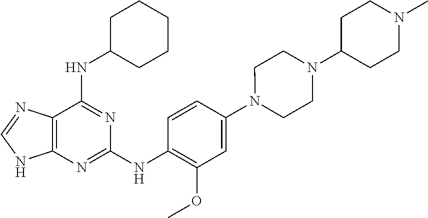 | Scheme 40 | Ret. Time (HPLC): 5.302 min; Mass Spec.: 520.34946 (M + 1) |
| C202 | N~6~- Cyclohexyl- N~2~-[4-(4- cyclohexyl- piperazin- 1-yl)-2- methoxy phenyl]- 3H-purine- 2,6-diamine | 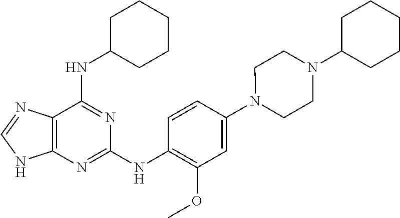 | Scheme 40 | Ret. Time (HPLC): 6.114 min; Mass Spec.: 505.33968 (M + 1) |
| C203 | N~6~- cyclohexyl- N~2~-[2- methoxy- 4-(4- phenyl- piperazin- 1-yl) phenyl]-9H- purine- 2,6-diamine | 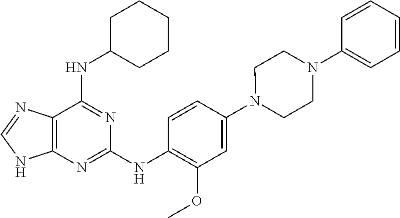 | Scheme 41 | Ret. Time (HPLC): 7.616 min; Mass Spec.: 499.29068 (M + 1) |
| C204 | N~2~-[2- Methoxy- 4-(4- phenyl- piperazin- 1-yl)phenyl]- N~6~- (tetrahydro- 2H-pyran- 4-yl)-3H- purine- 2,6-diamine | 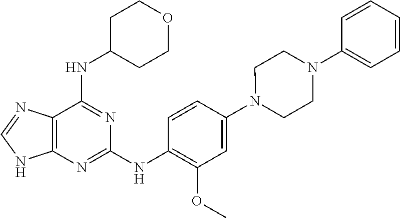 | Scheme 41 | Ret. Time (HPLC): 6.726 min; Mass Spec.: 501.27056 (M + 1) |
| C205 | N~6~- cyclohexyl- N~2~-(2- methoxy- 4-{4-[(2- methyl-1H- imidazol-1- yl)methyl] piperidin-1- yl} phenyl)-9H- purine- 2,6-diamine | 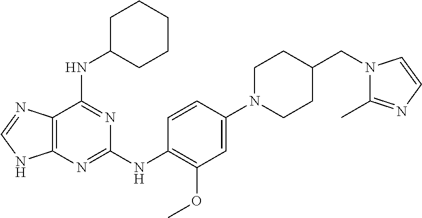 | Scheme 41 | Ret. Time (HPLC): 5.472 min; Mass Spec.: 516.31938 (M + 1) |
| C206 | N~6~- cyclohexyl- N~2~- {4-[3-(1H- imidazol-1- ylmethyl) piperidin- 1-yl]-2- methoxy- phenyl}- 9H-purine- 2,6-diamine | 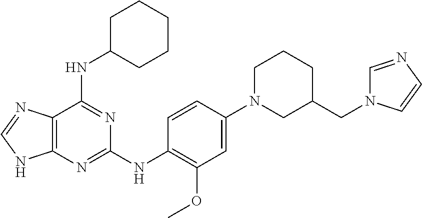 | Scheme 41 | Ret. Time (HPLC): 5.654 min; Mass Spec.: 502.30237 (M + 1 |
| C207 | N~2~- {4-[3-(1H- Imidazol-1- ylmethyl) piperidin- 1-yl]-2- methoxy- phenyl}- N~6~- (tetrahydro- 2H-pyran- 4-yl)-3H- purine-2,6- diamine | 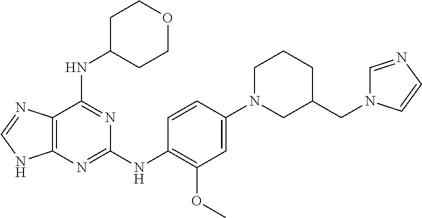 | Scheme 41 | Ret. Time (HPLC): 6.763 min; Mass Spec.: 504.28264 (M + 1) |
| C208 | N~2~-(2- Methoxy- 4-{4-[(2- methyl-1H- imidazol-1- yl)methyl] piperidin-1- yl}phenyl)- N~6~- (tetrahydro- 2H-pyran-4- yl)-3H- purine-2,6- diamine | 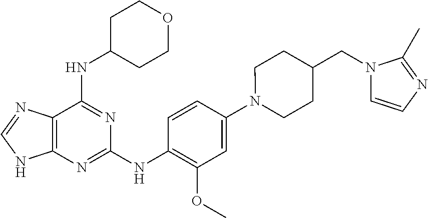 | Scheme 41 | Ret. Time (HPLC): 6.604 min; Mass Spec.: 518.29846 (M + l) |
| C209 | N-[1- (4-{[6- (cyclohexyl- amino)- 9H-purin-2- yl]amino}-3- methoxy- phenyl) piperidin-4- yl]methane- sulfonamide | 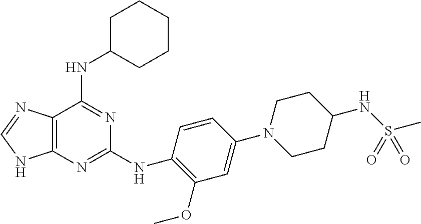 | Scheme 41 | Ret. Time (HPLC): 6.00 min; Mass Spec.: 515.25473 (M + 1) |
| C210 | N~6~- cyclohexyl- N~2~-[4- (hexahydro- pyrrolo [1,2-a] pyrazin- 2(1H)-yl)-2- methoxy- phenyl]- 9H-purine- 2,6-diamine | 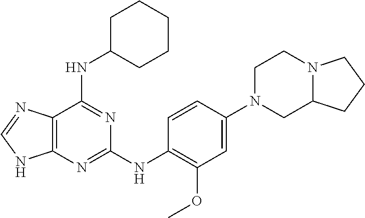 | Scheme 41 | Ret. Time (HPLC): 5.800 min; Mass Spec.: 463.29112 (M + 1) |
| C211 | N~2~-[4- (Hexahydro- pyrrolo [1,2-a] pyrazin- 2(1H)-yl)-2- methoxy- phenyl]- N~6~- (tetrahydro- 2H-pyran-4- yl)-3H- purine- 2,6-diamine | 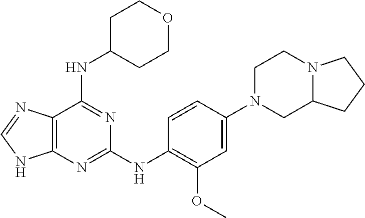 | Scheme 41 | Ret. Time (HPLC): 6.816 min; Mass Spec.: 465.27118 (M + 1) |
| C212 | [1-(4-{[6- (Cyclohexyl- amino)- 3H-purin-2- yl]amino}-3- methoxy- phenyl) pyrrolidin-2- yl]methanol | 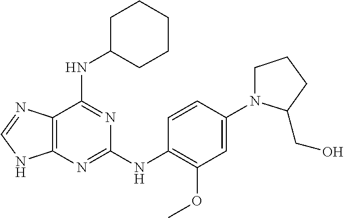 | Scheme 41 | Ret. Time (HPLC): 6.499 min; Mass Spec.: 438.25947 (M + 1) |
| C213 | [1-(3- Methoxy- 4-{[6- (tetrahydro- 2H-pyran-4- ylamino)-3H- purin-2- yl]amino} phenyl) piperidin-3- yl]methanol | 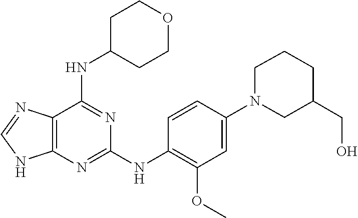 | Scheme 41 | Ret. Time (HPLC): 6.841 min; Mass Spec.: 454.25611 (M + 1) |
| C214 | 1-(3- Methoxy- 4-{[6- (tetrahydro- 2H-pyran-4- ylamino)-3H- purin-2- yl]amino} phenyl) piperidin- 4-ol | 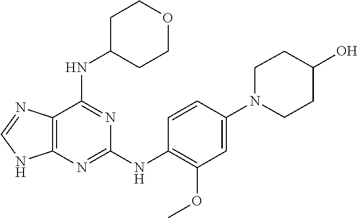 | Scheme 41 | Ret. Time (HPLC): 6.810 min; Mass Spec.: 440.24045 (M + 1) |
| C215 | (3S)-1-(3- Methoxy- 4-{[6- (tetrahydro- 2H- pyran-4- ylamino)-3H- purin-2- yl]amino} phenyl) pyrrolidin- 3-ol | 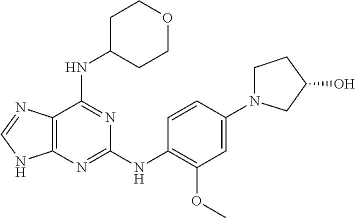 | Scheme 41 | Ret. Time (HPLC): 5.699 min; Mass Spec.: 426.22326 (M + 1) |
| C216 | N~2~- [4-(4- amino- piperidin- 1-yl)-2- methoxy- phenyl]- N~6~- cyclohexyl- 9H-purine- 2,6-diamine | 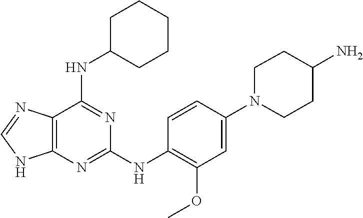 | Scheme 41 | Ret. Time (HPLC): 5.39 min; Mass Spec.: 437.27821 (M + 1) |
| C217 | N~2~- {4-[3- (dimethyl- amino) pyrrolidin- 1-yl]-2- methoxy- phenyl}- N~6~- (tetrahydro- 2H- pyran-4- yl)-9H- purine- 2,6-diamine | 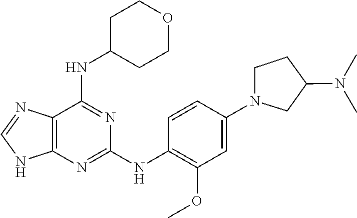 | Scheme 41 | Ret. Time (HPLC): 5.087 min; Mass Spec.: 453.27056 (M + 1) |
| C218 | 2-[4-(3- Methoxy- 4-{[6- (tetrahydro- 2H- pyran-4- ylamino)-3H- purin-2- yl]amino} phenyl)-1,4- diazepan-1- yl]ethanol | 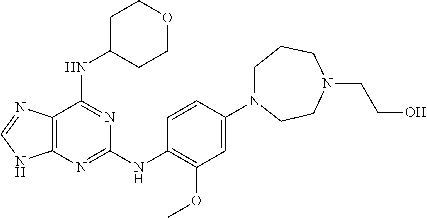 | Scheme 41 | Ret. Time (HPLC): 5.719 min; Mass Spec.: 483.28266 (M + 1) |
| C219 | N~2~-[2- methoxy-4- (morpholin- 4- yl)phenyl]- N~6~-[1- (methyl- sulfonyl) piperidin- 3-yl]- 9H-purine- 2,6-diamine | 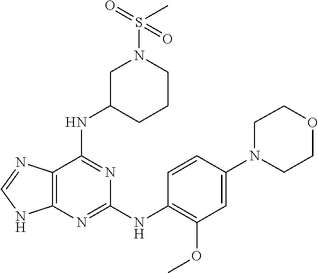 | Scheme 42 | Mas Spec.: 503.21727 (M + 1) |
| C220 | N~2~-{2- methoxy- 4-[4- (methyl- sulfonyl) piperazin-1- yl]phenyl}- N~6~-[1- (methyl- sulfonyl) piperidin- 4-yl]- 9H-purine- 2,6-diamine | 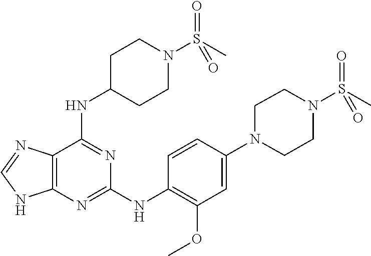 | Scheme 42 | Ret. Time (HPLC): 6.188 min; Mass Spec.: 580.21057 (M + 1) |
| C221 | N-ethyl-3- methoxy- 4-[(6-{[1- (methyl- sulfonyl) piperidin-4- yl]amino}- 9H-purin-2- yl)amino] benzamide | 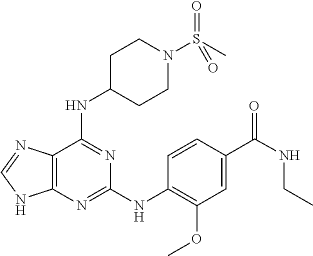 | Scheme 42 | Ret. Time (HPLC, MPI-0): 6.053 min; Mass Spec.: 489.20216 (M + 1) |
| C222 | N~2~-[2- Methoxy-4- (morpholin- 4-yl)phenyl]- N~6~-[1- (methyl- sulfonyl) piperidin- 4-yl]- 3H-purine- 2,6-diamine | 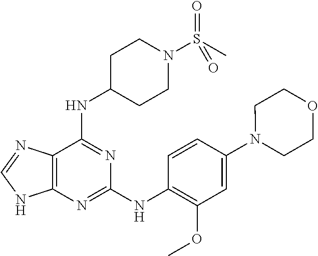 | Scheme 42 | Ret. Time (HPLC): 6.361 min; Mass Spec.: 503.21790 (M + 1) |
| C223 | N~6~- cyclohexyl- N~2~-(2- methoxy- 4-{1-[2- (morpholin-4- yl)ethyl]-1H- pyrazol-4- yl}phenyl)- 9H- purine-2,6- diamine | 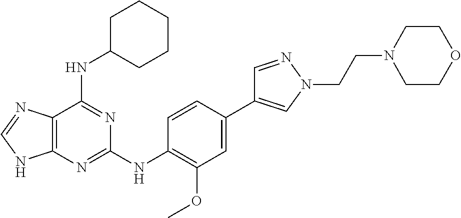 | Scheme 43 | Ret. Time (HPLC): 5.688 min; Mass Spec.: 518.3070 (M + 1). |
| C224 | N~2~-[3- methoxy-4′- (methyl- sulfonyl) biphenyl- 4-yl]-N~6~- (tetrahydro- 2H-pyran-4- yl)-9H- purine- 2,6-diamine | 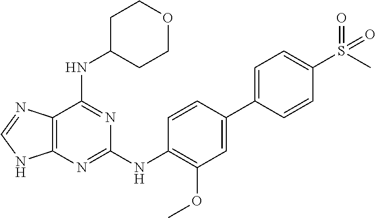 | Scheme 43 | Ret. Time (HPLC): 6.445 min; Mass Spec.: 495.18139 (M + 1). |
| C225 | N~6~- cyclohexyl- N~2~-[2- methoxy-4- (pyridin-4- yl)phenyl]- 9H-purine- 2,6-diamine | 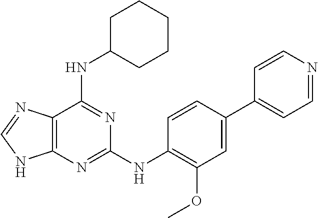 | Scheme 43 | Ret. Time (HPLC): 5.84 min; Mass Spec.: 416.2074 (M + 1) |
| C226 | (4′-{[6- (cyclohexyl- amino)- 9H-purin-2- yl]amino}-3′- methoxy- biphenyl- 4-yl) (morpholin- 4-yl) methanone | 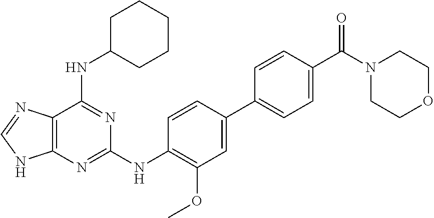 | Scheme 43 | Ret. Time (HPLC): 6.49 min; Mass Spec.: 528.2723 (M + l) |
| C227 | 3′-methoxy- N,N- dimethyl-4′- {[6- (tetrahydro- 2H-pyran-4- ylamino)- 9H-purin-2- yl]amino} biphenyl-3- sulfonamide | 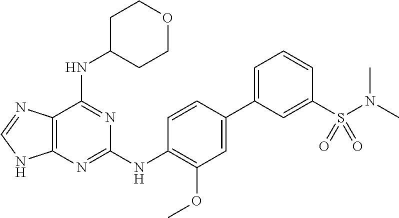 | Scheme 43 | Ret. Time (HPLC): 6.961 min; Mass Spec.: 524.21396 (M + 1) |
| C228 | N~6~- cyclohexyl- N~2~- [4-(3,6- dihydro- 2H-1,2′- bipyridin- 4-yl)-2- methoxy- phenyl]- 9H-purine- 2,6-diamine | 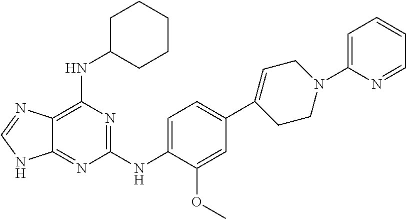 | Scheme 44 | Ret. Time (HPLC): 5.78 min; Mass Spec.: 497.27679 (M + 1) |
| C229 | [4-(4-{[6- (cyclohexyl- amino)- 9H-purin- 2-yl] amino}-3- methoxy- phenyl)- 3,6- dihydro- pyridin- 1(2H)-yl](1- methylcyclo- propyl) methanone | 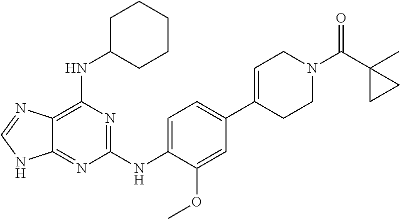 | Scheme 45 | Ret. Time (HPLC): 6.98 min; Mass Spec.: 502.29280 (M + 1) |
| C230 | N~6~- cyclohexyl- N~2~-{2- methoxy- 4-[1- (methyl- sulfonyl)- 1,2,3,6- tetrahydro- pyridin- 4-yl]phenyl}- 9H-purine- 2,6-diamine | 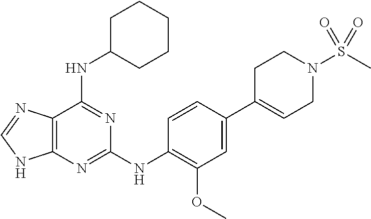 | Scheme 46 | Ret. Time (HPLC): 6.55 min; Mass Spec.: 498.22489 (M + 1) |
| C231 | 1-[4-(4-{[6- (cyclohexyl- amino)- 9H-purin-2- yl]amino}-3- methoxy- phenyl) piperidin- 1-yl]-2-(4- methyl- piperazin- 1-yl)ethane- 1,2-dione | 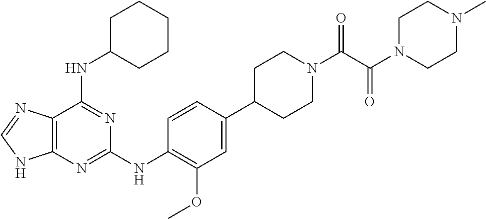 | Scheme 47 | Ret. Time (HPLC): 5.60 min; Mass Spec.: 576.34004 (M + 1) |
| C232 | 2-[4-(4-{[6- (cyclohexyl- amino)- 9H-purin-2- yl]amino}-3- methoxy- phenyl) piperidin- 1-yl]-N- methyl-2- oxoacetamide | 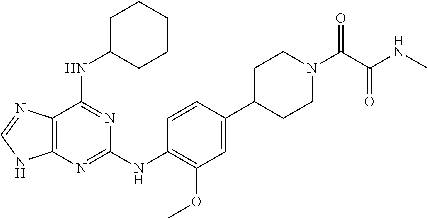 | Scheme 47 | Ret. Time (HPLC): 6.10 min; Mass Spec.: 507.28351 (M + 1) |
| C233 | 2-[4-(4-{[6- (cyclohexyl- amino)- 9H-purin-2- yl]amino}-3- methoxy- phenyl) piperidin- 1-yl]-N,N- dimethyl-2- oxoacetamide | 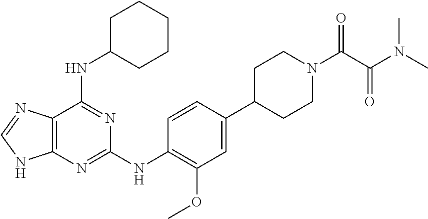 | Scheme 47 | Ret. Time (HPLC): 6.16 min; Mass Spec.: 521.29948 (M + 1) |
| C234 | [4-(4-{[6- (cyclohexyl- amino)- 9H-purin-2- yl]amino}-3- methoxy- phenyl) piperidin- 1-yl] (morpholin- 4-yl) methanone | 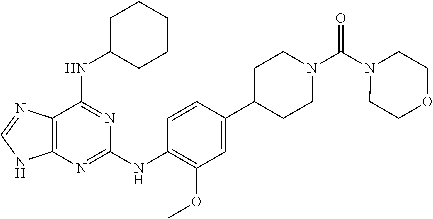 | Scheme 48 | Ret. Time (HPLC): 6.90 min; Mass Spec.: 535.31425 (M + 1) |
| C235 | N~6~- cyclohexyl- N~2~-{2- methoxy- 4-[1- (morpholin-4- ylsulfonyl) piperidin- 4-yl] phenyl}-9H- purine- 2,6-diamine | 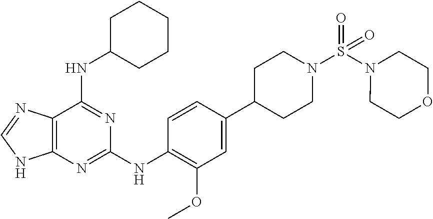 | Scheme 48 | Ret. Time (HPLC): 6.76 min; Mass Spec.: 571.28098 (M + 1) |
| C236 | N~6~- cyclohexyl- N~2~-[2- methoxy- 4-(1-{[3- (morpholin- 4-yl) propyl] sulfonyl} piperidin- 4-yl) phenyl]- 9H-purine- 2,6-diamine | 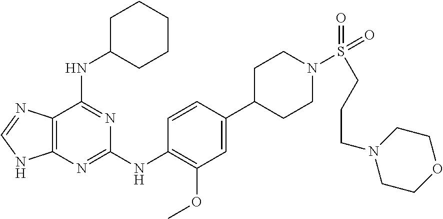 | Scheme 49 | Ret. Time (HPLC): 5.87 min; Mass Spec.: 613.3305 (M + 1) |
| C237 | N~2~-{2- methoxy- 4-[1-(methyl- sulfonyl) piperidin-4- yl]phenyl}- N~6~- (tetrahydro- 2H-pyran- 4-yl)-9H- purine- 2,6-diamine | 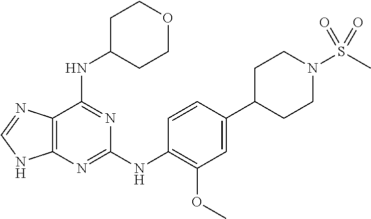 | Scheme 49 | Ret. Time (HPLC): 5.96 min; Mass Spec.: 502.22212 (M + 1) |
| C238 | N~6~- cyclohexyl- N~2~-{2- methoxy- 4-[1-(methyl- sulfonyl) piperidin-4- yl]phenyl}- 9H-purine- 2,6-diamine | 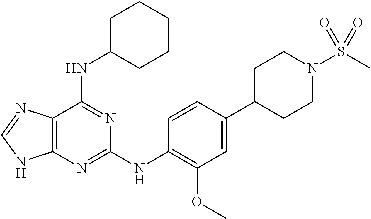 | Scheme 49 | Ret. Time (HPLC): 6.49 min; Mass Spec.: 500.24005 (M + 1) |
| C239 | N~6~- cyclohexyl- N~2~-(2- methoxy- 4-{1-[3- (morpholin-4- ylsulfonyl) propyl] piperidin-4- yl}phenyl)- 9H-purine- 2,6-diamine | 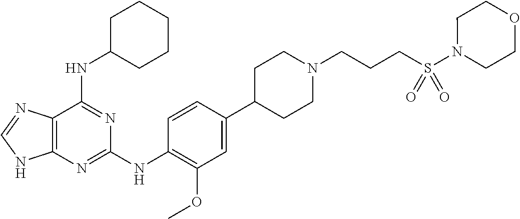 | Scheme 49 | Ret. Time (HPLC): 5.68 min; Mass Spec.: 613.32780 (M + 1) |
| C240 | [1-(4-{[6- (cyclohexyl- amino)- 9H-purin-2- yl]amino}-3- methoxy- phenyl) piperidin- 4-yl] (morpholin- 4-yl) methanone | 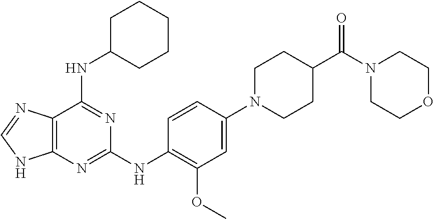 | Scheme 50 | Ret. Time (HPLC): 5.67 min; Mass Spec.: 535.31038 (M + 1) |
| C241 | N~6~- cyclohexyl- N~2~-{2- methoxy- 4-[4- (morpholin- 4-ylmethyl) piperidin- 1-yl] phenyl}- 9H-purine- 2,6-diamine | 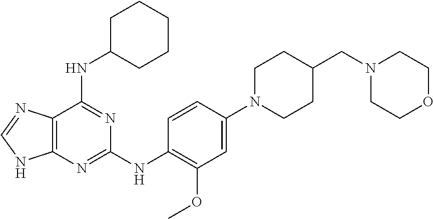 | Scheme 50 | Ret. Time (HPLC): 4.53 min; Mass Spec.: 521.33478 (M + 1) |
| C242 | [1-(4-{[6- (cyclohexyl- amino)- 9H-purin- 2-yl] amino}-3- methoxy- phenyl) piperidin- 4-yl][(2S)-2- (hydroxy- methyl) pyrrolidin- 1-yl] methanone | 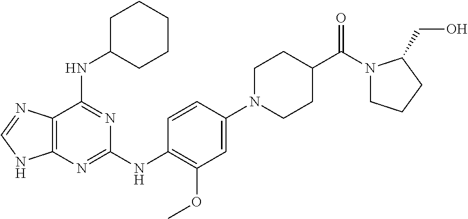 | Scheme 50 | Ret. Time (HPLC): 5.42 min; Mass Spec.: 549.32898 (M + 1) |
| C243 | 2-[1-(4-{[6- (cyclohexyl- amino)- 9H-purin- 2-yl] amino}-3- methoxy- phenyl) piperidin- 4-yl]-1- (morpholin- 4-yl) ethanone | 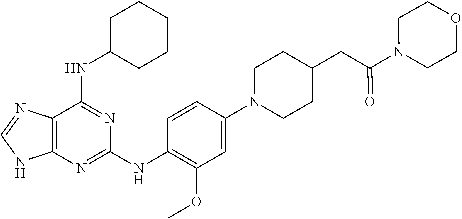 | Scheme 51 | Ret. Time (HPLC): 5.71 min; Mass Spec.: 549.3208 (M + 1) |
| C244 | 2-[1-(4-{[6- (cyclohexyl- amino)- 9H-purin- 2-yl] amino}-3- methoxy- phenyl) piperidin- 4-yl]-1- (pyrrolidin- 1-yl) ethanone | 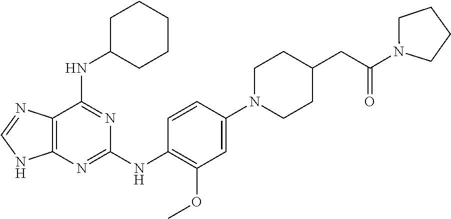 | Scheme 51 | Ret. Time (HPLC): 5.86 min; Mass Spec.: 533.33203 (M + 1) |
| C245 | N~6~- cyclohexyl- N~2~-[4-(1- ethyl- piperidin- 3-yl)-2- methoxy- phenyl] 9H-purine- 2,6-diamine | 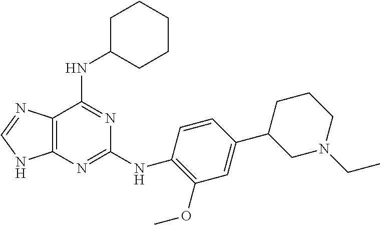 | Scheme 52 | Ret. Time (HPLC): 5.52 min; Mass Spec.: 450.29767 (M + 1) |
| C246 | N~2~-[4-(1- Ethyl- piperidin-3- yl)-2- methoxy- phenyl]- N~6~- (tetrahydro- 2H-pyran- 4-yl)-3H- purine- 2,6-diamine | 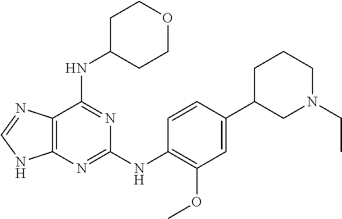 | Scheme 52 | Ret. Time (HPLC): 4.35 min; Mass Spec.: 452.27685 (M + 1) |
| C247 | N~6~- cyclohexyl- N~2~-{2- methoxy- 4-[1-(methyl sulfonyl) piperidin- 3-yl] phenyl}-9H- purine- 2,6-diamine | 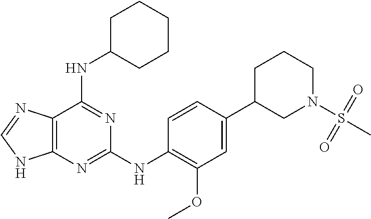 | Scheme 52 | Ret. Time (HPLC): 6.11 min; Mass Spec.: 500.24516 (M + 1) |
| C248 | N~6~- cyclohexyl- N~2~-{2- methoxy- 4-[3- (morpholin-4- ylmethyl) pyrrolidin- 1-yl] phenyl}- 9H-purine- 2,6-diamine | 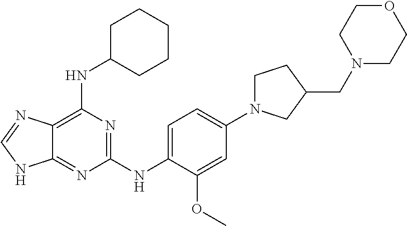 | Scheme 53 | Ret. Time (HPLC): 3.566 min; Mass Spec.: 507.3174 (M + 1) |
| C249 | 1-(4-{[6- (cyclohexyl- amino)- 9H-purin-2- yl]amino}-3- methoxy- phenyl)- 4-(morpholin- 4-ylmethyl) piperidin- 4-ol | 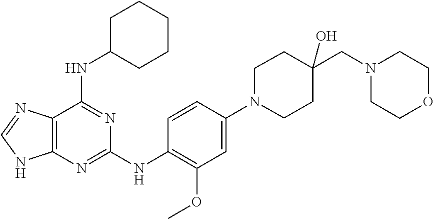 | Scheme 54 | Ret. Time (HPLC): 5.22 min; Mass Spec.: 537.32917 (M + 1) |
| C250 | 1-(4-{[6- (cyclohexyl- amino)- 9H-purin-2- yl]amino}-3- methoxy- phenyl)- 2- morpholin- 4-yl) ethanol | 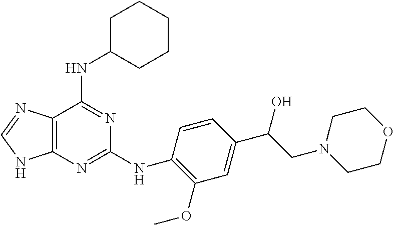 | Scheme 55 | Ret. Time (HPLC): 5.23 min; Mass Spec.: 468.27176 (M + 1) |
| C251 | 1-(3- methoxy-4- {[6- (tetrahydro- 2H-pyran-4- ylamino)-9H- purin-2- yl]amino} phenyl)-2- (morpholin- 4-yl) ethanol | 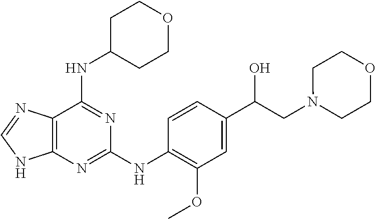 | Scheme 55 | Ret. Time (HPLC): 4.79 min; Mass Spec.: 470.25089 (M + 1) |
| C252 | N~2~-{2- Methoxy- 4-[1- (morpholin-4- yl)ethyl] phenyl}- N~6~- (tetrahydro- 2H-pyran-4- yl)-3H- purine-2,6- diamine | 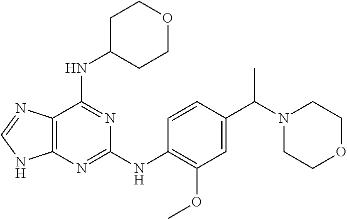 | Scheme 56 | Ret. Time (HPLC): 4.99 min; Mass Spec.: 454.25533 (M + 1) |
| C253 | N~2~-{2- methoxy- 4-[2- methyl-1- (morpholin- 4-yl)propan- 2-yl] phenyl}- N~6~- (tetrahydro- 2H-pyran- 4-yl)-9H- purine- 2,6-diamine | 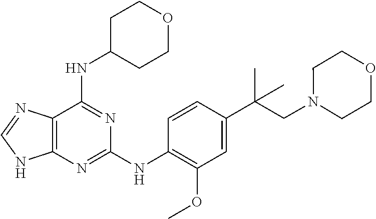 | Scheme 57 | Ret. Time (HPLC): 5.17 min; Mass Spec.: 482.28829 (M + 1) |
| C254 | N-[2- Chloro-4- (propan-2- yloxy) phenyl]- 6-[2- (morpholin- 4-yl) ethyl]-3H- purin-2- amine | 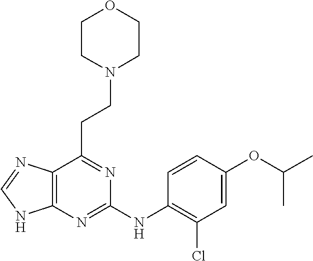 | Scheme 58 | Ret. Time (HPLC): 6.246 min; Mass Spec.: 417.18250 (M + 1) |
| C255 | N-[2- Methoxy-4- (morpholin- 4-yl) phenyl]- 6-[2- (morpholin- 4-yl)ethyl]- 3H-purin- 2-amine | 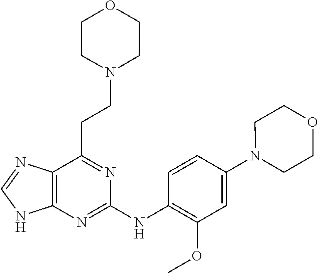 | Scheme 58 | Ret. Time (HPLC): 4.879 min; Mass Spec.: 440.24010 (M + 1) |
| C256 | N-[2- Methoxy-4- (morpholin- 4-yl) phenyl]- 6-[2- (piperidin- 1-yl) ethyl]-3H- purin-2- amine | 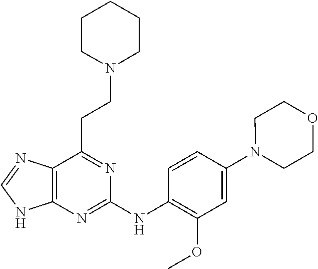 | Scheme 58 | Ret. Time (HPLC): 5.140 min; Mass Spec.: 438.26033 (M + 1) |
| C257 | N-[2- Chloro-4- (propan- 2-yloxy) phenyl]-6-[2- (piperidin- 1-yl) ethyl]-3H- purin-2- amine | 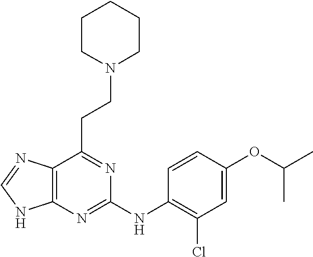 | Scheme 58 | Ret. Time (HPLC): 6.520 min; Mass Spec.: 415.20139 (M + 1) |
| C258 | N~6~- Cyclohexyl- N~2~- {2-methyl- 4-[(1E)-3- (morpholin- 4-yl)prop- 1-en-1-yl] phenyl}- 3H-purine- 2,6-diamine | 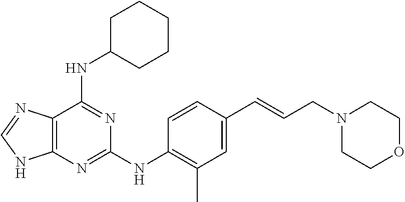 | Scheme 59 | Ret. Time (HPLC): 5.413 min; Mass Spec.: 448.28273 (M + 1) |
| C259 | N~6~- Cyclohexyl- N~2~-[4- (morpholin-4- yl)-2- (trifluoro- methoxy) phenyl]-3H- purine- 2,6-diamine | 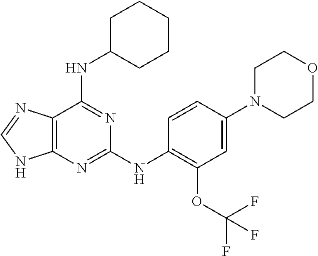 | Scheme 12 | Ret. Time (HPLC): 7.100 min; Mass Spec.: 478.20000 (M + 1) |
| C260 | 2-(3- Methoxy- 4-{[6- (tetrahydro- 2H-pyran-4- ylamino)- 3H-purin-2- yl]amino} phenyl)- 1-(morpholin- 4-yl) propan-1-one | 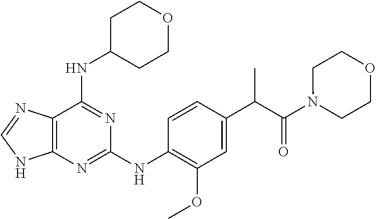 | Scheme 60 | Ret. Time (HPLC): 5.55 min; Mass Spec.: 482.25103 (M + 1) |
| C261 | 1-(3- Methoxy- 4-{[6- (tetrahydro- 2H-pyran-4- ylamino)- 3H-purin-2- yl]amino} phenyl) piperidine-4- carboxamide | 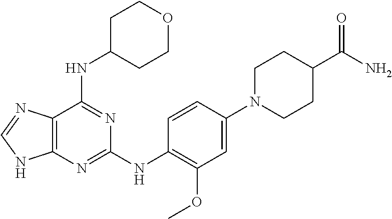 | Scheme 50 | Ret. Time (HPLC): 4.94 min; Mass Spec.: 467.25136 (M + 1) |
| C262 | N~6~- cyclohexyl- N~2~-(1H- indazol-6-yl)- 9H-purine- 2,6-diamine | 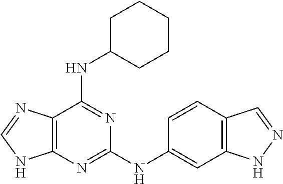 | Scheme 2 | Ret. Time (HPLC): 6.3 min; Mass Spec.: 349.1861 (M + 1) |
| C263 | N~6~- cyclohexyl- N~2~-(1H- indazol- 5-yl)- 9H-purine- 2,6-diamine | 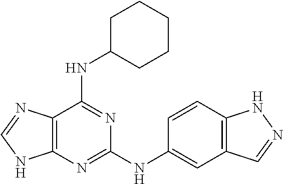 | Scheme 2 | Ret. Time (HPLC): 6.016 min; Mass Spec.: 349.1860 (M + 1) |
| C264 | N~6~- cyclohexyl- N~2~-(2,3- dihydro-1H- indol- 6-yl)-9H- purine-2,6- diamine | 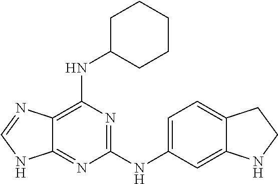 | Scheme 2 | Ret. Time (HPLC): 5.474 min; Mass Spec.: 350.2158 (M + 1) |
| C265 | 4-[[2- [(2-chloro-4- isopropoxy- phenyl) amino]- 9H-purin-6- yl]amino] cyclohexanol | 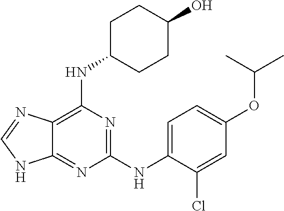 | Scheme 5 | Ret. Time (HPLC): 6.899 min; Mass Spec.: 417.18069 (M + 1) |

### Synthesis of N6-cyclohexyl-N2-(2-methyl-6-morpholino-3-pyridyl)-9H-purine-2,6-diamine (C266)

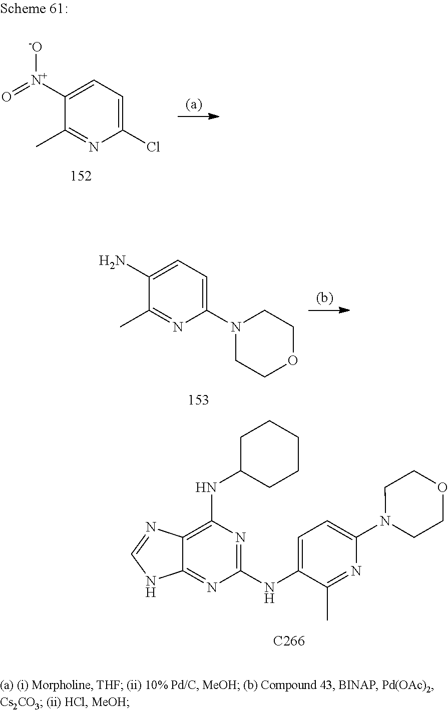

2-methyl-6-morpholino-pyridin-3-amine (153): 6-chloro-2-methyl-3-nitro-pyridine 152 (2.0 g, 11.6 mmol) and morpholine (2.02 mL, 22.2 mmol) were combined in tetrahydrofuran (5 mL) and stirred for 2 hours at room temperature. The intermediate nitro compound was isolated by filtration as a yellow solid. The yellow solid was taken up in methanol and hydrogenated in the presence of 10% Pd/C on a Parr hydrogenator at 30 psi for 2 hours. The reaction mixture was filtered over a bed of celite and concentrated. The residue was dissolved in methylene chloride and washed with saturated bicarbonate, water, and brine. The product was isolated after filtration as the HCl salt after precipitation with 4 mL 4 N HCl/dioxane. The product 152 was identified by ¹H-NMR.

N6-cyclohexyl-N2-(2-methyl-6-morpholino-3-pyridyl)-9H-purine-2,6-diamine: Compound 43 was coupled with 153 employing Buchwald reaction condition followed by hydrolysis of protecting group, as described in Scheme 12, furnished compound C266.

Compounds C267 and C268 were prepared according the procedure described above, using appropriately substituted starting materials.

### Synthesis of [4-[[2-[(2-methyl-6-morpholino-3-pyridyl)amino]-9H-purin-6-l]amino]cyclohexyl]methanol (C269)

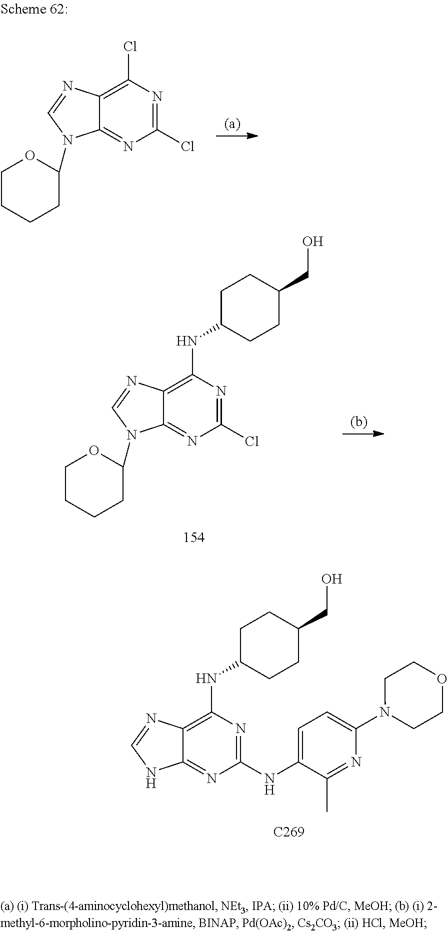

[4-[(2-chloro-9-tetrahydropyran-2-yl-purin-6-yl)amino]cyclohexyl]methanol (154): Trans-(4-aminocyclohexyl)methanol (1.51 g, 11.6 mmol), 2,6-dichloro-9-tetrahydropyran-2-yl-purine (3.16 g, 11.6 mmol) and triethylamine (4.8 mL, 34.8 mmol) were combined in isopropyl alcohol and heated at 80 C for 18 hours. Following solvent removal the reaction residue was purified by MPLC [silica: hexane/ethyl acetate] product was isolated as the free base (2.71 g, 7.4 mmol, 64%). [M+1]⁺=366.

[4-[[2-[(2-methyl-6-morpholino-3-pyridyl)amino]-9H-purin-6-yl]amino]cyclohexyl]methanol (C269): Compound 153 was coupled with 154 employing Buchwald reaction condition followed by hydrolysis of protecting group, as described in Scheme 12, to furnish compound C269.

In the Table 3, compound C270 was prepared in an analogous manner with appropriately substituted starting materials.

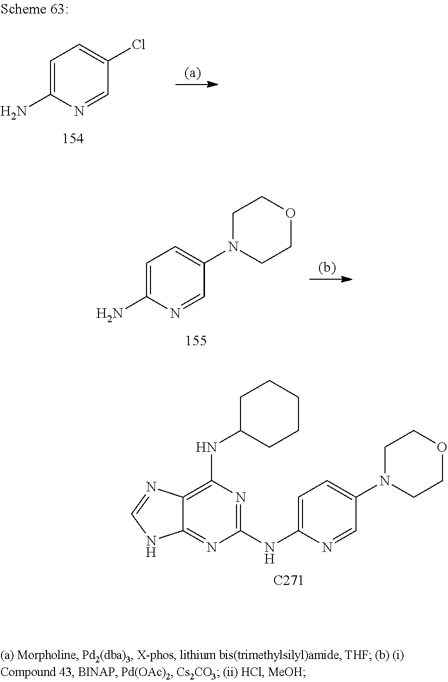

5-morpholinopyridin-2-amine (155): 2-amino-5-chloropyridine 154 (512 mg, 4 mmol), morpholine (1.04 mL, 12 mmol) Pd₂(dba)₃ (18.3 mg, 0.02 mmol) and X-phos (38.0 mg, 0.08 mmol) were combined in 9.0 mL of lithium bis(trimethylsilyl)amide (1.0 M in THF). The reaction vial was flushed with nitrogen and heated with magnetic stirring at 60° C. for 18 hours. The reaction mixture was filtered over Celite, diluted into methylene chloride, and washed 3× with water. The organic layer was dried over anhydrous sodium sulfate and concentrated to yield desired product (480 mg, 2.68 mmol, 67%). Product identified by GCMS with molecular ion at 179 amu.

N6-cyclohexyl-N2-(5-morpholino-2-pyridyl)-9H-purine-2,6-diamine (C271): Compound 155 was coupled with 43 employing Buchwald reaction condition followed by hydrolysis of protecting group, as described in Scheme 12, furnished compound C271. The product was purified by MPLC (C-18: MeOH/H₂O, 0.1% TFA. The ¹H-NMR is consistent with proposed structure.

In the Table 3, compound C272 was prepared in an analogous manner with appropriately substituted starting materials.

### Synthesis of N6-cyclohexyl-N2-[2-methyl-6-(3-morpholinoprop-1-ynyl)-3-pyridyl]-9H-purine-2,6-diamine (C273)

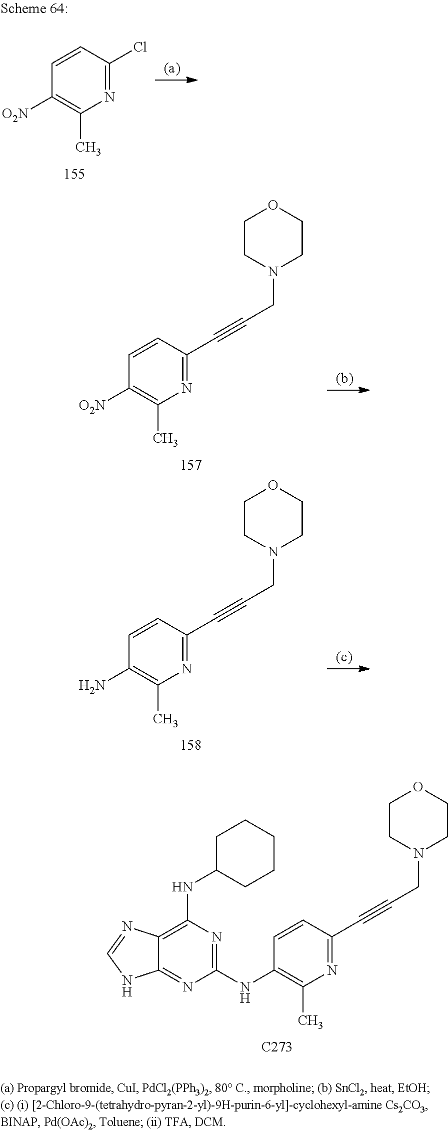

4-[3-(6-Methyl-5-nitro-2-pyridyl)prop-2-ynyl]morpholine (157): Compound 157 was synthesized using Sonagashira cross-coupling reaction conditions as described in Tetrahedron 2007, 63, 10671, starting from compound 155.

2-Methyl-6-(3-morpholinoprop-1-ynyl)pyridin-3-amine (158): To a stirred solution of 157 (1 equivalent) in ethanol (0.1 mmol solution) was added SnCl₂ (4 equivalents) and heated to 70° C. over 2 hours. Evaporated the solvent, added saturated solution of sodium bicarbonate, filtered solids and extracted repeatedly with ethyl acetate. Organic layer was washed with water, dried over anhydrous sodium sulfate and concentrated the product 158 and used as such in the next step.

N6-cyclohexyl-N2-[2-methyl-6-(3-morpholinoprop-1-ynyl)-3-pyridyl]-9H-purine-2,6-diamine (C273)Compound 158 was coupled with 43, employing Buchwald reaction condition followed by hydrolysis of protecting group as described in Scheme 12, to provide compound C273. The product was purified by reverse phase MPLC (C-18: MeOH/H₂O, 0.1% TFA). The ¹H-NMR is consistent with proposed structure.

### Synthesis of N6-cyclohexyl-N2-[2-methyl-6-(3-morpholinopropyl)-3-pyridyl]-9H-purine-2,6-diamine (C274)

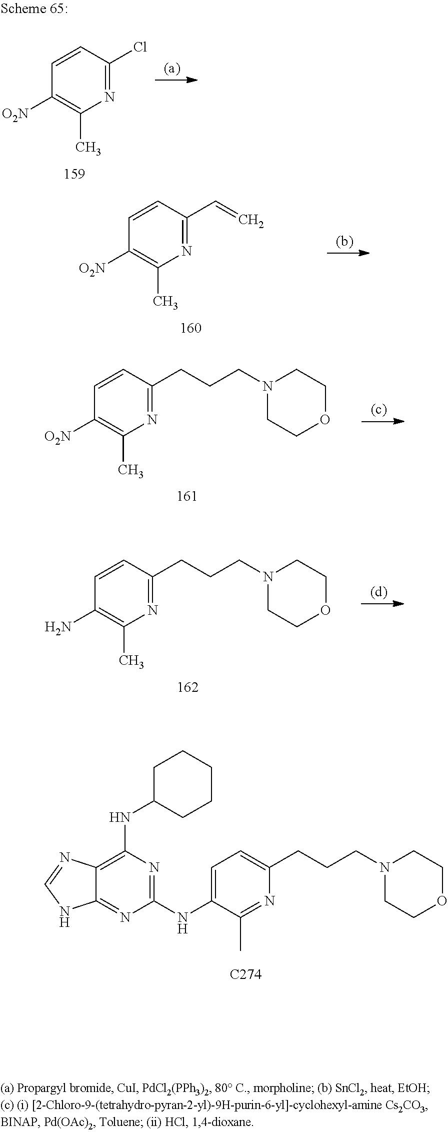

2-methyl-3-nitro-6-vinyl-pyridine (160): To a solution of 6-chloro-2-methyl-3-nitro-pyridine (1.0 g, 6.0 mmol) and tributyl(vinyl)stannane (2.1 mL, 7.0 mmol) in toluene (70 mL) under nitrogen was added Pd(PPh3)₄ (0.35 g, 0.3 mmol). The solution was degassed then heated to reflux for 18 hr under nitrogen atmosphere. The reaction was concentrated and the resulting residue purified by silica gel flash chromatography using EtOAc/Hexanes as eluent to afford 2-methyl-3-nitro-6-vinyl-pyridine obtained as an orange oil (0.58 g).

4-[3-(6-methyl-5-nitro-2-pyridyl)propyl]morpholine (161): To a solution of 2-methyl-3-nitro-6-vinyl-pyridine (0.1 g, 0.6 mmol) in DCM (6 mL) was added morpholine (0.09 mL, 0.1 mmol). The solution was allowed to stir for 72 hr under nitrogen atmosphere. The reaction was concentrated and to afford 4-[3-(3-methyl-4-nitro-phenyl)propyl]morpholine used without further purification.

2-methyl-6-(3-morpholinopropyl)pyridin-3-amine (162): To 4-[3-(3-methyl-4-nitro-phenyl)propyl]morpholine (150 mgs, 0.6 mmol) in MeOH (6 mLs) was added Pd/C (20 mgs, 10% Pd on cabon by wt.). The reaction was placed under a H2 balloon and allowed to stir for 6 hrs. Filtration over cellite and concentration in vacuo afforded 4-[3-(6-methyl-5-nitro-2-pyridyl)propyl]morpholine used without further purification.

N6-cyclohexyl-N2-[2-methyl-6-(3-morpholinopropyl)-3-pyridyl]-9H-purine-2,6-diamine (C274): Compound 162 was coupled with 43 employing Buchwald reaction condition followed by hydrolysis of protecting group, as described in Scheme 12, furnished compound C274. The product was purified by MPLC (C-18: MeOH/H₂O, 0.1% TFA). The ¹H-NMR is consistent with proposed structure.

### Synthesis of [5-[[6-(cyclohexylamino)-9H-purin-2-yl]amino]-6-methyl-2-pyridyl]-morpholino-methanone (C275)

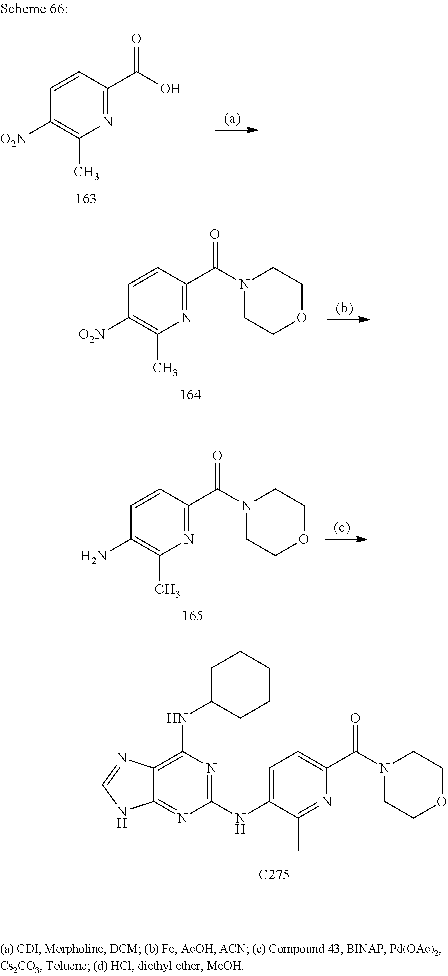

(6-Methyl-5-nitro-2-pyridyl)-morpholino-methanone (164): To a stirred solution of 163 (200 mg, 1.32 mmol) in DCM (13 mL) was added CDI (256 mg, 1.58 mmols) and after 1 hour morpholine (2 equivalent) stirred at room temperature overnight. Evaporated solvent and purification of the residue over silica gel column chromatography provided the desired amide 164.

(5-amino-6-methyl-2-pyridyl)-morpholino-methanone (165): Compound crude 164 (1 equivalent) was reduced using iron (5 equivalent) and acetic acid (10 equivalent) in acetonitrile (0.1 mmol) at room temperature over 4 hours. Solids were filtered off and concentrated to the desired intermediate used in the next step without further purification.

[5-[[6-(cyclohexylamino)-9H-purin-2-yl]amino]-6-methyl-2-pyridyl]-morpholino-methanone (C275): Compound 164 was coupled with 43 employing Buchwald reaction condition followed by hydrolysis of protecting group, as described in Scheme 12, furnished compound C275. The product was purified by MPLC (C-18: MeOH/H₂O, 0.1% TFA). The ¹H-NMR is consistent with proposed structure.

In the Table 3, compound C276 was prepared in an analogous manner with appropriately substituted starting materials.

The structures and characterization of the exemplary compounds made and the synthetic scheme of each (appropriate, commercially available starting materials are known to those skilled in the art) are provided in Table 3 below.

TABLE 3
Further Additional Exemplary Compounds of the Invention

| Compound # | IUPAC Name | Structure | Synthesis | Analytical data |
|---|---|---|---|---|
| C266 | N~6~- Cyclohexyl- N~2~-[2-methyl- 6-(morpholin-4- yl)pyridin-3-yl]- 3H-purine-2,6- diamine | 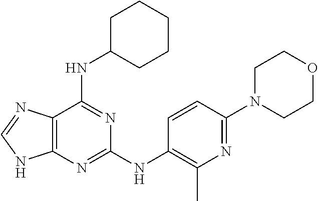 | Scheme 61 | Ret. Time (HPLC) 6.161 min; Mass Spec.: 409.24340 (M + 1) |
| C267 | N~6~- cyclohexyl- N~2~-{2-methyl- 6-[4- (methylsulfonyl) piperazin-1- yl]pyridin-3-yl}- 9H-purine-2,6- diamine | 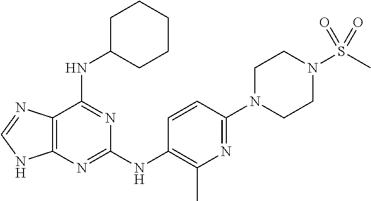 | Scheme 61 | Ret. Time (HPLC) 6.029 min; Mass Spec.: 486.23571 (M + 1) |
| C268 | N~6~- cyclohexyl- N~2~-{2-methyl- 6-[4-(morpholin- 4-yl)piperidin-1- yl]pyridin-3-yl}- 9H-purine-2,6- diamine | 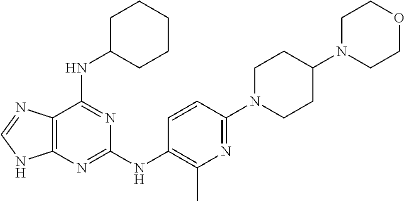 | Scheme 61 | Ret. Time (HPLC) 4.953 min; Mass Spec.: 492.32032 (M + 1) |
| C269 | {trans-4-[(2-{[2- methyl-6- (morpholin-4- yl)pyridin-3- yl]amino}-9H- purin-6- yl)amino] cyclohexyl}methanol | 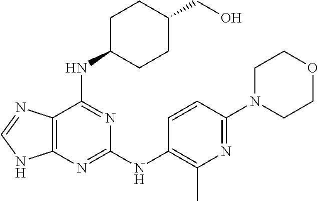 | Scheme 62 | Ret. Time (HPLC) 4.946 min; Mass Spec.: 438.25497 (M + 1) |
| C270 | (trans-4-{[2-({2- methyl-6-[4- (morpholin-4- yl)piperidin-1- yl]pyridin-3- yl}amino)-9H- purin-6- yl]amino} cyclohexyl)methanol | 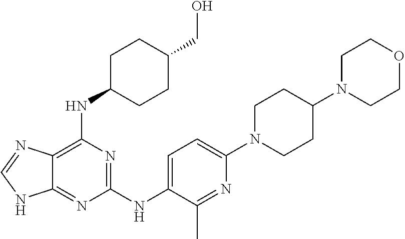 | Scheme 62 | Ret. Time (HPLC) 4.409 min; Mass Spec.: 522.32986 |
| C271 | N~6~- cyclohexyl- N~2~-[5- (morpholin-4- yl)pyridin-2-yl]- 9H-purine-2,6- diamine | 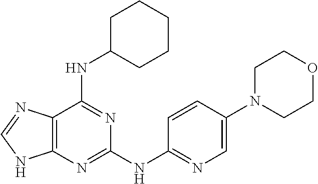 | Scheme 63 | Ret. Time (HPLC) 6.449 min; Mass Spec.: 395.22840 |
| C272 | N~6~- Cyclohexyl- N~2~-{5-[4- (morpholin-4- yl)piperidin-1- yl]pyridin-2-yl}- 3H-purine-2,6- diamine | 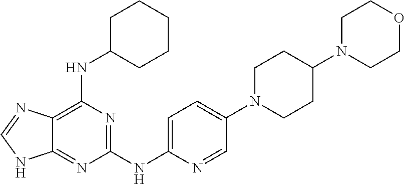 | Scheme 63 | Ret. Time (HPLC) 5.275 min; Mass Spec.: 478.30420 (M + 1) |
| C273 | N~6~- Cyclohexyl- N~2~-{2-methyl- 6-[3-(morpholin- 4-yl)prop-1-yn- 1-yl]pyridin-3- yl}-3H-purine- 2,6-diamine | 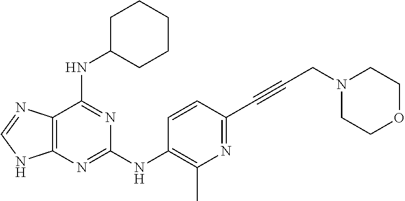 | Scheme 64 | Ret. Time (HPLC): 5.690 min; Mass Spec.: 462.26120 (M + 1) |
| C274 | N~6~- Cyclohexyl- N~2~-{2-methyl- 6-[2-(morpholin- 4- yl)ethyl]pyridin- 3-yl}-3H-purine- 2,6-diamine | 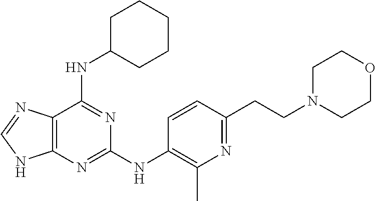 | Scheme 65 | Ret. Time (HPLC): 4.988 min; Mass Spec.: 437.27864 (M + 1) |
| C275 | (5-{[6- (cyclohexylamino)- 9H-purin-2- yl]amino}-6- methylpyridin-2- yl)(morpholin-4- yl)methanone | 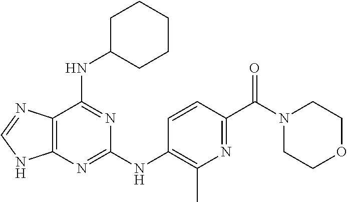 | Scheme 66 | Ret. Time (HPLC): 5.861 min; Mass Spec.: 437.21216 (M + 1) |
| C276 | 5-{[6- (Cyclohexylamino)- 3H-purin-2- yl]amino}-N- ethyl-6- methylpyridine- 2-carboxamide | 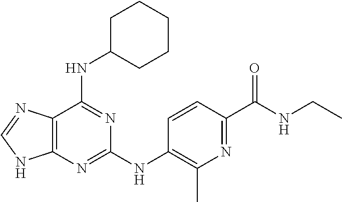 | Scheme 66 | Ret. Time (HPLC): 7.331 min; Mass Spec.: 395.23065 (M + 1) |
| C277 | trans-4-({2-[(2,6- Dimethoxypyridin- 3-yl)amino]- 3H-purin-6- yl}amino) cyclohexanol | 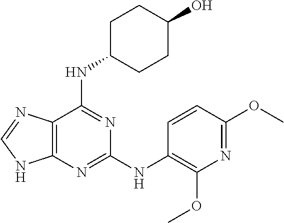 | Scheme 2 | Ret. Time (HPLC): 6.181 min; Mass Spec.: 386.19454 (M + 1) |
| C278 | N~6~- Cyclohexyl- N~2~-[2-ethoxy- 6-(morpholin-4- yl)pyridin-3-yl]- 3H-purine-2,6- diamine | 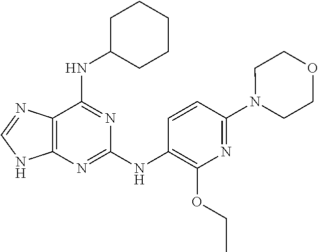 | Scheme 2 | Ret. Time (HPLC) 8.059 min; Mass Spec.: 439.25607 (M + 1) |
| C279 | N~6~- (Bicyclo[2.2.1] hept-2-yl)-N~2~- (2,6- dimethoxypyridin- 3-yl)-3H- purine-2,6- diamine | 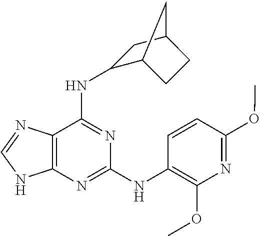 | Scheme 2 | Ret. Time (HPLC): 7.559 min; Mass Spec.: 382.2212 (M + 1) |
| C280 | N~6~- Cyclohexyl- N~2~-(2,6- dimethoxypyridin- 3-yl)-N~6~- methyl-3H- purine-2,6- diamine | 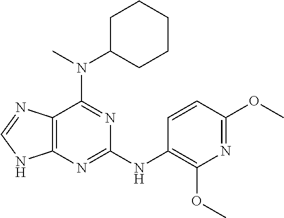 | Scheme 2 | Ret. Time (HPLC): 7.343 min; Mass Spec.: 384.21426 (M + 1) |
| C281 | N~6~- Cyclohexyl- N~2~-(1,3- dimethyl-1H- pyrazol-5-yl)- 3H-purine-2,6- diamine | 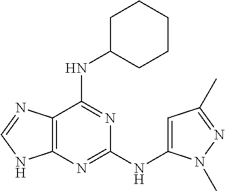 | Scheme 2 | Ret. Time (HPLC) 6.615 min; Mass Spec.: 327.20418 (M + 1) |
| C282 | N,N′-Bis(2,6- dimethoxypyridin- 3-yl)-3H- purine-2,6- diamine | 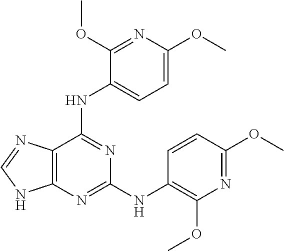 | Scheme 2 | Ret. Time (HPLC): 7.886 min; Mass Spec.: 425.16803 (M + 1) |
| C283 | N~6~- cyclohexyl- N~2~-(5-fluoro- 1-oxidopyridin- 2-yl)-9H-purine- 2,6-diamine | 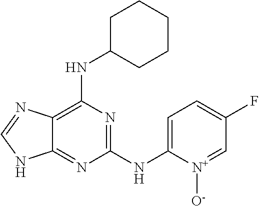 | Scheme 2 | Ret. Time (HPLC): 6.884 min; Mass Spec.: 344.16296 (M + 1) |
| C285 | N~6~- Cyclohexyl- N~2~-[4-methyl- 6-(morpholin-4- yl)pyridin-3-yl]- 3H-purine-2,6- diamine | 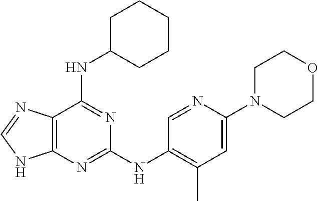 | Scheme 2 | Ret. Time (HPLC) 6.119 min; Mass Spec.: 409.24523 (M + 1) |
| C287 | 5-({6-[(4- Hydroxycyclohexyl) amino]-3H- purin-2- yl}amino)-4- methylpyridin- 2(1H)-one | 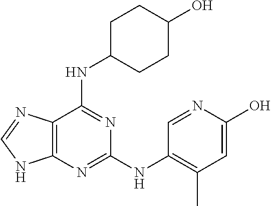 | Scheme 2 | Ret. Time (HPLC) 4.868 min; Mass Spec.: 356.18280 (M + 1) |
| C288 | 4-({2-[(3- Methyl-1H- pyrazol-5- yl)amino]-3H- purin-6- yl}amino) cyclohexanol | 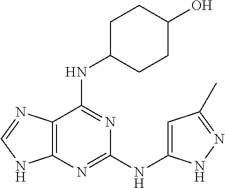 | Scheme 2 | Ret. Time (HPLC): 4.922 min; Mass Spec.: 329.18489 (M + 1) |

The HPLC conditions used to characterize each compound listed in Table 1 are as follows:
Flow Rate: 1.2 mL/minute
Solvents: A: Water+0.01% TFA
B: Acetonitrile (ACN)+0.01% trifluoroacetic acid (TFA)
Gradient: 5% B for 1 minute
5% B to 100% B in 9 minutes
at 100% B for 2.4 minutes
to 0% B in 0.1 minutes
at 0% for 0.5 minutes
Overall time: 13.00 minutes
Column: XTerra MS C18 3.5 um 4.6×150 mm

### Example 2

### Determination of Anti-Cancer Activity

The compounds of the invention were tested in the following cytotoxicity assay to detect anti-cancer activity. This assay determined viability of various cell lines, when treated with a test compound, by determining the metabolic activity of proliferating cells using the tetrazolium salt WST. While assays conducted with HCT116 colon cancer cell line cells were generally used to compare the relative efficacy of the compounds being tested, OVCAR8 and NCI/ADR-Res cell lines were also used according to the same procedure. In the HCT116-based Cytotoxicity assays, the compounds of the present invention show cytotoxicity with an IC₅₀ (i.e., concentration resulting in a 50% reduction in cell viability) of less than 10 μM.

MATERIALS:
A. 10% FBS/L-glutamine/NEAA/HEPES/Na-Pyruvate/RPMI, phenol-free;
B. HCT116, OVCAR8 or NCI/ADR-Res cell lines (new vial thawed every 8-10 weeks);
C. Test compounds, typically 50 mM stocks diluted in DMSO;
D. Clear, flat, TC-surface bottomed 96-well microplates (Corning #3595);
E. WST-1: Dojindo, W201 (powder).

METHODS:
1. HCT116, OVCAR8 or NCI/ADR-Res cell line cells were plated at a density of 5,000 cells in 100 μL/well in a 96-well plate, and were incubated overnight.
   a) Each compound, dissolved in dimethylsufoxide (DMSO) as shown below, was tested in triplicate, and two compounds were tested per plate.
   b) Blank Controls: A plate containing media only.
2. In the morning of the next day:
   a) 250× compound dilution plates were prepared as shown in Tables 4 and 5 below.

TABLE 4
Standard Dilution Series
Standard SAR compound dilution series (10 μM starting conc., 1:4 dilutions)
50 mM stock solution

| | | | | | | | | | | | |
|---|---|---|---|---|---|---|---|---|---|---|---|
| 250× | 2.5 | 0.625 | 0.156 | 0.04 | 0.01 | 0.002 | 0.0006 | 0.0002 | 4 × 10⁻⁵ | 0 | mM |
| cmpd | | | | | | | | | | | |
| final conc. | 10 | 2.5 | 0.625 | 0.156 | 0.039 | 0.01 | 0.002 | 0.0006 | 0.0002 | 0 | μM |
| Dilution | 1:20 | 1:04 | 1:04 | 1:04 | 1:04 | 1:04 | 1:04 | 1:04 | 1:04 | NA | cmpd: |
| | | | | | | | | | | | DMSO |
| dilution | 2 to 38 | 10 to 30 | 10 to 30 | 10 to 30 | 10 to 30 | 10 to 30 | 10 to 30 | 10 to 30 | 10 to 30 | 30 μl | cmpd: |
| volumes | | | | | | | | | | DMSO | DMSO |
| (μL) | | | | | | | | | | | |

*Add 0.4 μl diluted compound to 100 μl of cells + media

TABLE 5
Control Compound Dilution Series
Control Compound Dilution Series (10 μM starting conc., 1:5 dilutions)
50 mM stock solution

| | | | | | | | | | | | |
|---|---|---|---|---|---|---|---|---|---|---|---|
| 250× cmpd | 2.5 | 0.5 | 0.1 | 0.02 | 0.004 | 0.0008 | 0.0002 | 3 × 10⁻⁵ | 1 × 10⁻⁵ | 0 | mM |
| final conc. | 10 | 2 | 0.4 | 0.08 | 0.016 | 0.0032 | 0.0006 | 0.0001 | 3 × 10⁻⁵ | 0 | μM |
| Dilution | 1:20 | 1:05 | 1:05 | 1:05 | 1:05 | 1:05 | 1:05 | 1:05 | 1:05 | NA | cmpd: |
| | | | | | | | | | | | DMSO |
| dilution | 2 to 38 | 10 to 40 | 10 to 40 | 10 to 40 | 10 to 40 | 10 to 40 | 10 to 40 | 10 to 40 | 10 to 40 | 40 μL | cmpd: |
| volumes | | | | | | | | | | DMSO | DMSO |
| (μL) | | | | | | | | | | | |

*Add 0.4 μL diluted compound to 100 μl of cells + media

b) Without removing culture medium, 0.4 μl of a 250× concentrated solution of test compound in growth media was added to HCT116, OVCAR8 or NCI/ADR-Res plates. Plates were returned to incubator and left to incubate for 72 hours.
c) 10 μL of prepared WST-1 was added to treated cells and allowed to incubate at 37° C. for 90 minutes.
d) Absorbances at 490 and 650 nm were read and viability of treated cells relative to DMSO control was determined.

### Example 2B

The compounds of the present invention were tested against a panel of cancer cells to determine the effect on cell viability. Viability was determined by measuring cellular ATP levels using CellTiter Glo reagent (Promega, Madison, Wis.).

MATERIALS:
A. Cancer cell lines obtained from ATCC or DSMZ
B. RPMI supplemented with 10% FBS/L-glutamine/NEAA/HEPES/Na-Pyruvate
C. DMEM supplemented with 10% FBS and L-glutamine.
B. Compounds, typically 50 mM stocks diluted in DMSO;
C. Clear, flat, TC-surface bottomed 96-well microplates (Corning #3595)
D. CellTiter-Glo® Luminescent Cell Viability Assay (Promega #G7573)

METHODS:
1. Cells were plated at densities of 2000-5000 cells per well in 100 μl volume on a 96-well plate, and were incubated overnight.
2. Test compound(s) were serially diluted to the desired 250× concentration.
3. For each concentration of compound to be tested, 0.4 μl of 250× compound was added in triplicate to the cell plates. Plates were then incubated at 37° C. for 7 days.
4. 100 μL of prepared CellTiter Glo reagent was added to treated cells, and plates were gently mixed for 2 minutes, and equilibrated for 10 minutes at room temperature.
5. Luminescence was measured and the viability of treated cells relative to DMSO control was calculated.

TABLE 6
Cytotoxicity of N6-cyclohexyl-N2-(2-methyl-4-morpholino-phenyl)-9H-
purine-2,6-diamine—an exemplary TTK inhibitor of the present
invention—to aVariety of Cancer Cell Lines.

| Tissue | | 7 day IC50 |
|---|---|---|
| Type | Cell Line | (μM) |
| blood | OPM-2 | 0.099 |
| | KARPAS-299 | 0.096 |
| breast | DU4475 | 0.072 |
| | BT-474 | >30 |
| | MCF-7 | 0.061 |
| | MDA-MB-231 | 28 |
| | MDA-MB-436 | 8.7 |
| | SK-BR-3 | 0.056 |
| | ZR-75-1 | 0.056 |
| CNS | U-87MG | 0.67 |
| colon | Colo-205 | 0.09 |
| | DLD-1 | 0.11 |
| | HCT-15 | 0.28 |
| | SW-480 | 0.14 |
| gastric | NCI-N87 | 2.1 |
| kidney | 786-0 | 1.2 |
| lung | A549 | 0.11 |
| | HCC827 | 0.094 |
| | DMS-114 | 0.18 |
| melanoma | LOX-1MV1 | 1.1 |
| | MALME-3M | 0.11 |
| ovarian | IGR-OV1 | 0.58 |
| | OVCAR-3 | 0.15 |
| | OVCAR-5 | 0.15 |
| pancreas | PANC1 | 3.7 |
| | BxPC-3 | 0.12 |
| prostate | DU-145 | 0.37 |
| | PC-3 | 0.2 |

Cancer cell lines were treated with TTK inhibitor for 7 days and cell viability was measured using the CellTiter Glo assay (Promega, Madison, Wis.).

### Example 3

### Determination of TTK Inhibition

The compounds of the invention were also tested in the following assay to determine TTK-inhibitory activity. This assay measures transfer of radioactive phosphate from ATP to a protein substrate in the presence or absence of a test compound. The compounds of the invention show TTK inhibition in this assay with an IC₅₀ of less than 1 μM.

MATERIALS:
A. Recombinant TTK enzyme;
B. Phosphorylated myelin basic protein (MBP);
C. [γ-³³P]ATP: 1 mCi/100 μL;
D. Positive Control: a proprietary compound known to have no activity in this assay.

METHODS:

Final concentrations of reaction mixture components (50 μl total volume): 50 mM Tris-HCl, pH 7.5; 0.01% Triton X-100; 10 mM MgCl₂; 25 ng TTK (˜4 nM); 5 μM Phosphorylated MBP; 40 μM ATP (1 μCi/well, adjusted ATP specific activity according to strength of radiolabeled ATP).

In setting up the assay for the specific compounds to be tested, the following mixes were prepared as shown in Tables 7 & 8, below:

TABLE 7
Reaction Mix

| | | |
|---|---|---|
| Kinase reaction | per reaction (1X) | 110X (1 plate) |

| | | | | |
|---|---|---|---|---|
| 50 mM Tris-HCl, pH 7.5 | 2.5 | μL 1M Tris-HCl, pH 7.5 | 275 | μL |
| 0.01% Triton X-100 | 0.025 | μL 20% Triton X-100 | 2.75 | μL |
| 10 mM MgCl2 | 1 | μL 0.5M MgCl2 | 110 | μL |
| 25 ng TTK | 0.051 | μL 490 ng/μL TTK | 5.61 | μL |
| 5 μM Phosphorylated MBP | 0.92 | μL 271.7 μM Phosp. MBP | 101.2 | μL |
| | 34.504 | μL dH2O | 3795.44 | μL |
| | 39 | μL total | 4290 | μL total |

TABLE 8
ATP Mix (5x)

| Kinase reaction | per reaction (1X) | | 110X (1 plate) | |
|---|---|---|---|---|
| 40 μM ATP | 0.2 | μL of 10 mM ATP | 22 | μL |
| 1 μCi/well | 0.1 | μL of 10 μCi/μL [γ-³³P]ATP | 11 | μL |
| [γ-³³P]ATP | | | | |
| | 9.7 | μL dH2O | 1067 | μL |
| | 10 | μL total | 1100 | μL total |

Mixes were assembled in the wells of a 96-well polypropylene plate as follows: 1 μL 50× compound to be tested; 39 μL 1.282× Reaction Mix; 10 μL 5×ATP Mix.

The following reaction/assay steps were then performed:
1. Mix thoroughly and incubate at room temperature for 45 minutes;
2. Add 70 μL/well, except in background wells, of 3% phosphoric acid to terminate reaction, and transfer 100 μL to P81 filter plate;
3. Wash with 1% phosphoric acid, then dry the P81 filter plate completely;
4. Add 100 μL/well MicroSint-20 (PerkinElmer #6013621) and seal;
5. Read plate on TopCount (³³P/MicroSint) to determine amount of radiolabeled ³³P remaining (and hence the ability of the test compound to inhibit TTK-catalyzed transfer of ³³P, relative to negative controls).

### Example 4

### Determination of Mitotic Check-Point Escape (G₂/M Escape)

The compounds of the invention were tested in the following G₂/M Check-Point Escape assay. This assay determines the percentage of cells in mitosis in the presence or absence of TTK inhibitors by determining the phosphorylation state of the mitotic marker, histone H3.

MATERIALS:
A. Fetal Bovine Serum (Hyclone SV3-0096, Lot No. JRM28372)
B. Glutamax (Invitrogen 35050-061)
C. Sodium Pyruvate (Invitrogen 11360-070)
D. HEPES (Invitrogen 15630-080)
E. MEM non-essential amino acids (Invitrogen 11140-050)
F. DMEM (Invitrogen 31053-028)
G. HeLa cell line (ATCC CCL-2);
H. Compounds, typically 50 mM stocks diluted in DMSO;
I. Clear, flat, 96-well tissue culture plates (Greiner Bio-One μ-clear #655090);
J. Nocodazole (Sigma M1404);
K. Mouse monoclonal anti-β-tubulin antibody (Sigma T5293)
L. Rabbit polyclonal anti-phospho histone H3 Ser10 antibody (Upstate 06-570 1 mg/mL)
M. Alexa Fluor® 488-conjugated goat anti-Mouse IgG antibody (Invitrogen A11029, 2 mg/mL)
N. Alexa Fluor® 546-conjugated goat anti-rabbit IgG antibody (Invitrogen A11035, 2 mg/mL)
O. Hoechst 33342 DNA stain (Calbiochem 382065, 400 μg/mL (100×))
P. 35% Bovine Serum Albumin Solution in DPBS (Sigma A7979)
Q. Tween 20 (Sigma P5927)
R. Triton X-100 (Shelton Scientific, Inc IB07100)
S. 20% Paraformaldehyde (Ladd Research #20295)

METHODS:
1. HeLa cells were plated at densities of 5000 cells/well in 75 μL in a 96-well plate and incubated 7 hours.
2. 5 μL of a 10 mg/mL nocodazole stock was diluted into 2 mL of tissue culture media to prepare a 25 μg/mL nocodazole solution. 1 μl of the solution was added into the 75 μL of cell culture for a final concentration of 330 ng/mL nocodazole.
3. Cells were incubated with 330 ng/mL nocodazole for 17 hours.
4. The morning of the next day:
   a) 4× compound dilution plates were prepared as described in Table 9, below.

TABLE 9
Standard Compound Dilution Series
Standard SAR compound dilution series (10 μM starting conc., 1:4 dilutions)
50 mM stock solution (Dilution is into Tissue culture media containing 0.8% DMSO)

| | | | | | | | | | |
|---|---|---|---|---|---|---|---|---|---|
| 4× cmpd | 40 | 10 | 2.5 | 0.625 | 0.156 | 0.390 | 0.010 | 0 | mM |
| final conc. | 10 | 2.5 | 0.625 | 0.156 | 0.039 | 0.010 | 0.002 | 0 | μM |
| dilution | 1:1250 | 1:4 | 1:4 | 1:4 | 1:4 | 1:4 | 1:4 | NA | cmpd: |
| | | | | | | | | | DMSO |
| dilution | 1 to 1250 | 50 to 150 | 50 to 150 | 50 to 150 | 50 to 150 | 50 to 150 | 50 to 150 | | cmpd: |
| volumes | | | | | | | | | DMSO |
| (μL) | | | | | | | | | |

*Add 25 μl diluted compound to 75 μl of cells + media

b) Without removing culture medium, 25 μl of 4× compound was dispensed into HeLa cell plate wells such that each compound was tested in duplicate wells at every concentration. Plates were incubated at 37° C. for 4 hours.
c) Cells were fixed by adding 25 μL of 20% Paraformaldehyde (4% final) to the treated cells, and cells were incubated at 37° C. for 15 minutes.
d) The fixative was removed by aspiration and 50 μL/well of 0.2% Triton-X 100 in HBSS was added to permeabilize the cells. The mixture was incubated at 37° C. for 5 minutes.
e) The permeabilizing agent was removed by aspiration and the fixed, permabilized cells in each well were washed three times with 100 μL HBSS.
f) The final wash was removed by aspiration, and 50 μL/well blocking agent (1% BSA, 0.1% Tween 20) was added. Plates were incubated at 37° C. for 60 minutes.
g) The blocking agent was removed by aspiration, and 50 μL/well primary antibodies (mouse anti-β-tubulin 1:200 and rabbit anti-phospho histone H3 1:200) diluted in blocking agent were added. The mixture was incubated at 37° C. for 45 minutes.
h) The primary antibody solution was removed by aspiration and wells were washed three times with 100 μL HBSS.
i) The final wash was removed by aspiriation, and 50 μL/well of secondary antibodies (goat anti-mouse IgG-488 1:500 and goat anti-rabbit IgG-546 1:500) diluted in blocking agent were added. Plates were incubated at 37° C. for 45 minutes.
j) The secondary antibody solution was removed by aspiration and wells were washed three times with 100 μL HBSS.
k) 50 μL/well Hoechst DNA stain (4 μg/mL in HBSS) was added, and plates were incubated at 37° C. for 5 minutes.
l) The Hoechst DNA stain was removed by aspiration and wells were washed three times with 100 μL HBSS.
m) Plates were imaged using a BD Pathway 800 Bioimager. Cell numbers were scored based on the brightness of Hoechst-stained nuclei.
Numbers of mitotic cells were scored based on the brightness of the phospho-histone H3 staining. The percentage of mitotic cells was calculated based on the above staining scores. The percentage of mitotic cells was plotted versus test compound concentration to determine the EC₅₀.

### Example 5

### Effects of Aurora and TTK Inhibitors on Cell Cycle Progression

HeLa cells were synchronized in the G₁ phase of the cell cycle using a double thymidine block. The cells were released from the block by removing the media and replacing it with media containing DMSO, 500 nM VX-680 (Aurora inhibitor), or a TTK inhibitor. At the indicated time points, cells were fixed with methanol, permeabilized and stained with propidium iodide. DNA content was analyzed using a Guava EasyCyte flow cytometer.

Inhibitors of Aurora B kinase have been shown to induce polyploidy in various tumor cell lines. This is due to improper spindle formation and chromosome segregation followed by failed cytokinesis. Previously, it has been shown that inhibition of TTK using siRNA results in chromosome missegregation and mitotic defects. Using TTK inhibitors of the present invention, defects in chromosome segregation were observed. Therefore, the effect of the TTK inhibitors of the present invention were analyzed for their effect on cell cycle progression and/or DNA content. In the presence of TTK inhibitors, cells released from a G₁ block were able to progress through mitosis, divide and re-enter the G₁ phase of the cell cycle with a 2N DNA content, indicating that there is not a gross failure of cytokinesis resulting from TTK inhibition. This is in contrast to the Aurora inhibitor, VX-680, which causes cells to become tetraploid.

### Example 6

### Biomarker Examples

### Materials and Methods for Biomarker Examples

Image analysis: Cells were fixed and stained with Hoechst dye and anti-(3-tubulin, anti-pericentrin and anti-centromere antibodies. Images were taken on a BD Pathway high content imaging system. For cell cycle analysis, cells were fixed, stained with propidium iodide and analyzed by FACS. For Western blot analysis, cell lysates were run on SDS-polyacrylamide gels and proteins were transferred to PVDF membrane. Membranes were probed with the indicated antibodies.

Reagents: The exemplary TTK inhibitor used was one of the compounds of the present invention. Specifically, the compound was N6-cyclohexyl-N2-(2-methyl-4-morpholino-phenyl)-9H-purine-2,6-diamine, which is identified as C9 in Table 1, above. In the Figures and Examples included herein, N6-cyclohexyl-N2-(2-methyl-4-morpholino-phenyl)-9H-purine-2,6-diamine is referred to as alternatively as “Compound A” or simply “TTK inhibitor.” The Aurora kinase inhibitor used was VX-680. The PLK inhibitors used were thiophene benzimidazole (PLK1 and PLK3). The TTK, Aurora, and PLK inhibitors were synthesized in-house. KU-55933 and CGK 733 were purchased from Calbiochem. 4-Nitroquinoline 1-oxide (4-NQO), protease inhibitor cocktail I, and phosphatase inhibitor cocktails I and II were purchased from Sigma.

It should be noted that N6-cyclohexyl-N2-(2-methyl-4-morpholino-phenyl)-9H-purine-2,6-diamine (i.e., “Compound A”) is meant to serve as an example for all the compounds of the present invention, which all share the property of selectively inhibiting the protein kinase TTK.

Western blot analyses: Cells were lysed in 10 mM Tris-HCl pH 7.5, 1 mM EDTA, 150 mM NaCl, 10% glycerol, protease inhibitor cocktail, phosphatase 1, and phosphatase 2. Protein yields were determined by Bradford assay and equivalent amounts of protein were loaded for each sample.

Antibodies: All antibodies were purchased from Cell Signaling Technology.

For qRT-PCR: Primer probe sets for p21 and GAPDH were purchased from Applied Biosystems. qRT-PCR was performed using the Quantitect Probe RT-PCR Kit from Qiagen following the standard protocol.

### Example 6A

### Activity and Selectivity of Compound A

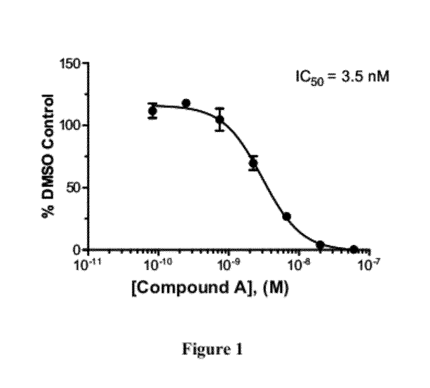

Inhibition of enzymatic activity was determined with a ³³P filter plate assay. For each enzyme reaction, ATP was used at a concentration equivalent to 2-fold the experimentally determined Kₘ for ATP. Compound A demonstrates potent activity towards TTK and is selective against 42 other kinases tested, as depicted in Table 10 below. Effect of Compound A on TTK in vitro kinase activity is shown in FIG. 1.

TABLE 10
Inhibition of Various Kinases by Compound A

| Kinase | IC₅₀ (μM) |
|---|---|
| FLT3 | 0.080 |
| JNK1 | 0.11 |
| ALK | 0.26 |
| INSR | 0.38 |
| FER | 0.59 |
| FAK1 | 2.7 |
| B-RAF | 3.2 |
| PLK4 | 3.3 |
| ERK2 | 3.9 |
| AUR-B | >5 |
| AUR-C | >5 |
| CHK1 | >5 |
| CHK2 | >5 |
| C-SRC | >5 |
| IKK-alpha | >5 |
| MEK1 | >5 |
| NEK2 | >5 |
| PLK1 | >5 |
| ROCK2 | >5 |
| RSK2 | >5 |
| TAK1-TAB1 | >5 |
| AKT3 | >10 |
| AUR-A | >10 |
| CDK1-cyclinB | >10 |
| CDK2-cyclinA | >10 |
| CDK6-cyclinD3 | >10 |
| CHK1 | >10 |
| C-MET | >10 |
| DYRK2 | >10 |
| HGK | >10 |
| IKK-beta | >10 |
| IKK-epsilon | >10 |
| JAK1 | >10 |
| PDK1 | >10 |
| PKA | >10 |
| PKC-delta | >10 |
| TAO1 | >10 |
| TBK1 | >10 |
| TBK1 | >10 |
| TRKB | >10 |
| YES1 | >10 |
| MST4 | >10 |

### Example 6B

### Compound A Cellular Effects

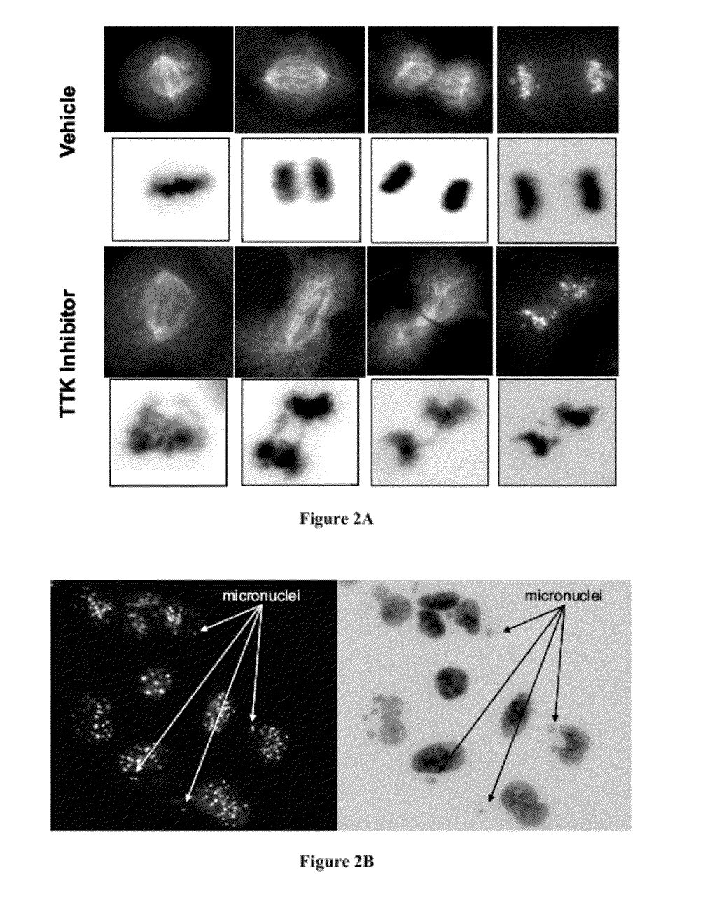

A549 cells were treated with vehicle or 10 μM Compound A for 24 hours. Cells were stained with Hoechst dye (blue) and anti-tubulin (green), anti-pericentrin (red) and anti-centromere (white) antibodies. Black and white images of representative metaphase and anaphase cells are shown in FIG. 2A.

A549 cells were treated with 10 μM Compound A for 24 hours and stained as above. Representative black and white images of interphase cells are shown in FIG. 2B. As revealed in the original full-color images of FIGS. 2A and 2B, inhibition of TTK causes defects in chromosome alignment, resulting in lagging chromosomes during anaphase and micronuclei during interphase. Virtually all the micronuclei contain centromeres, suggesting the presence of whole chromosomes. Therefore, Compound A causes chromosome segregation defects.

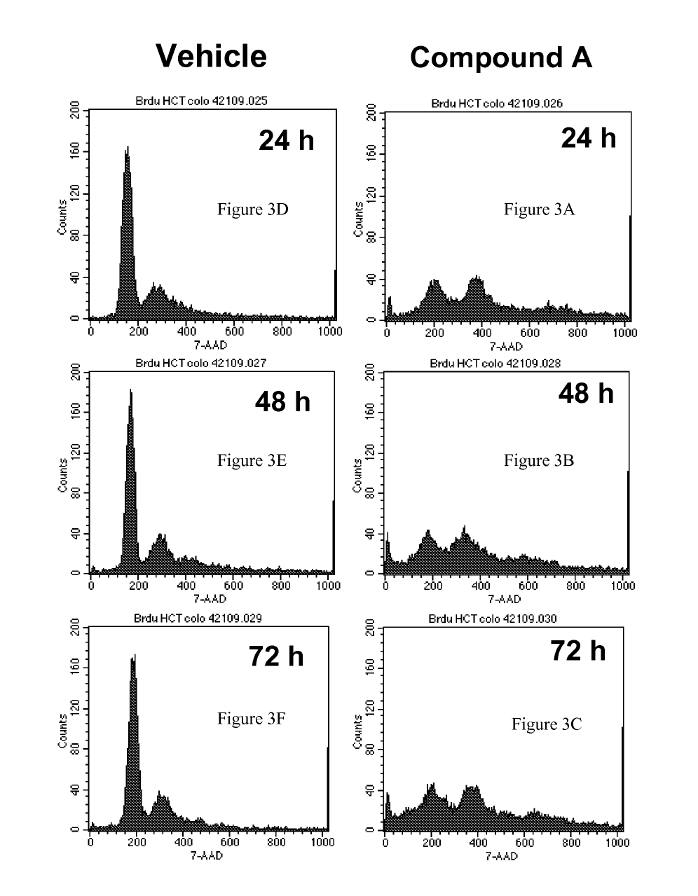

HCT-116 cells were treated with vehicle or 1 μM Compound A for 24, 48, or 72 hours. Cells were fixed, stained with propidium iodide and analyzed by fluorescence activated cell sorting (FACS) using a FACScan instrument. Inhibition of TTK induces both aneuploidy, as seen in FIG. 3 by a broadening of the 2N and 4N peaks, as well as the formation of tetraploid (8N) cells.

### Example 6C

### p53 Pathway Activation in Response to Compound A

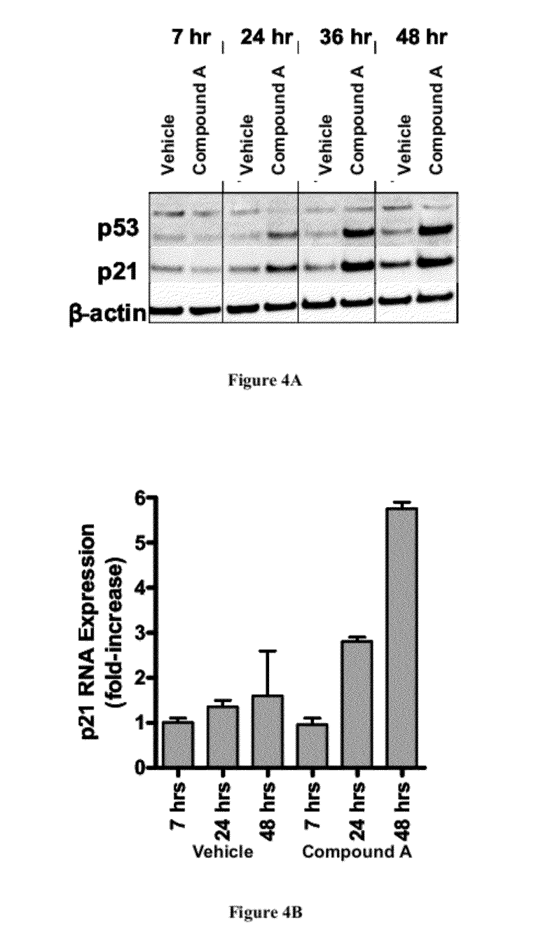

HCT-116 cells were treated with vehicle or 1 μM Compound A for the times indicated in FIG. 4A. Cell lysates were analyzed by Western blot with anti-p53, anti-p21 or anti-β-actin antibodies (see FIG. 4A). Compound A appears to activate the p53 pathway in a time- and dose-dependent manner, with maximal induction occurring by 36 hours.

p21 is known to be transcriptionally upregulated by p53. Therefore, p21 induction in response to Compound A was tested. Total mRNA was purified from HCT-116 cells treated with vehicle or 1 μM Compound A for the times indicated in FIG. 4B. qRT-PCR was performed with primers for either p21 or GAPDH. The p21 mRNA levels were normalized to the GAPDH mRNA levels from the same total RNA sample. Data are plotted as the fold-increase relative to the 7 hour DMSO control sample (see FIG. 4B). p21 mRNA is induced in a time-dependent manner in response to Compound A.

p53 was previously reported to suppress the expression of survivin. Therefore, survivin expression in response to treatment with Compound A was tested. HCT-116 cells were treated with increasing concentrations of Compound A for 48 hours. Cell lysates were analyzed by Western blot with anti-p53, anti-survivin or anti-β-actin antibodies (see FIG. 4C). Treatment with Compound A induced p53 in a dose- and time-dependent manner and this correlated with a decrease in survivin protein. Induction of p53 preceded the loss of survivin.

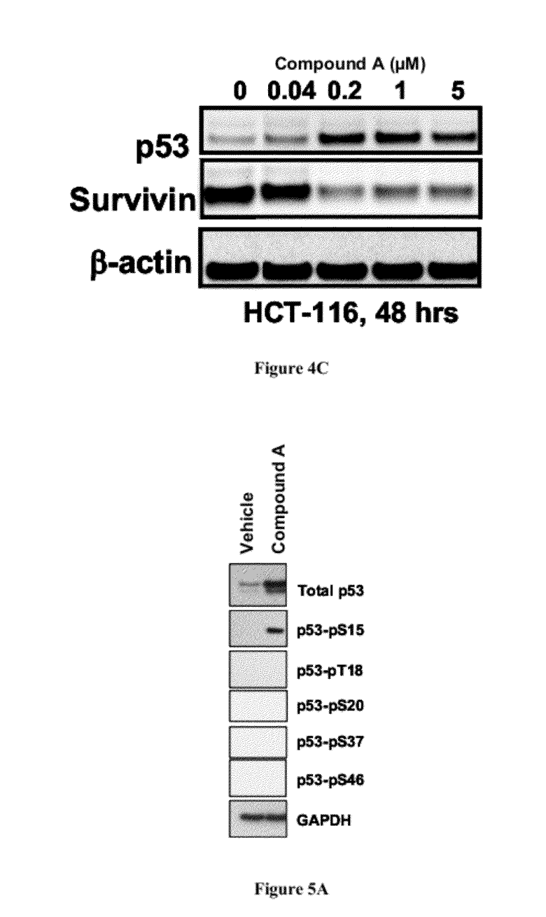

Phosphorylation of p53 at various sites on the N-terminus results in the stabilization of p53. Therefore, phospho-specific antibodies were used to determine phosphorylation location in response to treatment with Compound A. HCT-116 cells were treated with vehicle or 1 μM Compound A for 48 hours. Cell lysates were analyzed by Western blot with anti-GAPDH, anti-p53 or the phosphospecific anti-p53 antibodies. It was determined that p53 is phosphorylated on serine 15 in response to treatment with Compound A as indicated in FIG. 5A.

Phosphorylation and stabilization of p53 occurs in response to DNA damage. Phosphorylation of H2AX functions to recruit DNA repair enzymes to sites of DNA damage and serves as a marker for DNA double strand breaks. It was examined whether Compound A induced the phosphorylation of histone H2AX on serine 139. HCT-116 cells were treated with vehicle or 1 μM Compound A for 7, 24, 36, or 48 hours. Cell lysates were analyzed by Western blot with anti-H2AX-pS139 (gH2AX) or β-actin antibodies. Phosphorylation of serine139 on H2AX was induced by Compound A in a time-dependent manner that correlated with p53 induction, as depicted in FIG. 5B. Inhibition of TTK induces the phosphorylation of p53 on serine 15 and H2AX on serine 139.

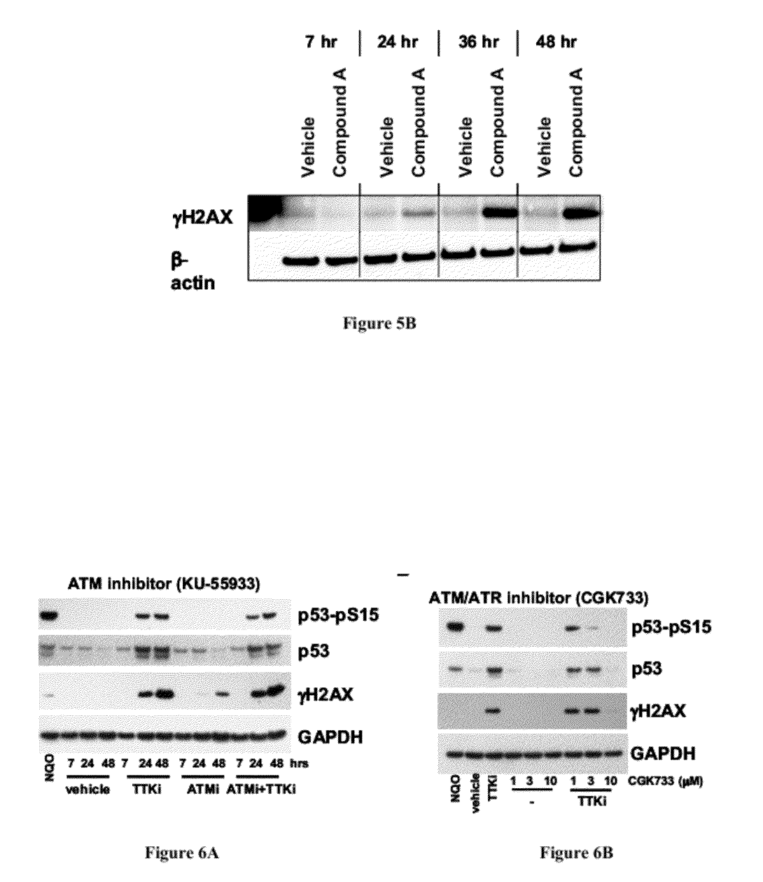

Phosphorylation of either p53 on serine 15 or H2AX on serine 139 is often mediated by the DNA damage checkpoint proteins, ATM or ATR. To determine whether these kinases mediated the phosphorylation of p53 or H2AX in response to TTK inhibitors, cells were treated with Compound A in the absence or presence of an ATM inhibitor or a dual ATM/ATR inhibitor. HCT-116 cells were treated with vehicle, 1 μM Compound A (referred to as TTKi in FIGS. 6A-6C), and/or 5 μM of either ATM inhibitor KU-55933 (see FIG. 6A), ATM/ATR inhibitor CGK733 (see FIG. 6B), or DNA-PK inhibitor NU7026 (see FIG. 6C). As a positive control, cells were treated with the UV mimetic 4-NQO (5 μM). Cell lysates were analyzed by Western blot with anti-p53, anti-p53-pS15, anti-H2AX-pS139 and anti-GAPDH antibodies (see FIGS. 6A-6C). Treatment with the dual ATM/ATR inhibitor blocked p53 and H2AX phosphorylation in response to Compound A. In contrast, ATM inhibitor had no effect, indicating that ATR is responsible for these phosphorylation events. Phosphorylation of p53 on serine 15 and H2AX on serine 139 appears mediated by ATR kinase in response to TTK inhibition.

### Example 6D

### Effect of Caspase Inhibition

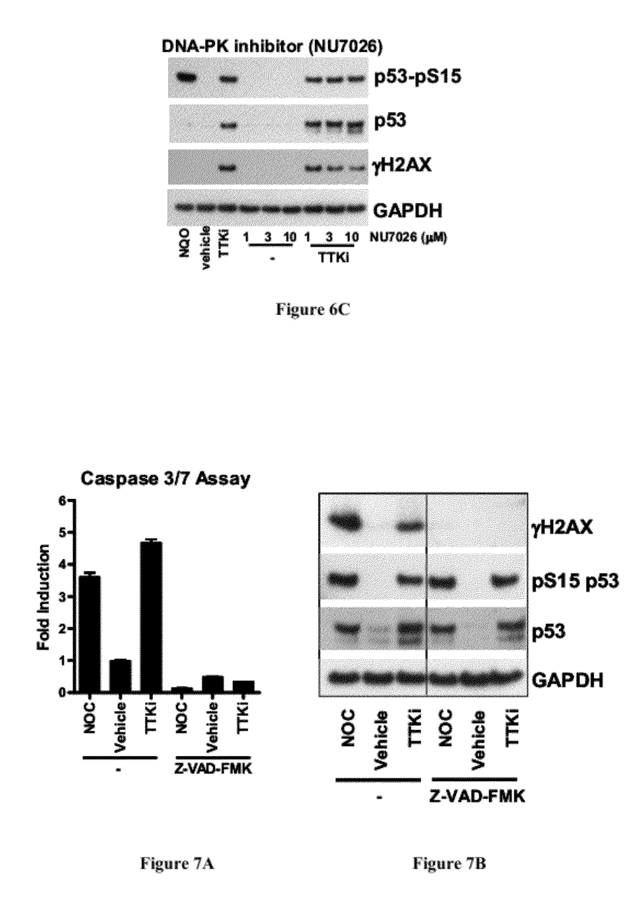

HCT-116 cells were pre-treated with 100 μM Z-VAD-FMK pan-caspase inhibitor for 1 hour prior to treatment with vehicle, 1 μM Compound A (referred to as TTKi in FIGS. 7A and 7B) or 250 ng/ml nocodazole for 48 hours. Caspase 3/7 activity was determined with a Caspase-Glo 3/7 assay (see FIG. 7A). Cell lysates were analyzed by Western blot with antibodies specific for H2AX-pS139, p53-pS15, p53 or GAPDH (see FIG. 7B). Cells were pretreated with vehicle or 100 μM Z-VAD-FMK for 1 hour followed by treatment with vehicle or increasing concentrations of Compound A. After 72 hours, cell viability was measured with a Cell Titer-Glo assay. Inhibition of caspase activity prevents TTK inhibitor-induced H2AX phosphorylation but has no effect on either p53 phosphorylation or cell viability.

### Example 6E

### Induction of the p53-p21 Pathway in Response to TTK Inhibitors

HCT-116 cells were treated with vehicle or 1 μM Compound A for the times indicated in FIG. 5A. Cell lysates were analyzed by Western blot with anti-p53, anti-p21 or anti-β-actin antibodies (see FIG. 5A). Compound A appears to activate the p53 pathway in a time-dependent manner, with maximal induction occurring by 36 hours.

Phosphorylation of p53 at various sites on the N-terminus results in the stabilization of p53. Therefore, phospho-specific antibodies were used to determine the site of phosphorylation on p53 in response to treatment with Compound A. HCT-116 cells were treated with vehicle or 1 μM Compound A for 48 hours. Cell lysates were analyzed by Western blot with anti-GAPDH, anti-p53 or the phosphospecific anti-p53 antibodies. It was determined that p53 is phosphorylated on serine 15 in response to treatment with Compound A as indicated in FIG. 5A.

Phosphorylation of p53 on serine 15 is often mediated by the DNA damage checkpoint proteins, ATM or ATR. To determine whether these kinases mediated the phosphorylation of p53 in response to TTK inhibitors, cells were treated with Compound A in the absence or presence of an ATM inhibitor or a dual ATM/ATR inhibitor. HCT-116 cells were treated with vehicle, 1 μM Compound A (referred to as TTKi in FIG. 5A), and/or 5 μM of either ATM inhibitor KU-55933 (see FIG. 5A), ATM/ATR inhibitor CGK733 (see FIG. 5A), or DNA-PK inhibitor NU7026 (see FIG. 5A). As a positive control, cells were treated with the UV mimetic 4-NQO (5 μM). Cell lysates were analyzed by Western blot with anti-p53, anti-p53-pS15 and anti-GAPDH antibodies (see FIG. 5A). Treatment with the dual ATM/ATR inhibitor blocked p53 phosphorylation in response to Compound A. In contrast, ATM inhibitor had no effect, indicating that ATR is responsible for this phosphorylation event. Phosphorylation of p53 on serine 15 appears to be mediated by ATR kinase in response to TTK inhibition.

p21 is known to be transcriptionally upregulated by p53. Therefore, p21 induction in response to Compound A was tested. Total mRNA was purified from HCT-116 cells treated with vehicle or 1 μM Compound A for the times indicated in FIG. 5A. qRT-PCR was performed with primers for either p21 or GAPDH. The p21 mRNA levels were normalized to the GAPDH mRNA levels from the same total RNA sample. Data are plotted as the fold-increase relative to the 7 hour DMSO control sample (see FIG. 5A). p21 mRNA is induced in a time-dependent manner in response to Compound A.

p53 was previously reported to suppress the expression of survivin. Therefore, survivin expression in response to treatment with Compound A was tested. HCT-116 cells were treated with increasing concentrations of Compound A for 48 hours. Cell lysates were analyzed by Western blot with anti-p53, anti-survivin or anti-β-actin antibodies (see FIG. 5A). Treatment with Compound A induced p53 in a dose- and time-dependent manner and this correlated with a decrease in survivin protein. Induction of p53 preceded the loss of survivin.

The discovery that inhibition of TTK induces the ATR-dependent phosphorylation and activation of p53 and its downstream effectors suggests that the monitoring of p53 and/or p21 expression at either the protein or mRNA level may provide important information as to the amount of TTK inhibition being achieved by the compounds of the present invention. Consequently, the level of expression of p53 and/or p21 may serve as a useful biomarker with which to evaluate the effects of TTK inhibition in both cellular and animal models in which p53 is not mutated.

### Example 6F

### Identification of Hsp90 as a Downstream Substrate for TTK

293T cells were transfected with plasmids overexpressing various forms of TTK using Lipofectamine-2000. Forty-eight hours later, cells lysates were prepared with RIPA buffer containing protease inhibitor cocktail (Sigma), phosphatase inhibitor 1 (Sigma) and phosphatase inhibitor 2 (Sigma). The amount of protein loaded for Western blots was normalized by Bradford assay.

Immunoprecipitations. Cells were lysed in Lysis/Wash Buffer (10 mM Tris-HCl pH 7.5, 1 mM EDTA, 150 mM NaCl, 10% glycerol, protease inhibitor cocktail, phosphatase 1, and phosphatase 2). Amounts of protein used for the immunoprecipitation were normalized by Bradford assay. Immunoprecipitations were carried out with anti-pT676-TTK or anti-Hsp90 antibody and protein G-Sepharose at 4° C. The immunoprecipitates were washed three times with 1 mL of Lysis/Wash Buffer and analyzed by Western blot.

In vitro TTK phosphorylation reaction. Assay mixes included 50 mM Tris-HCl pH 7.5, 0.01% Triton X-100, 10 mM MgCl₂, 40 μM ATP, 2 μM recombinant Hsp90a (Assay Designs), and 4 nM recombinant TTK (Invitrogen). The reaction was started with the addition of TTK and incubated for 50 minutes at room temperature. The reactions were terminated with the addition of LDS sample loading buffer. Samples were analyzed by Western blot analysis.

Construction of Hsp90 mutants. Hsp90 mutants were generated by PCR-based site-directed mutagenesis using Pfu turbo DNA polymerase (Stratagene).

Mass spectrometry analysis of in vitro phosphorylated Hsp90. In vitro phosphorylated Hsp90 was digested with trypsin or GluC protease, enriched with Immobilized Metal Affinity Columns (IMACs) followed by TiO₂ enrichment. Five phosphopeptides were identified from this sequential enrichment and analyzed by mass spectrometry. Only one phosphopeptide was found in the C-terminal region of Hsp90 between amino acids 669-732.

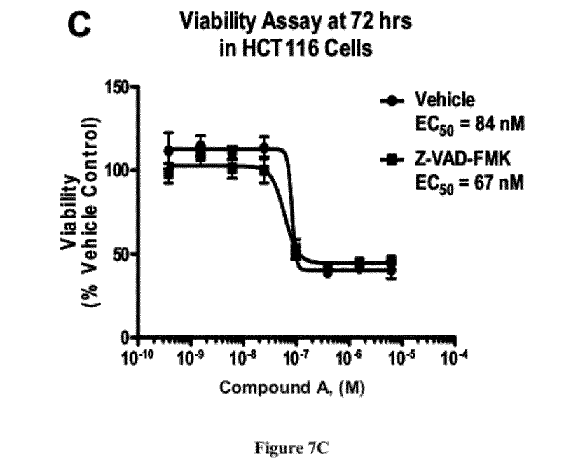

Two forms of GFP-tagged TTK were overexpressed in 293T cells and cell lysates were analyzed by Western blot with the pT676-TTK antibody (FIG. 8A). Overexpression of catalytically active TTK, but not a kinase-inactive mutant (D664A mutation) induced the appearance of the 90 kDa band. The intensity of this band increased with increasing levels of wild-type TTK, resulting from the transfection of higher amounts of TTK-expressing plasmid. Furthermore, if extracts from cells overexpressing wild-type TTK were pre-treated with lambda phosphatase, the 90 kDa band could no longer be detected with the anti-pT676-TTK antibody (FIG. 8B), indicating that this antibody recognizes only the phosphorylated form of the 90 kDa protein. If the TTK inhibitor Compound A was added to cells, the appearance of the 90 kDa band induced by TTK-WT was reduced in a dose-dependent manner (FIG. 8C). Together, these data supported the hypothesis that TTK directly or indirectly induced the phosphorylation of a ˜90 kDa protein.

FIG. 8. TTK overexpression induces phosphorylation of 90 kDa Band. A) 293T cells were transfected with plasmids encoding wild-type TTK (TTK-WT) or kinase-inactive TTK (TTK-KD). After 48 hours cell lysates were prepared and examined by Western blot using anti-pT676-TTK, anti-TTK or anti-GAPDH antibodies. B) Lysates from cells overexpressing wild-type TTK were treated with or without lambda phosphatase and analyzed by Western blot with anti-pT676-TTK or anti-GAPDH antibody. C) 293T cells were transfected with the plasmid expressing wild-type TTK. After 48 hours, cells were treated for 2 hours with the indicated concentrations of Compound A. Cell lysates were analyzed by Western blot using anti-pT676-TTK and anti-TTK antibodies.

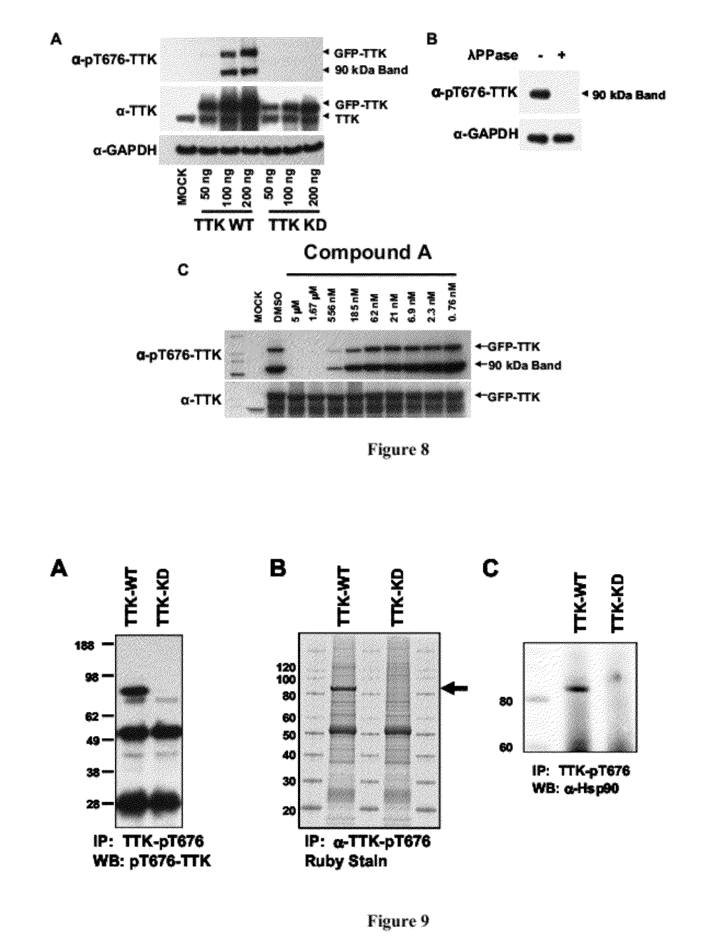

To determine the identity of the 90 kDa band, wild-type or kinase-inactive TTK was overexpressed in SW480 cells. Cell lysates were immunoprecipitated with the anti-pT676-TTK antibody and the eluate was analyzed by Western blot with anti-pT676-TTK antibody. The 90 kDa band was detected in the immunoprecipitate from cells expressing wild-type but not kinase-inactive TTK (FIG. 9A). Parallel samples were stained with Ruby red (FIG. 9B) and the 90 kDa band was isolated and analyzed by mass spectrometry. This band was identified as Hsp90a. Western blot analysis of the anti-pT676-TTK immunoprecipitation with anti-Hsp90 antibody confirmed that the 90 kDa band was Hsp90 (FIG. 9C). These data suggest that TTK functions upstream of Hsp90 and directly or indirectly phosphorylates Hsp90.

FIG. 9. Identification of the 90 kDa band detected by the anti-pT676-TTK antibody as Hsp90. Lysates from SW480 cells overexpressing wild-type or kinase-inactive TTK were immunoprecipitated with anti-pT676-TTK antibody (A-C) and analyzed by Western blot with anti-pT676-TTK antibody (A) or anti-Hsp90 antibody (C). Alternatively, the eluate from the immunoprecipitation was run on an SDS polyacrylamide gel and stained with Ruby Red (B).

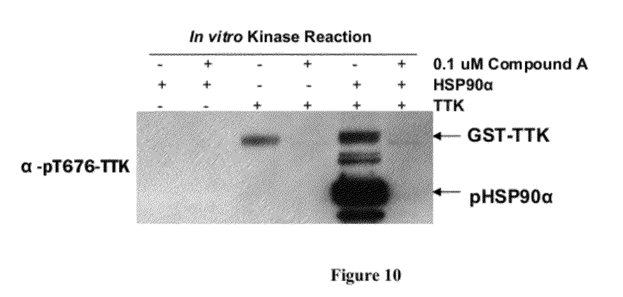

We examined TTK phosphorylation of the alpha isoform of Hsp90 in vitro to determine if Hsp90 is a direct substrate for TTK. Analysis of samples generated from the in vitro reaction revealed a robust induction of phosphorylated Hsp90α as measured with anti-pT676-TTK antibody by Western blot (FIG. 10). The TTK inhibitor, Compound A, completely perturbed the production of phospho-Hsp90α, demonstrating that TTK activity was required for Hsp90α phosphorylation.

FIG. 10. In vitro phosphorylation of Hsp90 by TTK. Recombinant TTK and HSP90α were incubated at room temperature for 50 minutes. The reaction was terminated with the addition of LDS sample loading buffer. Samples were analyzed by Western blot with anti-pT676-TTK antibody.

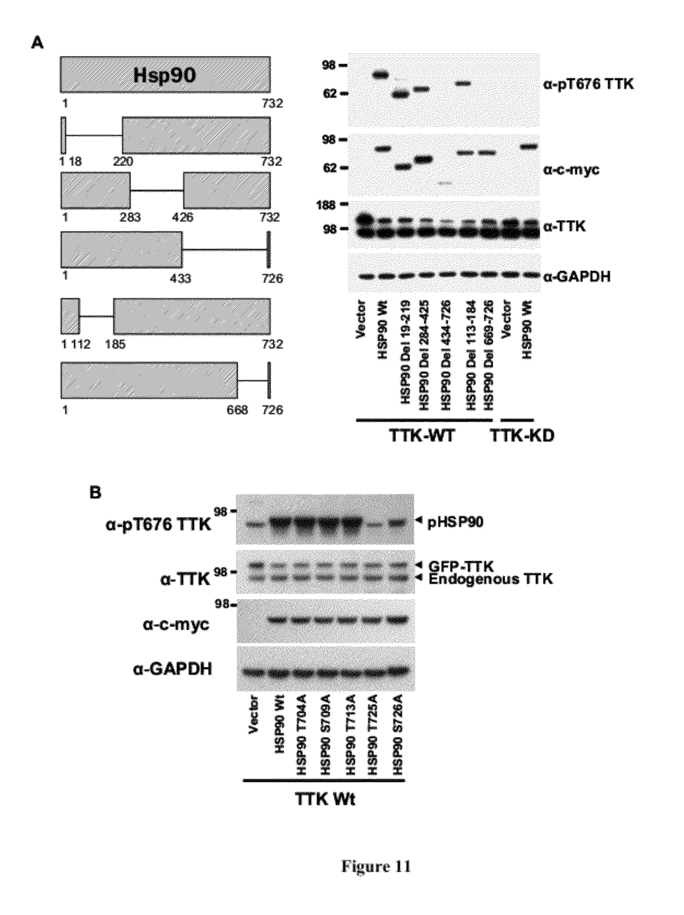

Several Hsp90 deletion mutants were constructed to map the Hsp90 phosphorylation site. Three rather large deletions were constructed: an N-terminal deletion of amino acids 19-219, a deletion of residues in the middle of the protein (284-425), and a C-terminal deletion of residues 434-726 (FIG. 11A). Two smaller deletions were also made in the N-terminus (113-184) and the C-terminus (669-726) (FIG. 11A). These Hsp90 deletion mutants were coexpressed with wild-type TTK to determine if the mutant Hsp90 proteins could be utilized as a substrate for TTK and phosphorylated. Only the two C-terminal deletion mutants were not phosphorylated by TTK as analyzed with the anti-pT676-TTK antibody by Western blot (FIG. 11A). This effectively mapped the Hsp90 phosphorylation site between amino acids 669 to 732.

Within this part of the C-terminus of Hsp90, there are a total of twelve threonine, serine, or tyrosine residues that can be phosphorylated by TTK. Of these twelve residues, six of them have been found to be phosphorylated by TTK in vitro. This observation was uncovered after in vitro phosphorylated Hsp90 was digested with purified protease and examined by mass spectrometry. Mass spectrometry analysis of one phosphorylated peptide had shown residues T704, T708, 5709, T713, T725, and 5726 are phosphorylated. We mutated theses sites in Hsp90 from a threonine/serine to alanine, an amino acid incapable of being phosphorylated. Only the T725A Hsp90 mutant was not phosphorylated by TTK (FIG. 11B), showing that T725 is a TTK phosphorylation site. Furthermore, the S726A Hsp90 mutant did show higher levels of pHsp90 than the vector control, showing that this mutant protein is phosphorylated by TTK (FIG. 11B). However, phosphorylated Hsp90 levels with the S726A Hsp90 protein are lower compared to those found with wild-type Hsp90 (FIG. 11B). The most likely explanation for this observation is that S726 is recognized by the pT676-TTK antibody as part of the epitope for antibody recognition of pT725 Hsp90.

FIG. 11. Identification of the Hsp90 phosphorylation site. A) 293T cells were transfected with plasmids encoding wild-type TTK or kinase-inactive TTK and wild-type Hsp90 or various Hsp90 deletion mutants. A map of the Hsp90 deletion mutants is shown in the left panel. After 48 hours cell lysates were prepared and examined by Western blot using anti-pT676-TTK, anti-c-myc, anti-TTK or anti-GAPDH antibodies (right panel). B) Plasmids expressing wild-type TTK and wild-type Hsp90 or Hsp90 C-terminal mutants were transfected in to 293T cells. Cell lysates were prepared and analyzed as in ‘A’.

Because TTK may modulate Hsp90 function, experiments were performed to determine whether TTK and Hsp90 are part of a complex. Lysates from cells overexpressing wild-type or kinase-inactive TTK were immunoprecipitated with Hsp90 antibody or pT676-TTK antibody. Eluates from immunoprecipitated samples were analyzed by Western blot with antibodies specific for Hsp90, pT676-TTK, or TTK. In Hsp90 immunoprecipitates from cells expressing wild-type TTK, Hsp90 co-precipitated with TTK protein. Finally, Western blot analysis of the pT676-TTK immunoprecipitation with the pT676-TTK antibody detected both TTK and Hsp90. This may be because the antibody recognizes both proteins independently or TTK may co-precipitate with phospho-Hsp90. Together, these data imply that TTK is in a complex with Hsp90.

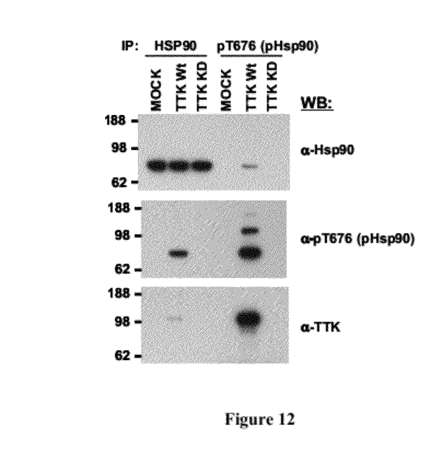

FIG. 12. Co-precipitation of TTK with Hsp90 in cells overexpressing TTK. 293T cells were transfected with empty vector or wild-type or kinase-inactive TTK. Cell lysates were immunoprecipitated with either anti-Hsp90 or anti-pT676-TTK antibody. Eluates from the precipitation were analyzed by Western blot with Hsp90, TTK or pT676-TTK antibodies.

The evidence has shown that TTK phosphorylates Hsp90 at T725 both in cells and in vitro. The two proteins also coimmunoprecipitate with one another showing TTK and Hsp90 have either a direct or closely connected protein-protein interaction. The biological significance of the phosphorylation of the C-terminus of Hsp90 is unclear. The C-terminal region of Hsp90 is important for both its dimerization as well as its association with proteins containing TPR motifs. Interestingly, TTK contains a TPR motif at its N-terminus. The C-terminus of Hsp90 is also important for the cellular localization of Hsp90. Together, these data suggest TTK may regulate Hsp90 dimerization, association with protein substrates and/or cellular trafficking of Hsp90 and its associated proteins.

### Example 7

### Relative Selectivity of TTK Inhibitors

Direct target affinity purification (DTAP) was used to investigate (1) the selectivity of compounds of the invention for TTK, relative to other kinases, and (2) the importance of a specific methyl substituent in providing that selectivity.

Specifically, two compounds of interest, differing by only a single methyl substituent on a phenyl group were synthesized. The two compounds were:

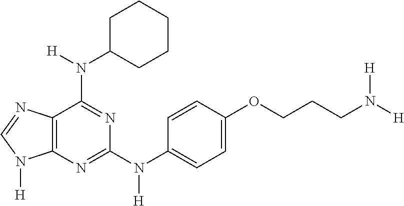

(N2-[4-(3-aminopropoxy)phenyl]-N-6-cyclohexyl-9H-purine-2,6-diamine), and

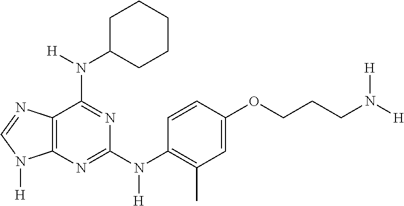

(N2-[4-(3-aminopropoxy)-2-methyl-phenyl]-N-6-cyclohexyl-9H-purine-2,6-diamine).

In preparation for DTAP studies, the two compounds depicted above were synthesized with an alkyl-amine linker to allow covalent coupling to epoxy-activated Sepharose 6B beads (GE Healthcare, Piscataway, N.J.). Sepharose beads were swollen and washed with water for 30 min followed by equilibration in coupling buffer (50% dimethylformamide, 50 mM Na₂CO₃). Beads were pelleted by centrifugation (15 sec at 2000×g) and the supernatant removed by aspiration. An equal volume of coupling buffer containing the linkered test compound was used to resuspend the beads. Compound concentrations in the coupling reaction ranged from 0.1 mM to 12.5 mM. The coupling reactions were incubated at 34° C. for 18 hrs on a rotator mixer. Ethanolamine was added to 1 M for the final 1 hr to quench the coupling reaction. Beads were washed extensively with binding buffer (1 M NaCl, 50 mM Hepes [pH 7.4], 1% Triton X-100, 1 mM EDTA and 1 mM dithiothreitol) to remove residual coupling reagents, and were then stored at 4° C.

Cellular proteins were prepared by mild sonication in lysis buffer (150 mM NaCl, 50 mM Hepes [pH 7.4], 1% Triton X-100, 1 mM EDTA and 2 mM dithiothrietol containing 1× Halt™ protease and phosphatase inhibitor cocktail [Thermo Fisher Scientific, Rockford, Ill.]). Lysates were centrifuged (20,000×g for 20 min) to remove debris, diluted to a protein concentration of ˜5 mg/ml, divided into aliquots, and stored at −80° C.

For DTAP reactions, cell lysates (˜0.5 ml per binding reaction) were thawed and the NaCl concentration adjusted to 1 M. Competitor compounds dissolved in DMSO (or a DMSO control) were then added to the lysate and incubated on ice for 5 min. The lysates were centrifuged at 20,000×g for 10 min and the cleared supernatant was transferred to a tube containing 50 μl of coupled beads. The binding reactions were incubated on a rotator mixer at 4° C. for 2 hrs, after which the beads were pelleted by centrifugation and the supernatant removed by aspiration. The beads were washed three times with 20 volumes of binding buffer, 2× with 20 volumes wash buffer (150 mM NaCl, 50 mM Hepes [pH 7.4], 1% Tween 20, 1 mM EDTA, 2 mM dithiothrietol) and finally twice with 10 volumes of 150 mM NaCl, 50 mM Hepes [pH 7.4].

During the final wash, an aliquot containing 10 μl of beads was transferred to a separate tube and resuspended with 15 μl of 2× SDS/PAGE loading buffer (Invitrogen Corporation, Carlsbad, Calif.) for 5 min at 90° C. The eluted proteins were resolved by electrophoresis on a NuPage 4-12% Bis-Tris Gel (Invitrogen Corporation, Carlsbad, Calif.) and visualized by staining with Ruby Red (Invitrogen Corporation, Carlsbad, Calif.). The remaining beads (40 μl) were processed for analysis by mass spectrometry.

Using the assays described above, N2-[4-(3-aminopropoxy)phenyl]-N-6-cyclohexyl-9H-purine-2,6-diamine was found to inhibit the protein kinase activity of TTK with an IC₅₀ of 8 nM and to be cytotoxic to HCT116 cells with an IC₅₀ of 1.2 μM. When coupled to beads, N2-[4-(3-aminopropoxy)phenyl]-N-6-cyclohexyl-9H-purine-2,6-diamine was found to bind TTK from cellular lysates with high affinity. However, it was also found to bind the kinases Aurora A, FER, JNK and JAK1. In vitro binding assays revealed that N2-[4-(3-aminopropoxy)phenyl]-N-6-cyclohexyl-9H-purine-2,6-diamine bound these four other kinases with IC₅₀ values of 420, 15, 57 and 320 nM, respectively.

Using the assay described above, N2-[4-(3-aminopropoxy)-2-methyl-phenyl]-N-6-cyclohexyl-9H-purine-2,6-diamine (which differs from N2-[4-(3-aminopropoxy)phenyl]-N-6-cyclohexyl-9H-purine-2,6-diamine by the addition of a single additional methyl substituent on its phenyl group) was found to inhibit the protein kinase activity of TTK with an IC₅₀ of 3 nM. However, when bound to beads, N2-[4-(3-aminopropoxy)-2-methyl-phenyl]-N-6-cyclohexyl-9H-purine-2,6-diamine failed to bind Aurora A from cell lysates, and showed reduced affinity for FER, JNK1 and JAK1, while maintaining a similar binding affinity for TTK. In vitro binding assays revealed that N2-[4-(3-aminopropoxy)-2-methyl-phenyl]-N-6-cyclohexyl-9H-purine-2,6-diamine bound Aurora A, FER, JNK and JAK1 with IC₅₀ values of >5,000,380, 120 and >5,000 nM, repectively.

These results indicate that the addition of the single methyl substituent on phenyl group of N2-[4-(3-aminopropoxy)-2-methyl-phenyl]-N-6-cyclohexyl-9H-purine-2,6-diamine significantly improved selectivity of this compound for TTK, relative to Aurora A, FER, JNK and JAK1.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound having a structure according to Formula I 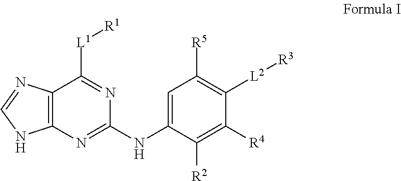
and pharmaceutically acceptable salts and solvates thereof, wherein:
R¹ is an optionally substituted carbocycle, heterocycle, aryl, or heteroaryl;
R² is chosen from the group consisting of: halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle;
R³ is chosen from: hydro, haloalkyl, —R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and lower alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle;
R⁴ and R⁵ are independently chosen from: hydro, halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycles; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl;
L¹ is a direct bond or a linker chosen from: —O—, —S—, —S(═O), S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl, alkoxyl, carboxyl, or carbocycle; and
L² is a direct bond or a linker chosen from: —O—, —S—, —(C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, —S(═O)₂—, -alkylene-, alkynylene, aryl, heteroaryl, heterocycle, arylalkyl, heteroarylalkyl, and heterocyclylalkyl and —O-alkylene-; wherein R^{f} is chosen from hydro and C₁₋₄ alkyl.

2. The compound of claim 1, wherein the structure is according to Formula Ia 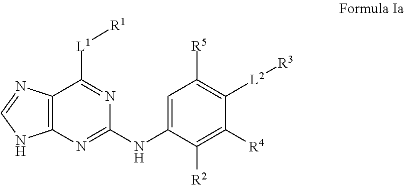
and pharmaceutically acceptable salts and solvates thereof, wherein:
R¹ is an optionally substituted carbocycle, heterocycle, aryl, or heteroaryl;
R² is chosen from the group consisting of: halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle;
R³ is a group chosen from: hydro, haloalkyl, —R^{c}, —NH(C═O)—R^{c}, -alkylene-NH(C═O)—R^{c}, —(C═O)NH—R^{c}, -alkylene-(C═O)NH—R^{c}, —NH—S(═O)₂—R^{c}, -alkylene-NH—S(═O)₂—R^{c}, —S(═O)₂NH—R^{c}, -alkylene-S(═O)₂NH—R^{c}, and —N(R^{d})(R^{e})—; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino; and wherein R^{d} and R^{e} are each independently chosen from hydro and C₁₋₄ alkyl;
R⁴ and R⁵ are independently chosen from: hydro, halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl;
L¹ is a direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═C)O—, —C(═O)NR^{a}—, wherein n is 0, 1, 2, 3, 4, or 5, and wherein R^{a} is hydrogen, hydroxyl, alkyl, alkoxyl, carboxyl, or carbocycle; and
L² is a direct bond or a linker chosen from: —O—, —S—, —(C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, —S(═O)₂—, -alkylene-, alkynylene, aryl, heteroaryl, heterocycle, arylalkyl, heteroarylalkyl, and heterocyclylalkyl and —O-alkylene-; wherein R^{f} is chosen from hydro and C₁₋₄ alkyl.

3. The compound of claim 1, wherein the structure is according to Formula Ia1 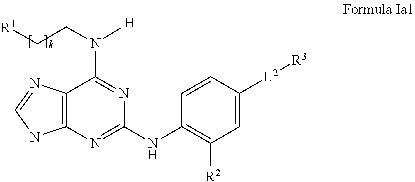
and pharmaceutically acceptable salts and solvates thereof; wherein:
R¹ is a substituted or unsubstituted C₃₋₆ cycloalkyl or heterocycle;
k is −1, 0, 1, or 2;
R² is chosen from the group consisting of: halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle;
R³ is chosen from the group consisting of: haloalkyl, —C₁₋₆ alkylene-NH(C═O)—R^{c}, —C₁₋₆ alkylene-(C═O)NH—R^{c}, —C₁₋₆ alkylene-NH—S(═O)₂—R^{c}, —C₁₋₆ alkylene-S(═O)₂NH—R^{c}, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, heterocycle, aryl, substituted or unsubstituted heteroaryl, nitro, hydroxy, and amino; and
L² is a direct bond, or a linker chosen from: —O—, —O-alkylene-, —C(═O)—, —C(═O)N(R^{a})— wherein R^{a} is hydro or C₁₋₃ alkyl (e.g., methyl or ethyl), alkylene, alkynylene.

4. The compound of claim 1, wherein the structure is according to Formula Ib 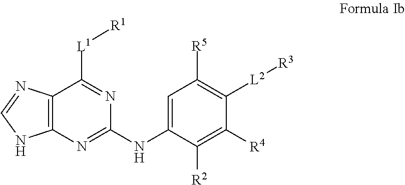
and pharmaceutically acceptable salts and solvates thereof; wherein:
R¹ is an optionally substituted carbocycle or heterocycle;
R² is chosen from the group consisting of: halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle;
R³ is a group chosen from: hydro, haloalkyl, —R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle; wherein, R⁴ and R⁵ are independently chosen from: hydro, halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl;
L¹ is a direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂)ₙ—N(—R^{a})—, —C(═O)—, —C(═O)O—, —C(═O)NR^{a}—, wherein n is 0, 1, 2, 3, 4, or 5, and wherein R^{a} is hydrogen, hydroxyl, alkyl, alkoxyl, carboxyl, or carbocycle; and
L² is a direct bond or a linker chosen from: alkynylene, aryl, heterocycle, heteroaryl, heteroarylalkyl, arylalkyl, and heterocyclylalkyl.

5. The compound of claim 1, wherein the structure is according to Formula Ib1 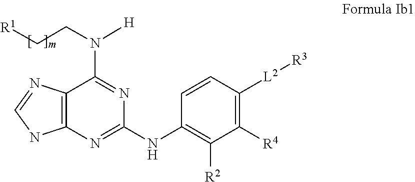
and pharmaceutically acceptable salts thereof; wherein:
R¹ is an optionally substituted carbocycle or heterocycle;
m is 0, 1, or −1;
R² halo, methyl optionally substituted with halo, ethyl optionally substituted with halo, methylthio, ethylthio, methoxy, or ethoxy;
R³ is a group chosen from: hydro, haloalkyl, —R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle; wherein, R⁴ and R⁵ are independently chosen from: hydro, halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl; and
L² is a direct bond or a linker chosen from: alkynylene, aryl, heterocycle, heteroaryl, heteroarylalkyl, arylalkyl, and heterocyclylalkyl.

6. The compound of claim 1, wherein the structure is according to Formula Ib2 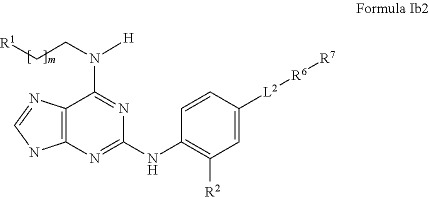
and pharmaceutically acceptable salts and solvates thereof; wherein
R¹ is an optionally substituted carbocycle or heterocycle;
m is 0, 1, or −1;
R² halo, methyl optionally substituted with halo, ethyl optionally substituted with halo, methylthio, ethylthio, methoxy, or ethoxy;
R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —N(R^{f})C(═O)—, -alkylene-N(R^{f})C(═O)—, —C(═O)N(R^{f})R^{f}—, -alkylene-C(═O)N(R^{f})R^{g}—, —N(R^{f})S(═O)₂—, -alkylene-N(R^{f})S(═O)₂—, —S(═O)₂N(R^{f})R^{g}—, -alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from: 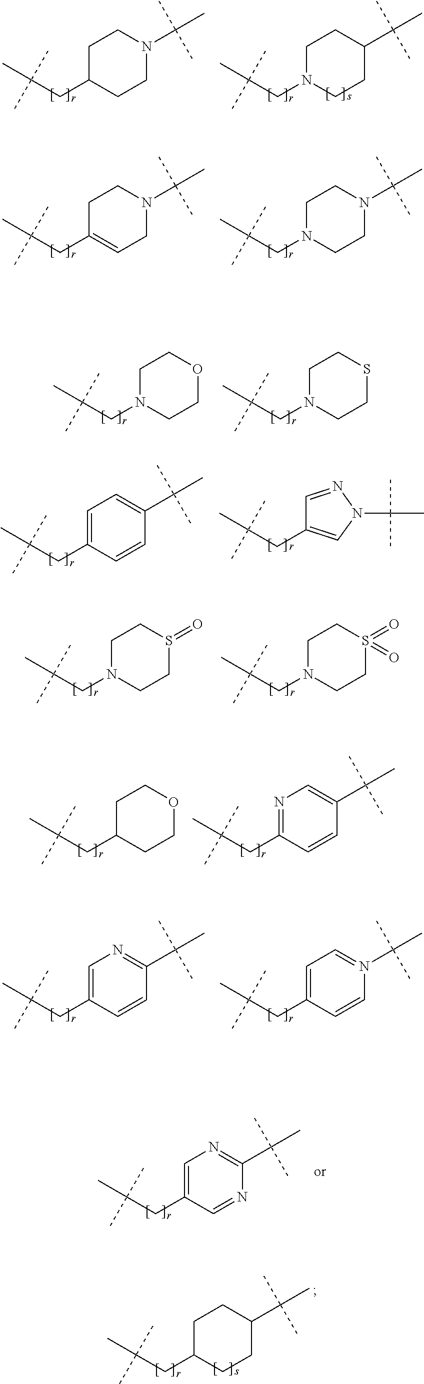
wherein r is 0, 1, or 2, and wherein s is 0, 1, −1;
R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, amino-C₁₋₃ alkylene, —N(R^{h})C(═O)—, —alkylene-N(R^{h})C(═O)—, —C(═O)N(R^{h})Rⁱ—, -alkylene-C(═O)N(R^{h})Rⁱ—, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle heteroaryl optionally substituted with methyl, hydroxyl, or amino; or R⁷ is one of: 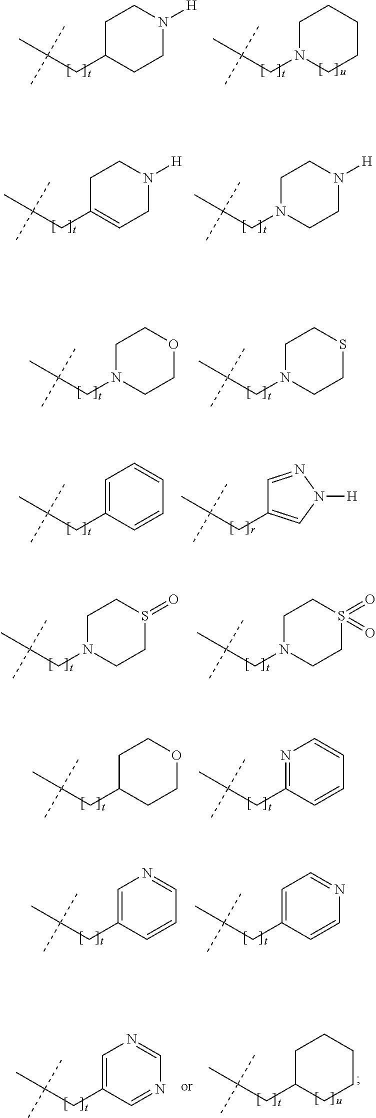
wherein t is 0, 1, or 2, and u is 0, 1, −1; and
L² is a direct bond or a linker chosen from: alkynylene, aryl, heterocycle, heteroaryl, heteroarylalkyl, arylalkyl, and heterocyclylalkyl.

7. The compound of claim 6, wherein R¹ is a C₃₋₆ carbocycle or heterocycle optionally substituted with one or more substituents independently chosen from the group consisting of: (1) halo; (2) hydroxyl; (3) cycloalkyl; (4) alkylthio; (5) C-carboxy; (6) carboxyalkoxy; (7) N-carbamyl; (8) amino; (9) N-amido; (10) sulfonamide; and (11) C₁₋₆ alkyl optionally substituted with N-carbamyl, sulfonamide or N-amido; (12) C₁₋₆ alkoxy optionally substituted with N-carbamyl or sulfonamide; (13) aminoalkyl optionally substituted with C-amido; and (14) heterocycle.

8. The compound of claim 6, wherein R⁶ is C₁₋₃ alkyl, hydroxy, hydroxy-C₁₋₃ alkylene, halo-C₁₋₃ alkylene, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from: 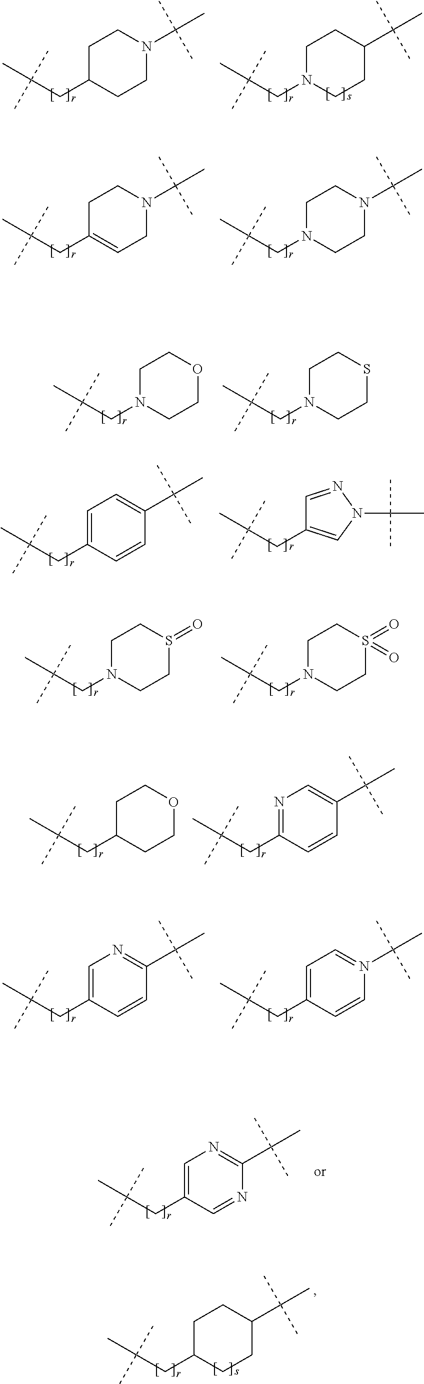
wherein r is 0, 1, or 2, and s is 0, 1, −1; and
R⁷ is not present, is hydro, or is one or more of: C₁₋₃ alkyl, C₃₋₆ cycloalkyl, hydroxy, hydroxy-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is one of: 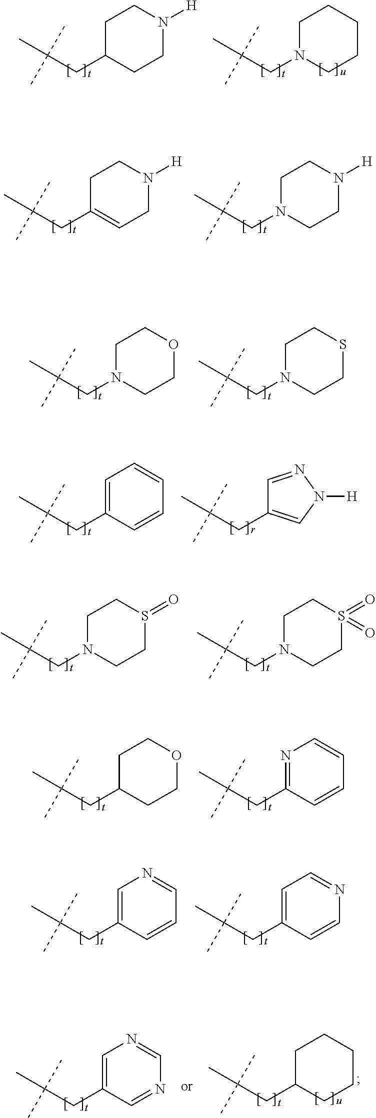
wherein t is 0, 1, or 2, and wherein u is 0, 1, −1.

9. The compound of claim 6, wherein R⁶ is C₁₋₃ alkyl, hydroxy, hydroxy-C₁₋₃ alkylene, halo-C₁₋₃ alkylene, —C₁₋₃ alkylene-C(═O)—, —N(R^{f})C(═O)—, -alkylene-N(R^{f})C(═O)—, —alkylene-C(═O)N(R^{f})R^{g}—, —N(R^{f})S(═O)₂—, -alkylene-N(R^{f})S(═O)₂—, —S(═O)₂N(R^{f})R^{g}—, —alkylene-S(═O)₂N(R^{f})R^{g}—, —S(═O)₂—, —N(R^{f})R^{g}—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; or R⁶ is selected from: 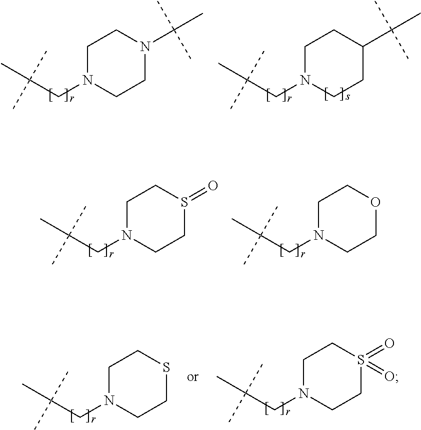
wherein r is 0, 1, or 2, and wherein s is 0, 1, −1;
R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is one of: 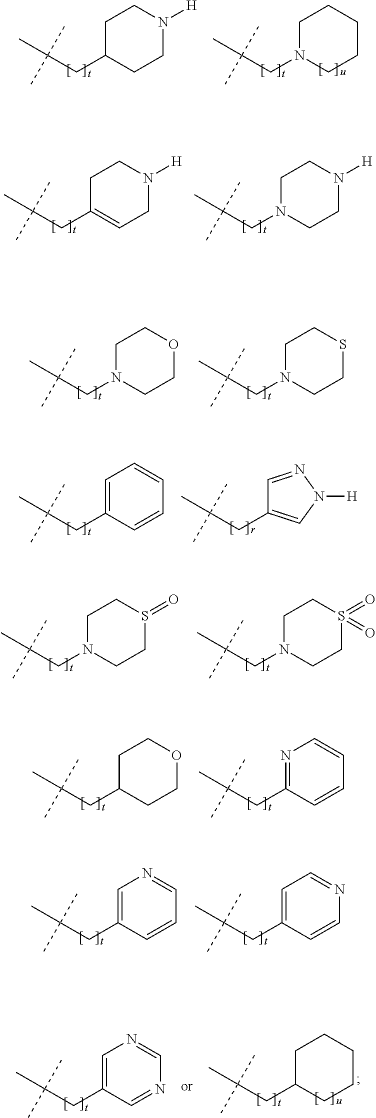
wherein t is 0, 1, or 2, and u is 0, 1, −1.

10. The compound of claim 6, wherein R⁶ is C₁₋₃ alkyl, hydroxy, hydroxy-C₁₋₃ alkylene, halo-C₁₋₃ alkylene, —C(═O)—, —C₁₋₃ alkylene-C(═O)—, —C(═O)N(R^{f})R^{f}—, -alkylene-C(═O)N(R^{f})R^{g}—, —S(═O)₂N(R^{f})R^{g}—, -alkylene-S(═O)₂N(R^{f})R^{g}—, —S(═O)₂—, wherein R^{f} and R^{g} are each independently chosen from hydro, hydroxyl, and C₁₋₃ alkyl, or R^{f} and R^{g} together with the nitrogen atom to which they are bound form a heterocycle linked with R⁷; wherein R⁷ is not present, is hydro, or is one or more of: C₁₋₃ alkyl, C₃₋₆ cycloalkyl, hydroxy, hydroxy-C₁₋₃ alkylene, halo-C₁₋₃ alkylene, amino, or amino-C₁₋₃ alkylene.

11. The compound of claim 6, wherein R⁶ is C₁₋₃ alkyl, hydroxy, hydroxy-C₁₋₃ alkylene, or halo-C₁₋₃ alkylene; and,
wherein R⁷ is not present, is hydro, or is —N(R^{h})Rⁱ—, wherein R^{h} and Rⁱ are each independently chosen from hydro, hydroxyl, C₁₋₃ alkyl, amino, and amino-C₁₋₃ alkylene-, or R^{h} and Rⁱ together with the nitrogen atom to which they are bound form a heterocycle or heteroaryl optionally substituted with methyl, hydroxyl, or amino; or R⁷ is: 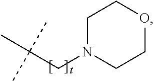
wherein t is 0, 1, or 2.

12. The compound of claim 6, wherein L² is alkynylene, aryl, arylalkyl, heteraryl, heteroarylalkyl, or 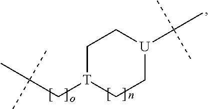
wherein T is carbon or nitrogen, U is carbon, nitrogen, sulfur, or oxygen, n is 0, 1, or −1, o is 0, 1, or 2, and there is optionally at least one ring carbon-ring carbon double bond.

13. The compound of claim 6, wherein, L² is alkynylene or one of: 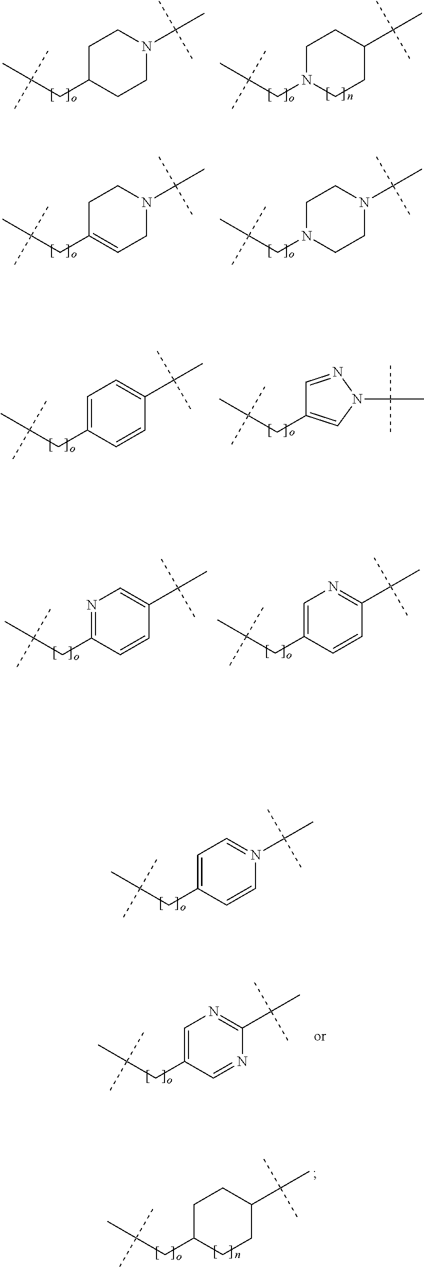
wherein o is 0, 1, or 2, and n is 0, 1, or −1.

14. A compound having a structure according to Formula II 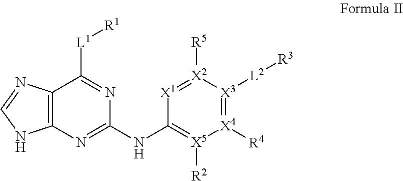
and pharmaceutically acceptable salts and solvates thereof; wherein:
R¹ is an optionally substituted carbocycle, heterocycle, aryl, or heteroaryl;
R² is chosen from the group consisting of: halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle;
R³ is a group chosen from: hydro, haloalkyl, —R^{c}, —N(R^{b})C(═O)R^{c}, -alkylene-N(R^{b})C(═O)R^{c}, —C(═O)N(R^{b})R^{c}, -alkylene-C(═O)N(R^{b})R^{c}, —N(R^{b})S(═O)₂R^{c}, -alkylene-N(R^{b})S(═O)₂R^{c}, —S(═O)₂N(R^{b})R^{c}, -alkylene-S(═O)₂N(R^{b})R^{c}, —S(═O)₂R^{c}, and —N(R^{d})(R^{e}); wherein R^{b} is a group chosen from hydro and C₁₋₄ alkyl; wherein R^{c} is a group chosen from: hydro, alkyl, alkenyl, alkynyl, cycloalkyl, heterocycle, aryl, heteroaryl, cycloalkylalkyl, heterocyclylalkyl, arylalkyl, heteroarylalkyl, hydroxyalkyl, aminoalkyl, alkoxyalkyl, and amino, wherein each group other than hydro may be optionally substituted at each position with one or more groups chosen from (═O), alkyl, alkenyl, alkynyl, cycloalkyl, substituted or unsubstituted heterocycle, aryl, substituted or unsubstituted heteroaryl, nitro, hydroxy, halo, and amino, or R^{b} and R^{c}, when attached to the same atom, together with the atom to which they are bound form an optionally substituted heterocycle or an optionally substituted carbocycle; and wherein R^{d} and R^{e} are each independently chosen from hydro and C₁₋₄ alkyl, or R^{d} and R^{e} together with the nitrogen atom to which they are bound form an optionally substituted heterocycle;
wherein, R⁴ and R⁵ are independently chosen from: hydro, halo, hydroxyl, alkyl, alkynyl, alkoxy, alkynyloxy, haloalkyl, haloalkoxy, cycloalkyloxy, heterocycle-alkoxy, cycloalkoxy, heterocycloxy, alkoxyalkyl, alkylthio, alkanoyl, amino, aminoalkyl, cyanyl, O-carboxy, C-carboxy ester, carboxyalkyl, carboxyalkynyl, carboxyalkoxy, carboxyalkanoyl, carboxyalkenoyl, carboxyalkoxyalkanoyl, O-carbamyl, N-carbamyl, C-amido, N-amido, aminothiocarbonyl, alkoxyaminocarbonyl, sulfonyl, cycloalkyl, and 4, 5 or 6-membered heterocycle; or R³ and either R⁴ or R⁵, together with the carbon atoms to which they are bound, form a carbocycle, heterocycle, aryl or heteroaryl; or R² and R⁴, together with the carbon atoms to which they are bound, form a substituted or unsubstituted carbocycle, substituted or unsubstituted heterocycle, substituted or unsubstituted aryl or substituted or unsubstituted heteroaryl;
X¹ is chosen from N, CH, or is not present;
X², X³, X⁴, and X⁵ are each independently chosen from N and C;
L¹ is a direct bond or a linker chosen from: —O—, —S—, —S(═O)—, —S(═O)₂—, —NR^{a}—, —CH(—R^{a})—, —(CH₂)ₙ—, —N(—R^{a})—(CH₂)ₙ—, —(CH₂), —N(—R^{a})—, —C(═O)—, —C(═C)O—, —C(═O)NR^{a}—, wherein n is 0, 1, 2, 3, 4, or 5, and wherein R^{a} is hydrogen, hydroxyl, alkyl (e.g., methyl), alkoxyl, carboxyl, or carbocycle; and
L² is a bond or a linker chosen from: —O—, —S—, —(C═O)—, —(C═S)—, —N(R^{f})—, —(C═O)N(R^{f})—, —N(R^{f})(C═O)—, —(C═S)N(R^{f})—, —N(R^{f})(C═S)—, —N(R^{f})S(═O)₂—, —S(═O)₂N(R^{f})—, —(C═O)O—, —O(C═O)—, —(C═S)O—, —O(C═S)—, —S(═O)₂—, -alkylene-, alkynylene, aryl, heteroaryl, heterocycle, arylalkyl, heteroarylalkyl, and heterocyclylalkyl and —O-alkylene-; wherein R^{f} is chosen from hydro and C₁₋₄ alkyl.

15. A pharmaceutical composition comprising a compound according to claim 1 and a pharmaceutically-acceptable carrier.

16. A method of treating cancer, said method comprising treating a mammal in need thereof with a therapeutically effective amount of a compound according to claim 1.

17. A method of making a compound according to claim 1, said method comprising:
reacting 2,6-disubstituted purine with -L¹-R¹ in a nucleophilic aromatic substitution reaction under suitable conditions and with suitable reactants to form a first intermediate substituted with -L¹-R¹ at the six position; and
reacting said first intermediate with a desired anilino derivative or analog to form a compound according to claim 1.

18. A method of monitoring the inhibition of TTK by a compound according to claim 1, comprising determining a level of p53 activation in a first biological sample that has been contacted with a compound according to claim 1, and comparing said level of p53 activation with a baseline level of p53 activation from a second biological sample that has not been contacted with said compound, wherein if the level of p53 activation is greater than the baseline level of p53 activation, then TTK has been at least partially inhibited by said compound.

19. A method of monitoring the inhibition of TTK by a compound according to claim 1, comprising determining a level of ATR activation in a first biological sample that has been contacted with a compound according to claim 1 and comparing said level of ATR activation with a baseline level of ATR activation from a second biological sample that has not been contacted with said compound, wherein if the level of ATR activation is greater than the baseline level of ATR activation, then TTK has been at least partially inhibited by said compound.

20. A method of monitoring the inhibition of TTK by a compound according to claim 1, comprising determining a level of Hsp90 phosphorylation in a first biological sample that has been contacted with a compound according to claim 1 and comparing said level of Hsp90 phosphorylation with a baseline level of Hsp90 phosphorylation from a second biological sample that has not been contacted with said compound, wherein if the level of Hsp90 phosphorylation is greater than the baseline level of Hsp90 phosphorylation, then TTK has been at least partially inhibited by said compound.
